SEQUENCE LISTING (44 sequences)

SEQ ID NO: 1 - protein, 897 aa, Homo sapiens
MERAGATSRGGQAPGFLLRLHTEGRAEAARVQEQDLRQWGLTGIHLRSYQLEGVNWLAQRFHCQNGCILGDEMGLGKTCQTIALFIYLAGRLNDEGPFLILCPLSVLSNWKEEMQRFAPGLSCVTYAGDKEERACLQQDLKQESRFHVLLTTYEICLKDASFLKSFPWSVLVVDEAHRLKNQSSLLHKTLSEFSVVFSLLLTGTPIQNSLQELYSLLSFVEPDLFSKEEVGDFIQRYQDIEKESESASELHKLLQPFLLRRVKAEVATELPKKTEVVIYHGMSALQKKYYKAILMKDLDAFENETAKKVKLQNILSQLRKCVDHPYLFDGVEPEPFEVGDHLTEASGKLHLLDKLLAFLYSGGHRVLLFSQMTQMLDILQDYMDYRGYSYERVDGSVRGEERHLAIKNFGQQPIFVFLLSTRAGGVGMNLTAADTVIFVDSDFNPQNDLQAAARAHRIGQNKSVKVIRLIGRDTVEEIVYRKAASKLQLTNMIIEGGHFTLGAQKPAADADLQLSEILKFGLDKLLASEGSTMDEIDLESILGETKDGQWVSDALPAAEGGSRDQEEGKNHMYLFEGKDYSKEPSKEDRKSFEQLVNLQKTLLEKASQEGRSLRNKGSVLIPGLVEGSTKRKRVLSPEELEDRQKKRQEAAAKRRRLIEEKKRQKEEAEHKKKMAWWESNNYQSFCLPSEESEPEDLENGEESSAELDYQDPDATSLKYVSGDVTHPQAGAEDALIVHCVDDSGHWGRGGLFTALEKRSAEPRKIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSAVLHSQSSSSSSRQLVP

SEQ ID NO: 2 - protein, 897 aa, Homo sapiens; identical to SEQ ID NO: 1

SEQ ID NO: 3 - protein, 897 aa, Homo sapiens; identical to SEQ ID NO: 1

SEQ ID NO: 4 - protein, 897 aa, Homo sapiens; identical to SEQ ID NO: 1

SEQ ID NO: 5 - protein, 897 aa, Homo sapiens; identical to SEQ ID NO: 1

SEQ ID NO: 6 - protein, 897 aa, Homo sapiens; identical to SEQ ID NO: 1

SEQ ID NO: 7 - protein, 897 aa, Homo sapiens; identical to SEQ ID NO: 1

SEQ ID NO: 8 - protein, 897 aa, Homo sapiens; identical to SEQ ID NO: 1

SEQ ID NO: 9 - protein, 897 aa, Homo sapiens; identical to SEQ ID NO: 1

SEQ ID NO: 10 - protein, 897 aa, Homo sapiens; identical to SEQ ID NO: 1

SEQ ID NO: 11 - protein, 897 aa, Homo sapiens; identical to SEQ ID NO: 1

SEQ ID NO: 12 - protein, 897 aa, Homo sapiens; identical to SEQ ID NO: 1

SEQ ID NO: 13 - protein, 897 aa, Homo sapiens; identical to SEQ ID NO: 1

SEQ ID NO: 14 - protein, 906 aa, Homo sapiens
ARGGASTGPMERAGATSRGGQAPGFLLRLHTEGRAEAARVQEQDLRQWGLTGIHLRSYQLEGVNWLAQRFHCQNGCILGDEMGLGKTCQTIALFIYLAGRLNDEGPFLILCPLSVLSNWKEEMQRFAPGLSCVTYAGDKEERACLQQDLKQESRFHVLLTTYEICLKDASFLKSFPWSVLVVDEAHRLKNQSSLLHKTLSEFSVVFSLLLTGTPIQNSLQELYSLLSFVEPDLFSKEEVGDFIQRYQDIEKESESASELHKLLQPFLLRRVKAEVATELPKKTEVVIYHGMSALQKKYYKAILMKDLDAFENETAKKVKLQNILSQLRKCVDHPYLFDGVEPEPFEVGDHLTEASGKLHLLDKLLAFLYSGGHRVLLFSQMTQMLDILQDYMDYRGYSYERVDGSVRGEERHLAIKNFGQQPIFVFLLSTRAGGVGMNLTAADTVIFVDSDFNPQNDLQAAARAHRIGQNKSVKVIRLIGRDTVEEIVYRKAASKLQLTNMIIEGGHFTLGAQKPAADADLQLSEILKFGLDKLLASEGSTMDEIDLESILGETKDGQWVSDALPAAEGGSRDQEEGKNHMYLFEGKDYSKEPSKEDRKSFEQLVNLQKTLLEKASQEGRSLRNKGSVLIPGLVEGSTKRKRVLSPEELEDRQKKRQEAAAKRRRLIEEKKRQKEEAEHKKKMAWWESNNYQSFCLPSEESEPEDLENGEESSAELDYQDPDATSLKYVSGDVTHPQAGAEDALIVHCVDDSGHWGRGGLFTALEKRSAEPRKIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSAVLHSQSSSSSSRQLVP

SEQ ID NO: 15 - protein, 897 aa, Homo sapiens
MERAGATSRGGQAPGFLLRLHTEGRAEAARVQEQDLRQWGLTGIHLRSYQLEGVNWLAQRFHCQNGCILGDEMGLGKTCQTIALFIYLAGRLNDEGPFLILCPLSVLSNWKEEMQRFAPGLSCVTYAGDKEERACLQQDLKQESRFHVLLTTYEICLKDASFLKSFPWSVLVVDEAHRLKNQSSLLHKTLSEFSVVFSLLLTGTPIQNSLQELYSLLSFVEPDLFSKEEVGDFIQRYQDIEKESESASELHKLLQPFLLRRVKAEVATELPKKTEVVIYHGMSALQKKYYKAILMKDLDAFENETAKKVKLQNILSQLRKCVDHPYLFDGVEPEPFEVGDHLTEASGKLHLLDKLLAFLYSGGHRVLLFSQMTQMLDILQDYMDYRGYSYERVDGSVRGEERHLAIKNFGQQPIFVFLLSTRAGGVGMNLTAADTVIFVDSDFNPQNDLQAAARAHRIGQNKSVKVIRLIGRDTVEEIVYRKAASKLQLTNMIIEGGHFTLGAQKPAADADLQLSEILKFGLDKLLASEGSTMDEIDLESILGETKDGQWVSDALPAAEGGSRDQEEGKNHMYLFEGKDYSKEPSKEDRKSFEQLVNLQKTLLEKASQEGRSLRNKGSVLIPGLVEGSTKRKRVLSPEELEDRQKKRQEAAAKRRRLIEEKKRQKEEAEHKKKMAWWESNNYQSFCLPSEESEPEDLENGEESSAELDYQDPDATSLKYVSGDVTHPQAGAEDALIVHCVDDSGHWGRGGLFTALEKRSAEPRKIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSAVLHAQSSSSSSRQLVP

SEQ ID NO: 16 - protein, 897 aa, Homo sapiens; identical to SEQ ID NO: 15

SEQ ID NO: 17 - protein, 897 aa, Homo sapiens; identical to SEQ ID NO: 15

SEQ ID NO: 18 - protein, 897 aa, Homo sapiens
MERAGATSRGGQAPGFLLRLHTEGRAEAARVQEQDLRQWGLTGIHLRSYQLEGVNWLAQRFHCQNGCILGDEMGLGKTCQTIALFIYLAGRLNDEGPFLILCPLSVLSNWKEEMQRFAPGLSCVTYAGDKEERACLQQDLKQESRFHVLLTTYEICLKDASFLKSFPWSVLVVDEAHRLKNQSSLLHKTLSEFSVVFSLLLTGTPIQNSLQELYSLLSFVEPDLFSKEEVGDFIQRYQDIEKESESASELHKLLQPFLLRRVKAEVATELPKKTEVVIYHGMSALQKKYYKAILMKDLDAFENETAKKVKLQNILSQLRKCVDHPYLFDGVEPEPFEVGDHLTEASGKLHLLDKLLAFLYSGGHRVLLFSQMTQMLDILQDYMDYRGYSYERVDGSVRGEERHLAIKNFGQQPIFVFLLSTRAGGVGMNLTAADTVIFVDSDFNPQNDLQAAARAHRIGQNKSVKVIRLIGRDTVEEIVYRKAASKLQLTNMIIEGGHFTLGAQKPAADADLQLSEILKFGLDKLLASEGSTMDEIDLESILGETKDGQWVSDALPAAEGGSRDQEEGKNHMYLFEGKDYSKEPSKEDRKSFEQLVNLQKTLLEKASQEGRSLRNKGSVLIPGLVEGSTKRKRVLSPEELEDRQKKRQEAAAKRRRLIEEKKRQKEEAEHKKKMAWWESNNYQSFCLPSEESEPEDLENGEESSAELDYQDPDATSLKYVSGDVTHPQAGAEDALIVHCVDDSGHWGRGGLFTALEKRSAEPRKIYELAGKMKDLSLGGVLLFPVDDKESRNKGQDLLALIVAQHRDRSNVLSGIKMAALEEGLKKIFLAAKKKKASVHLPRIGHATKGFNWYGTEQLIRKHLAARGIPTYIYYFPRSKSAVLHSQSSSSSSRQLVP

SEQ ID NO: 19 - protein, 897 aa, Homo sapiens [sequence omitted]

SEQ ID NO: 20 - protein, 897 aa, Homo sapiens [sequence omitted]

SEQ ID NO: 21 - protein, 900 aa, Homo sapiens [sequence omitted]

SEQ ID NO: 22 - protein, 900 aa, Homo sapiens [sequence omitted]

SEQ ID NO: 23 - protein, 896 aa, Homo sapiens
ERAGATSRGGQAPGFLLRLHTEGPAEAARVQEQDLRQWGLTGIHLRSYQLEGVNWLAQRFHCQNGCILGDEMGLGKTCQTIALFIYLAGRLNDEGPFLILCPLSVLSNWKEEMQRFAPGLSCVTYAGDKEERACLQQDLKQESRFHVLLTTYEICLKDASFLKSFPWSVLVVDEAHRLKNQSSLLHKTLSEFSVVFSLLLTGTPIQNSLQELYSLLSFVEPDLFSKEEVGDFIQRYQDIEKESESASELHKLLQPFLLRRVKAEVATELPKKTEVVIYHGMSALQKKYYKAILMKDLDAFENETAKKVKLQNILSQLRKCVDHPYLFDGVEPEPFEVGDHLTEASGKLHLLDKLLAFLYSGGHRVLLFSQMTQMLDILQDYMDYRGYSYERVDGSVRGEERHLAIKNFGQQPIFVFLLSTRAGGVGMNLTAADTVIFVDSDFNPQNDLQAAARAHRIGQNKSVKVIRLIGRDTVEEIVYRKAASKLQLTNMIIEGGHFTLGAQKPAADADLQLSEILKFGLDKLLASEGSTMDEIDLESILGETKDGQWVSDALPAAEGGSRDQEEGKNHMYLFEGKDYSKEPSKEDRKSFEQLVNLQKTLLEKASQEGRSLRNKGSVLIPGLVEGSTKRKRVLSPEELEDRQKKRQEAAAKRRRLIEEKKRQKEEAEHKKKMAWWESNNYQSFCLPSEESEPEDLENGEESSAELDYQDPDATSLKYVSGDVTHPQAGAEDALIVHCVDDCGHWGRGGLFTALEKRSAEPRKIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSAVLHSQSSSSSSRQLVP

SEQ ID NO: 24 - protein, 896 aa, Homo sapiens; identical to SEQ ID NO: 23

SEQ ID NO: 25 - protein, 896 aa, Homo sapiens; identical to SEQ ID NO: 23

SEQ ID NO: 26 - protein, 890 aa, Homo sapiens
SRGGQAPGFLLRLHTEGRAEAARVQEQDLRQWGLTGIHLRSYQLEGVNWLAQRFHCQNGCILGDEMGLGKTCQTIALFIYLAGRLNDEGPFLILCPLSVLSNWKEEMQRFAPGLSCVTYAGDKEERACLQQDLKQESRFHVLLTTYEICLKDASFLKSFPWSVLVVDEAHRLKNQSSLLHKTLSEFSVVFSLLLTGTPIQNSLQELYSLLSFVEPDLFSKEEVGDFIQRYQDIEKESESASELHKLLQPFLLRRVKAEVATELPKKTEVVIYHGMSALQKKYYKAILMKDLDAFENETAKKVKLQNILSQLRKCVDHPYLFDGVEPEPFEVGDHLTEASGKLHLLDKLLAFLYSGGHRVLLFSQMTQMLDILQDYMDYRGYSYERVDGSVRGEERHLAIKNFGQQPIFVFLLSTRAGGVGMNLTAADTVIFVDSDFNPQNDLQAAARAHRIGQNKSVKVIRLIGRDTVEEIVYRKAASKLQLTNMIIEGGHFTLGAQKPAADADLQLSEILKFGLDKLLASEGSTMDEIDLESILGETKDGQWVSDALPAAEGGSRDQEEGKNHMYLFEGKDYSKEPSKEDRKSFEQLVNLQKTLLEKASQEGRSLRNKGSVLIPGLVEGSTKRKRVLSPEELEDRQKKRQEAAAKRRRLIEEKKRQKEEAEHKKKMAWWESNNYQSFCLPSEESEPEDLENGEESSAELDYQDPDATSLKYVSGDVTHPQAGAEDALIVHCVDDSGHWGRGGLFTALEKRSAEPRKIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSAVLHSQSSSSSSRQLVP

SEQ ID NO: 27 - protein, 890 aa, Homo sapiens; identical to SEQ ID NO: 26

SEQ ID NO: 28 - protein, 890 aa, Homo sapiens; identical to SEQ ID NO: 26

SEQ ID NO: 29 - protein, 896 aa, Pan troglodytes
MERAGAASRGGQAPGFLLRLHTEGRAEAARVQEQDLRQWGLTGIHLRSYQLEGVNWLAQRFHCQNGCILGDEMGLGKTCQTIALFIYLAGRLNDEGPFLILCPLSVLSNWKEEMQRFAPGLSCVTYAGDKEERACLQQDLKQESRFHVLLTTYEICLKDASFLKSFPWSVLVVDEAHRLKNQSSLLHKTLSEFSVVFSLLLTGTPIQNSLQELYSLLSFVEPDLFSKEEVGDFIQRYQDIEKESESASELHKLLQPFLLRRVKAEVATELPKKTEVVIYHGMSALQKRYYKAILMKDLDAFENETAKKVKLQNILSQLRKCVDHPYLFDGVEPEPFEVGDHLIEASGKLHLLDKLLAFLYSGGHRVLLFSQMTQMLDILQDYMDYRGYSYERVDGSVRGEERHLAIKNFGRQPIFVFLLSTRAGGVGMNLTAADTVIFVDSDFNPQNDLQAAARAHRIGQNKSVKVIRLIGRDTVEEIVYRKAASKLQLTNMIIEGGHFTLGAQKPAADADLQLSEILKFGLDKLLASEGSTMDEIDLESILGETKDGQWVSDALPAAEGGSRDQEEGKNHMYLFEGKDYSKEPSKEDRKSFEQLVNLQKTLLEKTSQEGRSLRNKGSVLIPGLVEGSTKRKRVLSPEELEDRQKKRQEAAAKRRRLIEEKKRQKEEAEHKKKMAWWESNNYQSFCLPSEESEPEDLENGEESAAELDYQDPDATSLKYVSGDVTHPQAGAEDALIVHCVDDSGHWGRGGLFTALEKRSAEPRKIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSAVLHSQSSSSSRQLVP

SEQ ID NO: 30 - protein, 896 aa, Pan paniscus
MERAGAASRGGQAPGFLLRLHTEGRAEAARVQEQDLRQWGLTGIHLRSYQLEGVNWLAQRFHCQNGCILGDEMGLGKTCQTIALFIYLAGRLNDEGPFLILCPLSVLSNWKEEMQRFAPGLSCVTYAGDKEERACLQQDLKQESRFHVLLTTYEICLKDASFLKSFPWSVLVVDEAHRLKNQSSLLHKTLSEFSVVFSLLLTGTPIQNSLQELYSLLSFVEPDLFSKEEVGDFIQRYQDIEKESESASELHKLLQPFLLRRVKAEVATELPKKTEVVIYHGMSALQKKYYKAILMKDLDAFENETAKKVKLQNILSQLRKCVDHPYLFDGVEPEPFEVGDHLIEASGKLHLLDKLLAFLYSGGHRVLLFSQMTQMLDILQDYMDYRGYSYERVDGSVRGEERHLAIKNFGQQPIFIFLLSTRAGGVGMNLTAADTVIFVDSDFNPQNDLQAAARAHRIGQNKSVKVIRLIGRDTVEEIVYRKAASKLQLTNMIIEGGHFTLGAQKPAADADLQLSEILKFGLDKLLASEGSTIDEIDLESILGETKDGQWVSDALPAAEGGSRDQEEGKNHMYLFEGKDYSKEPSKEDRKSFEQLVNLQKTLLEKTSQEGRSLRNKGSVLIPGLVEGSTKRKRVLSPEELEDRQKKRQEAAAKRRRLIEEKKRQKEEAEHKKKMAWWESNNYQSFCLPSEESEPEDLENGEESAAELDYQDPDATSLKYVSGDVTHPQAGAEDALIVHCVDDSGHWGRGGLFTALEKRSAEPRKIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSAVLHSQSSSSSRQLVP

SEQ ID NO: 31 - protein, 896 aa, Pan troglodytes
MERAGAASRGGQAPGFLLRLHTEGRAEAARVQEQDLRQWGLTGIHLRSYQLEGVNWLAQRFHCQNGCILGDEMGLGKTCQTIALFIYLAGRLNDEGPFLILCPLSVLSNWKEEMQRFAPGLSCVTYAGDKEERACLQQDLKQESRFHVLLTTYEICLKDASFLKSFPWSVLVVDEAHRLKNQSSLLHKTLSEFSVVFSLLLTGTPIQNSLQELYSLLSFVEPDLFSKEEVGDFIQRYQDIEKEPESASELHKLLQPFLLRRVKAEVATELPKKTEVVIYHGMSALQKRYYKAILMKDLDAFENETAKKVKLQNILSQLRKCVDHPYLFDGVEPEPFEVGDHLIEASGKLHLLDKLLAFLYSGGHRVLLFSQMTQMLDILQDYMDYRGYSYERVDGSVRGEERHLAIKNFGQQPIFVFLLSTRAGGVGMNLTAADTVIFVDSDFNPQNDLQAAARAHRIGQNKSVKVIRLIGRDTVEEIVYRKAASKLQLTNMIIEGGHFTLGAQKPAADADLQLSEILKFGLDKLLASEGSTMDEIDLESILGETKDGQWVSDALPAAEGGSRDQEEGKNHMYLFEGKDYSKEPSKEDRKSFEQLVNLQKTLLEKTSQEGRSLRNKGSVLIPGLVEGSTKRKRVLSPEELEDRQKKRQEAAAKRRRLIEEKKRQKEEAEHKKKMAWWESNNYQSFCLPSEESEPEDLENGEESAAELDYQDPDATSLKYVSGDVTHPQAGAEDALIVHCVDDSGHWGRGGLFTALEKRSAEPRKIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSAVLHSQSSSSSRQLVP

SEQ ID NO: 32 - protein, 896 aa, Pan troglodytes
MERAGAASRGGQAPGFLLRLHTEGRAEAARVQEQDLRQWGLTGIHLRSYQLEGVNWLAQRFHCQNGCILGDEMGLGKTCQTIALFIYLAGRLNDEGPFLILCPLSVLSNWKEEMQRFAPGLSCVTYAGDKEERACLQQDLKQESRFHVLLTTYEICLKDASFLKSFPWSVLVVDEAHRLKNQSSLLHKTLSEFSVVFSLLLTGTPIQNSLQELYSLLSFVEPDLFSKEEVGDFIQRYQDIEKEPESASELHKLLQPFLLRRVKAEVATELPKKTEVVIYHGMSALQKRYYKAILMKDLDAFENETAKKVKLQNILSQLRKCVDHPYLFDGVEPEPFEVGDHLIEASGKLHLLDKLLAFLYSGGHRVLLFSQMTQMLDILQDYMDYRGYSYERVDGSVRGEERHLAIKNFGRQPIFVFLLSTRAGGVGMNLTAADTVIFVDSDFNPQNDLQAAARAHRIGQNKSVKVIRLIGRDTVEEIVYRKAASKLQLTNMIIEGGHFTLGAQKPAADADLQLSEILKFGLDKLLASEGSTMDEIDLESILGETKDGQWVSDALPAAEGGSRDQEEGKNHMYLFEGKDYSKEPSKEDRKSFEQLVNLQKTLLEKTSQEGRSLRNKGSVLIPGLVEGSTKRKRVLSPEELEDRQKKRQEAAAKRRRLIEEKKRQKEEAEHKKKMAWWESNNYQSFCLPSEESEPEDLENGEESAAELDYQDPDATSLKYVSGDVTHPQAGAEDALIVHCVDDSGHWGRGGLFTALEKRSAEPRKIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSAVLHSQSSSSSRQLVP

SEQ ID NO: 33 - protein, 898 aa, Gorilla gorilla
MERAGAASRGGQAPGFLVRLHTEGRAEAARMQEQDLRQWGLTGIHLRSYQLEGVNWLAQRFHCQNGCILGDEMGLGKTCQTIALFIYLVGRLNDEGPFLILCPLSVLSNWKEEMQRFAPGLSCVTYAGDKEERACLQQDLKQESCFHVLLTTYEICLKDASFLKSFPWSVLVVDEAHRLKNQSSLLHKTLSEFSVVFSLLLTGTPIQNSLQELYSLLSFVEPDLFSKEEVGDFIQRYQDIEKESESAGELHKLLQPFLLRRVKAEVATELPRKTEVVIYHGMSALQKKYYKAILMKDLDAFENETAKKVKLQNILSQLRKCVDHPYLFDGVEPEPFEVGDHLIEASGKLHLLDKLLAILYSGGHRVLLFSQMTQMLDILQDYMDYRGYSYERVDGSVRGEERHLAIKNFGQQPIFVFLLSTRAGGVGMNLTAADTVIFVDSDFNPQNDLQAAARAHRIGQNKSVKVIRLIGRDTVEEIVYRKAASKLQLTNMIIEGGHFTLGAQKPAADADLQLSEILKFGLDKLLASEGSTMDEIDLESILGETKDGQWVSDALPAAEGGSRDQEEGKNHMYLFEGKDYSKEPSKEDRKSFEQLVNLQKTLLEKTSQEGRSLRNRGSVLIPGLVEGSTKRKRVLSPEELEDRQKKRQEAAAKRRRLIEEKKRQKEEAEHKKKMAWWESNNYQSFCLPSEESEPEDLENGEDESSAELDYQDPDATSLKYVSGDVTHPQAGAEDALIVHCVDDSGRWGRGGLFTALEKRSAEPRRIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSAVLHSQSSSSSSRQLVP

SEQ ID NO: 34 - protein, 880 aa, Gorilla gorilla
MERAGATSRGGQAPGFLLRLHTEGRAEAARVQEQDLRQWGLTGIHLRSYQLEGVNWLAQRFHCQNGCILGDEMGLGKTCQTIALFIYLAGRLNDEGPFLILCPLSVLSNWKEEMQRFAPGLSCVTYAGDKEERACLQQDLKQESRFHVLLTTYEICLKDASFLKSFPWSVLVVDEAHRLKNQSSLLHKTLSEFSVVFSLLLTGTPIQNSLQELYSLLSFVEPDLFSKEEVGDFIQRYQDIEKESESASELHKLLQPFLLRRVKAEVATELPKKTEVVIYHGMSALQKKYYKAILMKDLDAFENETAKKVKLQNILSQLRKCVDHPYLFDGVEPEPFEVGDHLTEASGKLHLLDKLLAFLYSGGHRVLLFSQMTQMLDILQDYMDYRGYSYERVDGSVRGEERHLAIKNFGQQPIFVFLLSTRAGGVGMNLTAADTVIFVDSDFNPQNDLQAAARAHRIGQNKSVKVIRLIGRDTVEEIVYRKAASKLQLTNMIIEGGHFTLGAQKPAADADLQLSEILKFGLDKLLASEGSTMDEIDLESILGETKDGQWVSDALPAAEGGSRDQEEGKNHMYLFEGKDYSKEPSKEDRKSFEQLVNLQKTLLEKASQEGRSLRNKGSVLIPGLVEGSTKRKRVLSPEELEDRQKKRQEAAAKRRRLIEEKKRQKEEAEHKKKMAWWESNNYQSFCLPSEESEPEDLENGEESSAELDYQDPDATSLKYVSGDVTHPQAGAEDALIVHCVDDSGHWGRGGLFTALEKRSAEPRKIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKS

SEQ ID NO: 35 - protein, 897 aa, Gorilla gorilla
MERAGAASRGGQAPGFLVRLHTEGRAEAARMQEQDLRQWGLTGIHLRSYQLEGVNWLAQRFHCQNGCILGDEMGLGKTCQTIALFIYLVGRLNDEGPFLILCPLSVLSNWKEEMQRFAPGLSCVTYAGDKEERACLQQDLKQESCFHVLLTTYEICLKDASFLKSFPWSVLVVDEAHRLKNQSSLLHKTLSEFSVVFSLLLTGTPIQNSLQELYSLLSFVEPDLFSKEEVGDFIQRYQDIEKESESGELHKLLQPFLLRRVKAEVATELPRKTEVVIYHGMSALQKKYYKAILMKDLDAFENETAKKVKLQNILSQLRKCVDHPYLFDGVEPEPFEVGDHLIEASGKLHLLDKLLAILYSGGHRVLLFSQMTQMLDILQDYMDYRGYSYERVDGSVRGEERHLAIKNFGQQPIFVFLLSTRAGGVGMNLTAADTVIFVDSDFNPQNDLQAAARAHRIGQNKSVKVIRLIGRDTVEEIVYRKAASKLQLTNMIIEGGHFTLGAQKPAADADLQLSEILKFGLDKLLASEGSTMDEIDLESILGETKDGQWVSDALPAAEGGSRDQEEGKNHMYLFEGKDYSKEPSKEDRKSFEQLVNLQKTLLEKTSQEGRSLRNRGSVLIPGLVEGSTKRKRVLSPEELEDRQKKRQEAAAKRRRLIEEKKRQKEEAEHKKKMAWWESNNYQSFCLPSEESEPEDLENGEDESSAELDYQDPDATSLKYVSGDVTHPQAGAEDALIVHCVDDSGRWGRGGLFTALEKRSAEPRRIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSAVLHSQSSSSSSRQLVP

SEQ ID NO: 36 - protein, 898 aa, Gorilla gorilla
MERAGAASRGGQAPGFLVRLHTEGRAEAARMQEQDLRQGGGTGIHLRSYQLEGVNWLAQRFHCQNGCILGDEMGLGKTCQTIALFIYLVGRLNDEGPFLILCPLSVLSNWKEEMQRFAPGLSCVTYAGDKEERACLQQDLKQESCFHVLLTTYEICLKDASFLKSFPWSVLVVDEAHRLKNQSSLLHKTLSEFSVVFSLLLTGTPIQNSLQELYSLLSFVEPDLFSKEEVGDFIQRYQDIEKESESAGELHKLLQPFLLRRVKAEVATELPRKTEVVIYHGMSALQKKYYKAILMKDLDAFENETAKKVKLQNILSQLRKCVDHPYLFDGVEPEPFEVGDHLIEASGKLHLLDKLLAILYSGGHRVLLFSQMTQMLDILQDYMDYRGYSYERVDGSVRGEERHLAIKNFGQQPIFVFLLSTRAGGVGMNLTAADTVIFVDSDFNPQNDLQAAARAHRIGQNKSVKVIRLIGRDTVEEIVYRKAASKLQLTNMIIEGGHFTLGAQKPAADADLQLSEILKFGLDKLLASEGSTMDEIDLESILGETKDGQWVSDALPAAEGGSRDQEEGKNHMYLFEGKDYSKEPSKEDRKSFEQLVNLQKTLLEKTSQEGRSLRNRGSVLIPGLVEGSTKRKRVLSPEELEDRQKKRQEAAAKRRRLIEEKKRQKEEAEHKKKMAWWESNNYQSFCLPSEESEPEDLENGEDESSAELDYQDPDATSLKYVSGDVTHPQAGAEDALIVHCVDDSGRWGRGGLFTALEKRSAEPRRIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSAVLHSQSSSSSSRQLVP

SEQ ID NO: 37 - protein, 900 aa, Mandrillus leucophaeus
MERAGAASRGGQAPGFLLRLHTEGRAEAARARVQEQDLRQWGLTGIHLRSYQLEGVNWLAQRFHCQNGCILGDEMGLGKTCQTIALLIYLAGRLNDEGPFLILCPLSVLSNWKEEMQRFAPGLSCVTYAGDKEERACLQQDLKQESHFHVLLTTYEICLKDASFLKSFPWSVLVVDEAHRLKNQSSLLHKTLSEFSVVFSLLLTGTPIQNSLQELYSLLSFVEPDLFSKEEVGDFVQRYQDIEKESESASELHKLLQPFLLRRVKADVATELPKKTEVVIYHGMSALQKKYYKAILMKDLDAFENETAKKVKLQNILSQLRKCVDHPYLFDGVEPEPFEVGDHLIEASGKLHLLDKLLAFLYSGGHRVLLFSQMTQMLDILQDYMDYRGYSYERVDGSVRGEERHLAIKNFGQQPIFVFLLSTRAGGVGMNLTAADTVIFVDSDFNPQNDLQAAARAHRIGQNKSVKVIRLIGRDTVEEIVSRKAASKLQLTNMIIEGGHFTLGAQKPSADADLQLSEILKFGLDKLLASEGSTMDEIDLESILGETKDGQWVSDALPAAEGGIREQEEGKNHMYLFEGKDYSKEPSKEDRKSFEQLVNLQKTLLEKTSQEGRSLRNKGSVLIPGLVEGSTKRKQVLSPEELEDRQKKRQEAAAKRRRLIEEKKRQKEEAEHKKKMAWWESNHYQSFCLPSEESEPEDLENGEDESSAELDYQDPDATSLKYVSGDVTHPQAGAEDALIVHCVDDSGHWGRGGLFTALEKRSAEPRKIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSSVLHSQSSSSSSRQLVP

SEQ ID NO: 38 - protein, 900 aa, Chlorocebus sabaeus
MERAGAASRGGQAPGFLLRLHTEGRAEAARARVQEQDLRQWGLTGIHLRSYQLEGVNWLAQRFHCQNGCILGDEMGLGKTCQTIALLIYLAGRLNDEGPFLILCPLSVLSNWKEEMQRFAPGLSCVTYAGDKEERACLQQDLKQESHFHVLLTTYEICLKDASFLKSFPWSVLVVDEAHRLKNQSSLLHKTLSEFSVVFSLLLTGTPIQNSLQELYSLLSFVEPDLFSKEEVGDFVQRYQDIEKESESASELHKLLQPFLLRRVKADVATELPKKTEVVIYHGMSALQKKYYKAILMKDLDAFENETAKKVKLQNILSQLRKCVDHPYLFDGVEPEPFEVGDHLIEASGKLHLLDKLLAFLYSGGHRVLLFSQMTQMLDILQDYMDYRGYSYERVDGSVRGEERHLAIKNFGQQPIFVFLLSTRAGGVGMNLTAADTVIFVDSDFNPQNDLQAAARAHRIGQNKSVKVIRLIGRDTVEEIVYRKAASKLQLTNMIIEGGHFTLGAQKPSADADLQLSEILKFGLDKLLASEGSTMDEIDLESILGETKDGQWVSDALPAAQGGIREQEEGKNHMYLFEGKDYSKEPSKEDRKSFEQLVNLQKTLLEKTSQEGRSLRNKGSVLIPGLVEGSTKRKQVLSPEELEDRQKKRQEAAAKRRRLIEEKKRQKEEAEHKKKMAWWESNHYQSFCLPSEESEPEDLENGEDESSAELDYQDPDATSLKYVSGDVTHPQAGAEDALIVHCVDDSGHWGRGGLFTALEKRSAEPRKIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSSVLYSQSSSSSSRQLVP

SEQ ID NO: 39 - protein, 909 aa, Gorilla gorilla
MERAGAASRGGQAPGFLVRLHTEGRAEAARMQEQDLRQWGLTGIHLRSYQLEGVNWLAQRFHCQNGCILGDEMGLGKTCQTIALFIYLVGRLNDEGPFLILCPLSVLSNWKEEMQRFAPGLSCVTYAGDKEERACLQQDLKQESCFHVLLTTYEICLKDASFLKSFPWSVLVVDEAHRLKNQSSLLHKTLSEFSVVFSLLLTGTPIQNSLQELYSLLSFVEPDLFSKEEVGDFIQRYQDIEKESESAGELHKLLQPFLLRRVKAEVATELPRKTEVVIYHGMSALQKKYYKAILMKDLDAFENETAKKVKLQNILSQLRKCVDHPYLFDGVEPEPFEVGDHLIEASGKLHLLDKLLAILYSGGHRVLLFSQMTQMLDILQDYMDYRGYSYERVDGSVRGEERHLAIKNFGQQPIFVFLLSTRAGGVGMNLTAADTVIFVDSDFNPQNDLQAAARAHRIGQNKSVKVIRLIGRDTVEEIVYRKAASKLQLTNMIIEGGHFTLGAQKPAADADLQNLLDFFVPLFKLSEILKFGLDKLLASEGSTMDEIDLESILGETKDGQWVSDALPAAEGGSRDQEEGKNHMYLFEGKDYSKEPSKEDRKSFEQLVNLQKTLLEKTSQEGRSLRNRGSVLIPGLVEGSTKRKRVLSPEELEDRQKKRQEAAAKRRRLIEEKKRQKEEAEHKKKMAWWESNNYQSFCLPSEESEPEDLENGEDESSAELDYQDPDATSLKYVSGDVTHPQAGAEDALIVHCVDDSGRWGRGGLFTALEKRSAEPRRIYELAGKMKDLSLGGVLLFPVDDKESRNKGQDLLALIVAQHRDRSNVLSGIKMAALEEGLKKIFLAAKKKKASVHLPRIGHATKGFNWYGTERLIRKHLAARGIPTYIYYFPRSKSAVLHSQSSSSSSRQLVP

SEQ ID NO: 40 - protein, 17 aa, Homo sapiens
RRVKAEVATELPKKTEV

SEQ ID NO: 41 - protein, 5 aa, Homo sapiens
SDFNP

SEQ ID NO: 42 - protein, 4 aa, Homo sapiens
KVIR

SEQ ID NO: 43 - protein, 11 aa, Homo sapiens
IVYRKAASKLQ

SEQ ID NO: 44 - protein, 12 aa, Homo sapiens
MYLFEGKDYSKE

(19) United States
(12) Patent Application Publication
(10) Pub. No.: US 2026/0240844 A1
(43) Pub. Date: Aug. 20, 2026

(54) INHIBITORS OF CHD1L AND USES THEREOF

(71) Applicant: The Regents of the University of Colorado, a body corporate, Denver, CO (US)

(72) Inventors: Daniel V. LaBarbera, Denver, CO (US); Qiong Zhou, Denver, CO (US); Hector Esquer, Denver, CO (US); Paul Awolade, Denver, CO (US); Rita Sala-Faig, Denver, CO (US)

(21) Appl. No.: 19/471,971

(22) PCT Filed: Apr. 2, 2024
(86) PCT No.: PCT/US2024/022678

§ 371 Date: Oct. 2, 2025

Related U.S. Application Data
(60) Provisional application No. 63/493,974, filed on Apr. 3, 2023.
(60) Provisional application No. 63/614,302, filed on Dec. 22, 2023.

(51) Int. Cl. A61K 31/506; A61K 31/337; A61K 31/421; A61K 31/4745; A61K 31/502; A61K 31/513; A61K 31/704; A61P 35/00; G01N 21/76; G01N 33/50

(52) U.S. Cl. CPC A61K 31/506; A61K 31/337; A61K 31/421; A61K 31/4745; A61K 31/502; A61K 31/513; A61K 31/704; A61P 35/00; G01N 21/76; G01N 33/5011

(57) ABSTRACT

Provided are inhibitors and combinations of inhibitors and chemotherapies for the treatment of cancer. The inhibitors may be CHD1L inhibitors and may bind to an allosteric binding site of CHD1L. The inhibitors may inhibit CHD1L autoactivation, CHD1L ATP-ase activity, inhibit tumor growth, trap CHD1L onto chromatin or a combination thereof. The combinations of CHD1L inhibitors and chemotherapies may improve the efficacy of the chemotherapies against drug-resistant cancers.

### CROSS-REFERENCE

This application claims the benefit of U.S. Provisional Application Nos. 63/493,974, filed Apr. 3, 2023, and 63/614,302, filed Dec. 22, 2023, each of which is incorporated herein in its entirety by reference.

### STATEMENT AS TO FEDERALLY SPONSORED RESEARCH

This invention was made with government support under R01-CA251361-01 awarded by the National Cancer Institute of the National Institutes of Health. The government has certain rights in this invention.

### INCORPORATION BY REFERENCE OF SEQUENCE LISTING

The present application is being filed along with a Sequence Listing in electronic format. The Sequence Listing is provided as a file entitled 65194-703_601-SequenceListing.XML, created Mar. 30, 2024, which is 71.6 kilobytes in size. The information in the electronic format of the Sequence Listing is incorporated by reference in its entirety.

### BACKGROUND

Despite the advancement of cutting-edge technologies for cancer diagnosis and treatment, cancer continues to stand as one of the most devastating diseases, impacting millions across the globe. The challenges posed by tumor progression, metastasis, and multidrug resistance (MDR) remain formidable obstacles in the realm of cancer treatment, accounting for a staggering 90% of patient distress and mortality across all types of cancer. Conventional approaches such as standard-of-care (SOC) chemotherapy and targeted therapy have proven inadequate in tackling these critical issues.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### SUMMARY

In some aspects, provided herein is a method of treating cancer, the method comprising: administering to a subject with the cancer a composition comprising: a therapeutically effective amount of a chromodomain-helicase-DNA-binding protein 1-like (CHD1L) inhibitor having the structure of Formula (I):

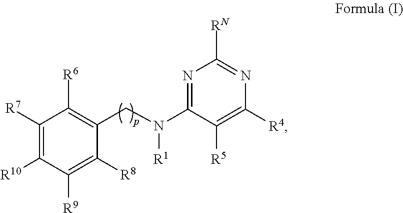

or a pharmaceutically acceptable salt thereof, wherein:
R^{N} is an optionally substituted 5- to 7-membered heterocycle optionally containing a second heteroatom selected from the of group N, S, and O;
R¹ is selected from hydrogen or C₁₋₃ alkyl;
each R⁴ and R⁵ is selected from hydrogen, C₁₋₃ alkyl, and halogen;
R⁶ is hydrogen or a halogen;
each R⁷, R⁸, and R⁹ is independently selected from hydrogen and C₁₋₃ alkyl, optionally substituted with C₁₋₃ alkyl or aryl;
R¹⁰ is NH—C(O)—(CH₂)ₘ—Ar, wherein
   m is 0, 1, 2, 3, 4, or 5, and
   Ar is aryl or heteroaryl optionally substituted with R^{a},
wherein each R^{a} is selected from hydrogen, C₁₋₃ alkyl, C₁₋₃ alkoxy, C₁₋₃ haloalkyl, or halogen, or two R^{a} together with the carbon to which they are bound form a 5- or 6-membered carbocycle, aryl, or heteroaryl optionally substituted with R^{a}; and
   p is 0, 1, or 2,
   wherein the cancer is breast cancer, melanoma, osteosarcoma, lung cancer, or pancreatic cancer. In some embodiments, p is 0 or 1. In some embodiments, R¹ is hydrogen. In some embodiments,
Ar is a 5- to 10-membered heterocycle. In some embodiments, the 5- to 10-membered heterocycle is thiophenyl, furanyl, pyranyl, pyrrolyl, benzofuranyl, isobenzofuranyl, oxazolyl, indolyl, benzo[b]thiophenyl, or benzo[c]thiophenyl. In some embodiments, wherein at least one of R⁶, R⁷, R⁸, or R⁹ is hydrogen. In some embodiments, Ar is substituted with at least one R^{a}. In some embodiments, R¹⁰ has the structure:

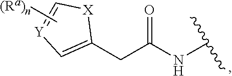

wherein X is N, S, or O; Y is N, S, or C; R^{a} is selected from: hydrogen, C₁₋₃ alkyl, C₁₋₃ alkoxy, C₁₋₃ haloalkyl, or halogen; and
n is 0, 1, 2, or 3. In some embodiments, the CHD1Li has the structure of Formula (II):

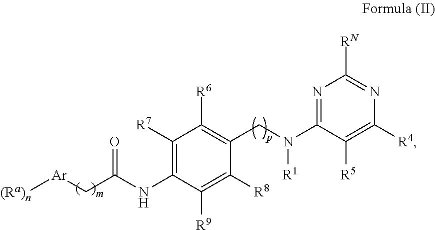

or a pharmaceutically acceptable salt thereof, wherein:
R^{N} is an optionally substituted 5- to 7-membered heterocycle optionally containing a second heteroatoms selected from the group N, S, or O;
R¹ is hydrogen or methyl;
R⁴ and R⁵ are each independently hydrogen, C₁₋₃ alkyl group, or halogen;
R⁶ is hydrogen or halogen;
R⁷, R⁸, and R⁹ are each independently hydrogen, C₁₋₃ alkyl, C₁₋₃ alkoxy, optionally substituted C₁₋₃ alkyl, or aryl;
Ar is an aryl or heteroaryl optionally substituted R^{a}, wherein
each R^{a} is selected from hydrogen, C₁₋₃ alkyl, C₁₋₃ alkoxy, C₁₋₃ haloalkyl, or halogen, or two R^{a} together with the carbon to which they are bound form a 5- or 6-membered carbocycle, aryl, or heteroaryl optionally substituted with R^{b}, and
   each R^{b} is selected from C₁₋₃ alkyl, C₁₋₃ alkoxy, C₁₋₃ haloalkyl, or halogen;
   p is 0 or 1;
   m is 0, 1, 2, 3, 4, or 5; and
   n is an integer from 0 to 9.
   In some embodiments, Ar is a 5- to 10-membered heterocycle. In some embodiments, the 5- to 10-membered heterocycle is thiophenyl, furanyl, pyranyl, pyrrolyl, benzofuranyl, isobenzofuranyl, oxazolyl, indolyl, benzo[b]thiophenyl, or benzo[c]thiophenyl. In some embodiments, R⁴ is methyl. In some embodiments, R¹ is hydrogen. In some embodiments, at least two of R⁵, R⁶, R⁷, R⁸, and R⁹ are hydrogen. In some embodiments, at least one of R⁵, R⁶, R⁷, R⁸, or R⁹ is halogen or C₁₋₃ alkoxy. In some embodiments, the CHD1L inhibitor has the structure of Formula (IIIA):

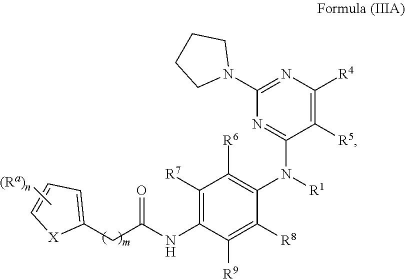

or a pharmaceutically acceptable salt thereof, wherein:
R¹ is hydrogen or methyl;
R⁴ and R⁵ are each independently hydrogen, C₁₋₃ alkyl group, or halogen;
R⁶ is hydrogen or a halogen;
R⁷, R⁸, and R⁹ are each independently hydrogen, C₁₋₃ alkyl, optionally substituted C₁₋₃ alkyl, or aryl;
each R^{a} is selected from hydrogen, C₁₋₃ alkyl, C₁₋₃ alkoxy, C₁₋₃ haloalkyl, or halogen, or two R^{a} together with the carbon to which they are bound form a 5- or 6-membered carbocycle, aryl, or heteroaryl optionally substituted with R^{b};
each R^{b} is selected from C₁₋₃ alkyl, C₁₋₃ alkoxy, C₁₋₃ haloalkyl, or halogen;
X is N, S, or O;
m is 0, 1, 2, 3, 4, or 5; and
n is 0, 1, 2, 3, or 4.

In some embodiments, R¹ is hydrogen. In some embodiments, at least one of R⁶, R⁷, R⁸, or R⁹ is hydrogen. In some embodiments, m is 1, 2, 3, 4, or 5. In some embodiments, n is 1, 2, 3, or 4. In some embodiments, when n is 0, m is at least 1. In some embodiments, R^{a} is a C₁₋₃ alkyl or halogen. In some embodiments, the halogen is bromo or chloro. In some embodiments, X is N. In some embodiments, X is S. In some embodiments, X is O. In some embodiments, at least one of R⁴ or R⁵ is methyl. In some embodiments, CHD1L inhibitor having the structure of Formula (IIIA) is:

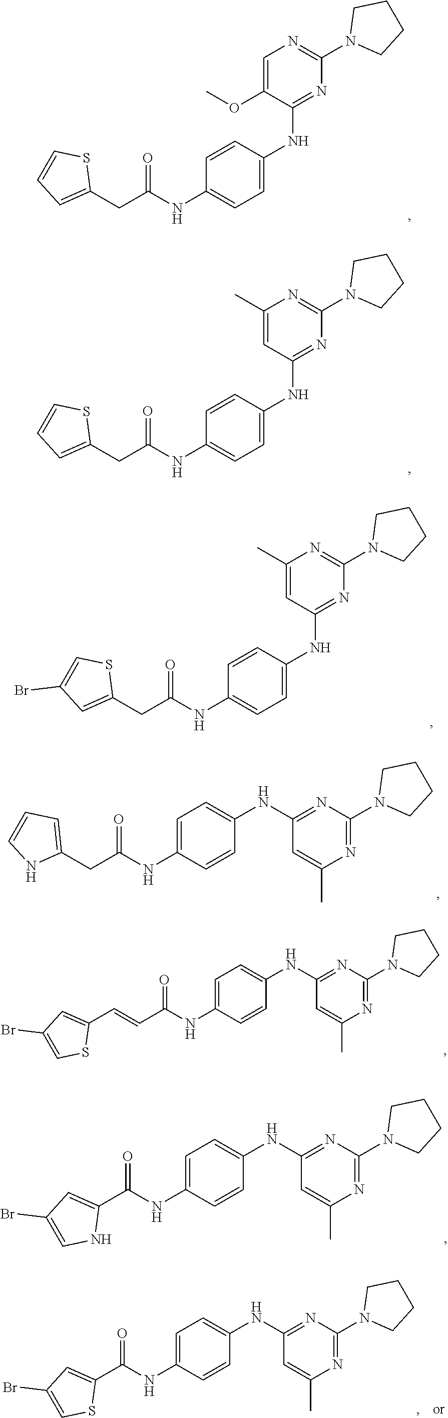

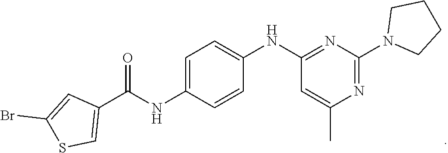 [SMILES: Cc1cc(Nc2ccc(NC(=O)c3csc(Br)c3)cc2)nc(N2CCCC2)n1]

In some embodiments, the CHD1L inhibitor has the structure of Formula (IIIB):

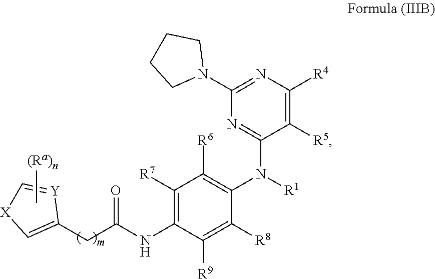

or a pharmaceutically acceptable salt thereof, wherein:
R¹ is hydrogen or methyl;
R⁴ and R⁵ are each independently hydrogen, C₁₋₃ alkyl group, or halogen;
R⁶ is hydrogen or a halogen;
R⁷, R⁸, and R⁹ are each independently hydrogen, C₁₋₃ alkyl, optionally substituted C₁₋₃ alkyl, or aryl;
each R^{a} is selected from hydrogen, C₁₋₃ alkyl, C₁₋₃ alkoxy, C₁₋₃ haloalkyl, or halogen, or two R^{a} together with the carbon to which they are bound form a 5- or 6-membered carbocycle, aryl, or heteroaryl optionally substituted with R^{b};
each R^{b} is selected from C₁₋₃ alkyl, C₁₋₃ alkoxy, C₁₋₃ haloalkyl, or halogen;
X is N, S, or O;
Y is C, N, or S;
m is 1, 2, 3, 4, or 5; and
n is 1, 2, 3, or 4.
   In some embodiments, R¹ is hydrogen. In some embodiments, at least one of R⁶, R⁷, R⁸, or R⁹ is hydrogen. In some embodiments, m is 1, 2, or 3. In some embodiments, n is 1, 2, 3, or 4. In some embodiments, R^{a} is a C₁₋₃ alkyl or halogen. In some embodiments, the halogen is bromo or chloro. In some embodiments, X is N. In some embodiments, X is O. In some embodiments, X is S. In some embodiments, Y is N, n is 1, and R^{a} is halogen. In some embodiments, at least one or R⁴ or R⁵ is methyl. In some embodiments, the CHD1L inhibitor of Formula (IIIB) is:

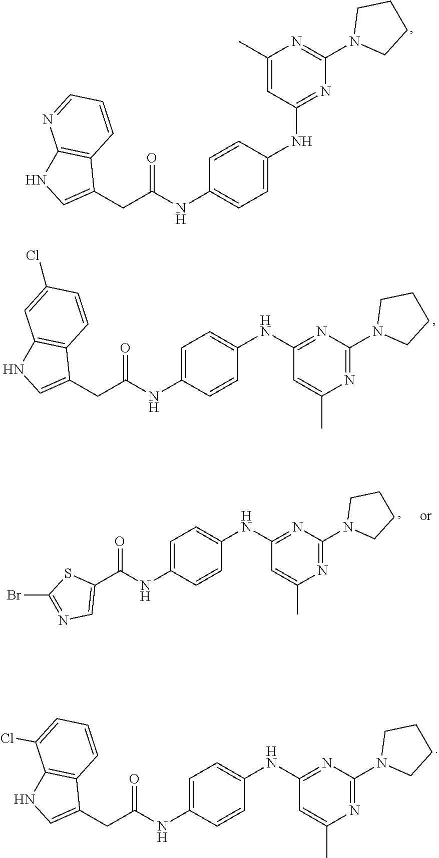 [SMILES: Cc1cc(Nc2ccc(NC(=O)Cc3c[nH]c4ncccc34)cc2)nc(N2CCCC2)n1 ; Cc1cc(Nc2ccc(NC(=O)Cc3c[nH]c4cc(Cl)ccc34)cc2)nc(N2CCCC2)n1 ; Cc1cc(Nc2ccc(NC(=O)c3cnc(Br)s3)cc2)nc(N2CCCC2)n1 ; Cc1cc(Nc2ccc(NC(=O)Cc3c[nH]c4c(Cl)cccc34)cc2)nc(N2CCCC2)n1]

In some embodiments, the CHD1L inhibitor is a compound selected from Table 1. In some embodiments, the CHD1L inhibitor binds to an allosteric binding site comprising an N-terminus of a C-terminal ATP-ase domain of CHD1L. In some embodiments, the CHD1L inhibitor reduces an ATP-ase activity of CHD1L. In some embodiments, the CHD1L inhibitor interacts with a conserved lysine in the allosteric binding site. In some embodiments, the CHD1L comprises a sequence having at least 80% sequence identity SEQ ID NOs: 1-40 or Table 2. In some embodiments, the binding of CHD1L to an allosteric site of CHD1L inhibits chromatin remodeling. In some embodiments, the CHD1L inhibitor modulates poly-ADP-ribosylation (PAR) in a cancer cell. In some embodiments, administering the CHD1L inhibitor induces PAR-mediated programmed cell death (PARthanatos). In some embodiments, administering the CHD1L inhibitor prevents cell DNA repair in the tumor cell. In some embodiments, administering the CHD1L inhibitor induces PARthanatos and prevents cell DNA repair in the tumor cell. In some embodiments, administering the CHD1L inhibitor traps CHD1L within a nucleus of the tumor cell. In some embodiments, administering the CHD1L inhibitor inhibits a cell cycle of the tumor cell. In some embodiments, administering the CHD1L inhibitor inhibits chromatin remodeling. In some embodiments, administering the CHD1L inhibitor reduces tumor cell viability by about 20%, about 30%, about 40%, about 50%, about 60%, about 70%, about 80%, about 90%, about 95%, about 97% about 99% or about 100%, when the tumor cell viability is measured using an assay that measures ATP as an indicator of the tumor cell viability and generates a luminescence readout. In some embodiments, assay comprises: (a) introducing the CHD1L inhibitor and a detection reagent to organoids in a culture medium, wherein the detection reagent becomes luminescent when ATP in the organoids is extracted from the organoids; (b) mixing the CHD1L inhibitor, the detection reagent and the organoids to produce a mixture under conditions sufficient to induce cell lysis and extract the ATP from the organoids; (c) incubating the mixture to stabilize a luminescent signal; and (d) recording luminescence to produce the luminescence readout. In some embodiments, the cancer displays multidrug resistance (MDR). In some embodiments, the cancer is breast cancer. In some embodiments, the cancer is lung cancer. In some embodiments, the cancer is melanoma. In some embodiments, the cancer is osteosarcoma. In some embodiments, the cancer is pancreatic cancer. In some embodiments, the method further comprises administering a chemotherapy to the subject. In some embodiments, a combination of the CHD1L inhibitor and the chemotherapy is therapeutically effective to treat the cancer. In some embodiments, a combination of the CHD1L inhibitor potentiates or is synergistic with the chemotherapy. In some embodiments, the cancer is resistant to the chemotherapy. In some embodiments, the chemotherapy is a PARP inhibitor. In some embodiments, the chemotherapy is a standard of care (SOC) chemotherapy for the cancer. In some embodiments, the chemotherapy comprises irinotecan, olaparib, doxorubicin, docetaxel, AZD5305, or 5-fluorouracil (5-FU), or a combination of two or more thereof. In some embodiments, the administration of the CHD1L inhibitor and the chemotherapy provides a Bliss synergy score of at least about 10, wherein the Bliss energy score is determined using a Bliss model. In some embodiments, the administration of the CHD1L inhibitor and the chemotherapy provides a Bliss synergy score of at least about 10 to about 60 using a Bliss model. In some embodiments, the administering the CHD1L inhibitor and the administering the chemotherapy are performed simultaneously. In some embodiments, the administration of the CHD1L inhibitor localizes apoptosis inducing factor (AIF) in the nucleus of the tumor cell. In some embodiments, the administration of the CHD1L inhibitor provides a higher concentration of AIF in the nucleus of the tumor cell relative to a concentration of AIF in a cytoplasm of the tumor cell. In some embodiments, the CHD1L inhibitor induces localization of PAR in the nucleus of the tumor cell.

In some aspects, provided herein is a pharmaceutical composition for treating a cancer comprising: a CHD1L inhibitor having the structure:

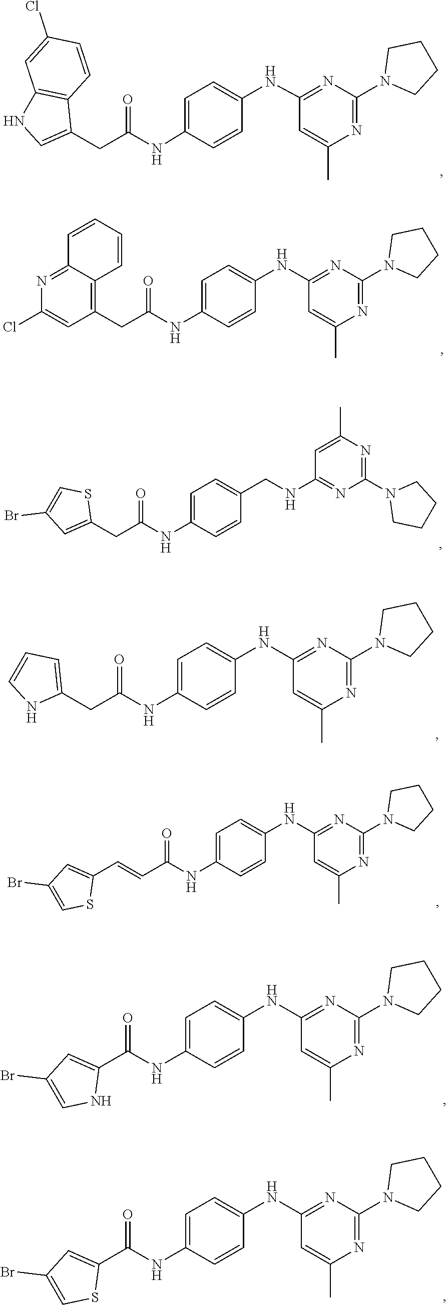

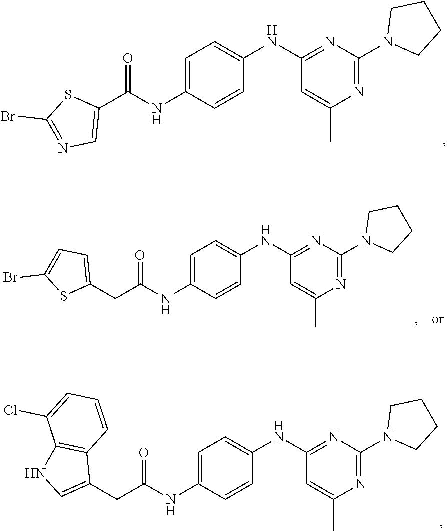 [SMILES: Cc1cc(Nc2ccc(NC(=O)c3cnc(Br)s3)cc2)nc(N2CCCC2)n1 ; Cc1cc(Nc2ccc(NC(=O)Cc3ccc(Br)s3)cc2)nc(N2CCCC2)n1 ; Cc1cc(Nc2ccc(NC(=O)Cc3c[nH]c4c(Cl)cccc34)cc2)nc(N2CCCC2)n1]

or a pharmaceutically acceptable salt thereof. In some embodiments, the pharmaceutical composition further comprises a chemotherapy. In some embodiments, the chemotherapy comprises irinotecan, olaparib, doxorubicin, docetaxel, AZD5305, or 5-fluorouracil (5-FU), or a combination of two or more thereof. In some embodiments, the chemotherapy is olaparib. In some embodiments, the chemotherapy is irinotecan. In some embodiments, the chemotherapy is doxorubicin. In some embodiments, the chemotherapy is docetaxel. In some embodiments, the chemotherapy is AZD5305. In some embodiments, the chemotherapy is 5-fluorouracil (5-FU). In some embodiments, the chemotherapy is effective against breast cancer. In some embodiments, the chemotherapy is effective against colorectal cancer. In some embodiments, the CHD1L inhibitor is a compound of Table 1. In some embodiments, the CHD1L inhibitor is 2-(6-chloro-1H-indol-3-yl)-N-(4-((6-methyl-2-(pyrrolidin-1-yl)pyrimidin-4-yl)amino)phenyl)acetamide.

In some embodiments, the CHD1L inhibitor is 2-(2-chloroquinolin-4-yl)-N-(4-((6-methyl-2-(pyrrolidin-1-yl)pyrimidin-4-yl)amino)phenyl)acetamide. In some embodiments, the CHD1L inhibitor is 2-(4-bromothiophen-2-yl)-N-(4-(((6-methyl-2-(pyrrolidin-1-yl)pyrimidin-4-yl)amino)methyl)phenyl)acetamide. In some embodiments, the CHD1L inhibitor is N-(4-((6-methyl-2-(pyrrolidin-1-yl)pyrimidin-4-yl)amino)phenyl)-2-(1H-pyrrol-2-yl)acetamide. In some embodiments, the CHD1L inhibitor is (E)-3-(4-bromothiophen-2-yl)-N-(4-((6-methyl-2-(pyrrolidin-1-yl)pyrimidin-4-yl)amino)phenyl)acrylamide. In some embodiments, the CHD1L inhibitor is 4-bromo-N-(4-((6-methyl-2-(pyrrolidin-1-yl)pyrimidin-4-yl)amino)phenyl)-1H-pyrrole-2-carboxamide. In some embodiments, the CHD1L inhibitor is 4-bromo-N-(4-((6-methyl-2-(pyrrolidin-1-yl)pyrimidin-4-yl)amino)phenyl)thiophene-2-carboxamide. In some embodiments, the CHD1L inhibitor is 2-bromo-N-(4-((6-methyl-2-(pyrrolidin-1-yl)pyrimidin-4-yl)amino)phenyl)thiazole-5-carboxamide. In some embodiments, the CHD1L inhibitor is 2-(5-bromothiophen-2-yl)-N-(4-((6-methyl-2-(pyrrolidin-1-yl)pyrimidin-4-yl)amino)phenyl)acetamide. In some embodiments, the CHD1L inhibitor is 2-(7-chloro-1H-indol-3-yl)-N-(4-((6-methyl-2-(pyrrolidin-1-yl)pyrimidin-4-yl)amino)phenyl)acetamide. In some embodiments, the CHD1L inhibitor and the chemotherapy are formulated as separate dosages. In some embodiments, the CHD1L inhibitor and the chemotherapy are formulated to be administered simultaneously. In some embodiments, the CHD1L inhibitor and the chemotherapy are formulated to be administered sequentially. In some embodiments, the CHD1L inhibitor is formulated to be administered prior to administration of the chemotherapy. In some embodiments, the CHD1L inhibitor is formulated to be administered after administration of the chemotherapy. In some embodiments, the pharmaceutical composition is formulated to be administered orally or parenterally. In some embodiments, the pharmaceutical composition is formulated to be administered orally. In some embodiments, the pharmaceutical composition is formulated to be parenterally administered as an intradermal injection, subcutaneous injection, intramuscular injection, intraperitoneal injection, intraperitoneal infusion, or intravenous injection.

In some aspects, provided herein is a method for treating cancer comprising administering a pharmaceutical composition as described herein. In some embodiments, the cancer is colorectal cancer, breast cancer, osteosarcoma, melanoma, or lung cancer. In some embodiments, the cancer is colorectal cancer. In some embodiments, the cancer is breast cancer. In some embodiments, the cancer is osteosarcoma. In some embodiments, the cancer is melanoma. In some embodiments, the cancer is lung cancer. In some embodiments, the pharmaceutical composition further comprises a chemotherapy. In some embodiments, a combination of the CHD1L inhibitor and the chemotherapy is therapeutically effective to treat the cancer. In some embodiments, the CHD1L inhibitor potentiates or is synergistic with the chemotherapy. In some embodiments, the cancer is resistant to the chemotherapy, when the cancer is not treated with the CHD1L inhibitor. In some embodiments, the chemotherapy is a PARP inhibitor. In some embodiments, the chemotherapy is a standard of care (SOC) chemotherapy for the cancer. In some embodiments, the chemotherapy comprises irinotecan, olaparib, doxorubicin, docetaxel, AZD5305, or 5-fluorouracil (5-FU), or a combination of two or more thereof. In some embodiments, the administration of the CHD1L inhibitor and the chemotherapy provides a Bliss synergy score of at least about 10, the Bliss energy score is determined using a Bliss model. In some embodiments, the administration of the CHD1L inhibitor and the chemotherapy provides a Bliss synergy score of at least about 10 to about 60 using a Bliss model. In some embodiments, the administration of the CHD1L inhibitor localizes apoptosis inducing factor (AIF) in the nucleus of the tumor cell. In some embodiments, the administration of the CHD1L inhibitor provides a higher concentration of AIF in the nucleus of the tumor cell relative to a concentration of AIF in a cytoplasm of the tumor cell. In some embodiments, the CHD1L inhibitor induces localization of PAR in the nucleus of the tumor cell.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A-FIG. 1G shows an illustration of CHD1L. FIG. 1A provides a comparison of full length CHD1L and catalytic CHD1L (cat-CHD1L), which lacks a portion of the linker domain and macro domain. FIG. 1B provides a PDB structure of CHD1L showing the ATP binding site in the N-ATPase domain and the allosteric binding site in the C-ATPase site. FIG. 1C provides a picture of an allosteric binding site made of Lys-263, Lys-272, Lys-273, and other residues in CHD1L (SEQ ID NO: 1; PDB:7EPU) with Compound 6.11 bound. FIG. 1D provides an inhibition curve of cat-CHD1L (wildtype) at varying concentrations of Compound 6.11. FIG. 1E provides an inhibition curve of cat-CHD1L-K272A at varying concentrations of Compound 6.11. FIG. 1F provides a dose-dependent increase in CHD1L trapping in breast cancer as a function of Compound 6.11 concentration (μM). FIG. 1G provides a dose-dependent increase in CHD1L trapping in colorectal cancer as a function of Compound 6.11 concentration (μM).

FIG. 2 shows a plot of efficacy/safety of various standard of care therapies for CRC, which are limited in safety and efficacy and do not meet the 3-year survival rate demand by CRC patients. Compound 6.11 combined with CRC chemo/targeted therapy has the potential to exceed the 3-year survival demand. Modified from Global-Data 2010. CRC drugs/combinations: FOLFOX (5FU/LV+oxaliplatin), FOLFIRI (5FU/LV+irinotecan), irinotecan, Xeloda (capecitabine), Erbitux and Avastin (biologics).

FIG. 3 shows chemotherapy and targeted therapy frontline and standard of care regimens for mCRC. Source: University of Colorado Cancer Center and Global-Data 2017.

FIG. 4A-FIG. 4D shows an illustration of the CHD1L-mediated blockade of PARthanatos programmed cell death, according to some embodiments, herein. FIG. 4A provides a picture showing CHD1L blocks PARG while promoting PARP1-mediated PARylation in the nucleus, resulting in DNA repair, tumor cell survival, and MDR to chemotherapy. Events in panel A using SUM149PT HR-deficient cells were measured. FIG. 4B provides a graph showing doxorubicin DNA damage induces nuclear PARylation via CHD1L/PARP. Compound 6.11 inhibits CHD1L/PARP-mediated PARylation as part of the mechanism preventing DNA repair and MDR. FIG. 4C provides a graph showing CHD1L and PARP both inhibit PARG-mediated PAR cytoplasm translocation. Compound 6.11 and PARPi inhibit PARG blockade, resulting in FIG. 4D provides a graph showing AIF translocation to the nucleus and PARthanatos. PAR and AIF localization were measured by immunofluorescent staining using an Opera Phenix Plus high-content imaging and analysis system.

FIG. 5A-FIG. 5B shows graphs of tumor organoid viability on the y-axis and log-concentration of irinotecan (SN38) in combination with CDH1Li on the x-axis. FIG. 5A provides graphs showing isolated quasi EMT SW620 CRC cells E (red), E/M (yellow), and M (green) were cultured as tumor organoids and treated with SN38 combined with Compound 6 over 72 h as indicated. M-organoids were significantly more resistant to SN38 (6=0 μM) compared to E and E/M organoids but synergized with Compound 6 at all doses against each EMT phenotype. FIG. 5B Isolated E (red), E/M (yellow), and M (green) HCT116 tumor organoids were treated with olaparib combined with Compound 6 over 72 h. Like panel A, M-organoids were more resistant to olaparib (6=0 μM) compared to E and E/M organoids.

FIG. 6A-FIG. 6B show graphs illustrating the synergy score of Compound 6.11 and irinotecan (SN38) as a three-dimensional diagram (left) or as a 2-dimensional contour plot (right). The plots indicate that Compound 6.11 synergizes with SN38. FIG. 6A shows a plot of synergy scores for different combinations of Compound 6.11 and SN-38 and a corresponding contour plot of a combination Compound 6.11 and SN-38, which shows significant DNA damage at nM concentrations of SN-38. FIG. 6B shows a plot of synergy scores for different combinations of Compound 6.11 and 5-FU and a corresponding contour plot of a combination Compound 6.11 and 5-FU.

FIG. 7A-FIG. 7C provide a series of plots illustrating the effect of combinations of Compound 6.11 and (A) SN-38, (B) 5-FU, and (C) Olaparib on colorectal cancer organoids. FIG. 7A shows a graph illustrating the synergy score of Compound 6.11 and SN-38, a contour plot corresponding to % cytotoxicity of organoids treated with SN-38 (μM) and Compound 6.11 (μM), and a plot of % viability (y-axis) as a function of SN-38 concentration (nM) (x-axis) as a function of Compound 6.11 concentration (μM). FIG. 7B shows a graph illustrating the synergy score of Compound 6.11 and 5-FU, a contour plot corresponding to % cytotoxicity of organoids treated with 5-FU (μM) and Compound 6.11 (μM), and a plot of % viability (y-axis) as a function of 5-FU concentration (μM) (x-axis) as a function of Compound 6.11 concentration (μM). FIG. 7C shows a graph illustrating the synergy score of Compound 6.11 and Olaparib, a contour plot corresponding to % cytotoxicity of organoids treated with Olaparib (μM) and Compound 6.11 (μM), and a plot of % viability (y-axis) as a function of Olaparib concentration (nM) (x-axis) as a function of Compound 6.11 concentration (μM).

FIG. 8A-FIG. 8B show graphs of synergy of Compound 6.11 and SN38 as a treatment in ATM-mutant drug resistant SW948 tumor organoids. FIG. 8A shows the results of Compound 6.11 and SN-38 in decreasing tumor cell viability as a function of Compound 6.11 and SN-38 administration. FIG. 8B shows the results of Compound 6.11 and SN-38 in decreasing tumor cell viability as a function of Compound 6.11 and 5-FU administration.

FIG. 9A-9E show graphs illustrating the downregulation of CHD1L-mediated TCF-transcription in M-phenotype CRC cells by CHD1L inhibitors (CHD1Li). FIG. 9A provides a bar graph of % TCF-transcription in SW620 cells and HCT116 cells in the presence of Compound 6.11. FIG. 9B provides % TCF-transcription as a function of a log of CHD1L inhibitor concentration (μM) and illustrates the different CHD1Li compounds can decrease % TCF-transcription by inhibiting CHD1L in SW620 cells. FIG. 9C provides % TCF-transcription as a function of a log of CHD1L inhibitor concentration (μM) and illustrates the different CHD1Li compounds can decrease % TCF-transcription by inhibiting CHD1L in HCT116 cells. FIG. 9D provides a plot of clonogenic colonies as a function of CHD1L inhibitor concentration (μM) in SW620 cells and shows CHD1L inhibitors of the instant disclosure are able to decrease the number of clonogenic colonies in SW620 cells through inhibition of CHD1L. FIG. 9E provides a plot of clonogenic colonies as a function of CHD1L inhibitor concentration (μM) in HCT116 cells and shows CHD1L inhibitors of the instant disclosure are able to decrease the number of clonogenic colonies in HCT116 cells through inhibition of CHD1L.

FIG. 10 shows graphs of tumor volume and percent (%) survival of irinotecan drug-resistant SW620 M-phenotype mouse xenografts after treatment with Compound 6.11 and irinotecan and irinotecan alone.

FIG. 11 shows histology images of bone marrow, cardiomyocytes, and hepatocytes in healthy mice that were given a vehicle, Compound 6.11, and a combination of Compound 6.11 and irinotecan.

FIG. 12 shows a schematic of a mouse with a catheter and VAB port according to embodiments of the disclosure.

FIG. 13 shows a synthesis scheme of compounds according to embodiments of the disclosure.

FIG. 14 shows an alternate synthesis scheme of compounds according to embodiments of the disclosure.

FIG. 15A-FIG. 15B shows graphs illustrating off-target considerations for Compound 6.11. FIG. 15A Compound 6.11 does not cause DNA damage, measured by immunofluorescence of γ-H2AX foci, a clinical biomarker for DNA damage. FIG. 15B A panel of 200 kinase inhibitors were screened against recombinant cat-CHD1L ATPase. None of the kinase inhibitors show inhibition at 20 μM.

FIG. 16A-FIG. 16D shows graph illustrating induction PARthanatos cell death by Compound 6.11. FIG. 16A provides a graph showing Compound 6.11 cytotoxicity dose response IC₅₀ values in a panel of cancer cell lines. FIG. 16B provides a series of graphs showing PARthanatos biomarker events in SUM149PT breast cancer cells were measured using chemotherapies with and without Compound 6.11. Compound 6.11 induces PAR translocation to the cytoplasm and AIF translocation to the nucleus alone and when combined with olaparib and doxorubicin. FIG. 16C provides a series of graphs showing Compound 6.11 induces PARthanatos in HCT116 CRC cells alone and when combined with SN-38. SN-38 does not induce PARthanatos. FIG. 16D provides a series of graphs showing Compound 6.11 does not induce caspase 3 activation, which is consistent with its induction of PARthanatos. SN-38 induces caspase 3 activation. Compound 6.11 combined with SN-38 potentiates SN-38's apoptosis induction, which is attributed to Compound 6.11's ability to inhibit CHD1L-mediated DNA repair. Compound 6.11's synergizes with chemotherapy to potentiate cell death. ANOVA significance where *P<0.05, **P<0.001, ***P<0.0001.

FIG. 17A-FIG. 17B shows Compound 6 does not cause DNA damage alone but trapping of CHD1L prevents DNA repair, synergizing with chemotherapy to enhance DNA damage. 3D contour plots show dose dependent synergy where a score 10≤is synergistic. DNA damage γ-H2AX foci were measured by immunofluorescent imaging over 4 h. ANOVA significance is shown where *P<0.05, **P<0.001, ***P<0.0001. FIG. 17A provides a series of plots (top) illustrating a synergy score (z-axis) as a function of chemotherapy (μM; y-axis) and Compound 6.11 (μM; x-axis) concentrations. A set of corresponding contour plots of the combinations are provided (bottom). The chemotherapies tested include Olaparib, doxorubicin, and 5-FU. FIG. 17B provides a series of plots (left) illustrating a synergy score (z-axis) as a function of chemotherapy (μM; y-axis) and Compound 6.11 (μM; x-axis) concentrations, and the corresponding contour plots of the combinations are presented (right). The chemotherapies tested include SN-38 and 5-FU.

FIG. 18 shows Compound 6 synergizes with chemotherapy to kill BC tumor organoids. Tables display fixed Compound 6 doses and combination IC₅₀ values. Organoid viability was measured by 3D CellTiter-Glo. 3D synergy plots show dose-dependent synergy (score ≥10).

FIG. 19 shows Compound 6 synergizes with chemotherapy to kill CRC tumor organoids. Tables display fixed Compound 6 doses and combination IC₅₀ values. Organoid viability was measured by 3D CellTiter-Glo. 3D synergy plots show dose-dependent synergy (score ≥10).

FIG. 20A-FIG. 20C shows CHD1Li synergizes with TNBC therapies in SUM149PT organoids. FIG. 20A provides Bliss Synergy 3D plots showing synergy scores for each dose combination of Compound 6.11 (1.7-1.9 μM) and PARPi and SOC chemotherapy. FIG. 20B provides dose response matrices representing the percentage of cell death caused by Compound 6, PARPi and SOC therapy, and their combinations. FIG. 20C provides dose response curves showing Compound 6.11's synergistic effect when combined with PARPi and SOC chemotherapy as measured by IC50 values. SUM149PT organoids were treated with drug combinations for 72 h. Bliss Synergy score values were calculated using the SynergyFinder R package. To evaluate synergy, Compound 6.11 was treated at sub-lethal doses. Synergy score values above 10 are considered a synergistic interaction between drugs. Data are presented as the mean of two independent experiments ±S.E.M.

FIG. 21A-FIG. 21B shows CHD1Li synergizes with TNBC therapies in MDA-MB-231 and HCC1937 tumor organoids. FIG. 21A provides dose response curves showing Compound 6.11's synergistic effect when combined with PARPi and SOC chemotherapy as measured by IC50 values in MDA-MB-231 tumor organoids treated for 72 h. FIG. 21B provides dose response curves showing Compound 6's synergistic effect when combined with PARPi and SOC chemotherapy as measured by IC₅₀ values in HCC1937 tumor organoids treated for 72 h. Data are presented as the mean of two independent experiments ±S.E.M.

FIG. 22A-FIG. 22C shows inhibition of CHD1L enhances chemotherapy and PARPi-mediated DNA damage. FIG. 22A provides Bliss Synergy 3D plots showing synergy scores for each dose combination of Compound 6.11 (1.5-2 μM) and PARPi or SOC chemotherapy. FIG. 22B provides dose response matrices representing the percentage of DNA damage measured by y-H2AX immunofluorescence, for doses of Compound 6.11, PARPi and SOC chemotherapy, and their combinations. Bliss Synergy scores were generated using the SynergyFinder R package. A synergistic drug interaction is considered when values are above 10. Data are presented as the mean of two independent experiments ±S.E.M. FIG. 22C Representative images of γ-H2AX immunofluorescence in SUM149PT cells treated for 4 hours with Compound 6, PARPi and SOC chemotherapy, and their combinations. Scale bar=100 μm.

FIG. 23A-FIG. 23B shows inhibition of CHD1L enhances chemotherapy and PARPi-mediated cell cycle arrest. FIG. 23A provides representative flow cytometry profiles of SUM149PT cells treated for 24 h with Compound 6.11, PARPi and SOC chemotherapy (Olaparib or 5-FU), and their combinations. After treatment, cells were fixed and stained with DAPI. FIG. 23B provides the distribution of cells in G1, S, or G2/M is indicated. The experiment was performed in two independent experimental replicates.

FIG. 24A-FIG. 24E shows inhibition of CHD1L traps PARP1, PARP2, and CHD1L at DNA damage sites. FIG. 24A provides trapping profiles of PARP1, PARP2, and CHD1L measured after dose-response treatment with olaparib. FIG. 24B provides trapping profiles of PARP1, PARP2, and CHD1L measured after dose-response treatment with Compound 6.11. FIG. 24C provides trapping profiles of PARP1, PARP2, and CHD1L measured after dose-response treatment with AZD5305. FIG. 24D provides trapping profiles of PARP1, PARP2, and CHD1L measured after dose-response treatment with doxorubicin. Doxorubicin was used as a negative control of trapping. FIG. 24E provides trapping profiles of PARP1, PARP2, and CHD1L measured after dose-response treatment with olaparib combined with Compound 6.11 or vice versa. For all the conditions, SUM149PT cells were treated with the drug of interest in combination and 0.01% MMS for 4 hours. All data were normalized to MMS treated cells and expressed as the mean of two independent experiments ±S.E.M.

FIG. 25A-FIG. 25F shows CHD1Li-mediated PAR translocation to the cytoplasm activates PARthanatos. FIG. 25A provides a graph showing intensity of cytoplasmic PAR in SUM149PT cells treated for 6 h with Compound 6.11, olaparib, AZD5305 or doxorubicin, and their combinations. PAR localization is measured as the sum intensity in the cytoplasm and normalized by the number of nuclei per field. FIG. 25B provides a graph of intensity of nuclear PAR in SUM149PT cells treated for 4 h with Compound 6.11, olaparib, AZD5305 or doxorubicin, and their combinations. PAR mean intensity is normalized by the number of nuclei per field. Data expressed as mean of three independent experiments ±S.E.M. FIG. 25C provides representative images of PAR immunofluorescence showing changes in nuclear PAR and its translocation to the cytoplasm with Compound 6.11 treatment. Scale bar=50 μm. FIG. 25D provides a graph of intensity of cytoplasmic AIF in SUM149PT cells treated for 18 hours with Compound 6.11, olaparib, AZD5305 or doxorubicin, and their combinations. FIG. 25E provides a graph of intensity of nuclear AIF in SUM149PT cells treated for 18 h with Compound 6, olaparib, AZD5305, or doxorubicin, and their combinations. AIF mean intensity is normalized by the number of nuclei per field and expressed as mean of two independent experiments ±S.E.M. FIG. 25F provides representative images of AIF immunofluorescence showing changes in cytoplasmic AIF and its translocation to the nucleus with Compound 6 treatment. Scale bar=50 μm.

FIG. 26A-FIG. 26D provide graphs showing the ability of CHD1L inhibitors to trap PARP1 and CHD1L as single agents. FIG. 26A shows a graph of PARP1 trapping relative to a standard (immunofluorescence of cells exposed to doxorubicin as control) as a function of Compound 6.11 or Compound 7 concentration in micromolar. Compounds 6.11 and 7 were able to trap PARP1 by at least 5-fold. FIG. 26B illustrates a graph of CHD1L trapping relative to a standard (immunofluorescence of cells exposed to doxorubicin as control) relative to Compound 6.11 and Compound 7 concentration in micromolar. Compound 7 was able to trap CHD1L by about 5-fold relative to the baseline, and Compound 6.11 was able to trap CHD1L by about 3-fold relative to the baseline. FIG. 26C shows a graph of PARP1 trapping relative to a standard (immunofluorescence of cells exposed to doxorubicin as control) as a function of a concentration (μM) of Compounds 13, 14, and 16. FIG. 26D illustrates a graph of CHD1L trapping relative to a standard (immunofluorescence of cells exposed to doxorubicin as control) relative to the concentration (μM) of Compounds 13, 14, and 16.

FIG. 27 shows a graph of % nucleosome sliding as a function of time for different samples of CHD1L, PARP1, ATP, and Compound 6.11, where (a) represents the presence of CHD1L and PARP1; (b) represents the presence of CHD1L, PARP1, and ATP; (c) represents the presence of CHD1L and ATP; (d) represents the presence of PARP1 and ATP; and (e) represents CHD1L, PARP1, ATP, and CHD1L inhibitor (Compound 6.11).

FIG. 28 shows a series of bar graphs comparing AIR localization in the nucleus or cytoplasm in SUM149 and MDA-MB-231 cell lines (breast cancer cell lines) in the presence or Compounds 12, 15, and 16.

FIG. 29 shows bar graphs illustrating the activation of factors involved in homologous recombination or nonhomologous end-joining in the presence of (a) vehicle, (b) etoposide, (c), Compound 6.11, (d) Compound 12, (e) Compound 14, and (f) Compound 16 in Colo679 cells, MDA-MB-231, and SUM149PT cells. The factors monitored and assayed include pATM, RAD51, 53BP1, and γ-H2AX.

FIG. 30 shows a diagram illustrating the relationship of the factors assessed in FIG. 29 in the two different DNA Damage Repair (DDR) pathways.

FIG. 31 shows a graph of % tumor organoid viability on the y-axis and concentration of Compound 6.11 on the x-axis, where organoid viability in the presence of Compound 6.11 (as a single agent) was tested in SUM149PT, MDA-MB-231, HCC1937, and MCF-7 cell lines.

FIG. 32 shows contour plots (top) and plots of synergy scores, chemotherapy (docetaxel, 5-FU, doxorubicin, and Olaparib), and Compound 6.11.

FIG. 33 shows graphs of tumor cell viability % of Colo678 cells as a function of SN-38 or 5-FU concentration and Compound 6.11 concentration, and the mean synergy scores are presented.

FIG. 34 shows graphs of tumor cell viability % of Colo678 cells as a function of SN-38 or 5-FU concentration and Compound 12 concentration, and the mean synergy scores are presented.

FIG. 35 shows graphs of tumor cell viability % of Colo678 cells as a function of SN-38 or 5-FU concentration and Compound 13 concentration, and the mean synergy scores are presented.

FIG. 36 shows graphs of tumor cell viability % of Colo678 cells as a function of SN-38 or 5-FU concentration and Compound 20 concentration, and the mean synergy scores are presented.

FIG. 37 shows graphs of tumor cell viability % of Colo678 cells as a function of SN-38 or 5-FU concentration and Compound 14 concentration, and the mean synergy scores are presented.

FIG. 38 shows graphs of tumor cell viability % of Colo678 cells as a function of SN-38 concentration and Compound 16 concentration, and the mean synergy scores are presented.

FIG. 39A-FIG. 39D shows graphs of tumor cell viability % of HCT116 and SW620 cells as a function of SN-38 or 5-FU concentration and Compound 11 concentration, and the mean synergy scores are presented. FIG. 39A provides a plot of % HCT116 cell viability as a function of both SN-38 concentration (in μM) and Compound 11 concentration (in μM) showing a decrease in the IC₅₀ of SN-38 with increasing Compound 11 concentration, and the synergy score suggests synergy between SN-38 and Compound 11. FIG. 39B provides a plot of % HCT116 cell viability as a function of both 5-FU concentration (in μM) and Compound 11 concentration (in μM) showing a decrease in the IC₅₀ of 5-FU with increasing Compound 11 concentration, and the synergy score suggests synergy between SN-38 and Compound 11, FIG. 39C provides a plot of % SW620 cell viability as a function of both SN-38 concentration (in μM) and Compound 11 concentration (in μM) showing a decrease in the IC₅₀ of SN-38 with increasing Compound 11 concentration, and the synergy score suggests synergy between SN-38 and Compound 11. FIG. 39D provides a plot of % SW620 cell viability as a function of both 5-FU concentration (in μM) and Compound 11 concentration (in μM) showing a decrease in the IC₅₀ of 5-FU with increasing Compound 11 concentration, and the synergy score suggests synergy between SN-38 and Compound 11.

FIG. 40A-FIG. 40D shows graphs of tumor cell viability % of HCT116 and SW620 cells as a function of SN-38 or 5-FU concentration and Compound 12 concentration, and the mean synergy scores are presented. FIG. 40A provides a plot of % HCT116 cell viability as a function of both SN-38 concentration (in μM) and Compound 12 concentration (in μM) showing a decrease in the IC₅₀ of SN-38 with increasing Compound 12 concentration, and the synergy score suggests synergy between SN-38 and Compound 12. FIG. 40B provides a plot of % HCT116 cell viability as a function of both 5-FU concentration (in μM) and Compound 12 concentration (in μM) showing a decrease in the IC₅₀ of 5-FU with increasing Compound 12 concentration, and the synergy score suggests synergy between SN-38 and Compound 12. FIG. 40C provides a plot of % SW620 cell viability as a function of both SN-38 concentration (in μM) and Compound 12 concentration (in μM) showing a decrease in the IC₅₀ of SN-38 with increasing Compound 12 concentration, and the synergy score suggests synergy between SN-38 and Compound 12. FIG. 40D provides a plot of % SW620 cell viability as a function of both 5-FU concentration (in μM) and Compound 12 concentration (in μM) showing a decrease in the IC₅₀ of 5-FU with increasing Compound 12 concentration, and the synergy score suggests synergy between SN-38 and Compound 12.

FIG. 41A-FIG. 41B shows graphs of tumor cell viability % of MiaPaca2 as a function of SN-38 concentration and Compound 6.11 concentrations, and the mean synergy scores are presented. FIG. 41A provides a plot of % MiaPaca2 cell viability as a function of both SN-38 concentration (in μM) and Compound 6.11 concentration (in μM) showing a decrease in the IC₅₀ of SN-38 with increasing Compound 6.11 concentration, and the synergy score suggests synergy between SN-38 and Compound 6.11. FIG. 41B provides a plot of % MiaPaca2 cell viability as a function of both 5-FU concentration (in μM) and Compound 6.11 concentration (in μM) showing a decrease in the IC₅₀ of 5-FU with increasing Compound 6.11 concentration, and the synergy score suggests synergy between SN-38 and Compound 6.11.

FIG. 42A-FIG. 42B shows graphs of tumor cell viability % of MiaPaca2 as a function of SN-38 concentration and Compound 7 concentrations, and the mean synergy scores are presented. FIG. 42A provides a plot of % MiaPaca2 cell viability as a function of both SN-38 concentration (in μM) and Compound 7 concentration (in μM) showing a decrease in the IC₅₀ of SN-38 with increasing Compound 7 concentration, and the synergy score suggests synergy between SN-38 and Compound 7. FIG. 42B provides a plot of % MiaPaca2 cell viability as a function of both 5-FU concentration (in μM) and Compound 7 concentration (in μM) showing a decrease in the IC₅₀ of 5-FU with increasing Compound 7 concentration, and the synergy score suggests synergy between SN-38 and Compound 7.

FIG. 43A-FIG. 43B shows graphs of tumor cell viability % of MiaPaca2 as a function of SN-38 concentration and Compound 8 concentrations, and the mean synergy scores are presented. FIG. 43A provides a plot of % MiaPaca2 cell viability as a function of both SN-38 concentration (in μM) and Compound 8 concentration (in μM) showing a decrease in the IC₅₀ of SN-38 with increasing Compound 8 concentration, and the synergy score suggests synergy between SN-38 and Compound 8. FIG. 43B provides a plot of % MiaPaca2 cell viability as a function of both 5-FU concentration (in μM) and Compound 8 concentration (in μM) showing a decrease in the IC₅₀ of 5-FU with increasing Compound 8 concentration, and the synergy score suggests synergy between SN-38 and Compound 8.

FIG. 44A-FIG. 44B shows graphs of tumor cell viability % of MiaPaca2 as a function of SN-38 concentration and Compound 12 concentrations, and the mean synergy scores are presented. FIG. 44A provides a plot of % MiaPaca2 cell viability as a function of both SN-38 concentration (in μM) and Compound 12 concentration (in μM) showing a decrease in the IC₅₀ of SN-38 with increasing Compound 12 concentration, and the synergy score suggests synergy between SN-38 and Compound 12. FIG. 44B provides a plot of % MiaPaca2 cell viability as a function of both 5-FU concentration (in μM) and Compound 12 concentration (in μM) showing a decrease in the IC₅₀ of 5-FU with increasing Compound 12 concentration, and the synergy score suggests synergy between SN-38 and Compound 12.

FIG. 45A-FIG. 45B shows graphs of tumor cell viability % of MiaPaca2 as a function of SN-38 concentration and Compound 13 concentrations, and the mean synergy scores are presented. FIG. 45A provides a plot of % MiaPaca2 cell viability as a function of both SN-38 concentration (in μM) and Compound 13 concentration (in μM) showing a decrease in the IC₅₀ of SN-38 with increasing Compound 13 concentration, and the synergy score suggests synergy between SN-38 and Compound 13. FIG. 45B provides a plot of % MiaPaca2 cell viability as a function of both 5-FU concentration (in μM) and Compound 13 concentration (in μM) showing a decrease in the IC₅₀ of 5-FU with increasing Compound 13 concentration, and the synergy score suggests synergy between SN-38 and Compound 13.

FIG. 46A-FIG. 46D shows graphs of tumor cell viability % of MDA-MB-231 and SUM149PT cells as a function of SN-38 or 5-FU concentration and Compound 13 concentration, and the mean synergy scores are presented. FIG. 46A provides a plot of % MDA-MB-231 cell viability as a function of both SN-38 concentration (in μM) and Compound 13 concentration (in μM) showing a decrease in the IC₅₀ of SN-38 with increasing Compound 13 concentration, and the synergy score suggests synergy between SN-38 and Compound 13. FIG. 46B provides a plot of % SUM149PT cell viability as a function of both 5-FU concentration (in μM) and Compound 13 concentration (in μM) showing a decrease in the IC₅₀ of 5-FU with increasing Compound 13 concentration, and the synergy score suggests synergy between SN-38 and Compound 13. FIG. 46C provides a plot of % MDA-MB-231 cell viability as a function of both SN-38 concentration (in μM) and Compound 13 concentration (in μM) showing a decrease in the IC₅₀ of SN-38 with increasing Compound 13 concentration, and the synergy score suggests synergy between SN-38 and Compound 13. FIG. 46D provides a plot of % SUM149PT cell viability as a function of both 5-FU concentration (in μM) and Compound 13 concentration (in μM) showing a decrease in the IC₅₀ of 5-FU with increasing Compound 13 concentration, and the synergy score suggests synergy between SN-38 and Compound 13.

FIG. 47A-FIG. 47B shows graphs of tumor cell viability % of MDA-MB-231 and SUM149PT as a function of Olaparib concentration and Compound 13 concentrations, and the mean synergy scores are presented. FIG. 47A provides a plot of % MDA-MB-231 cell viability as a function of both Olaparib concentration (in μM) and Compound 13 concentration (in μM) showing a decrease in the IC₅₀ of Olaparib with increasing Compound 13 concentration, and the synergy score suggests synergy between Olaparib and Compound 13. FIG. 47B provides a plot of % SUM149PT cell viability as a function of both 5-FU concentration (in μM) and Compound 13 concentration (in μM) showing a decrease in the IC₅₀ of 5-FU with increasing Compound 13 concentration, and the synergy score suggests synergy between Olaparib and Compound 13.

FIG. 48 shows a bar graph of tumor volume (in millimeters³, mm³) in a colon cancer mouse xenograft model treated with vehicle (left) and with Compound 6.11, and the results indicate treatment with Compound 6.11 reduced a tumor volume by about 50% over 30 days.

FIG. 49 shows a bar graph of plasma area under the curve (AUC) over the course of 8 hours (ng/mL/hr) of Compound 6.11 in plasma in different oral or intravenous formulations.

FIG. 50A-FIG. 50D illustrates the efficacy of Compound 6.11 as a single agent and in combination with irinotecan in a colon cancer mouse xenograft model. FIG. 50A shows a graph of tumor volume (mm³; y-axis) as a function of days post treatment (x-axis) and provides a comparison of tumor volume in mice administered (a) vehicle and (b) Compound 6.11 (125 mg/kg; 5×/week; oral). FIG. 50B shows a graph of tumor volume days after the colon cancer mouse xenograft model was given (a) vehicle, (b) irinotecan, and (c) Compound 6.11 and irinotecan. FIG. 50C shows a percent survival of the xenograft model in the days following treatment with (a) vehicle, (b) irinotecan, and (c) Compound 6.11 and irinotecan. FIG. 50D shows a graph of bioluminescence imaging of the model in the days following treatment, and the results show the combination of Compound 6.11 and irinotecan significantly reduced metastatic tumor burden.

### DETAILED DESCRIPTION

Breast, lung and bronchus, prostate, and colorectal cancers are becoming increasingly prevalent and account for about 50% of new cancer diagnoses in the United States. Tumors and metastatic tumors may become resistant to standard of care chemotherapies, which may be treated with higher doses of the chemotherapy and thereby increasing the likelihood of undesirable side effects. Further, remission success varies depending on the type of cancer and initial rounds of treatment, and acquired drug resistance results in fewer options when the cancer returns and lower survival rates.

For instance, CRC is the third most prevalent cancer diagnosed each year and CRC patients have the second highest mortality rate worldwide. Early detection, surgery, and chemo/targeted therapy such as FOLFIRI (FOLFIRI (FOLinic acid aka leucovorin (LV), 5FU, IRInotecan)), FOLFOX (LV (FOLinic acid), 5-FU, OXaliplatin), and Avastin have minimally improved CRC overall survival. Despite the emergence of targeted therapies such as Avastin, which have not proven effective alone, chemotherapy is still the safer, more effective and affordable treatment for CRC. Chemotherapy will remain a frontline treatment for the foreseeable future. Still, only about half of CRC patients respond to FOLFIRI-based chemotherapy and almost all display MDR at some point in the treatment. This poor response rate is attributed to the high tumor heterogeneity of CRC. In some cancers, PARP inhibitors may be used to block DNA Damage Repair (DDR), but cancers that become resistant to PARP inhibitors may become deathly. Thus, there is a need for a cancer therapies and treatments that overcome drug resistance or chemotherapy resistance, such as treatments that can decrease a dosage of the chemotherapy.

Provided herein are compositions and methods for treating drug-resistant cancers, such as breast cancer, lung cancer, pancreatic cancer, melanoma, osteosarcoma, and colorectal cancer, and metastatic forms thereof. The compositions and methods comprise target Chromodomain Helicase DNA Binding Protein 1 Like (CHD1L) (also known as amplified in liver cancer 1, ALC1), which is a protein expressed by an oncogene that promotes tumor progression, metastasis, and multidrug resistance (MDR) in many cancers such as lung cancer, breast cancer, pancreatic cancer, melanoma, osteosarcoma, and colorectal cancer (CRC), among others. CHD1L is an oncogene and its amplification and overexpression in patients is a marker of metastatic cancer, poor prognosis, low survival, and multidrug resistance (MDR). CHD1L functions at the interface of malignant gene expression and tumor cell survival. CHD1L expression is upregulated in cancer patients. In some cases, such as metastatic CRC, surgery is not typical, and chemo/targeted therapy is the standard of care, yet these therapies are ineffective, evidenced by a low 11% 5-year overall survival.

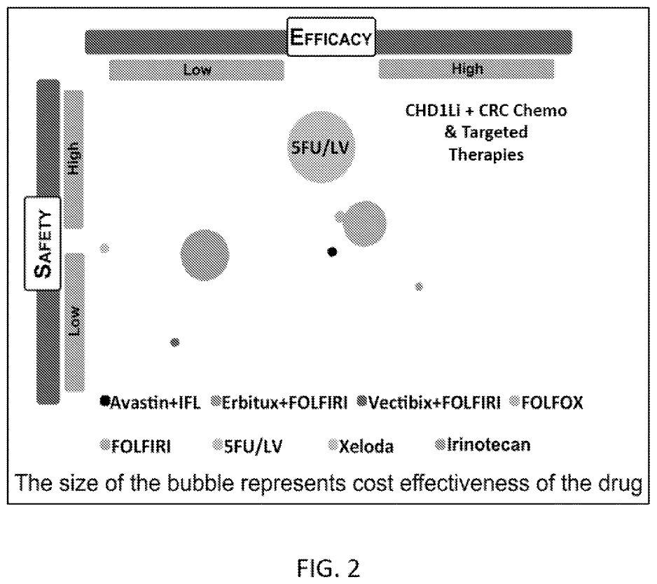

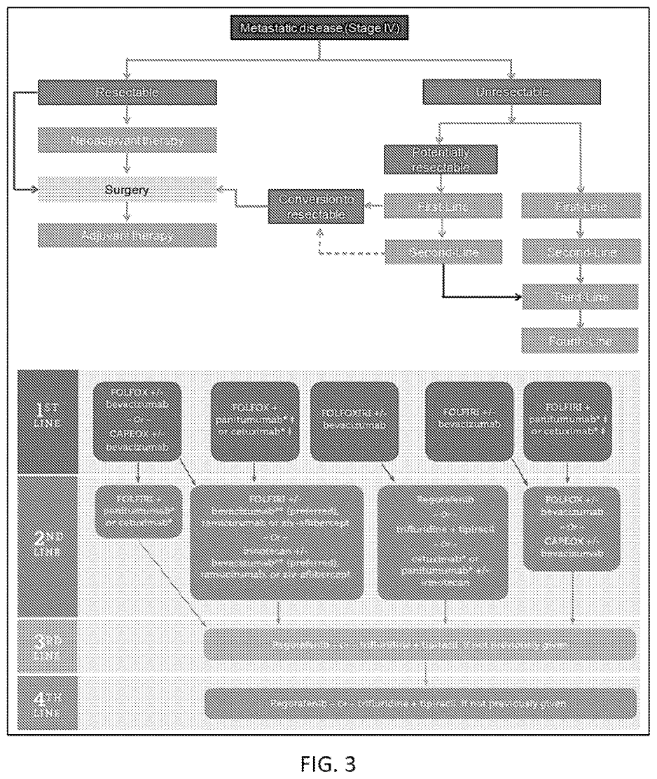

CRC is the third most prevalent cancer diagnosed each year and CRC patients have the second highest mortality rate worldwide. Early detection, surgery, and chemo/targeted therapy such as FOLFIRI, FOLFOX, and Avastin have minimally improved CRC overall survival. Despite the emergence of targeted therapies such as Avastin, which have not proven effective alone, chemotherapy is still the safer, more effective and affordable treatment for CRC (FIG. 2). Chemotherapy will remain a frontline treatment for the foreseeable future. Still, only about half of CRC patients respond to FOLFIRI-based chemotherapy and almost all display MDR at some point in the treatment. This poor response rate is attributed to the high tumor heterogeneity of CRC. CHD1L promotes CRC tumor progression, heterogeneity, and MDR. CHD1L expression is higher in stage IV metastatic CRC (mCRC). Surgery is not typical for mCRC, and chemo/targeted therapy is the standard of care (FIG. 3), yet these therapies are ineffective, evidenced by a low 11% 5-year overall survival.

The CRC market is expected to grow from $8B (2015) to $11B by 2025. There is a significant market demand to improve cure rates in neoadjuvant and adjuvant chemotherapy treatments for resected CRC patients (Stages II, III, and IV). Currently, there is no pipeline of drugs to address this unmet need, and chemotherapy such as FOLFIRI is projected to be the mainstay treatment of early stage and high-risk tumor resectable CRC patients. A significant portion of CRC patients that receive surgical resection and chemotherapy are projected to have tumor recurrence and/or progress to mCRC.

Despite new technologies to diagnose and treat cancer it remains to be a devastating disease, affecting millions of people worldwide. Tumor progression, metastasis, and multidrug resistance (MDR) remain the most difficult clinical challenges in the treatment of cancer and are the main cause of 90% of patient mortality for all cancer types.

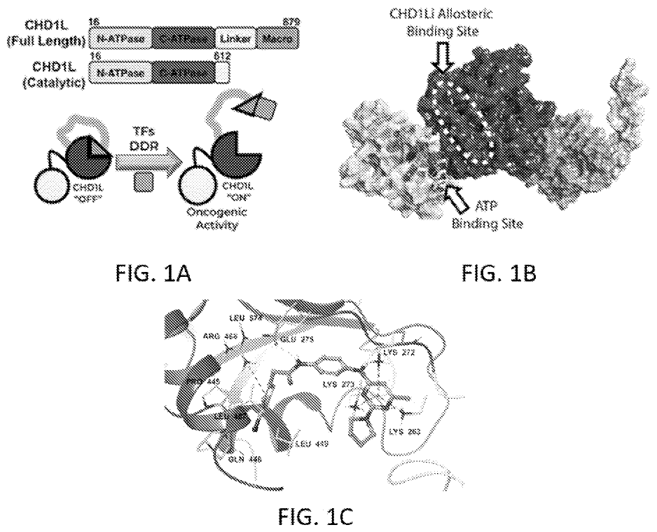

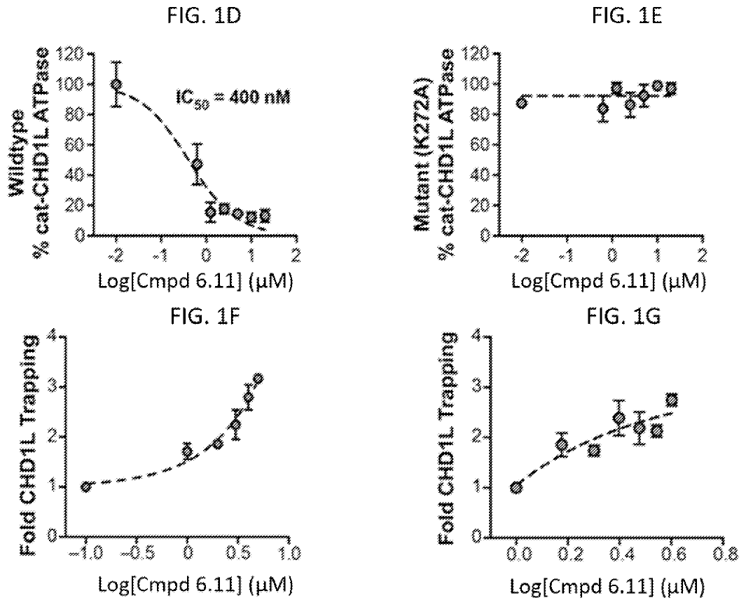

CHD1L is a chromatin remodeling enzyme that was first reported in 2008. CHD1L is unique from other chromatin remodelers in that it contains a macro domain (FIG. 1A-1C). The macro domain regulates CHD1L activity by autoinhibition. In normal cells that are slow growing or non-dividing CHD1L is off. However, in tumor cells it is activated by transcription factors (TFs), DNA damage response/repair (DDR) proteins (e.g., PARP1/2), and cell survival proteins. CHD1L has emerged as an oncogene that is amplified (Chr1q21) and over expressed in many cancers. CHD1L overexpression is a marker for poor prognosis and metastasis in numerous cancers, including colorectal cancer (CRC). Oncogenic potential by analyses of transcriptome data from 585 CRC patients over 17 years (GEOdataset, GSE40967) was determined. Low CHD1L-patients live 9-years longer compared to high-CHD1L patients. CHD1L is overexpressed in CRC patients with increased lymph node metastasis and patients that display multidrug resistance (MDR). CHD1L as a druggable molecular target in CRC and other cancers and discovered the first CHD1L inhibitors (CHD1Li) was validated. CHD1Li proved to be effective antitumor agents in vitro and in vivo by reversing epithelial-mesenchymal transition (EMT), inhibiting cancer stem cell (CSC) sternness, invasive potential, and tumor growth in CRC mouse xenografts.

Compound 6.11 likely binds to the CHD1L N-terminal allosteric binding site in the C-ATPase domain (FIG. 1B). This is substantiated with enzyme inhibition studies using cat-CHD1L, which is missing the C-terminal macro domain. Thus, CHD1Li does not inhibit CHD1L via the macro domain. In addition, molecular dynamics studies have identified the most stable binding site to be allosteric to the N-ATPase domain ATP binding site (FIGS. 1B & 1C). Importantly, the N-ATPase domain has the only active site for ATP hydrolysis. Compound 6.11 inhibition of CHD1L ATPase causes inhibition of CHD1L-mediated gene expression, epithelial-mesenchymal transition (EMT), cancer stem cell (CSC) sternness, invasive potential, PARP-mediated DNA repair, cell cycle progression, and cell survival. Compound 6.11 displays potent in vitro and in vivo antitumor activity (alone and in combination with chemotherapy) and is orally bioavailable with a plasma half-life of 8 hours in mice. Compound 6.11 shows no toxicity in mice as measured by complete blood count (CBC) panel and H&E histopathology of liver, heart, and bone marrow organs after treatment with 100 mg/kg Compound 6.11 daily for 30 days.

Identification of novel antitumor agents that target and inhibit tumor progression and synergize with standard of care chemotherapy can overcome IDR and improve the overall disease-free survival cure rates in many cancers including CRC. Compound 6.11 is a product that addresses the CRC unmet needs and market demand (FIGS. 1 & 2). Compound 6.11 combined with irinotecan or 5FU improves the potency of each drug ranging up to hundreds- to thousands-of-fold in cell-based models and tumor organoids. Furthermore, low doses of Compound 6.11 combined with irinotecan strongly synergize in vivo, significantly inhibiting tumor growth while increasing mouse survival by 30 days compared to irinotecan alone in drug resistant CRC nude mouse xenografts.

Provided herein are compounds, such as cancer drugs, that target and inhibit CHD1L (FIG. 1). In some embodiments, the compounds may be formulated for administration to a subject for treating a cancer. In some embodiments, the cancer is breast cancer, pancreatic cancer, melanoma, osteosarcoma, and lung cancer. In some embodiments, the subject may have a cancer that is drug resistant. A combination of a CHD1L inhibitor as described herein and a chemotherapy, such as a chemotherapy to which the cancer has become resistant, may be administered to the subject in need thereof. The chemotherapy may be a standard of care chemotherapy. In some instances, the CHD1L inhibitors described herein may be prescribed to a subject with the drug-resistant cancer in advance to prime the subject before receiving standard of care chemotherapy. In some embodiments, the combination of a CHD1L inhibitor and the chemotherapy may be maintained as a combination therapy to synergize in antitumor efficacy. In some instances, after completing therapies, subjects may stay on the CHD1L inhibitor or the compounds described herein as maintenance therapy to prevent tumor recurrence and promote disease-free overall survival extending the lives of patients beyond the current expectancies.

Compounds described herein may be effective with other therapeutic modalities such as biologics, emerging immune therapies, radiation therapy. Compounds described herein may be administered to patients as part of a diagnostic program, such as analyzing circulating tumor DNA (ctDNA) diagnostics (liquid biopsy). For example, ctDNA is proving to be an effective prognostic biomarker for CRC and other cancers, including measurable residual disease (MRD) that predicts if a cancer patient will likely relapse post-surgery/frontline therapy if no additional treatment is offered. Thus, compounds described herein when combined with chemo/targeted therapies could be deployed after ctDNA MDR detection in CRC and other cancer patients to prevent tumor relapse after surgery and frontline therapy. In addition, ctDNA identification combined with chemotherapy (e.g., FOLFIRI) and other therapies is in phase III clinical trials for treating CRC micro-metastatic disease (ClinicalTrials.gov Identifier: NCT03803553). Accordingly, compounds described herein may be combined with ctDNA identification for the treatment of micro-metastasis because the compounds of the present disclosure inhibit tumor progression and metastatic potential and may synergize with chemotherapy and other therapies.

CHD1L has been validated through clinical and basic science research as an oncogene, promoting tumor progression, metastasis, and MDR in the most prominent cancers, including lung, breast, and CRC. CHD1L is a chromatin remodeling enzyme that functions at the interface of tumor progression and tumor cell survival. It promotes malignant gene expression leading to tumor heterogeneity and increased metastatic potential. Further, it is involved in DDR and DNA repair, and is a direct inhibitor of programmed cell death mechanisms, facilitating tumor cell survival and MDR. Taken together, the aberrant function of CHD1L in tumor cells is a significant factor that leads to an increase in cancer patient mortality. CHD1Li Compound 6.11 may display single agent antitumor efficacy in tumor cell models, tumor organoids, and in mouse models of cancer, including the ability to inhibit CHD1L-mediated malignant gene expression, DNA repair, and tumor cell survival. In some embodiments, Compound 6.11 synergizes with the most broadly used frontline and standard of care chemotherapies, including but not limited to irinotecan, 5FU, doxorubicin, and the PARP inhibitor (PARPi) olaparib.

In some aspects, provided herein is a 4-step synthesis of Compound 6 and analogs. Using this synthetic approach combined with Schrödinger molecular modeling with the CHD1L crystal structure (PDB:7EPU) (FIG. 1C), drug design and medicinal chemistry generated a pipeline of >80 analogs. Structure activity relationships (SAR) using CHD1L recombinant enzyme, tumor cell models, tumor organoids, and tumor xenograft models were determined. In addition, in vitro and in vivo drug-like properties, drug metabolism and pharmacokinetics (DMPK), including aqueous solubility, mouse and human liver microsomal stability studies, in vivo PK/PD, oral bioavailability, and in vivo toxicology have been measured.

Without being bound by any particular theory, CHD1Li likely bind to the CHD1L N-terminal allosteric binding site in the C-ATPase domain (FIG. 1B). This is substantiated with enzyme inhibition studies using cat-CHD1L, which is missing the C-terminal macro domain. Thus, CHD1Li do not inhibit CHD1L via the macro domain. In addition, molecular dynamics studies have identified the most stable binding site to be allosteric to the N-ATPase domain ATP binding site (FIGS. 1B & 1C). Importantly, the N-ATPase domain has the only active site for ATP hydrolysis. Compound 6.11 inhibition of CHD1L ATPase causes inhibition of CHD1L-mediated gene expression, EMT, CSC stemness, invasive potential, PARP-mediated DNA repair, cell cycle progression, and cell survival. Compound 6.11 displays potent in vitro and in vivo antitumor activity (alone and in combination with chemotherapy) and is orally bioavailable with a plasma half-life of 8 hours in mice. Compound 6.11 shows no toxicity in mice as measured by complete blood count (CBC) panel and H&E histopathology of liver, heart, and bone marrow organs after treatment with 100 mg/kg Compound 6.11 daily for 30 days.

CHD1L is involved in PARP-mediated DNA repair and knockdown of CHD1L sensitizes tumor cells to DNA damaging agents. More recent papers validate CHD1L as a significant factor promoting MDR to DNA damaging chemotherapy and PARPi. Knockout of CHD1L sensitizes BRCA1/2 mutant tumor cells to olaparib in vitro and in vivo. Irinotecan generates single stranded DNA breaks via stabilizing the cleavage complex of topoisomerase 1 (TOP1) causing tumor cell death. CHD1L/PARP recruit TDP1, to excise the irinotecan/TOP1 complex for DNA repair, which may cause MDR to irinotecan among other CHD1L-mediated cell survival mechanisms (e.g. PARthanatos). Consistent with this mechanism of MDR, PARPi are also reported to synergize with irinotecan. Unlike CHD1Li, PARPi are ineffective against HR-proficient tumor cells and cause bone marrow toxicity. Thus, increasing PARPi administration to a patient with a drug-resistant cancer will increase undesirable side effects, such as bone marrow toxicity. As described herein, administration of an CHD1L inhibitor with a PARPi (such as Olaparib) unexpectedly and advantageously lowered the IC50 (i.e., dosage) of the PARPi. Lowering the IC₅₀ of a chemotherapy, such as a PARPi, allows for a subject to continue treatment while minimizing undesirable side effects that accompany higher dosages of the chemotherapy.

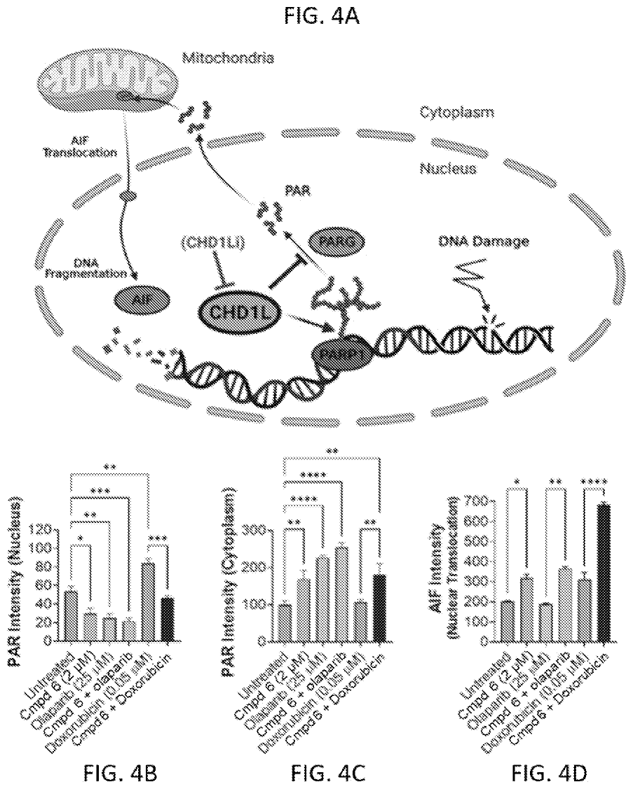

CHD1L may function as an anti-apoptotic factor by inhibiting pro-apoptotic proteins such as Nur77 and TP53 while activating other anti-apoptotic proteins like SPOCK1, TCTP, and MDM2. Additionally, CHD1L may regulate poly-ADP-ribosylation (PAR & PARylation) in cells and PAR-mediated programmed cell death (PARthanatos) (FIG. 4A). During PARthanatos, PAR is translocated to the cytoplasm and mitochondria, releasing apoptosis inducing factor (AIF, aka AIFM1), which is a biomarker for PARthanatos. AIF translocates to the nucleus promoting PARthanatos. CHD1L inhibitors may inhibit CHD1L/PARP1 mediated PARylation in the nucleus (FIG. 4B). DNA damage induced by doxorubicin significantly increases nuclear PARylation, promoting DNA repair, and MDR. In some instances, CHD1L inhibitors combined with doxorubicin inhibit nuclear PARylation. CHD1L inhibitors inhibit CHD1L blockade of PARglycohydrolase (PARG), increasing cytoplasmic PAR (FIG. 4C) and release of AIF, which translocates to the nucleus to induce PARthanatos (FIG. 4D). Of note, the combination of CHD1L inhibitors and olaparib or doxorubicin increases PARthanatos, revealing a unique synergistic mechanism of tumor cell death.

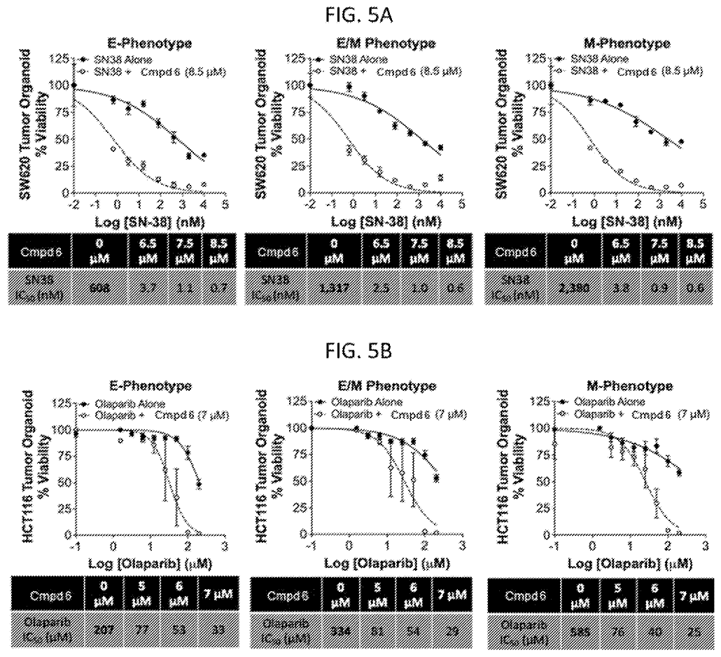

CHD1L also regulates DNA repair via CHD1L/TCF-driven EMT by upregulating ZEB1 in CRC. ZEB1 promotes mesenchymal cell survival by upregulating the DDR. CHD1Li may sensitize isolated quasi EMT phenotypes by inhibiting EMT and DNA repair. To test this, combination studies with Compound 6 and chemotherapy measuring the viability of isolated EMT dual-reporter quasi-E, -E/M, and -M SW620 (HR-proficient) cells and HCT116 (HR-deficient) cells cultured as tumor organoids were tested. MDR to SN38 (the active pharmacophore of the prodrug irinotecan chemotherapy) or olaparib increased with the increasing mesenchymal character (M>E/M>E). However, Compound 6 combined with SN38 sensitized each EMT phenotype the same, increasing the SN38 cytotoxic potency by hundreds-of-fold to thousands-of-fold in the respective EMT phenotypes (FIG. 5A). Similarly, Compound 6 combined with olaparib sensitized each EMT phenotype, increasing the potency of olaparib from very high UM (IC₅₀~600) to low μM (IC₅₀~30) (FIG. 5B). Taken together, CHD1Li synergized with chemotherapy and targeted therapy to overcome EMT-mediated drug resistance in CRC.

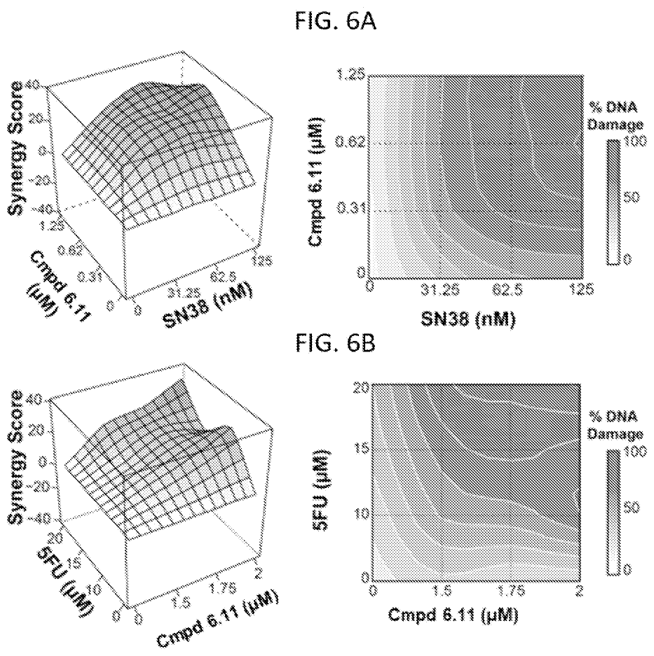

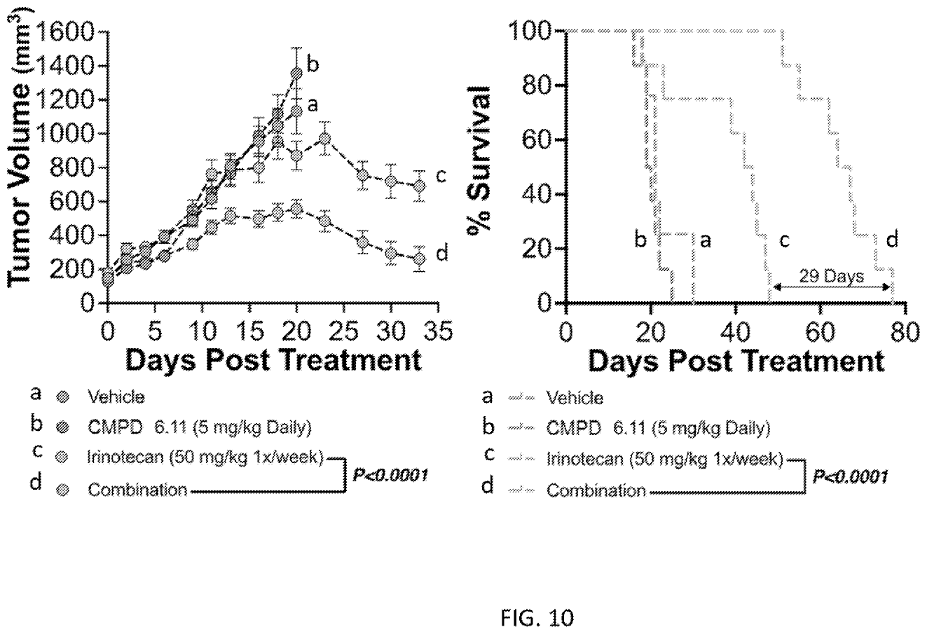

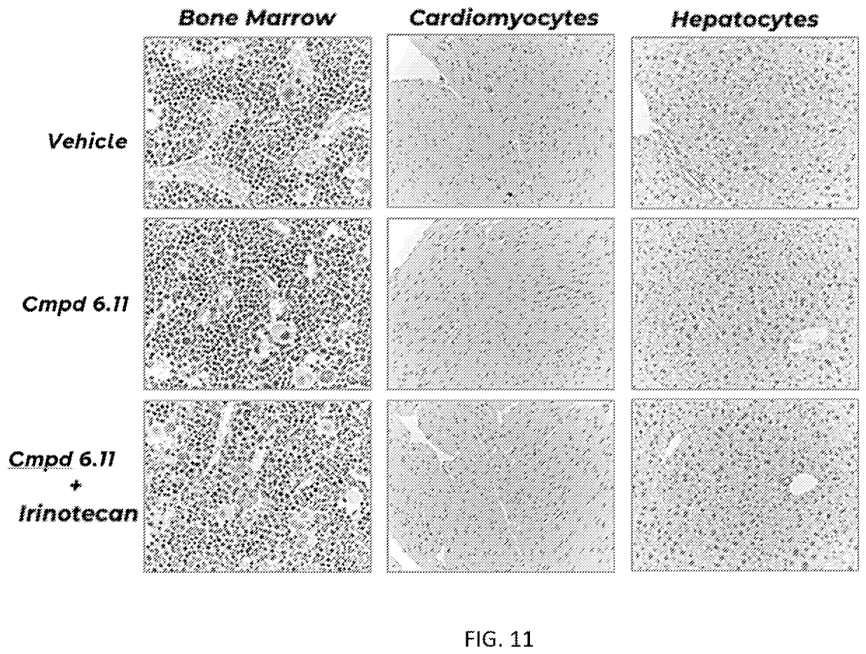

Without being bound by any particular theory, CHD1L inhibitors may be an effective inhibitor of tumor progression, may display single agent efficacy, and may synergize with broadly used front line chemotherapy for CRC and other cancers. CHD1L inhibitors may synergize with the active drug components of FOLFIRI (FOLinic acid aka leucovorin (LV), 5FU, IRInotecan). For example, Compound 6.11 may increase the potency of irinotecan/SN38 by hundreds to thousands-of-fold (FIGS. 5-8). This synergy was also measured in vivo using irinotecan drug resistant SW620 M-phenotype mouse tumor xenografts, in which the combination significantly inhibited tumor growth and increased survival compared to irinotecan alone (FIG. 10). 5FU has a complex mechanism of action (MOA) with the major target being thymidylate synthase (TS), affecting RNA processing, DNA replication and repair, and promoting cell death. In addition, LV is used with 5FU to enhance its binding to TS improving patient response rate but not overall survival. Like irinotecan/SN38, Compound 6.11 may synergize with 5FU, increasing its cytotoxic potency by hundreds to thousands-of-fold even in the absence of IV (FIGS. 6, 7, and 8). Recent literature shows that 5FU has an adverse therapy-induced MDR effect by promoting CSC stemness in CRC via the Wnt-pathway. CHD1L may be a component of the TCF/LEF complex that is recruited to open target promoter sites for transcription, and Compound 6.11 inhibits this action, which in turn inhibits EMT, CSC stemness, and invasive potential. Compound 6.11 may synergize with 5FU by inhibiting CHD1L-mediated DNA replication and repair and CHD1L-mediated TCF-driven EMT. In addition, Compound 6.11 alone induces tumor cell death via PARthanatos without causing significant DNA damage but increases PARthanatos when combined with chemotherapy (FIG. 4A). Also, Compound 6.11 synergizes with a variety of chemo and targeted therapies having broadly different MOAs. Compound 6.11 has excellent drug-like properties and the potential for clinical translation. A 4-step total synthesis of Compound 6.11 and demonstrate the scale up to gram quantity was developed. Optimize the scale up synthesis of Compound 6.11 from 5 g to 1 kg of non-GLP material (at least 97% purity). Deliver a kg scale synthesis to be transferred to a CRO for GMP synthesis to be used in IND studies.

CHD1L is a novel and contemporary oncogene that functions at the interface of tumor progression, metastasis, tumor cell survival, and MDR. Compound 6.11 represents an antitumor pharmacophore from a pipeline of CHD1Li that are currently the first- and best-in-class drugs targeting CHD1L (FIG. 3). In addition to regulating malignant gene expression and DNA repair, CHD1L is essential for PAR-mediated PARthanatos (FIG. 4). Furthermore, CHD1L inhibitors may synergize with broadly used chemotherapy and targeted therapy, making the market landscape less of a competition and more of an opportunity to develop more effective combination therapies to improve patient response rates and survival while decreasing tumor progression and recurrence. For example, Compound 6.11 and other CHD1Li described herein are the only known compounds that synergize with PARPi, improving their potency and efficacy. PARPi have not shown effectiveness in BRCA wildtype HR-proficient cancers. Data described herein demonstrates that Compound 6.11 combinations with olaparib significantly improve the potency in HR-deficient and HR-proficient CRC tumor organoids (FIGS. 5B & 7C). Thus, Compound 6.11 may help to expand the indications for PARPi. Compound 6.11 is also unique in that it inhibits mechanisms of tumor progression, metastasis, CSC stemness, and cell survival, and strongly synergizes with broadly used chemotherapies, such as 5FU and irinotecan (FOLFIRI), doxorubicin, docetaxel, and olaparib.

Provided herein are CHD1L inhibitors (CHD1Li). Further provided herein are therapeutic compositions comprising a CHD1Li and a therapeutic agent. CHD1L inhibitors (CHD1Li) are used as targeted therapies for the treatment of multiple cancer types, including CRC, breast cancer, lung cancer, pancreatic cancer, melanoma, and osteosarcoma. CHD1Li are effective antitumor agents that synergize with broadly used standard of care chemotherapy and targeted therapy. In some embodiments, the CHD1L inhibitors synergize with the chemotherapy and can reduce a dosage of the chemotherapy for treating cancer, such as drug-resistant cancers.

In some aspects, provided herein are methods of treating a cancer, such as colorectal cancer (CRC), breast cancer, lung cancer, pancreatic cancer, melanoma, and osteosarcoma. In some embodiments, the breast cancer is triple negative breast cancer (TNBC). In some embodiments, the breast cancer is luminal breast cancer.

In some aspects, provided herein are methods of treating a cancer comprising administering a CHD1L inhibitor. In some embodiments, the CHD1L inhibitor is selected from Table 1. In some embodiments, treating the cancer comprises administering a composition comprising a CHD1L inhibitor described herein.

### Methods of Treatment

Provided herein are methods for treating cancer in a subject with a therapeutically effective amount of a composition (e.g., composition, pharmaceutical composition, pharmaceutical formulation) of the present disclosure. In some embodiments, the methods comprise administering to the subject a therapeutically effective amount of a compound described herein, or a pharmaceutically acceptable salt or solvate thereof. In some embodiments, the compound is provided in Table 1. In some embodiments, the compound has a structure of Formula (I), (II), (IIIA), or (IIIB) (see “Compositions” Section). In some embodiments, the methods comprise administering to the subject a combination of the compound or the pharmaceutically acceptable salt or solvate thereof, and an additional therapeutic agent (e.g., chemotherapy). In some embodiments, the administration of the compound or a pharmaceutically acceptable salt or solvate thereof potentiates or is synergistic with the chemotherapy. In some embodiments, the cancer is resistant to the chemotherapy when the cancer is treated without the compound or the pharmaceutically acceptable salt or solvate thereof. In some embodiments, the combination therapies comprising a chemotherapy and compound of the present disclosure are therapeutically effective to treat the cancer in a subject, even if the cancer is resistant to treatment with the chemotherapy alone.

In some embodiments, the compound described herein, or a pharmaceutically acceptable salt or solvate thereof, modulates activity of CHD1L in a subject in need thereof. In some embodiments, the compound described herein, or a pharmaceutically acceptable salt or solvate thereof, inhibits CHD1L in a subject in need thereof. In some embodiments, the cancer is colorectal cancer, breast cancer, melanoma, osteosarcoma, or lung cancer. In some embodiments, the cancer is colorectal cancer or breast cancer. In some embodiments, the breast cancer is triple negative breast cancer (TNBC). In some embodiments, the cancer is pancreatic cancer. In some embodiments, the cancer is lunch cancer. In some embodiments, the cancer is melanoma. In some embodiments, the cancer is osteosarcoma. In some embodiments, the subject is a mammal. In some embodiments, the subject is a human.

In some embodiments, the methods comprise administering an inhibitor of CHD1L (also referred to as “CHD1Li”) to a subject in need thereof. In some embodiments, method comprise treating a cancer in the subject. In some embodiments, the method comprises binding a CHD1L inhibitor to an allosteric binding site of CHD1L. In some embodiments, binding a CHD1L inhibitor comprises binding the CHD1L inhibitor to a CHD1L having at least about 70% sequence identity to SEQ ID NO: 1. In some embodiments, binding a CHD1L inhibitor comprises binding the CHD1L inhibitor to a lysine in the allosteric binding site. In some instances, the lysine is lysine-272 (K272). In some embodiments, binding a CHD1L inhibitor comprises binding the CHD1L inhibitor to a glutamate in the allosteric binding site. In some instances, the glutamate is glutamate-275 (E275). In some embodiments, the CHD1L has a sequence selected from Table 2. In some embodiments, the CHD1L inhibitor binds to a region of CHD1L comprising SEQ ID NO: 40, SEQ ID NO: 41, SEQ ID NO: 42, SEQ ID NO: 43, or SEQ ID NO: 44, or a combination of two or more thereof. In some embodiments, the CHD1L inhibitor binds to a region of CHD1L comprising SEQ ID NO: 40. In some embodiments, the CHD1L inhibitor binds to a region of CHD1L comprising SEQ ID NO: 41. In some embodiments, the CHD1L inhibitor binds to a region of CHD1L comprising SEQ ID NO: 42. In some embodiments, the CHD1L inhibitor binds to a region of CHD1L comprising SEQ ID NO: 43. In some embodiments, the CHD1L inhibitor binds to a region of CHD1L comprising SEQ ID NO: 44.

In some aspects, disclosed herein is a method of treating cancer, the method comprising: administering to a subject with the cancer a composition comprising: a therapeutically effective amount of a chromodomain-helicase-DNA-binding protein 1-like (CHD1L) inhibitor having the structure of Formula (I):

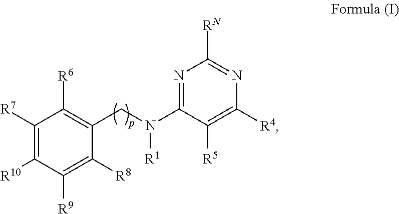

or a pharmaceutically acceptable salt thereof, wherein:
   R^{N} is an optionally substituted 5- to 7-membered heterocycle optionally containing a second heteroatom selected from the of group N, S, and O;
   R¹ is selected from hydrogen or C₁₋₃ alkyl;
   each R⁴ and R⁵ is selected from hydrogen, C₁₋₃ alkyl, and halogen;
   R⁶ is hydrogen or a halogen;
   each R⁷, R⁸, and R⁹ is independently selected from hydrogen and C₁₋₃ alkyl, optionally substituted with C₁₋₃ alkyl or aryl;
   R¹⁰ is NH—C(O)—(CH₂)ₘ—Ar, wherein
      m is 0, 1, 2, 3, 4, or 5, and
      Ar is aryl or heteroaryl optionally substituted with R^{a},
         wherein each R^{a} is selected from hydrogen, C₁₋₃ alkyl, C₁₋₃ alkoxy, C₁₋₃ haloalkyl, or halogen, or two R^{a} together with the carbon to which they are bound form a 5- or 6-membered carbocycle, aryl, or heteroaryl optionally substituted with R^{a}; and
   p is 0, 1, or 2,
      wherein the cancer is breast cancer, melanoma, osteosarcoma, lung cancer, or pancreatic cancer.

In some embodiments, p is 0 or 1. In some embodiments, R¹ is hydrogen. In some embodiments, Ar is a 5- to 10-membered heterocycle. In some embodiments, the 5- to 10-membered heterocycle is thiophenyl, furanyl, pyranyl, pyrrolyl, benzofuranyl, isobenzofuranyl, oxazolyl, indolyl, benzo[b]thiophenyl, or benzo[c]thiophenyl. In some embodiments, at least one of R⁶, R⁷, R⁸, or R⁹ is hydrogen. In some embodiments, Ar is substituted with at least one R^{a}. In some embodiments, R¹⁰ has the structure:

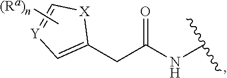

wherein X is N, S, or O; Y is N, S, or C; R^{a} is selected from: hydrogen, C₁₋₃ alkyl, C₁₋₃ alkoxy, C₁₋₃ haloalkyl, or halogen; and n is 0, 1, 2, or 3.

In some embodiments, the CHD1Li has the structure of Formula (II):

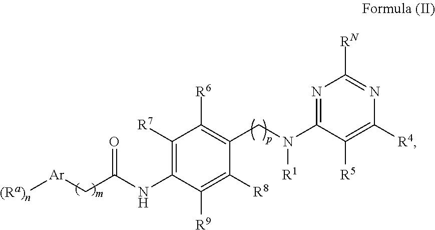

or a pharmaceutically acceptable salt thereof, wherein:
   R^{N} is an optionally substituted 5- to 7-membered heterocycle optionally containing a second heteroatoms selected from the group N, S, or O;
   R¹ is hydrogen or methyl;
   R⁴ and R⁵ are each independently hydrogen, C₁₋₃ alkyl group, or halogen;
   R⁶ is hydrogen or halogen;
   R⁷, R⁸, and R⁹ are each independently hydrogen, C₁₋₃ alkyl, C₁₋₃ alkoxy, optionally substituted C₁₋₃ alkyl, or aryl;
   Ar is an aryl or heteroaryl optionally substituted R^{a}, wherein
      each R^{a} is selected from hydrogen, C₁₋₃ alkyl, C₁₋₃ alkoxy, C₁₋₃ haloalkyl, or halogen, or two R^{a} together with the carbon to which they are bound form a 5- or 6-membered carbocycle, aryl, or heteroaryl optionally substituted with R^{b}, and
      each R^{b} is selected from C₁₋₃ alkyl, C₁₋₃ alkoxy, C₁₋₃ haloalkyl, or halogen;
   p is 0 or 1;
   m is 0, 1, 2, 3, 4, or 5; and
   n is an integer from 0 to 9.

In some embodiments, Ar is a 5- to 10-membered heterocycle. In some embodiments, the 5- to 10-membered heterocycle is thiophenyl, furanyl, pyranyl, pyrrolyl, benzofuranyl, isobenzofuranyl, oxazolyl, indolyl, benzo[b]thiophenyl, or benzo[c]thiophenyl. In some embodiments, R⁴ is methyl. In some embodiments, R¹ is hydrogen. In some embodiments, at least two of R⁵, R⁶, R⁷, R⁸, and R⁹ are hydrogen. In some embodiments, at least one of R⁵, R⁶, R⁷, R⁸, or R⁹ is halogen or C₁₋₃ alkoxy.

In some embodiments, the CHD1L inhibitor has the structure of Formula (IIIA):

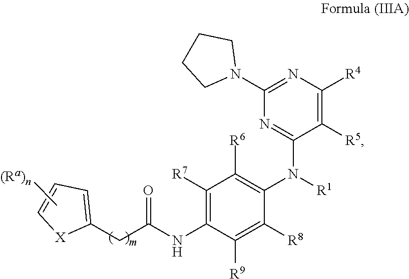

or a pharmaceutically acceptable salt thereof, wherein:
   R¹ is hydrogen or methyl;
   R⁴ and R⁵ are each independently hydrogen, C₁₋₃ alkyl group, or halogen;
   R⁶ is hydrogen or a halogen;
   R⁷, R⁸, and R⁹ are each independently hydrogen, C₁₋₃ alkyl, optionally substituted C₁₋₃ alkyl, or aryl;
   each R^{a} is selected from hydrogen, C₁₋₃ alkyl, C₁₋₃ alkoxy, C₁₋₃ haloalkyl, or halogen, or two R^{a} together with the carbon to which they are bound form a 5- or 6-membered carbocycle, aryl, or heteroaryl optionally substituted with R^{b};
   each R^{b} is selected from C₁₋₃ alkyl, C₁₋₃ alkoxy, C₁₋₃ haloalkyl, or halogen;
   X is N, S, or O;
   m is 0, 1, 2, 3, 4, or 5; and
   n is 0, 1, 2, 3, or 4.

In some embodiments, R¹ is hydrogen. In some embodiments, at least one of R⁶, R⁷, R⁸, or R⁹ is hydrogen. The In some embodiments, m is 1, 2, 3, 4, or 5. In some embodiments, n is 1, 2, 3, or 4. In some embodiments, when n is 0, m is at least 1. In some embodiments, R^{a} is a C₁₋₃ alkyl or halogen. In some embodiments, the halogen is bromo or chloro. In some embodiments, X is N. In some embodiments, X is S. In some embodiments, X is O. In some embodiments, at least one of R⁴ or R⁵ is methyl. In some embodiments, CHD1L inhibitor having the structure of Formula (IIIA) is:

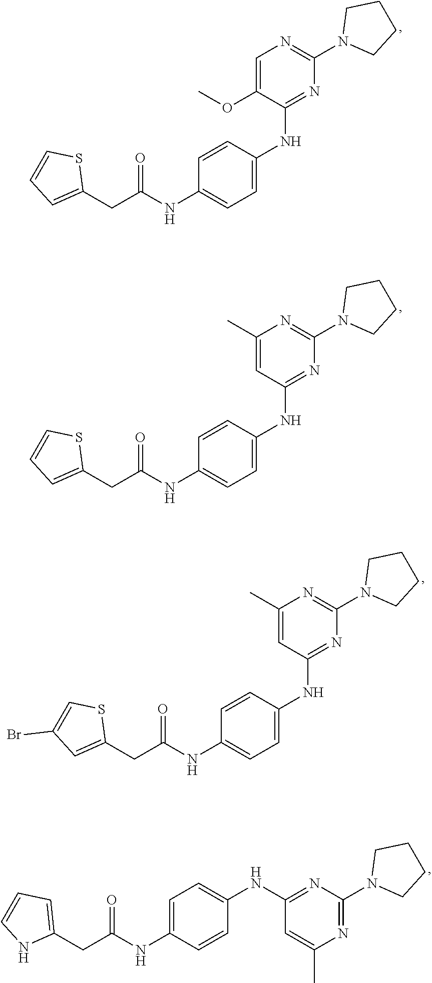 [SMILES: COc1cnc(N2CCCC2)nc1Nc1ccc(NC(=O)Cc2cccs2)cc1 ; Cc1cc(Nc2ccc(NC(=O)Cc3cccs3)cc2)nc(N2CCCC2)n1 ; Cc1cc(Nc2ccc(NC(=O)Cc3cc(Br)cs3)cc2)nc(N2CCCC2)n1 ; Cc1cc(Nc2ccc(NC(=O)Cc3ccc[nH]3)cc2)nc(N2CCCC2)n1]

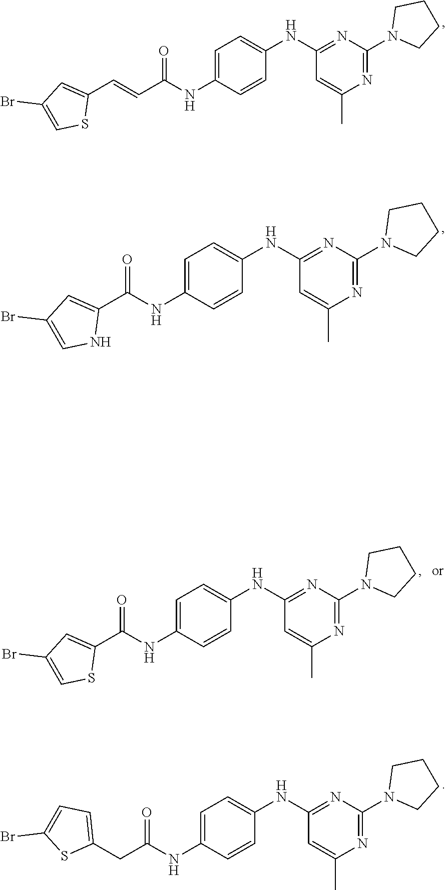 [SMILES: Cc1cc(Nc2ccc(NC(=O)/C=C/c3cc(Br)cs3)cc2)nc(N2CCCC2)n1 ; Cc1cc(Nc2ccc(NC(=O)c3cc(Br)c[nH]3)cc2)nc(N2CCCC2)n1 ; Cc1cc(Nc2ccc(NC(=O)c3cc(Br)cs3)cc2)nc(N2CCCC2)n1 ; Cc1cc(Nc2ccc(NC(=O)Cc3ccc(Br)s3)cc2)nc(N2CCCC2)n1]

In some embodiments, the CHD1L inhibitor has the structure of Formula (IIIB):

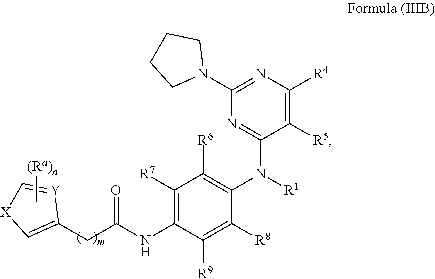

or a pharmaceutically acceptable salt thereof, wherein:
   R¹ is hydrogen or methyl;
   R⁴ and R⁵ are each independently hydrogen, C₁₋₃ alkyl group, or halogen;
   R⁶ is hydrogen or a halogen;
   R⁷, R⁸, and R⁹ are each independently hydrogen, C₁₋₃ alkyl, optionally substituted C₁₋₃ alkyl, or aryl;
   each R^{a} is selected from hydrogen, C₁₋₃ alkyl, C₁₋₃ alkoxy, C₁₋₃ haloalkyl, or halogen, or two R^{a} together with the carbon to which they are bound form a 5- or 6-membered carbocycle, aryl, or heteroaryl optionally substituted with R^{b};
   each R^{b} is selected from C₁₋₃ alkyl, C₁₋₃ alkoxy, C₁₋₃ haloalkyl, or halogen;
   X is N, S, or O;
   Y is C, N, or S;
   m is 1, 2, 3, 4, or 5; and
   n is 1, 2, 3, or 4.

In some embodiments, R¹ is hydrogen. In some embodiments, at least one of R⁶, R⁷, R⁸, or R⁹ is hydrogen. In some embodiments, m is 1, 2, or 3. In some embodiments, n is 1, 2, 3, or 4. In some embodiments, R^{a} is a C₁₋₃ alkyl or halogen. In some embodiments, the halogen is bromo or chloro. In some embodiments, X is N. In some embodiments, X is O. In some embodiments, X is S. In some embodiments, Y is N, n is 1, and R^{a} is halogen. In some embodiments, at least one or R⁴ or R⁵ is methyl. In some embodiments, the CHD1L inhibitor of Formula (IIIB) is:

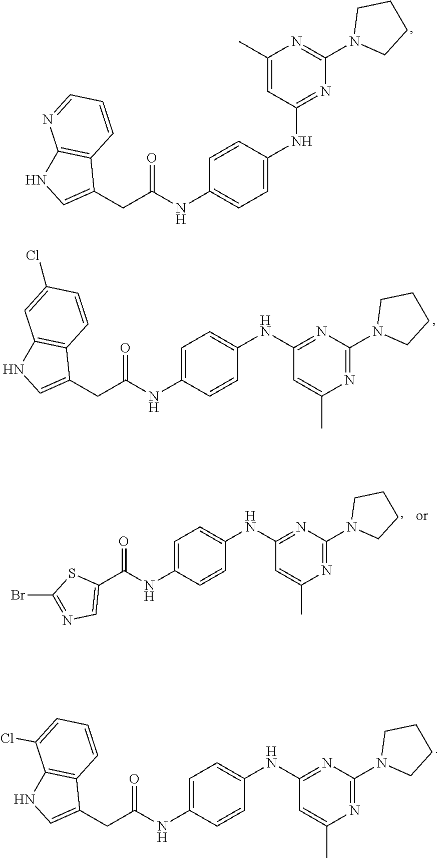 [SMILES: Cc1cc(Nc2ccc(NC(=O)Cc3c[nH]c4ncccc34)cc2)nc(N2CCCC2)n1 ; Cc1cc(Nc2ccc(NC(=O)Cc3c[nH]c4cc(Cl)ccc34)cc2)nc(N2CCCC2)n1 ; Cc1cc(Nc2ccc(NC(=O)c3cnc(Br)s3)cc2)nc(N2CCCC2)n1 ; Cc1cc(Nc2ccc(NC(=O)Cc3c[nH]c4c(Cl)cccc34)cc2)nc(N2CCCC2)n1]

In some embodiments, the CHD1L inhibitor is a compound selected from Table 1.

TABLE 1
Compounds.

| Compound | | |
|---|---|---|
| No. | Structure | Name |
| 1 | 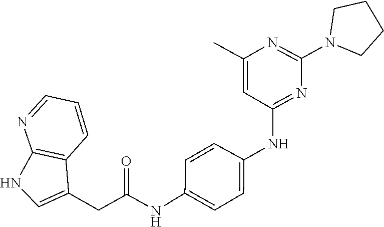 [SMILES: Cc1cc(Nc2ccc(NC(=O)Cc3c[nH]c4ncccc34)cc2)nc(N2CCCC2)n1] | N-(4-((6-methyl-2-(pyrrolidin-1- yl)pyrimidin-4-yl)amino)phenyl)- 2-(1H-pyrrolo[2,3-b]pyridin-3- yl)acetamide |
| 2 | 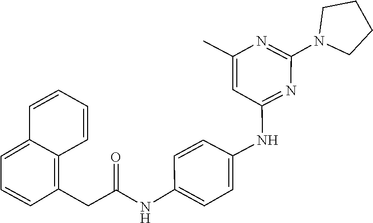 [SMILES: Cc1cc(Nc2ccc(NC(=O)Cc3cccc4ccccc34)cc2)nc(N2CCCC2)n1] | N-(4-((6-methyl-2-(pyrrolidin-1- yl)pyrimidin-4-yl)amino)phenyl)- 2-(naphthalen-1-yl)acetamide |
| 3 | 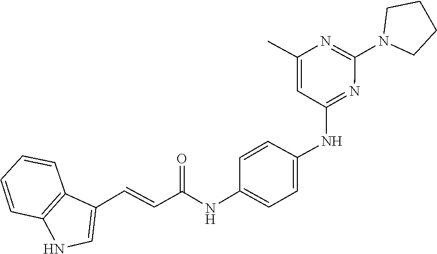 [SMILES: Cc1cc(Nc2ccc(NC(=O)/C=C/c3c[nH]c4ccccc34)cc2)nc(N2CCCC2)n1] | (E)-3-(1H-indol-3-yl)-N-(4-((6- methyl-2-(pyrrolidin-1- yl)pyrimidin-4- yl)amino)phenyl)acrylamide |
| 4 | 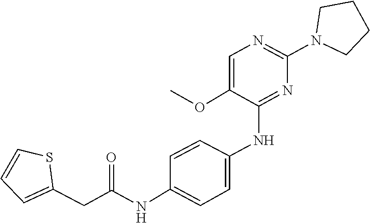 [SMILES: COc1cnc(N2CCCC2)nc1Nc1ccc(NC(=O)Cc2cccs2)cc1] | N-(4-((5-methoxy-2-(pyrrolidin- 1-yl)pyrimidin-4- yl)amino)phenyl)-2-(thiophen-2- yl)acetamide |
| 5 | 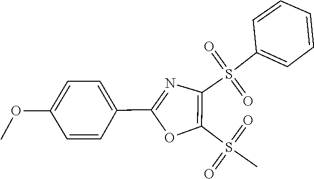 [SMILES: COc1ccc(-c2nc(S(=O)(=O)c3ccccc3)c(S(C)(=O)=O)o2)cc1] | 2-(4-methoxyphenyl)-5- (methylsulfonyl)-4- (phenylsulfonyl)oxazole |
| 6 | 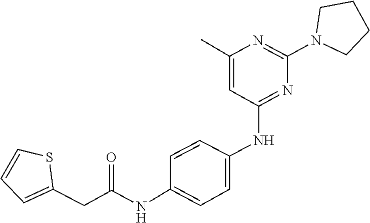 [SMILES: Cc1cc(Nc2ccc(NC(=O)Cc3cccs3)cc2)nc(N2CCCC2)n1] | N-(4-((6-methyl-2-(pyrrolidin-1- yl)pyrimidin-4-yl)amino)phenyl)- 2-(thiophen-2-yl)acetamide |
| 6.11 | 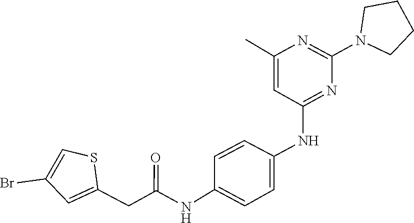 [SMILES: Cc1cc(Nc2ccc(NC(=O)Cc3cc(Br)cs3)cc2)nc(N2CCCC2)n1] | 2-(4-bromothiophen-2-yl)-N-(4- ((6-methyl-2-(pyrrolidin-1- yl)pyrimidin-4- yl)amino)phenyl)acetamide |
| 7 | 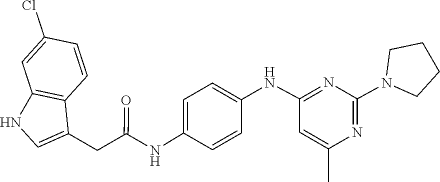 [SMILES: Cc1cc(Nc2ccc(NC(=O)Cc3c[nH]c4cc(Cl)ccc34)cc2)nc(N2CCCC2)n1] | 2-(6-chloro-1H-indol-3-yl)-N-(4- ((6-methyl-2-(pyrrolidin-1- yl)pyrimidin-4- yl)amino)phenyl)acetamide |
| 8 | 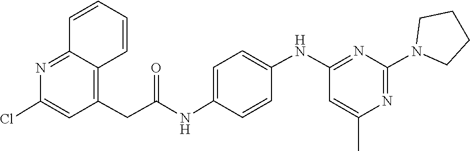 [SMILES: Cc1cc(Nc2ccc(NC(=O)Cc3cc(Cl)nc4ccccc34)cc2)nc(N2CCCC2)n1] | 2-(2-chloroquinolin-4-yl)-N-(4- ((6-methyl-2-(pyrrolidin-1- yl)pyrimidin-4- yl)amino)phenyl)acetamide |
| 9 | 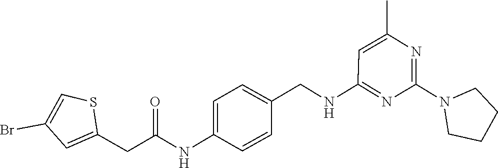 [SMILES: Cc1cc(NCc2ccc(NC(=O)Cc3cc(Br)cs3)cc2)nc(N2CCCC2)n1] | 2-(4-bromothiophen-2-yl)-N-(4- (((6-methyl-2-(pyrrolidin-1- yl)pyrimidin-4- yl)amino)methyl)phenyl) acetamide |
| 10 | 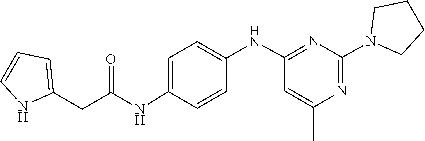 [SMILES: Cc1cc(Nc2ccc(NC(=O)Cc3ccc[nH]3)cc2)nc(N2CCCC2)n1] | N-(4-((6-methyl-2-(pyrrolidin-1- yl)pyrimidin-4-yl)amino)phenyl)- 2-(1H-pyrrol-2-yl)acetamide |
| 11 | 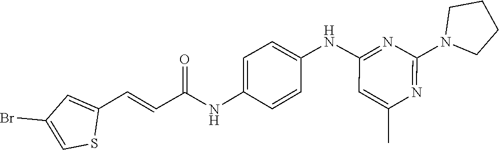 [SMILES: Cc1cc(Nc2ccc(NC(=O)/C=C/c3cc(Br)cs3)cc2)nc(N2CCCC2)n1] | (E)-3-(4-bromothiophen-2-yl)-N- (4-((6-methyl-2-(pyrrolidin-1- yl)pyrimidin-4- yl)amino)phenyl)acrylamide |
| 12 | 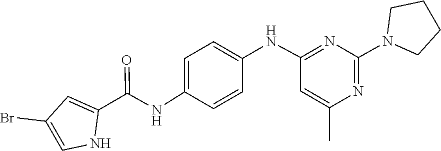 [SMILES: Cc1cc(Nc2ccc(NC(=O)c3cc(Br)c[nH]3)cc2)nc(N2CCCC2)n1] | 4-bromo-N-(4-((6-methyl-2- (pyrrolidin-1-yl)pyrimidin-4- yl)amino)phenyl)-1H-pyrrole-2- carboxamide |
| 13 | 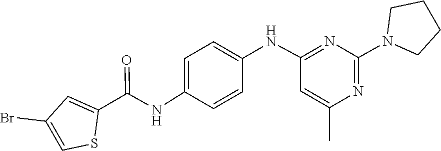 [SMILES: Cc1cc(Nc2ccc(NC(=O)c3cc(Br)cs3)cc2)nc(N2CCCC2)n1] | 4-bromo-N-(4-((6-methyl-2- (pyrrolidin-1-yl)pyrimidin-4- yl)amino)phenyl)thiophene-2- carboxamide |
| 14 | 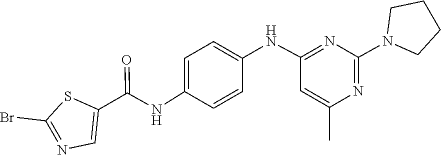 [SMILES: Cc1cc(Nc2ccc(NC(=O)c3cnc(Br)s3)cc2)nc(N2CCCC2)n1] | 2-bromo-N-(4-((6-methyl-2- (pyrrolidin-1-yl)pyrimidin-4- yl)amino)phenyl)thiazole-5- carboxamide |
| 15 | 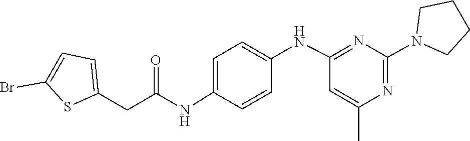 [SMILES: Cc1cc(Nc2ccc(NC(=O)Cc3ccc(Br)s3)cc2)nc(N2CCCC2)n1] | 2-(5-bromothiophen-2-yl)-N-(4- ((6-methyl-2-(pyrrolidin-1- yl)pyrimidin-4- yl)amino)phenyl)acetamide |
| 16 | 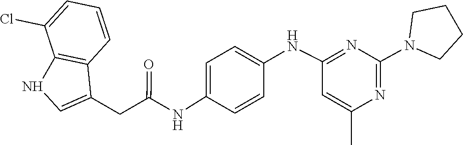 [SMILES: Cc1cc(Nc2ccc(NC(=O)Cc3c[nH]c4c(Cl)cccc34)cc2)nc(N2CCCC2)n1] | 2-(7-chloro-1H-indol-3-yl)-N-(4- ((6-methyl-2-(pyrrolidin-1- yl)pyrimidin-4- yl)amino)phenyl)acetamide |
| 17 | 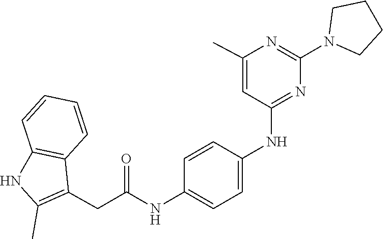 [SMILES: Cc1cc(Nc2ccc(NC(=O)Cc3c(C)[nH]c4ccccc34)cc2)nc(N2CCCC2)n1] | 2-(2-methyl-1H-indol-3-yl)-N-(4- ((6-methyl-2-(pyrrolidin-1- yl)pyrimidin-4- yl)amino)phenyl)acetamide |

Described herein, in some embodiments, is a compound of Formula (I), (II), (IIIA), or (IIIB), or a pharmaceutically acceptable salt or solvate thereof, for use in treating or preventing a cancer. In some embodiments, the methods comprise treating or preventing the cancer by administering the compound of Formula (I), (II), (IIIA), or (IIIB), or a pharmaceutically acceptable salt or solvate thereof, to the subject. In some embodiments, the compound is any one of compounds 1-17 provided in Table 1. In some embodiments, the cancer is osteosarcoma, breast cancer, lung cancer, pancreatic cancer, colorectal cancer (CRC), or metastasis of any of the foregoing. In some embodiments, the cancer is osteosarcoma. In some embodiments, the cancer is breast cancer. In some embodiments, the cancer is lung cancer. In some embodiments, the cancer is pancreatic cancer. In some embodiments, the cancer is colorectal cancer (CRC). In some embodiments, the cancer is colorectal cancer, breast cancer, melanoma, osteosarcoma, lung cancer, or pancreatic cancer that has metastasized elsewhere in the subject.

Described herein, in some embodiments, is a compound 1, or a pharmaceutically acceptable salt or solvate thereof, for use in treating or preventing a cancer. In some embodiments, the methods comprise treating cancer in a subject, the method comprising administering compound 1, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

Described herein, in some embodiments, is a compound 2, or a pharmaceutically acceptable salt or solvate thereof, for use in treating or preventing a cancer. Provided herein are methods of treating cancer in a subject, the method comprising administering compound 2, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

Described herein, in some embodiments, is a compound 3, or a pharmaceutically acceptable salt or solvate thereof, for use in treating or preventing a cancer. In some embodiments, the methods comprise treating cancer in a subject, the method comprising administering compound 3, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

Described herein, in some embodiments, is a compound 4, or a pharmaceutically acceptable salt or solvate thereof, for use in treating or preventing a cancer. In some embodiments, the methods comprise treating cancer in a subject, the method comprising administering compound 4, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

Described herein, in some embodiments, is a compound 5, or a pharmaceutically acceptable salt or solvate thereof, for use in treating or preventing a cancer. In some embodiments, the methods comprise treating cancer in a subject, the method comprising administering compound 5, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

Described herein, in some embodiments, is a compound 6, or a pharmaceutically acceptable salt or solvate thereof, for use in treating or preventing a cancer. In some embodiments, the methods comprise treating cancer in a subject, the method comprising administering compound 6, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

Described herein, in some embodiments, is a compound 6.11, or a pharmaceutically acceptable salt or solvate thereof, for use in treating or preventing a cancer. In some embodiments, the methods comprise treating cancer in a subject, the method comprising administering compound 6, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

Described herein, in some embodiments, is a compound 7, or a pharmaceutically acceptable salt or solvate thereof, for use in treating or preventing a cancer. In some embodiments, the methods comprise treating cancer in a subject, the method comprising administering compound 7, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

Described herein, in some embodiments, is a compound 8, or a pharmaceutically acceptable salt or solvate thereof, for use in treating or preventing a cancer. In some embodiments, the methods comprise treating cancer in a subject, the method comprising administering compound 8, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

Described herein, in some embodiments, is a compound 9, or a pharmaceutically acceptable salt or solvate thereof, for use in treating or preventing a cancer. In some embodiments, the methods comprise treating cancer in a subject, the method comprising administering compound 9, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

Described herein, in some embodiments, is a compound 10, or a pharmaceutically acceptable salt or solvate thereof, for use in treating or preventing a cancer. In some embodiments, the methods comprise treating cancer in a subject, the method comprising administering compound 10, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

Described herein, in some embodiments, is a compound 11, or a pharmaceutically acceptable salt or solvate thereof, for use in treating or preventing a cancer. In some embodiments, the methods comprise treating in a subject, the method comprising administering compound 11, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

Described herein, in some embodiments, is a compound 12, or a pharmaceutically acceptable salt or solvate thereof, for use in treating or preventing a cancer. In some embodiments, the methods comprise treating cancer in a subject, the method comprising administering compound 12, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

Described herein, in some embodiments, is a compound 13, or a pharmaceutically acceptable salt or solvate thereof, for use in treating or preventing a cancer. In some embodiments, the methods comprise treating cancer in a subject, the method comprising administering compound 13, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

Described herein, in some embodiments, is a compound 14, or a pharmaceutically acceptable salt or solvate thereof, for use in treating or preventing a cancer. In some embodiments, the methods comprise treating cancer in a subject, the method comprising administering compound 14, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

Described herein, in some embodiments, is a compound 15, or a pharmaceutically acceptable salt or solvate thereof, for use in treating or preventing a cancer. In some embodiments, the methods comprise treating cancer in a subject, the method comprising administering compound 15, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

Described herein, in some embodiments, is a compound 16, or a pharmaceutically acceptable salt or solvate thereof, for use in treating or preventing a cancer. In some embodiments, the methods comprise treating cancer in a subject, the method comprising administering compound 16, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

Described herein, in some embodiments, is a compound 17, or a pharmaceutically acceptable salt or solvate thereof, for use in treating or preventing a cancer. In some embodiments, the methods comprise treating cancer in a subject, the method comprising administering compound 17, or a pharmaceutically acceptable salt or solvate thereof, to the subject. In some embodiments, the cancer is osteosarcoma, breast cancer, lung cancer, pancreatic cancer, colorectal cancer (CRC), or metastasis of any of the foregoing.

In some embodiments, the subject has osteosarcoma, breast cancer, lung cancer, pancreatic cancer, colorectal cancer (CRC), or metastasis of any of the foregoing. In some embodiments, the subject is suspected of having a severe form of osteosarcoma, breast cancer, lung cancer, pancreatic cancer, colorectal cancer (CRC), or metastasis of any of the foregoing. In some embodiments, the subject is suspected of having or has a form of osteosarcoma, breast cancer, lung cancer, pancreatic cancer, colorectal cancer (CRC), or metastasis of any of the foregoing, that is resistant to treatment by a chemotherapy of the present disclosure.

In some embodiments, a compound described herein, or a pharmaceutically acceptable salt or solvate thereof, is administered to a patient already suffering from cancer, in an amount sufficient to treat the cancer or synergize with a chemotherapy. Amounts effective for this use will depend on the severity and course of the disease or condition, previous therapy, the patient's health status, weight, and response to the drugs, and the judgment of the treating physician.

A compound described herein, or a pharmaceutically acceptable salt or solvate thereof, may be administered to the subject using different administration routes, including oral, transmucosal, topical, transdermal, inhalation, intravenous, subcutaneous, intradermal, intramuscular, intra-articular, perineural, intraventricular, intravenous, intraperitoneal, intranasal, and intraocular. In some embodiments, the compound is formulated in a pharmaceutical composition for oral administration. In some embodiments, the compound is formulated in a pharmaceutical composition for intravenous administration.

In some embodiments, the CHD1L inhibitor binds to an allosteric binding site comprising an N-terminus of a C-terminal ATP-ase domain of CHD1L. In some embodiments, the CHD1L inhibitor binds to an allosteric binding site of CHD1L, as shown in FIGS. 1A and 1B. In some embodiments, the CHD1L inhibitor reduces an ATP-ase activity of CHD1L. In some embodiments, the CHD1L inhibitor interacts with a conserved lysine in the allosteric binding site. In some embodiments, the CHD1L comprises a sequence having at least 80% sequence identity SEQ ID NOs: 1-40 or Table 2. In some embodiments, the CHD1L inhibitor binds to a site of CHD1L comprising a sequence having at least about 70% sequence identity to SEQ ID NO: 40, SEQ ID NO: 41, SEQ ID NO: 42, SEQ ID NO: 43, SEQ ID NO: 44, or a combination thereof. In some embodiments, the CHD1L inhibitor binds to a site of CHD1L comprising a sequence having at least about 70% sequence identity to SEQ ID NO: 40, SEQ ID NO: 41, SEQ ID NO: 42, SEQ ID NO: 43, SEQ ID NO: 44, or a combination thereof. In some embodiments, the CHD1L inhibitor binds to a site of CHD1L comprising a sequence having at least about 80% sequence identity to SEQ ID NO: 40, SEQ ID NO: 41, SEQ ID NO: 42, SEQ ID NO: 43, SEQ ID NO: 44, or a combination thereof. In some embodiments, the CHD1L inhibitor binds to a site of CHD1L comprising a sequence having at least about 90% sequence identity to SEQ ID NO: 40, SEQ ID NO: 41, SEQ ID NO: 42, SEQ ID NO: 43, SEQ ID NO: 44, or a combination thereof. In some embodiments, the CHD1L inhibitor binds to a site of CHD1L comprising a sequence having at least about 95% sequence identity to SEQ ID NO: 40, SEQ ID NO: 41, SEQ ID NO: 42, SEQ ID NO: 43, SEQ ID NO: 44, or a combination thereof.

TABLE 2
CHD1L Sequences.

| SEQ ID NO | Sequence | Organism |
|---|---|---|
| 1 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDFNPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 2 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDFNPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 3 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDENPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 4 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDFNPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 5 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDENPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 6 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDENPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 7 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDFNPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 8 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDENPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 9 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDENPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 10 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDENPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 11 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDENPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 12 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDENPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 13 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDENPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 14 | ARGGASTGPMERAGATSRGGQAPGFLLRLHTEGRAEA | Homo sapiens |
| | ARVQEQDLRQWGLTGIHLRSYQLEGVNWLAQRFHCQ | |
| | NGCILGDEMGLGKTCQTIALFIYLAGRLNDEGPFLILCP | |
| | LSVLSNWKEEMQRFAPGLSCVTYAGDKEERACLQQDL | |
| | KQESRFHVLLTTYEICLKDASFLKSFPWSVLVVDEAHR | |
| | LKNQSSLLHKTLSEFSVVFSLLLTGTPIQNSLQELYSLLS | |
| | FVEPDLFSKEEVGDFIQRYQDIEKESESASELHKLLQPF | |
| | LLRRVKAEVATELPKKTEVVIYHGMSALQKKYYKAIL | |
| | MKDLDAFENETAKKVKLQNILSQLRKCVDHPYLFDGV | |
| | EPEPFEVGDHLTEASGKLHLLDKLLAFLYSGGHRVLLF | |
| | SQMTQMLDILQDYMDYRGYSYERVDGSVRGEERHLAI | |
| | KNFGQQPIFVFLLSTRAGGVGMNLTAADTVIFVDSDEN | |
| | PQNDLQAAARAHRIGQNKSVKVIRLIGRDTVEEIVYRK | |
| | AASKLQLTNMIIEGGHFTLGAQKPAADADLQLSEILKF | |
| | GLDKLLASEGSTMDEIDLESILGETKDGQWVSDALPAA | |
| | EGGSRDQEEGKNHMYLFEGKDYSKEPSKEDRKSFEQL | |
| | VNLQKTLLEKASQEGRSLRNKGSVLIPGLVEGSTKRKR | |
| | VLSPEELEDRQKKRQEAAAKRRRLIEEKKRQKEEAEH | |
| | KKKMAWWESNNYQSFCLPSEESEPEDLENGEESSAEL | |
| | DYQDPDATSLKYVSGDVTHPQAGAEDALIVHCVDDSG | |
| | HWGRGGLFTALEKRSAEPRKIYELAGKMKDLSLGGVL | |
| | LFPVDDKESRNKGQDLLALIVAQHRDRSNVLSGIKMA | |
| | ALEEGLKKIFLAAKKKKASVHLPRIGHATKGFNWYGT | |
| | ERLIRKHLAARGIPTYIYYFPRSKSAVLHSQSSSSSSRQL | |
| | VP | |
| 15 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDFNPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHAQSSSSSSRQLVP | |
| 16 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDENPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHAQSSSSSSRQLVP | |
| 17 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDFNPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHAQSSSSSSRQLVP | |
| 18 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDENPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTEQLIRKHLAA | |
| | RGIPTYIYYFPRSKSAVLHSQSSSSSSRQLVP | |
| 19 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLQLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDFNPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 20 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESGNELHKLLQPFLLRRVKAE | |
| | VATELPKKTEVVIYHGMSALQKKYYKAILMKDLDAFE | |
| | NETAKKVKLQNILSQLRKCVDHPYLFDGVEPEPFEVGD | |
| | HLTEASGKLHLLDKLLAFLYSGGHRVLLFSQMTQMLD | |
| | ILQDYMDYRGYSYERVDGSVRGEERHLAIKNFGQQPIF | |
| | VFLLSTRAGGVGMNLTAADTVIFVDSDFNPQNDLQAA | |
| | ARAHRIGQNKSVKVIRLIGRDTVEEIVYRKAASKLQLT | |
| | NMIIEGGHFTLGAQKPAADADLQLSEILKFGLDKLLAS | |
| | EGSTMDEIDLESILGETKDGQWVSDALPAAEGGSRDQE | |
| | EGKNHMYLFEGKDYSKEPSKEDRKSFEQLVNLQKTLL | |
| | EKASQEGRSLRNKGSVLIPGLVEGSTKRKRVLSPEELE | |
| | DRQKKRQEAAAKRRRLIEEKKRQKEEAEHKKKMAW | |
| | WESNNYQSFCLPSEESEPEDLENGEESSAELDYQDPDA | |
| | TSLKYVSGDVTHPQAGAEDALIVHCVDDSGHWGRGG | |
| | LFTALEKRSAEPRKIYELAGKMKDLSLGGVLLFPVDDK | |
| | ESRNKGQDLLALIVAQHRDRSNVLSGIKMAALEEGLK | |
| | KIFLAAKKKKASVHLPRIGHATKGFNWYGTERLIRKHL | |
| | AARGIPTYIYYFPRSKSAVLHSQSSSSSSRQLVP | |
| 21 | MERAGATSRGGQAPGFLLRLHTEGRAEAARVQEQDLR | Homo sapiens |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEVFEFSVVFSLLLTGTPIQNSLQELYSLLSFVEPDL | |
| | FSKEEVGDFIQRYQDIEKESESASELHKLLQPFLLRRVK | |
| | AEVATELPKKTEVVIYHGMSALQKKYYKAILMKDLDA | |
| | FENETAKKVKLQNILSQLRKCVDHPYLFDGVEPEPFEV | |
| | GDHLTEASGKLQLLDKLLAFLYSGGHRVLLFSQMTQM | |
| | LDILQDYMDYRGYSYERVDGSVRGEERHLAIKNFGQQ | |
| | PIFVFLLSTRAGGVGMNLTAADTVIFVDSDFNPQNDLQ | |
| | AAARAHRIGQNKSVKVIRLIGRDTVEEIVYRKAASKLQ | |
| | LTNMIIEGGHFTLGAQKPAADADLQLSEILKFGLDKLL | |
| | ASEGSTMDEIDLESILGETKDGQWVSDALPAAEGGSRD | |
| | QEEGKNHMYLFEGKDYSKEPSKEDRKSFEQLVNLQKT | |
| | LLEKASQEGRSLRNKGSVLIPGLVEGSTKRKRVLSPEEL | |
| | EDRQKKRQEAAAKRRRLIEEKKRQKEEAEHKKKMAW | |
| | WESNNYQSFCLPSEESEPEDLENGEESSAELDYQDPDA | |
| | TSLKYVSGDVTHPQAGAEDALIVHCVDDSGHWGRGG | |
| | LFTALEKRSAEPRKIYELAGKMKDLSLGGVLLFPVDDK | |
| | ESRNKGQDLLALIVAQHRDRSNVLSGIKMAALEEGLK | |
| | KIFLAAKKKKASVHLPRIGHATKGFNWYGTERLIRKHL | |
| | AARGIPTYIYYFPRSKSAVLHSQSSSSSSRQLVP | |
| 22 | ERAGATSRGGQAPGFLLRLHTEGPAEAARVQEQDLRQ | Homo sapiens |
| | WGLTGIHLRSYQLEGVNWLAQRFHCQNGCILGDEMG | |
| | LGKTCQTIALFIYLAGRLNDEGPFLILCPLSVLSNWKEE | |
| | MQRFAPGLSCVTYAGDKEERACLQQDLKQESRFHVLL | |
| | TTYEICLKDASFLKSFPWSVLVVDEAHRLKNQSSLLHK | |
| | TLSEFSVVFSLLLTGTPIQNSLQELYSLLSFVEPDLFSKE | |
| | EVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDENPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDCGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 23 | ERAGATSRGGQAPGFLLRLHTEGPAEAARVQEQDLRQ | Homo sapiens |
| | WGLTGIHLRSYQLEGVNWLAQRFHCQNGCILGDEMG | |
| | LGKTCQTIALFIYLAGRLNDEGPFLILCPLSVLSNWKEE | |
| | MQRFAPGLSCVTYAGDKEERACLQQDLKQESRFHVLL | |
| | TTYEICLKDASFLKSFPWSVLVVDEAHRLKNQSSLLHK | |
| | TLSEFSVVFSLLLTGTPIQNSLQELYSLLSFVEPDLFSKE | |
| | EVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDENPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDCGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 24 | ERAGATSRGGQAPGFLLRLHTEGPAEAARVQEQDLRQ | Homo sapiens |
| | WGLTGIHLRSYQLEGVNWLAQRFHCQNGCILGDEMG | |
| | LGKTCQTIALFIYLAGRLNDEGPFLILCPLSVLSNWKEE | |
| | MQRFAPGLSCVTYAGDKEERACLQQDLKQESRFHVLL | |
| | TTYEICLKDASFLKSFPWSVLVVDEAHRLKNQSSLLHK | |
| | TLSEFSVVFSLLLTGTPIQNSLQELYSLLSFVEPDLFSKE | |
| | EVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDFNPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDCGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 25 | ERAGATSRGGQAPGFLLRLHTEGPAEAARVQEQDLRQ | Homo sapiens |
| | WGLTGIHLRSYQLEGVNWLAQRFHCQNGCILGDEMG | |
| | LGKTCQTIALFIYLAGRLNDEGPFLILCPLSVLSNWKEE | |
| | MQRFAPGLSCVTYAGDKEERACLQQDLKQESRFHVLL | |
| | TTYEICLKDASFLKSFPWSVLVVDEAHRLKNQSSLLHK | |
| | TLSEFSVVFSLLLTGTPIQNSLQELYSLLSFVEPDLFSKE | |
| | EVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDFNPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDCGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKSAVLHSQSSSSSSRQLVP | |
| 26 | SRGGQAPGFLLRLHTEGRAEAARVQEQDLRQWGLTGI | Homo sapiens |
| | HLRSYQLEGVNWLAQRFHCQNGCILGDEMGLGKTCQ | |
| | TIALFIYLAGRLNDEGPFLILCPLSVLSNWKEEMQRFAP | |
| | GLSCVTYAGDKEERACLQQDLKQESRFHVLLTTYEICL | |
| | KDASFLKSFPWSVLVVDEAHRLKNQSSLLHKTLSEFSV | |
| | VFSLLLTGTPIQNSLQELYSLLSFVEPDLFSKEEVGDFIQ | |
| | RYQDIEKESESASELHKLLQPFLLRRVKAEVATELPKK | |
| | TEVVIYHGMSALQKKYYKAILMKDLDAFENETAKKV | |
| | KLQNILSQLRKCVDHPYLFDGVEPEPFEVGDHLTEASG | |
| | KLHLLDKLLAFLYSGGHRVLLFSQMTQMLDILQDYMD | |
| | YRGYSYERVDGSVRGEERHLAIKNFGQQPIFVFLLSTR | |
| | AGGVGMNLTAADTVIFVDSDFNPQNDLQAAARAHRIG | |
| | QNKSVKVIRLIGRDTVEEIVYRKAASKLQLTNMIIEGG | |
| | HFTLGAQKPAADADLQLSEILKFGLDKLLASEGSTMDE | |
| | IDLESILGETKDGQWVSDALPAAEGGSRDQEEGKNHM | |
| | YLFEGKDYSKEPSKEDRKSFEQLVNLQKTLLEKASQEG | |
| | RSLRNKGSVLIPGLVEGSTKRKRVLSPEELEDRQKKRQ | |
| | EAAAKRRRLIEEKKRQKEEAEHKKKMAWWESNNYQS | |
| | FCLPSEESEPEDLENGEESSAELDYQDPDATSLKYVSG | |
| | DVTHPQAGAEDALIVHCVDDSGHWGRGGLFTALEKRS | |
| | AEPRKIYELAGKMKDLSLGGVLLFPVDDKESRNKGQD | |
| | LLALIVAQHRDRSNVLSGIKMAALEEGLKKIFLAAKKK | |
| | KASVHLPRIGHATKGFNWYGTERLIRKHLAARGIPTYI | |
| | YYFPRSKSAVLHSQSSSSSSRQLVP | |
| 27 | SRGGQAPGFLLRLHTEGRAEAARVQEQDLRQWGLTGI | Homo sapiens |
| | HLRSYQLEGVNWLAQRFHCQNGCILGDEMGLGKTCQ | |
| | TIALFIYLAGRLNDEGPFLILCPLSVLSNWKEEMQRFAP | |
| | GLSCVTYAGDKEERACLQQDLKQESRFHVLLTTYEICL | |
| | KDASFLKSFPWSVLVVDEAHRLKNQSSLLHKTLSEFSV | |
| | VFSLLLTGTPIQNSLQELYSLLSFVEPDLFSKEEVGDFIQ | |
| | RYQDIEKESESASELHKLLQPFLLRRVKAEVATELPKK | |
| | TEVVIYHGMSALQKKYYKAILMKDLDAFENETAKKV | |
| | KLQNILSQLRKCVDHPYLFDGVEPEPFEVGDHLTEASG | |
| | KLHLLDKLLAFLYSGGHRVLLFSQMTQMLDILQDYMD | |
| | YRGYSYERVDGSVRGEERHLAIKNFGQQPIFVFLLSTR | |
| | AGGVGMNLTAADTVIFVDSDFNPQNDLQAAARAHRIG | |
| | QNKSVKVIRLIGRDTVEEIVYRKAASKLQLTNMIIEGG | |
| | HFTLGAQKPAADADLQLSEILKFGLDKLLASEGSTMDE | |
| | IDLESILGETKDGQWVSDALPAAEGGSRDQEEGKNHM | |
| | YLFEGKDYSKEPSKEDRKSFEQLVNLQKTLLEKASQEG | |
| | RSLRNKGSVLIPGLVEGSTKRKRVLSPEELEDRQKKRQ | |
| | EAAAKRRRLIEEKKRQKEEAEHKKKMAWWESNNYQS | |
| | FCLPSEESEPEDLENGEESSAELDYQDPDATSLKYVSG | |
| | DVTHPQAGAEDALIVHCVDDSGHWGRGGLFTALEKRS | |
| | AEPRKIYELAGKMKDLSLGGVLLFPVDDKESRNKGQD | |
| | LLALIVAQHRDRSNVLSGIKMAALEEGLKKIFLAAKKK | |
| | KASVHLPRIGHATKGFNWYGTERLIRKHLAARGIPTYI | |
| | YYFPRSKSAVLHSQSSSSSSRQLVP | |
| 28 | SRGGQAPGFLLRLHTEGRAEAARVQEQDLRQWGLTGI | Homo sapiens |
| | HLRSYQLEGVNWLAQRFHCQNGCILGDEMGLGKTCQ | |
| | TIALFIYLAGRLNDEGPFLILCPLSVLSNWKEEMQRFAP | |
| | GLSCVTYAGDKEERACLQQDLKQESRFHVLLTTYEICL | |
| | KDASFLKSFPWSVLVVDEAHRLKNQSSLLHKTLSEFSV | |
| | VFSLLLTGTPIQNSLQELYSLLSFVEPDLFSKEEVGDFIQ | |
| | RYQDIEKESESASELHKLLQPFLLRRVKAEVATELPKK | |
| | TEVVIYHGMSALQKKYYKAILMKDLDAFENETAKKV | |
| | KLQNILSQLRKCVDHPYLFDGVEPEPFEVGDHLTEASG | |
| | KLHLLDKLLAFLYSGGHRVLLFSQMTQMLDILQDYMD | |
| | YRGYSYERVDGSVRGEERHLAIKNFGQQPIFVFLLSTR | |
| | AGGVGMNLTAADTVIFVDSDFNPQNDLQAAARAHRIG | |
| | QNKSVKVIRLIGRDTVEEIVYRKAASKLQLTNMIIEGG | |
| | HFTLGAQKPAADADLQLSEILKFGLDKLLASEGSTMDE | |
| | IDLESILGETKDGQWVSDALPAAEGGSRDQEEGKNHM | |
| | YLFEGKDYSKEPSKEDRKSFEQLVNLQKTLLEKASQEG | |
| | RSLRNKGSVLIPGLVEGSTKRKRVLSPEELEDRQKKRQ | |
| | EAAAKRRRLIEEKKRQKEEAEHKKKMAWWESNNYQS | |
| | FCLPSEESEPEDLENGEESSAELDYQDPDATSLKYVSG | |
| | DVTHPQAGAEDALIVHCVDDSGHWGRGGLFTALEKRS | |
| | AEPRKIYELAGKMKDLSLGGVLLFPVDDKESRNKGQD | |
| | LLALIVAQHRDRSNVLSGIKMAALEEGLKKIFLAAKKK | |
| | KASVHLPRIGHATKGFNWYGTERLIRKHLAARGIPTYI | |
| | YYFPRSKSAVLHSQSSSSSSRQLVP | |
| 29 | MERAGAASRGGQAPGFLLRLHTEGRAEAARVQEQDLR | Pan troglodytes |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKRYYKAILMKDLDAFEN | |
| | ETAKKVKLQNILSQLRKCVDHPYLFDGVEPEPFEVGDH | |
| | LIEASGKLHLLDKLLAFLYSGGHRVLLFSQMTQMLDIL | |
| | QDYMDYRGYSYERVDGSVRGEERHLAIKNFGRQPIFV | |
| | FLLSTRAGGVGMNLTAADTVIFVDSDFNPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KTSQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESAAELDYQDPDAT | |
| | SLKYVSGDVTHPQAGAEDALIVHCVDDSGHWGRGGL | |
| | FTALEKRSAEPRKIYELAGKMKDLSLGGVLLFPVDDKE | |
| | SRNKGQDLLALIVAQHRDRSNVLSGIKMAALEEGLKKI | |
| | FLAAKKKKASVHLPRIGHATKGFNWYGTERLIRKHLA | |
| | ARGIPTYIYYFPRSKSAVLHSQSSSSSRQLVP | |
| 30 | MERAGAASRGGQAPGFLLRLHTEGRAEAARVQEQDLR | Pan paniscus |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LIEASGKLHLLDKLLAFLYSGGHRVLLFSQMTQMLDIL | |
| | QDYMDYRGYSYERVDGSVRGEERHLAIKNFGQQPIFIF | |
| | LLSTRAGGVGMNLTAADTVIFVDSDFNPQNDLQAAAR | |
| | AHRIGQNKSVKVIRLIGRDTVEEIVYRKAASKLQLTNM | |
| | IIEGGHFTLGAQKPAADADLQLSEILKFGLDKLLASEGS | |
| | TIDEIDLESILGETKDGQWVSDALPAAEGGSRDQEEGK | |
| | NHMYLFEGKDYSKEPSKEDRKSFEQLVNLQKTLLEKT | |
| | SQEGRSLRNKGSVLIPGLVEGSTKRKRVLSPEELEDRQ | |
| | KKRQEAAAKRRRLIEEKKRQKEEAEHKKKMAWWESN | |
| | NYQSFCLPSEESEPEDLENGEESAAELDYQDPDATSLK | |
| | YVSGDVTHPQAGAEDALIVHCVDDSGHWGRGGLFTA | |
| | LEKRSAEPRKIYELAGKMKDLSLGGVLLFPVDDKESRN | |
| | KGQDLLALIVAQHRDRSNVLSGIKMAALEEGLKKIFLA | |
| | AKKKKASVHLPRIGHATKGFNWYGTERLIRKHLAARG | |
| | IPTYIYYFPRSKSAVLHSQSSSSSRQLVP | |
| 31 | MERAGAASRGGQAPGFLLRLHTEGRAEAARVQEQDLR | Pan troglodytes |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKEPESASELHKLLQPFLLRRVKAEV | |
| | ATELPKKTEVVIYHGMSALQKRYYKAILMKDLDAFEN | |
| | ETAKKVKLQNILSQLRKCVDHPYLFDGVEPEPFEVGDH | |
| | LIEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDENPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KTSQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESAAELDYQDPDAT | |
| | SLKYVSGDVTHPQAGAEDALIVHCVDDSGHWGRGGL | |
| | FTALEKRSAEPRKIYELAGKMKDLSLGGVLLFPVDDKE | |
| | SRNKGQDLLALIVAQHRDRSNVLSGIKMAALEEGLKKI | |
| | FLAAKKKKASVHLPRIGHATKGFNWYGTERLIRKHLA | |
| | ARGIPTYIYYFPRSKSAVLHSQSSSSSRQLVP | |
| 32 | MERAGAASRGGQAPGFLLRLHTEGRAEAARVQEQDLR | Pan troglodytes |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKEPESASELHKLLQPFLLRRVKAEV | |
| | ATELPKKTEVVIYHGMSALQKRYYKAILMKDLDAFEN | |
| | ETAKKVKLQNILSQLRKCVDHPYLFDGVEPEPFEVGDH | |
| | LIEASGKLHLLDKLLAFLYSGGHRVLLFSQMTQMLDIL | |
| | QDYMDYRGYSYERVDGSVRGEERHLAIKNFGRQPIFV | |
| | FLLSTRAGGVGMNLTAADTVIFVDSDENPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KTSQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESAAELDYQDPDAT | |
| | SLKYVSGDVTHPQAGAEDALIVHCVDDSGHWGRGGL | |
| | FTALEKRSAEPRKIYELAGKMKDLSLGGVLLFPVDDKE | |
| | SRNKGQDLLALIVAQHRDRSNVLSGIKMAALEEGLKKI | |
| | FLAAKKKKASVHLPRIGHATKGFNWYGTERLIRKHLA | |
| | ARGIPTYIYYFPRSKSAVLHSQSSSSSRQLVP | |
| 33 | MERAGAASRGGQAPGFLVRLHTEGRAEAARMQEQDL | Gorilla gorilla |
| | RQWGLTGIHLRSYQLEGVNWLAQRFHCQNGCILGDE | |
| | MGLGKTCQTIALFIYLVGRLNDEGPFLILCPLSVLSNW | |
| | KEEMQRFAPGLSCVTYAGDKEERACLQQDLKQESCFH | |
| | VLLTTYEICLKDASFLKSFPWSVLVVDEAHRLKNQSSL | |
| | LHKTLSEFSVVFSLLLTGTPIQNSLQELYSLLSFVEPDLF | |
| | SKEEVGDFIQRYQDIEKESESAGELHKLLQPFLLRRVK | |
| | AEVATELPRKTEVVIYHGMSALQKKYYKAILMKDLDA | |
| | FENETAKKVKLQNILSQLRKCVDHPYLFDGVEPEPFEV | |
| | GDHLIEASGKLHLLDKLLAILYSGGHRVLLFSQMTQML | |
| | DILQDYMDYRGYSYERVDGSVRGEERHLAIKNFGQQP | |
| | IFVFLLSTRAGGVGMNLTAADTVIFVDSDFNPQNDLQA | |
| | AARAHRIGQNKSVKVIRLIGRDTVEEIVYRKAASKLQL | |
| | TNMIIEGGHFTLGAQKPAADADLQLSEILKFGLDKLLA | |
| | SEGSTMDEIDLESILGETKDGQWVSDALPAAEGGSRDQ | |
| | EEGKNHMYLFEGKDYSKEPSKEDRKSFEQLVNLQKTL | |
| | LEKTSQEGRSLRNRGSVLIPGLVEGSTKRKRVLSPEELE | |
| | DRQKKRQEAAAKRRRLIEEKKRQKEEAEHKKKMAW | |
| | WESNNYQSFCLPSEESEPEDLENGEDESSAELDYQDPD | |
| | ATSLKYVSGDVTHPQAGAEDALIVHCVDDSGRWGRG | |
| | GLFTALEKRSAEPRRIYELAGKMKDLSLGGVLLFPVDD | |
| | KESRNKGQDLLALIVAQHRDRSNVLSGIKMAALEEGL | |
| | KKIFLAAKKKKASVHLPRIGHATKGFNWYGTERLIRK | |
| | HLAARGIPTYIYYFPRSKSAVLHSQSSSSSSRQLVP | |
| 34 | MERAGATSRGGQAPGFLLRLHTEGRAEAARVQEQDLR | Gorilla gorilla |
| | QWGLTGIHLRSYQLEGVNWLAQRFHCQNGCILGDEM | |
| | GLGKTCQTIALFIYLAGRLNDEGPFLILCPLSVLSNWKE | |
| | EMQRFAPGLSCVTYAGDKEERACLQQDLKQESRFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESASELHKLLQPFLLRRVKAEV | |
| | ATELPKKTEVVIYHGMSALQKKYYKAILMKDLDAFEN | |
| | ETAKKVKLQNILSQLRKCVDHPYLFDGVEPEPFEVGDH | |
| | LTEASGKLHLLDKLLAFLYSGGHRVLLFSQMTQMLDIL | |
| | QDYMDYRGYSYERVDGSVRGEERHLAIKNFGQQPIFV | |
| | FLLSTRAGGVGMNLTAADTVIFVDSDENPQNDLQAAA | |
| | RAHRIGQNKSVKVIRLIGRDTVEEIVYRKAASKLQLTN | |
| | MIIEGGHFTLGAQKPAADADLQLSEILKFGLDKLLASE | |
| | GSTMDEIDLESILGETKDGQWVSDALPAAEGGSRDQEE | |
| | GKNHMYLFEGKDYSKEPSKEDRKSFEQLVNLQKTLLE | |
| | KASQEGRSLRNKGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEESSAELDYQDPDATS | |
| | LKYVSGDVTHPQAGAEDALIVHCVDDSGHWGRGGLF | |
| | TALEKRSAEPRKIYELAGKMKDLSLGGVLLFPVDDKES | |
| | RNKGQDLLALIVAQHRDRSNVLSGIKMAALEEGLKKIF | |
| | LAAKKKKASVHLPRIGHATKGFNWYGTERLIRKHLAA | |
| | RGIPTYIYYFPRSKS | |
| 35 | MERAGAASRGGQAPGFLVRLHTEGRAEAARMQEQDL | Gorilla gorilla |
| | RQWGLTGIHLRSYQLEGVNWLAQRFHCQNGCILGDE | |
| | MGLGKTCQTIALFIYLVGRLNDEGPFLILCPLSVLSNW | |
| | KEEMQRFAPGLSCVTYAGDKEERACLQQDLKQESCFH | |
| | VLLTTYEICLKDASFLKSFPWSVLVVDEAHRLKNQSSL | |
| | LHKTLSEFSVVFSLLLTGTPIQNSLQELYSLLSFVEPDLF | |
| | SKEEVGDFIQRYQDIEKESESGELHKLLQPFLLRRVKAE | |
| | VATELPRKTEVVIYHGMSALQKKYYKAILMKDLDAFE | |
| | NETAKKVKLQNILSQLRKCVDHPYLFDGVEPEPFEVGD | |
| | HLIEASGKLHLLDKLLAILYSGGHRVLLFSQMTQMLDI | |
| | LQDYMDYRGYSYERVDGSVRGEERHLAIKNFGQQPIF | |
| | VFLLSTRAGGVGMNLTAADTVIFVDSDENPQNDLQAA | |
| | ARAHRIGQNKSVKVIRLIGRDTVEEIVYRKAASKLQLT | |
| | NMIIEGGHFTLGAQKPAADADLQLSEILKFGLDKLLAS | |
| | EGSTMDEIDLESILGETKDGQWVSDALPAAEGGSRDQE | |
| | EGKNHMYLFEGKDYSKEPSKEDRKSFEQLVNLQKTLL | |
| | EKTSQEGRSLRNRGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEDESSAELDYQDPDA | |
| | TSLKYVSGDVTHPQAGAEDALIVHCVDDSGRWGRGG | |
| | LFTALEKRSAEPRRIYELAGKMKDLSLGGVLLFPVDDK | |
| | ESRNKGQDLLALIVAQHRDRSNVLSGIKMAALEEGLK | |
| | KIFLAAKKKKASVHLPRIGHATKGFNWYGTERLIRKHL | |
| | AARGIPTYIYYFPRSKSAVLHSQSSSSSSRQLVP | |
| 36 | MERAGAASRGGQAPGFLVRLHTEGRAEAARMQEQDL | Gorilla gorilla |
| | RQGGGTGIHLRSYQLEGVNWLAQRFHCQNGCILGDEM | |
| | GLGKTCQTIALFIYLVGRLNDEGPFLILCPLSVLSNWKE | |
| | EMQRFAPGLSCVTYAGDKEERACLQQDLKQESCFHVL | |
| | LTTYEICLKDASFLKSFPWSVLVVDEAHRLKNQSSLLH | |
| | KTLSEFSVVFSLLLTGTPIQNSLQELYSLLSFVEPDLFSK | |
| | EEVGDFIQRYQDIEKESESAGELHKLLQPFLLRRVKAE | |
| | VATELPRKTEVVIYHGMSALQKKYYKAILMKDLDAFE | |
| | NETAKKVKLQNILSQLRKCVDHPYLFDGVEPEPFEVGD | |
| | HLIEASGKLHLLDKLLAILYSGGHRVLLFSQMTQMLDI | |
| | LQDYMDYRGYSYERVDGSVRGEERHLAIKNFGQQPIF | |
| | VFLLSTRAGGVGMNLTAADTVIFVDSDENPQNDLQAA | |
| | ARAHRIGQNKSVKVIRLIGRDTVEEIVYRKAASKLQLT | |
| | NMIIEGGHFTLGAQKPAADADLQLSEILKFGLDKLLAS | |
| | EGSTMDEIDLESILGETKDGQWVSDALPAAEGGSRDQE | |
| | EGKNHMYLFEGKDYSKEPSKEDRKSFEQLVNLQKTLL | |
| | EKTSQEGRSLRNRGSVLIPGLVEGSTKRKRVLSPEELED | |
| | RQKKRQEAAAKRRRLIEEKKRQKEEAEHKKKMAWW | |
| | ESNNYQSFCLPSEESEPEDLENGEDESSAELDYQDPDA | |
| | TSLKYVSGDVTHPQAGAEDALIVHCVDDSGRWGRGG | |
| | LFTALEKRSAEPRRIYELAGKMKDLSLGGVLLFPVDDK | |
| | ESRNKGQDLLALIVAQHRDRSNVLSGIKMAALEEGLK | |
| | KIFLAAKKKKASVHLPRIGHATKGFNWYGTERLIRKHL | |
| | AARGIPTYIYYFPRSKSAVLHSQSSSSSSRQLVP | |
| 37 | MERAGAASRGGQAPGFLLRLHTEGRAEAARARVQEQ | Mandrillus leucophaeus |
| | DLRQWGLTGIHLRSYQLEGVNWLAQRFHCQNGCILGD | |
| | EMGLGKTCQTIALLIYLAGRLNDEGPFLILCPLSVLSNW | |
| | KEEMQRFAPGLSCVTYAGDKEERACLQQDLKQESHFH | |
| | VLLTTYEICLKDASFLKSFPWSVLVVDEAHRLKNQSSL | |
| | LHKTLSEFSVVFSLLLTGTPIQNSLQELYSLLSFVEPDLF | |
| | SKEEVGDFVQRYQDIEKESESASELHKLLQPFLLRRVK | |
| | ADVATELPKKTEVVIYHGMSALQKKYYKAILMKDLD | |
| | AFENETAKKVKLQNILSQLRKCVDHPYLFDGVEPEPFE | |
| | VGDHLIEASGKLHLLDKLLAFLYSGGHRVLLFSQMTQ | |
| | MLDILQDYMDYRGYSYERVDGSVRGEERHLAIKNFGQ | |
| | QPIFVFLLSTRAGGVGMNLTAADTVIFVDSDFNPQNDL | |
| | QAAARAHRIGQNKSVKVIRLIGRDTVEEIVSRKAASKL | |
| | QLTNMIIEGGHFTLGAQKPSADADLQLSEILKFGLDKL | |
| | LASEGSTMDEIDLESILGETKDGQWVSDALPAAEGGIR | |
| | EQEEGKNHMYLFEGKDYSKEPSKEDRKSFEQLVNLQK | |
| | TLLEKTSQEGRSLRNKGSVLIPGLVEGSTKRKQVLSPEE | |
| | LEDRQKKRQEAAAKRRRLIEEKKRQKEEAEHKKKMA | |
| | WWESNHYQSFCLPSEESEPEDLENGEDESSAELDYQDP | |
| | DATSLKYVSGDVTHPQAGAEDALIVHCVDDSGHWGR | |
| | GGLFTALEKRSAEPRKIYELAGKMKDLSLGGVLLFPVD | |
| | DKESRNKGQDLLALIVAQHRDRSNVLSGIKMAALEEG | |
| | LKKIFLAAKKKKASVHLPRIGHATKGFNWYGTERLIRK | |
| | HLAARGIPTYIYYFPRSKSSVLHSQSSSSSSRQLVP | |
| 38 | MERAGAASRGGQAPGFLLRLHTEGRAEAARARVQEQ | Chlorocebus sabaeus |
| | DLRQWGLTGIHLRSYQLEGVNWLAQRFHCQNGCILGD | |
| | EMGLGKTCQTIALLIYLAGRLNDEGPFLILCPLSVLSNW | |
| | KEEMQRFAPGLSCVTYAGDKEERACLQQDLKQESHFH | |
| | VLLTTYEICLKDASFLKSFPWSVLVVDEAHRLKNQSSL | |
| | LHKTLSEFSVVFSLLLTGTPIQNSLQELYSLLSFVEPDLF | |
| | SKEEVGDFVQRYQDIEKESESASELHKLLQPFLLRRVK | |
| | ADVATELPKKTEVVIYHGMSALQKKYYKAILMKDLD | |
| | AFENETAKKVKLQNILSQLRKCVDHPYLFDGVEPEPFE | |
| | VGDHLIEASGKLHLLDKLLAFLYSGGHRVLLFSQMTQ | |
| | MLDILQDYMDYRGYSYERVDGSVRGEERHLAIKNFGQ | |
| | QPIFVFLLSTRAGGVGMNLTAADTVIFVDSDENPQNDL | |
| | QAAARAHRIGQNKSVKVIRLIGRDTVEEIVYRKAASKL | |
| | QLTNMIIEGGHFTLGAQKPSADADLQLSEILKFGLDKL | |
| | LASEGSTMDEIDLESILGETKDGQWVSDALPAAQGGIR | |
| | EQEEGKNHMYLFEGKDYSKEPSKEDRKSFEQLVNLQK | |
| | TLLEKTSQEGRSLRNKGSVLIPGLVEGSTKRKQVLSPEE | |
| | LEDRQKKRQEAAAKRRRLIEEKKRQKEEAEHKKKMA | |
| | WWESNHYQSFCLPSEESEPEDLENGEDESSAELDYQDP | |
| | DATSLKYVSGDVTHPQAGAEDALIVHCVDDSGHWGR | |
| | GGLFTALEKRSAEPRKIYELAGKMKDLSLGGVLLFPVD | |
| | DKESRNKGQDLLALIVAQHRDRSNVLSGIKMAALEEG | |
| | LKKIFLAAKKKKASVHLPRIGHATKGFNWYGTERLIRK | |
| | HLAARGIPTYIYYFPRSKSSVLYSQSSSSSSRQLVP | |
| 39 | MERAGAASRGGQAPGFLVRLHTEGRAEAARMQEQDL | Gorilla gorilla |
| | RQWGLTGIHLRSYQLEGVNWLAQRFHCQNGCILGDE | |
| | MGLGKTCQTIALFIYLVGRLNDEGPFLILCPLSVLSNW | |
| | KEEMQRFAPGLSCVTYAGDKEERACLQQDLKQESCFH | |
| | VLLTTYEICLKDASFLKSFPWSVLVVDEAHRLKNQSSL | |
| | LHKTLSEFSVVFSLLLTGTPIQNSLQELYSLLSFVEPDLF | |
| | SKEEVGDFIQRYQDIEKESESAGELHKLLQPFLLRRVK | |
| | AEVATELPRKTEVVIYHGMSALQKKYYKAILMKDLDA | |
| | FENETAKKVKLQNILSQLRKCVDHPYLFDGVEPEPFEV | |
| | GDHLIEASGKLHLLDKLLAILYSGGHRVLLFSQMTQML | |
| | DILQDYMDYRGYSYERVDGSVRGEERHLAIKNFGQQP | |
| | IFVFLLSTRAGGVGMNLTAADTVIFVDSDFNPQNDLQA | |
| | AARAHRIGQNKSVKVIRLIGRDTVEEIVYRKAASKLQL | |
| | TNMIIEGGHFTLGAQKPAADADLQNLLDFFVPLFKLSEI | |
| | LKFGLDKLLASEGSTMDEIDLESILGETKDGQWVSDAL | |
| | PAAEGGSRDQEEGKNHMYLFEGKDYSKEPSKEDRKSF | |
| | EQLVNLQKTLLEKTSQEGRSLRNRGSVLIPGLVEGSTK | |
| | RKRVLSPEELEDRQKKRQEAAAKRRRLIEEKKRQKEE | |
| | AEHKKKMAWWESNNYQSFCLPSEESEPEDLENGEDES | |
| | SAELDYQDPDATSLKYVSGDVTHPQAGAEDALIVHCV | |
| | DDSGRWGRGGLFTALEKRSAEPRRIYELAGKMKDLSL | |
| | GGVLLFPVDDKESRNKGQDLLALIVAQHRDRSNVLSGI | |
| | KMAALEEGLKKIFLAAKKKKASVHLPRIGHATKGFNW | |
| | YGTERLIRKHLAARGIPTYIYYFPRSKSAVLHSQSSSSSS | |
| | RQLVP | |
| 40 | RRVKAEVATELPKKTEV | Homo sapiens |
| 41 | SDFNP | Homo sapiens |
| 42 | KVIR | Homo sapiens |
| 43 | IVYRKAASKLQ | Homo sapiens |
| 44 | MYLFEGKDYSKE | Homo sapiens |

In some embodiments, the binding of CHD1L to an allosteric site of CHD1L inhibits chromatin remodeling. In some embodiments, the CHD1L inhibitor modulates poly-ADP-ribosylation (PAR) in a cancer cell. In some embodiments, administering the CHD1L inhibitor induces PAR-mediated programmed cell death (PARthanatos).

In some embodiments, administering the CHD1L inhibitor prevents cell DNA repair in the tumor cell. In some embodiments, administering the CHD1L inhibitor reduces DNA repair in a tumor cell by about 10%, about 20%, about 30%, about 40%, about 50%, about 60%, about 70%, about 80%, about 90%, or about 95%. In some embodiments, administering the CHD1L inhibitor induces PARthanatos and prevents cell DNA repair in the tumor cell.

In some embodiments, administering the CHD1L inhibitor traps CHD1L in a nucleus of the tumor cell. In some embodiments, administering the CHD1L inhibitor inhibits chromatin remodeling. In some embodiments, the amount of CHD1L trapping is determined using immunofluorescence. In some embodiments, administering the CHD1L inhibitor increases CHD1L trapping by about 1-fold, about 2-fold, about 5-fold, about 10-fold, or about 15-fold relative to a control, such as administration of a chemotherapy that does not trap CHD1L. In some instances, the control that does not trap CHD1L is doxorubicin. In some embodiments, administration of the CHD1L inhibitor and a chemotherapy traps CHD1L, PARP1, PARP2, or a combination thereof. In some embodiments, administration of the CHD1L inhibitor traps CHD1L, PARP1, and PARP2. In some embodiments, administration of the CHD1L inhibitor traps CHD1L and PARP1. In some embodiments, administration of the CHD1L inhibitor traps CHD1L and PARP2. In some embodiments, administration of CHD1L and the chemotherapy synergizes to treat the cancer by trapping PARP1 and CHD1L onto chromatin and nucleosomes. In some embodiments, administration of CHD1L and PARP1 increases CHD1L trapping by about 1-fold, about 2-fold, about 3-fold, or about 4-fold. In some embodiments, administration of CHD1L and PARP1 increases PARP1 trapping by about 1-fold, about 2-fold, about 5-fold, about 10-fold, about 15-fold, or about 20-fold. In some embodiments, CHD1L trapping is determined using an immunofluorescence assay. In some embodiments, the immunofluorescence assay comprises: (a) incubating a cell with a CHD1L inhibitor to provide a mixture; (b) adding a fluorescent anti-CHD1L antibody; (c) fixing the mixture comprising the cell, the CHD1L inhibitor, and the fluorescent anti-CHD1L antibody; (d) irradiating the mixture with light sufficient to generate a fluorescence signal by the fluorescent anti-CHD1L antibody. In some embodiments, the immunofluorescence assay further comprises quantifying a fluorescence signal generated from the fluorescent anti-CHD1L antibody. In some embodiments, the immunofluorescence assay further comprises providing a control comprising incubating a cell with Hoechst stain.

In some embodiments, administering the CHD1L inhibitor inhibits a cell cycle of the tumor cell. In some embodiments, the tumor or cancer is treated with a CHD1Li or with a combination of the CHD1Li and a chemotherapy. In some embodiments, administering the CHD1L inhibitor induces the tumor or cancer cell to remain in the G1 or S phase. In some embodiments, administering the CHD1L inhibitor prevents the tumor or cancer cell from entering into the G2 or M phase. In some embodiments, administration of the CHD1L inhibitor and the chemotherapy induces about 20%, about 30%, about 40%, about 50%, about 60%, about 70%, about 80%, or about 90% of the tumor or cancer cells to remain in the G1 phase. In some embodiments, administration of the CHD1L inhibitor and the chemotherapy induces about 20%, about 30%, about 40%, about 50%, about 60%, about 70%, about 80%, or about 90% of the tumor or cancer cells to remain in the S phase. In some embodiments, administration of the CHD1L inhibitor and the chemotherapy induces about 20%, about 30%, about 40%, about 50%, about 60%, about 70%, about 80%, or about 90% of the tumor or cancer cells to remain in the G2 or M phase. In some embodiments, the ability of CHD1L to modulate the cell cycle of the tumor or cancer cell is determined using the tetraploid DNA content. In some embodiments, the ability of a CHD1L inhibitor to inhibit a cell cycle or a phase within the cell cycle is determined using flow cytometry profiles of the tumor or cancer stained with DAPI. In some embodiments, an assay for determining the distribution of cells in a phase of the cell cycle comprises: (a) treating a tumor cell with a CHD1L inhibitor or a CHD1L in combination with a chemotherapy, including but not limited to, Olaparib, 5-FU, or docetaxel; (b) harvesting the tumor cell; (c) fixing the tumor cell; (d) staining the tumor cell with DAPI; (5) analyzing the cell size distribution using flow cytometry.

In some embodiments, administering the CHD1L inhibitor reduces tumor cell viability by about 20%, about 30%, about 40%, about 50%, about 60%, about 70%, about 80%, about 90%, about 95%, about 97%, about 99%, or about 100%, when the tumor cell viability is measured using an assay that measures ATP as an indicator of the tumor cell viability and generates a luminescence readout. In some embodiments, the tumor cell viability of colorectal cancer, breast cancer, lung cancer, pancreatic cancer, osteosarcoma, and melanoma is determined using the assay. In some embodiments, assay comprises: (a) introducing the CHD1L inhibitor and a detection reagent to organoids in a culture medium, wherein the detection reagent becomes luminescent when ATP in the organoids is extracted from the organoids; (b) mixing the CHD1L inhibitor, the detection reagent and the organoids to produce a mixture under conditions sufficient to induce cell lysis and extract the ATP from the organoids; (c) incubating the mixture to stabilize a luminescent signal; and (d) recording luminescence to produce the luminescence readout.

In some embodiments, the cancer displays multidrug resistance (MDR). In some embodiments, the cancer is breast cancer, lung cancer, pancreatic cancer, melanoma, or osteosarcoma. In some embodiments the cancer is breast cancer. In some embodiments, the cancer is lung cancer. In some embodiments, the cancer is melanoma. In some embodiments, the cancer is osteosarcoma. In some embodiments, the cancer is pancreatic cancer.

In some embodiments, the method for treating cancer further comprises administering a chemotherapy to the subject. In some embodiments, the cancer is resistant to the chemotherapy. In some embodiments, a combination of the CHD1L inhibitor and the chemotherapy is therapeutically effective to treat the cancer. In some embodiments, a combination of the CHD1L inhibitor potentiates or is synergistic with the chemotherapy. In some embodiments, the chemotherapy is a PARP inhibitor (PARPi). In some embodiments, the chemotherapy is a standard of care (SOC) chemotherapy for the cancer. In some embodiments, the chemotherapy comprises irinotecan, olaparib, doxorubicin, docetaxel, AZD5305, or 5-fluorouracil (5-FU), or a combination of two or more thereof.

In some embodiments, the synergy of the CHD1L inhibitor and the chemotherapy is determined by quantify DNA damage via an assay. In some embodiments, the assay comprises using immunofluorescence to quantify the number of γH2AX foci in a tumor cell in the presence and absence of a CHD1L inhibitor. In some embodiments, the assay comprises using immunofluorescence to quantify the number of γH2AX foci in a tumor cell in the presence and absence of a CHD1L inhibitor and a chemotherapy. In some embodiments, assay comprises: (a) introducing the CHD1L inhibitor and a γH2AX-specific detection reagent to organoids in a culture medium, wherein the detection γH2AX-specific reagent becomes luminescent when in contact with γH2AX; (b) mixing the CHD1L inhibitor, the γH2AX-specific detection reagent and the organoids; (c) incubating the mixture to stabilize a fluorescence signal; and (d) recording luminescence to produce the fluorescence readout. In some embodiments, an increase in fluorescence corresponds to an increase in γH2AX, which corresponds to DNA damage.

In some embodiments, administration of the CHD1L inhibitor localizes apoptosis inducing factor (AIF) in the nucleus of the tumor cell. In some embodiments, the administration of the CHD1L inhibitor provides a higher concentration of apoptosis inducing factor (AIF) in the nucleus of the tumor cell relative to a concentration of AIF in a cytoplasm of the tumor cell. In some embodiments, the administration of the CHD1L inhibitor localizes AIF in the nucleus of a tumor cell relative to a concentration of AIF in a cytoplasm of the tumor cell. In some embodiments, determining a relative distribution of AIF in a nucleus and AIF in a cytoplasm of a cell comprises quantifying AIF using immunofluorescence, wherein the cell is treated with Hoechst stain and an anti-AIF antibody. In some embodiments, the relative concentration of AIF in the nucleus to AIF in the cytoplasm is about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 15, about 20, about 25, or about 30. In some embodiments, the relative concentration is a relative ratio of the average intensity of AIF in the nucleus divided by the average intensity of AIF in the cytoplasm. In some embodiments, assay comprises: (a) introducing the CHD1L inhibitor and a AIF-specific detection reagent to organoids in a culture medium, wherein the detection AIF-specific reagent becomes luminescent when in contact with AIF; (b) mixing the CHD1L inhibitor, the AIF-specific detection reagent and the organoids; (c) incubating the mixture to stabilize a fluorescence signal; and (d) recording luminescence to produce the fluorescence readout. In some embodiments, the administration of the CHD1L inhibitor induces localization of PAR in the nucleus of the tumor cell. In some embodiments, the administration of the CHD1L inhibitor induces localization of PARP1 onto nucleosomes in the nucleus of the tumor cell. In some embodiments, the administration of the CHD1L inhibitor traps PARP1 onto a nucleosome of a tumor cell. In some embodiments, determining an amount of PARP1 trapping comprises using an immunofluorescence assay. In some embodiments, the immunofluorescence assay comprises treating the cell with Hoechst stain and a fluorescent anti-AIF antibody. In some embodiments, the relative concentration of AIF in the nucleus to AIF in the cytoplasm is about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 15, about 20, about 25, or about 30. In some embodiments, the relative concentration is a relative ratio of the average intensity of AIF in the nucleus divided by the average intensity of AIF in the cytoplasm. In some embodiments, assay comprises: (a) introducing the CHD1L inhibitor and a AIF-specific detection reagent to organoids in a culture medium, wherein the detection AIF-specific reagent becomes luminescent when in contact with AIF; (b) mixing the CHD1L inhibitor, the AIF-specific detection reagent and the organoids; (c) incubating the mixture to stabilize a fluorescence signal; and (d) recording luminescence to produce the fluorescence readout.

In some embodiments, the administration of the CHD1L inhibitor and the chemotherapy provides a synergy score of at least about 10, when the Bliss energy score is determined using a Bliss model. In some embodiments, calculation of the Bliss synergy scores is described in De Veroli et al. In some embodiments, administering the CHD1L inhibitor and the chemotherapy provides a synergy score of at least about 10 to about 100 using a Bliss model, thus showing synergy between the CHD1L inhibitor and the chemotherapy. In some embodiments, administering the CHD1L inhibitor and the chemotherapy provides a synergy score of at least about 10 to about 90 using the Bliss model. In some embodiments, administering the CHD1L inhibitor and the chemotherapy provides a synergy score of at least about 10 to about 80 using the Bliss model. In some embodiments, administering the CHD1L inhibitor and the chemotherapy provides a synergy score of at least about 10 to about 70 using the Bliss model. In some embodiments, administering the CHD1L inhibitor and the chemotherapy provides a synergy score of at least about 10 to about 60 using the Bliss model. In some embodiments, administering the CHD1L inhibitor and the chemotherapy provides a synergy score of at least about 10 to about 50 using the Bliss model. In some embodiments, administering the CHD1L inhibitor and the chemotherapy provides a synergy score of at least about 10 to about 40 using the Bliss model. In some embodiments, administering the CHD1L inhibitor and the chemotherapy provides a synergy score of at least about 10 to about 30 using the Bliss model. In some embodiments, administering the CHD1L inhibitor and the chemotherapy provides a synergy score of at least about 10 to about 20 using the Bliss model. In some embodiments, administering the CHD1L inhibitor and the chemotherapy provides a synergy score of at least about 10, at least about 20, at least about 30, at least about 40, at least about 50, at least about 60, at least about 70, at least about 80, at least about 90, at least about 95, or at least about 100.

In some embodiments, administering the CHD1L inhibitor and administering the chemotherapy are performed simultaneously. In some embodiments, the CHD1L inhibitor and the chemotherapy are co-administered. In some embodiments, administering the CHD1L inhibitor is prior to administering the chemotherapy. In some embodiments, administering the CHD1L inhibitor is after administering the chemotherapy. In some embodiments, the CHD1L inhibitor is administered orally, parenterally, intravenously, intraperitoneally, subcutaneously, or a combination thereof. In some embodiments, the chemotherapy is administered orally, parenterally, intravenously, intraperitoneally, subcutaneously, or a combination thereof. In some embodiments, the CHD1L inhibitor is administered orally, and the chemotherapy is administered intravenously.

In some embodiments, the CHD1L inhibitor is administered at least once per week. In some embodiments, the CHD1L inhibitor is administered at least twice per week. In some embodiments, the CHD1L inhibitor is administered at least three times per week. In some embodiments, the CHD1L inhibitor is administered at least four times per week. In some embodiments, the CHD1L inhibitor is administered at least five times per week. In some embodiments, the CHD1L inhibitor is administered six times per week. In some embodiments, the chemotherapy is administered once per week. In some embodiments, the chemotherapy is administered twice per week. In some embodiments, the chemotherapy is administered three times per week. In some embodiments, the chemotherapy is administered four times per week. In some embodiments, the chemotherapy is administered five times per week.

In some embodiments, the CHD1L inhibitor is administered more frequently than the chemotherapy. In some embodiments, the CHD1L inhibitor is administered at least three times per week, and chemotherapy is administered once per week. In some embodiments, the CHD1L inhibitor is administered at least four times per week, and chemotherapy is administered once per week. In some embodiments, the CHD1L inhibitor is administered at least five times per week, and chemotherapy is administered once per week. In some embodiments, the CHD1L inhibitor is administered at least six times per week, and chemotherapy is administered once per week.

### Compositions

Disclosed herein, in some embodiments, are compositions comprising a compound of Formula (I), (II), (IIIA), or (IIIB), or a pharmaceutically acceptable salt or solvate thereof. In some embodiments, the compound is any one of compounds 1-17 provided in Table 1. In some embodiments, the compound is compound 1, or a pharmaceutically acceptable salt or solvate thereof. In some embodiments, the compound is compound 2, or a pharmaceutically acceptable salt or solvate thereof. In some embodiments, the compound is compound 3, or a pharmaceutically acceptable salt or solvate thereof. In some embodiments, the compound is compound 4, or a pharmaceutically acceptable salt or solvate thereof. In some embodiments, the compound is compound 5, or a pharmaceutically acceptable salt or solvate thereof. In some embodiments, the compound is compound 6, or a pharmaceutically acceptable salt or solvate thereof. In some embodiments, the compound is compound 6.11, or a pharmaceutically acceptable salt or solvate thereof. In some embodiments, the compound is compound 7, or a pharmaceutically acceptable salt or solvate thereof. In some embodiments, the compound is compound 8, or a pharmaceutically acceptable salt or solvate thereof. In some embodiments, the compound is compound 9, or a pharmaceutically acceptable salt or solvate thereof. In some embodiments, the compound is compound 10, or a pharmaceutically acceptable salt or solvate thereof. In some embodiments, the compound is compound 11, or a pharmaceutically acceptable salt or solvate thereof. In some embodiments, the compound is compound 12, or a pharmaceutically acceptable salt or solvate thereof. In some embodiments, the compound is compound 13, or a pharmaceutically acceptable salt or solvate thereof. In some embodiments, the compound is compound 14, or a pharmaceutically acceptable salt or solvate thereof. In some embodiments, the compound is compound 15, or a pharmaceutically acceptable salt or solvate thereof. In some embodiments, the compound is compound 16, or a pharmaceutically acceptable salt or solvate thereof. In some embodiments, the compound is compound 17, or a pharmaceutically acceptable salt or solvate thereof. In some embodiments, compositions are pharmaceutical compositions comprising the compound or a pharmaceutically acceptable salt or solvate thereof or pharmaceutically acceptable salt or solvate thereof; and a pharmaceutically acceptable: excipient, diluent or carrier. In some embodiments, the compositions are pharmaceutical formulations comprising a compound, or a pharmaceutically acceptable salt or solvate thereof, or pharmaceutical composition of the present disclosure, wherein the pharmaceutical formulation is formulated for treating cancer. In some embodiments, the cancer is osteosarcoma, breast cancer, lung cancer, pancreatic cancer, colorectal cancer (CRC), or metastasis of any of the foregoing. In some embodiments, the cancer is osteosarcoma. In some embodiments, the cancer is breast cancer. In some embodiments, the cancer is lung cancer. In some embodiments, the cancer is pancreatic cancer. In some embodiments, the cancer is colorectal cancer (CRC). The pharmaceutical formulations of the present disclosure may be formulated for administration to a subject orally, parenterally, intravenously, intraperitoneally, subcutaneously, or a combination thereof.

In some embodiments, a compound described herein, or a pharmaceutically acceptable salt or solvate thereof, is a therapeutic agent. In some embodiments, the compound described herein, or a pharmaceutically acceptable salt or solvate thereof, is a CHD1L modulator. In some embodiments, the compound described herein, or a pharmaceutically acceptable salt or solvate thereof, modulates activity of a CHD1L protein. In some embodiments, the compound described herein, or a pharmaceutically acceptable salt or solvate thereof, is a CHD1L antagonist. In some embodiments, the compound described herein, or a pharmaceutically acceptable salt or solvate thereof, is an inhibitor of CHD1L activity.

The methods of the present disclosure comprise administration of a compound described herein, or a pharmaceutically acceptable salt or solvate thereof, that may modulate activity of a CHD1L protein. In some embodiments, at a therapeutically effective concentration, a compound, such as a CHD1L inhibitor, described herein, or a pharmaceutically acceptable salt or solvate thereof, modulates CHD1L protein activity by at least about 0.01%, at least about 0.1%, at least about 1%, at least about 2%, at least about 3%, at least about 4%, at least about 5%, at least about 10%, at least about 20%, at least about 30%, at least about 40%, or at least about 50% relative to CHD1L protein activity under conditions without the CHD1L inhibitor present.

In some embodiments, the compounds are inhibitors of CHD1L, wherein the compounds have the structure of Formula (I):

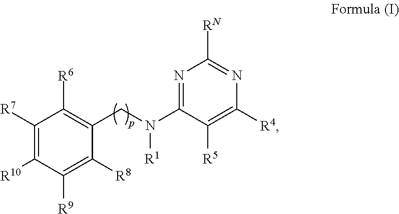

or a pharmaceutically acceptable salt thereof, wherein:
   R^{N} is an optionally substituted 5- to 7-membered heterocycle optionally containing a second heteroatom selected from the of group N, S, and O;
   R¹ is selected from hydrogen or C₁₋₃ alkyl;
   each R⁴ and R⁵ is selected from hydrogen, C₁₋₃ alkyl, and halogen;
   R⁶ is hydrogen or a halogen;
   each R⁷, R⁸, and R⁹ is independently selected from hydrogen and C₁₋₃ alkyl, optionally substituted with C₁₋₃ alkyl or aryl;
   R¹⁰ is NH—C(O)—(CH₂)ₘ—Ar, wherein
      m is 0, 1, 2, 3, 4, or 5, and
      Ar is aryl or heteroaryl optionally substituted with R^{a},
         wherein each R^{a} is selected from hydrogen, C₁₋₃ alkyl, C₁₋₃ alkoxy, C₁₋₃ haloalkyl, or halogen, or two R^{a} together with the carbon to which they are bound form a 5- or 6-membered carbocycle, aryl, or heteroaryl optionally substituted with R^{a}; and
   p is 0, 1, or 2.

In some embodiments, the cancer is breast cancer, melanoma, osteosarcoma, or lung cancer.

In some embodiments, p is 0 or 1. In some embodiments, R¹ is hydrogen. In some embodiments, Ar is a 5- to 10-membered heterocycle. In some embodiments, the 5- to 10-membered heterocycle is thiophenyl, furanyl, pyranyl, pyrrolyl, benzofuranyl, isobenzofuranyl, oxazolyl, indolyl, benzo[b]thiophenyl, or benzo[c]thiophenyl. In some embodiments, at least one of R⁶, R⁷, R⁸, or R⁹ is hydrogen. In some embodiments, Ar is substituted with at least one R^{a}. In some embodiments, R¹⁰ has the structure:

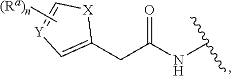

wherein X is N, S, or O; Y is N, S, or C; R^{a} is selected from: hydrogen, C₁₋₃ alkyl, C₁₋₃ alkoxy, C₁₋₃ haloalkyl, or halogen; and n is 0, 1, 2, or 3.

In some embodiments, p is 0. In some embodiments, p is 0 and R¹ is H. In some embodiments, R^{N} is pyrrolidine or piperidine. In some embodiments, one or R⁴ or R⁵ is methyl and the other is hydrogen. In some embodiments, Ar is a 5- to 10-membered aryl or heteroaryl. In some embodiments, the 5- or 6-membered aryl or heteroaryl is imidazolyl, thiazolyl, furanyl, thiophenyl, pyridinyl, pyrimidinyl, pyrazinyl, pyranyl, indolyl, naphthyl, or isoquinolinyl. In some embodiments, Ar is substituted with at least one R^{a}. In some embodiments, R^{a} is a halogen. In some embodiments, the halogen is bromo or chloro.

In some embodiments, p is 1. In some embodiments, p is 1 and R¹ is H. In some embodiments, R^{N} is pyrrolidine or piperidine. In some embodiments, one or R⁴ or R⁵ is methyl and the other is hydrogen. In some embodiments, Ar is a 5- to 10-membered aryl or heteroaryl. In some embodiments, the 5- to 10-membered aryl or heteroaryl is imidazolyl, thiazolyl, furanyl, thiophenyl, pyridinyl, pyrimidinyl, pyrazinyl, pyranyl, indolyl, naphthyl, or isoquinolinyl. In some embodiments, Ar is substituted with at least one R^{a}. In some embodiments, R^{a} is a halogen. In some embodiments, the halogen is bromo or chloro.

In some embodiments, the CHD1Li has the structure of Formula (II):

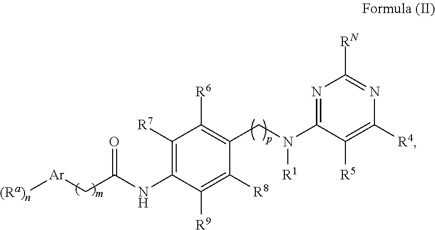

or a pharmaceutically acceptable salt thereof, wherein:
   R^{N} is an optionally substituted 5- to 7-membered heterocycle optionally containing a second heteroatoms selected from the group N, S, or O;
   R¹ is hydrogen or methyl;
   R⁴ and R⁵ are each independently hydrogen, C₁₋₃ alkyl group, or halogen;
   R⁶ is hydrogen or halogen;
   R⁷, R⁸, and R⁹ are each independently hydrogen, C₁₋₃ alkyl, C₁₋₃ alkoxy, optionally substituted C₁₋₃ alkyl, or aryl;
   Ar is an aryl or heteroaryl optionally substituted R^{a}, wherein
      each R^{a} is selected from hydrogen, C₁₋₃ alkyl, C₁₋₃ alkoxy, C₁₋₃ haloalkyl, or halogen, or two R^{a} together with the carbon to which they are bound form a 5- or 6-membered carbocycle, aryl, or heteroaryl optionally substituted with R^{b}, and
      each R^{b} is selected from C₁₋₃ alkyl, C₁₋₃ alkoxy, C₁₋₃ haloalkyl, or halogen;
   p is 0 or 1;
   m is 0, 1, 2, 3, 4, or 5; and
   n is an integer from 0 to 9.

In some embodiments, Ar is a 5- to 10-membered heterocycle. In some embodiments, the 5- to 10-membered heterocycle is thiophenyl, furanyl, pyranyl, pyrrolyl, benzofuranyl, isobenzofuranyl, oxazolyl, indolyl, benzo[b]thiophenyl, or benzo[c]thiophenyl. In some embodiments, the 5- to 10-membered aryl or heteroaryl is imidazolyl, thiazolyl, furanyl, thiophenyl, pyridinyl, pyrimidinyl, pyrazinyl, pyranyl, indolyl, naphthyl, or isoquinolinyl. In some embodiments, R⁴ is methyl. In some embodiments, R¹ is hydrogen. In some embodiments, at least two of R⁵, R⁶, R⁷, R⁸, and R⁹ are hydrogen. In some embodiments, at least one of R⁵, R⁶, R⁷, R⁸, or R⁹ is halogen or C₁₋₃ alkoxy.

In some embodiments, m is 1. In some embodiments, R^{N} is pyrrolidine or piperidine. In some embodiments, R¹ is hydrogen. In some embodiments, one of R⁴ or R⁵ is hydrogen. In some embodiments, R⁶ is hydrogen. In some embodiments, one of R⁷, R⁸, or R⁹ is hydrogen. In some embodiments, Ar is a 5- to 10-membered aryl or heteroaryl. In some embodiments, n is an integer from 1 to 9. In some embodiments, R^{a} is hydrogen, C₁₋₃ alkyl, C₁₋₃ haloalkyl, or halogen. In some embodiments, the 5- to 10-membered heterocycle is thiophenyl, furanyl, pyranyl, pyrrolyl, benzofuranyl, isobenzofuranyl, oxazolyl, indolyl, benzo[b]thiophenyl, or benzo[c]thiophenyl. In some embodiments, the 5- to 10-membered aryl or heteroaryl is imidazolyl, thiazolyl, furanyl, thiophenyl, pyridinyl, pyrimidinyl, pyrazinyl, pyranyl, indolyl, naphthyl, or isoquinolinyl.

In some embodiments, m is 2. In some embodiments, R^{N} is pyrrolidine or piperidine. In some embodiments, R¹ is hydrogen. In some embodiments, one of R⁴ or R⁵ is hydrogen. In some embodiments, R⁶ is hydrogen. In some embodiments, one of R⁷, R⁸, or R⁹ is hydrogen. In some embodiments, Ar is a 5- to 10-membered aryl or heteroaryl. In some embodiments, n is an integer from 1 to 9. In some embodiments, R^{a} is hydrogen, C₁₋₃ alkyl, C₁₋₃ haloalkyl, or halogen. In some embodiments, the 5- to 10-membered heterocycle is thiophenyl, furanyl, pyranyl, pyrrolyl, benzofuranyl, isobenzofuranyl, oxazolyl, indolyl, benzo[b]thiophenyl, or benzo[c]thiophenyl. In some embodiments, the 5- to 10-membered aryl or heteroaryl is imidazolyl, thiazolyl, furanyl, thiophenyl, pyridinyl, pyrimidinyl, pyrazinyl, pyranyl, indolyl, naphthyl, or isoquinolinyl.

In some embodiments, the CHD1L inhibitor has the structure of Formula (IIIA):

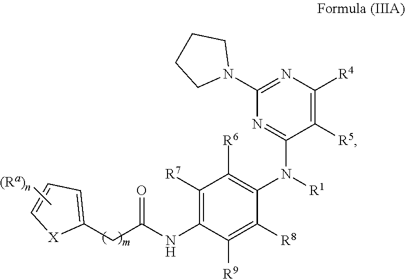

or a pharmaceutically acceptable salt thereof, wherein:
   R¹ is hydrogen or methyl;
   R⁴ and R⁵ are each independently hydrogen, C₁₋₃ alkyl group, or halogen;
   R⁶ is hydrogen or a halogen;
   R⁷, R⁸, and R⁹ are each independently hydrogen, C₁₋₃ alkyl, optionally substituted C₁₋₃ alkyl, or aryl;
   each R^{a} is selected from hydrogen, C₁₋₃ alkyl, C₁₋₃ alkoxy, C₁₋₃ haloalkyl, or halogen, or two R^{a} together with the carbon to which they are bound form a 5- or 6-membered carbocycle, aryl, or heteroaryl optionally substituted with R^{b};
   each R^{b} is selected from C₁₋₃ alkyl, C₁₋₃ alkoxy, C₁₋₃ haloalkyl, or halogen;
   X is N, S, or O;
   m is 0, 1, 2, 3, 4, or 5; and
   n is 0, 1, 2, 3, or 4.

In some embodiments, R¹ is hydrogen. In some embodiments, at least one of R⁶, R⁷, R⁸, or R⁹ is hydrogen. The In some embodiments, m is 1, 2, 3, 4, or 5. In some embodiments, n is 1, 2, 3, or 4. In some embodiments, when n is 0, m is at least 1. In some embodiments, R^{a} is a C₁₋₃ alkyl or halogen. In some embodiments, the halogen is bromo or chloro. In some embodiments, X is N. In some embodiments, X is S. In some embodiments, X is O. In some embodiments, at least one of R⁴ or R⁵ is methyl. In some embodiments, CHD1L inhibitor having the structure of Formula (IIIA) is:

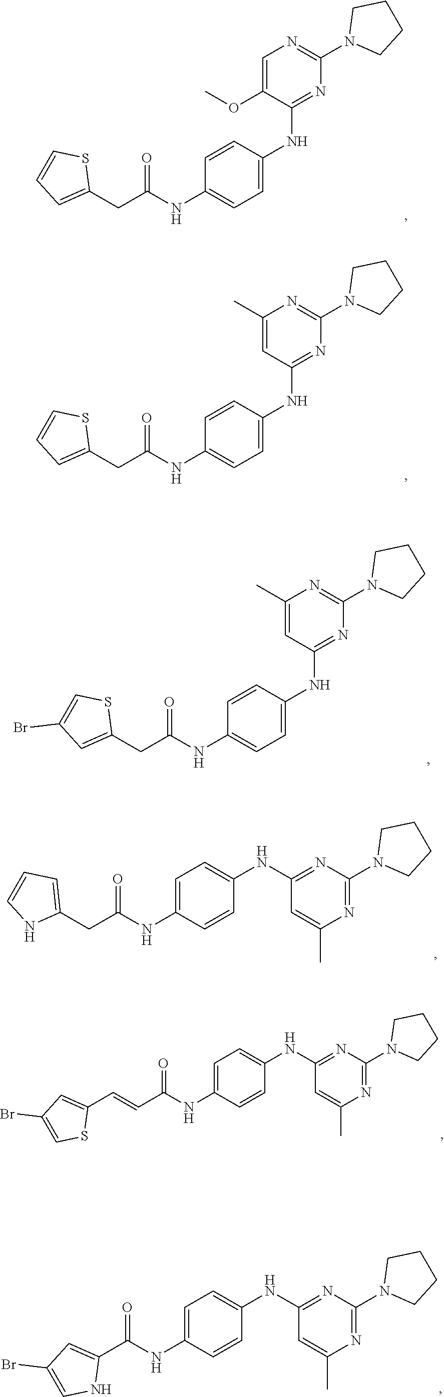 [SMILES: COc1cnc(N2CCCC2)nc1Nc1ccc(NC(=O)Cc2cccs2)cc1 ; Cc1cc(Nc2ccc(NC(=O)Cc3cccs3)cc2)nc(N2CCCC2)n1 ; Cc1cc(Nc2ccc(NC(=O)Cc3cc(Br)cs3)cc2)nc(N2CCCC2)n1 ; Cc1cc(Nc2ccc(NC(=O)Cc3ccc[nH]3)cc2)nc(N2CCCC2)n1 ; Cc1cc(Nc2ccc(NC(=O)/C=C/c3cc(Br)cs3)cc2)nc(N2CCCC2)n1 ; Cc1cc(Nc2ccc(NC(=O)c3cc(Br)c[nH]3)cc2)nc(N2CCCC2)n1]

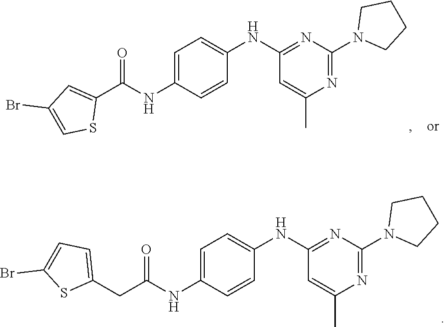 [SMILES: Cc1cc(Nc2ccc(NC(=O)c3cc(Br)cs3)cc2)nc(N2CCCC2)n1 ; Cc1cc(Nc2ccc(NC(=O)Cc3ccc(Br)s3)cc2)nc(N2CCCC2)n1]

In some embodiments, the CHD1L inhibitor has the structure of Formula (IIIB):

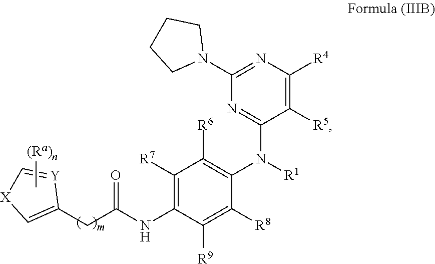

or a pharmaceutically acceptable salt thereof, wherein:
   R¹ is hydrogen or methyl;
   R⁴ and R⁵ are each independently hydrogen, C₁₋₃ alkyl group, or halogen;
   R⁶ is hydrogen or a halogen;
   R⁷, R⁸, and R⁹ are each independently hydrogen, C₁₋₃ alkyl, optionally substituted C₁₋₃ alkyl, or aryl;
   each R^{a} is selected from hydrogen, C₁₋₃ alkyl, C₁₋₃ alkoxy, C₁₋₃ haloalkyl, or halogen, or two R^{a} together with the carbon to which they are bound form a 5- or 6-membered carbocycle, aryl, or heteroaryl optionally substituted with R^{b};
   each R^{b} is selected from C₁₋₃ alkyl, C₁₋₃ alkoxy, C₁₋₃ haloalkyl, or halogen;
   X is N, S, or O;
   Y is C, N, or S;
   m is 1, 2, 3, 4, or 5; and
   n is 1, 2, 3, or 4.

In some embodiments, R¹ is hydrogen. In some embodiments, at least one of R⁶, R⁷, R⁸, or R⁹ is hydrogen. In some embodiments, m is 1, 2, or 3. In some embodiments, n is 1, 2, 3, or 4. In some embodiments, R^{a} is a C₁₋₃ alkyl or halogen. In some embodiments, the halogen is bromo or chloro. In some embodiments, X is N. In some embodiments, X is O. In some embodiments, X is S. In some embodiments, Y is N, n is 1, and R^{a} is halogen. In some embodiments, at least one or R⁴ or R⁵ is methyl. In some embodiments, the CHD1L inhibitor of Formula (IIIB) is:

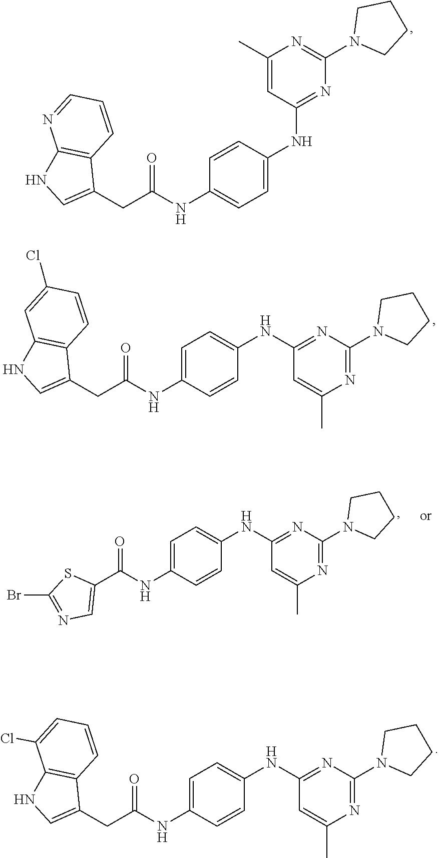 [SMILES: Cc1cc(Nc2ccc(NC(=O)Cc3c[nH]c4ncccc34)cc2)nc(N2CCCC2)n1 ; Cc1cc(Nc2ccc(NC(=O)Cc3c[nH]c4cc(Cl)ccc34)cc2)nc(N2CCCC2)n1 ; Cc1cc(Nc2ccc(NC(=O)c3cnc(Br)s3)cc2)nc(N2CCCC2)n1 ; Cc1cc(Nc2ccc(NC(=O)Cc3c[nH]c4c(Cl)cccc34)cc2)nc(N2CCCC2)n1]

In some embodiments, the CHD1L inhibitor is a compound selected from Table 1.

In some aspects, the CHD1L inhibitor, or a pharmaceutically acceptable salt thereof, is formulated as a pharmaceutical composition. In some embodiments, the pharmaceutical composition for treating a cancer comprises a CHD1L inhibitor having the structure:

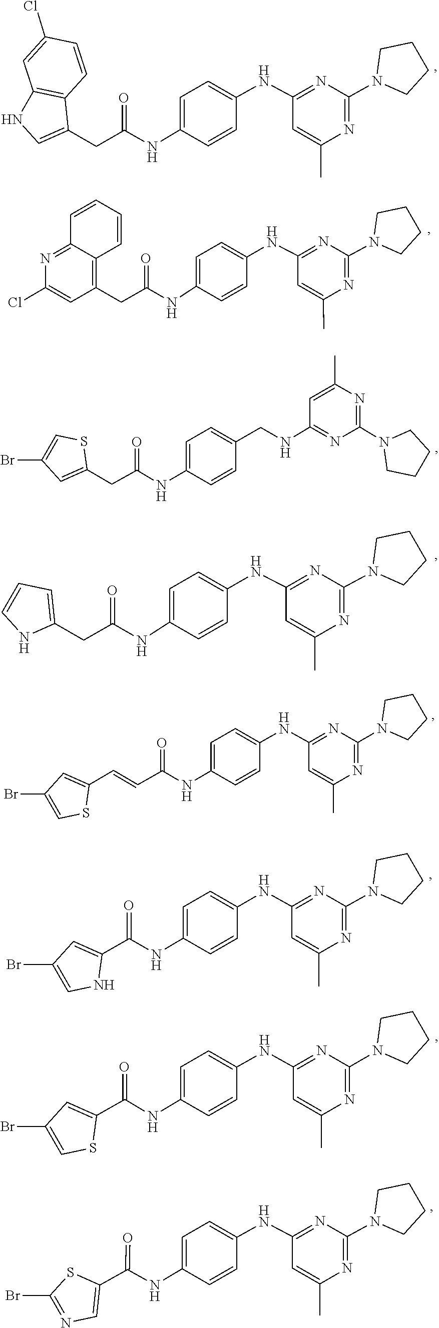

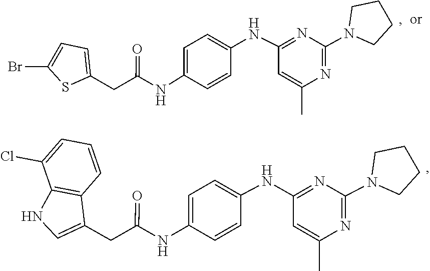 [SMILES: Cc1cc(Nc2ccc(NC(=O)Cc3ccc(Br)s3)cc2)nc(N2CCCC2)n1 ; Cc1cc(Nc2ccc(NC(=O)Cc3c[nH]c4c(Cl)cccc34)cc2)nc(N2CCCC2)n1]

or a pharmaceutically acceptable salt thereof. In some embodiments, the CHD1L inhibitor of the pharmaceutical composition is: 2-(6-chloro-1H-indol-3-yl)-N-(4-((6-methyl-2-(pyrrolidin-1-yl)pyrimidin-4-yl)amino)phenyl)acetamide, 2-(2-chloroquinolin-4-yl)-N-(4-((6-methyl-2-(pyrrolidin-1-yl)pyrimidin-4-yl)amino)phenyl)acetamide, 2-(4-bromothiophen-2-yl)-N-(4-(((6-methyl-2-(pyrrolidin-1-yl)pyrimidin-4-yl)amino)methyl)phenyl)acetamide, N-(4-((6-methyl-2-(pyrrolidin-1-yl)pyrimidin-4-yl)amino)phenyl)-2-(1H-pyrrol-2-yl)acetamide, (E)-3-(4-bromothiophen-2-yl)-N-(4-((6-methyl-2-(pyrrolidin-1-yl)pyrimidin-4-yl)amino)phenyl)acrylamide, 4-bromo-N-(4-((6-methyl-2-(pyrrolidin-1-yl)pyrimidin-4-yl)amino)phenyl)-1H-pyrrole-2-carboxamide, 4-bromo-N-(4-((6-methyl-2-(pyrrolidin-1-yl)pyrimidin-4-yl)amino)phenyl)thiophene-2-carboxamide, 2-bromo-N-(4-((6-methyl-2-(pyrrolidin-1-yl)pyrimidin-4-yl)amino)phenyl)thiazole-5-carboxamide, 2-(5-bromothiophen-2-yl)-N-(4-((6-methyl-2-(pyrrolidin-1-yl)pyrimidin-4-yl)amino)phenyl)acetamide, 2-(7-chloro-1H-indol-3-yl)-N-(4-((6-methyl-2-(pyrrolidin-1-yl)pyrimidin-4-yl)amino)phenyl)acetamide.

In some embodiments, the pharmaceutical composition further comprises a chemotherapy. In some embodiments, the chemotherapy comprises irinotecan, olaparib, doxorubicin, docetaxel, AZD5305 (or, or 5-fluorouracil (5-FU), or a combination of two or more thereof. In some embodiments, the chemotherapy is olaparib. In some embodiments, the chemotherapy is irinotecan. In some embodiments, wherein the chemotherapy is doxorubicin. In some embodiments, the chemotherapy is docetaxel. In some embodiments, the chemotherapy is AZD5305. In some embodiments, the chemotherapy is 5-fluorouracil (5-FU). In some embodiments, the chemotherapy is effective against breast cancer. In some embodiments, the chemotherapy is effective against colorectal cancer. In some embodiments, the CHD1L inhibitor is a compound of Table 1. In some embodiments, the CHD1L inhibitor is a compound of Formula (I), (II), (IIIA), or (IIIB), or a pharmaceutically acceptable salt or solvate thereof. In some embodiments, a combination of the CHD1L inhibitor, or the pharmaceutically acceptable salt or solvate thereof, and the chemotherapy is therapeutically effective to treat cancer.

Also provided are pharmaceutical formulations comprising the CHD1L inhibitor, or a pharmaceutically acceptable salt or solvate thereof, for administration in combination with a chemotherapy. In some embodiments, the CHD1L inhibitor is a compound of Table 1. In some embodiments, the CHD1L inhibitor is a compound of Formula (I), (II), (IIIA), or (IIIB), or a pharmaceutically acceptable salt or solvate thereof. In some embodiments, the pharmaceutical formulation comprises the CHD1L inhibitor, or a pharmaceutically acceptable salt or solvate thereof; and the chemotherapy. In some embodiments, the CHD1L inhibitor and the chemotherapy are formulated as separate dosages. In some embodiments, the CHD1L inhibitor and the chemotherapy are formulated to be administered simultaneously. In some embodiments, the CHD1L inhibitor and the chemotherapy are formulated to be administered sequentially. In some embodiments, the CHD1L inhibitor is formulated to be administered prior to administration of the chemotherapy. In some embodiments, the CHD1L inhibitor is formulated to be administered after administration of the chemotherapy. In some embodiments, the pharmaceutical composition is formulated to be administered orally or parenterally. In some embodiments, the pharmaceutical composition is formulated to be administered orally. In some embodiments, the pharmaceutical composition is formulated to be administered intraperitoneally. In some embodiments, the pharmaceutical formulation is formulated to be parenterally administered as an intradermal injection, subcutaneous injection, intramuscular injection, intraperitoneal injection, intraperitoneal infusion, or intravenous injection.

In some embodiments, the CHD1L inhibitor, or the pharmaceutically acceptable salt or solvate thereof, potentiates the chemotherapy. In some embodiments, the CHD1L inhibitor, or the pharmaceutically acceptable salt or solvate thereof, is synergistic with the chemotherapy. In some embodiments, the cancer is resistant to the chemotherapy, when the cancer is not treated with the CHD1L inhibitor. In some embodiments, the chemotherapy is a PARP inhibitor. In some embodiments, the chemotherapy is a standard of care (SOC) chemotherapy for the cancer. In some embodiments, the chemotherapy comprises irinotecan, olaparib, doxorubicin, docetaxel, AZD5305, or 5-fluorouracil (5-FU), or a combination of two or more thereof. In some embodiments, the administration of the CHD1L inhibitor and the chemotherapy provides a Bliss synergy score of at least about 10, wherein the Bliss energy score is determined using a Bliss model. In some embodiments, the administration of the CHD1L inhibitor and the chemotherapy provides a Bliss synergy score of at least about 10 to about 60 using a Bliss model. In some embodiments, the administration of the CHD1L inhibitor localizes apoptosis inducing factor (AIF) in the nucleus of the tumor cell. In some embodiments, the administration of the CHD1L inhibitor provides a higher concentration of AIF in the nucleus of the tumor cell relative to a concentration of AIF in a cytoplasm of the tumor cell. In some embodiments, the CHD1L inhibitor induces localization of PAR in the nucleus of the tumor cell.

### Certain Definitions

Unless defined otherwise, all technical and scientific terms used herein have the same meaning as is commonly understood by one of skill in the art to which this invention belongs. All patents and publications referred to herein are incorporated by reference.

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulas, all combinations and sub-combinations of ranges and specific embodiments therein are intended to be included.

The term “about” when referring to a number or a numerical range means that the number or numerical range referred to is an approximation within experimental variability (or within statistical experimental error), and thus the number or numerical range, in some instances, will vary between 1% and 15% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below:

As used herein, “C₁-Cₓ” includes C₁-C₂, C₁-C₃ . . . C₁-Cₓ. By way of example only, a group designated as “C₁-C₄” indicates that there are one to four carbon atoms in the moiety, i.e., groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

“Alkyl” refers to an optionally substituted straight-chain, or optionally substituted branched-chain saturated hydrocarbon monoradical having from one to about ten carbon atoms, or more preferably, from one to six carbon atoms, wherein an sp³-hybridized carbon of the alkyl residue is attached to the rest of the molecule by a single bond. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted as described below by one or more of the following substituents: halo, cyano, nitro, oxo, thioxo, imino, oximo, trimethylsilanyl, —OR^{a′}, —SR^{a′}, —OC(O)R^{a′}, —OC(O)—OR^{f′}, —N(R^{a′})₂, —N⁺(R^{a′})₃, —C(O)R^{a′}, —C(O)OR^{a′}, —C(O)N(R^{a′})₂, —N(R^{a′})C(O)OR^{f′}, —OC(O)—N(R^{a′})₂, —N(R^{a′})C(O)R^{f′}, —N(R^{a′})S(O)ₜR^{f′} (where t is 1 or 2), —S(O)ₜOR^{a′} (where t is 1 or 2), —S(O)ₜR^{f′} (where t is 1 or 2) and —S(O)ₜN(R^{a′})₂ (where t is 1 or 2) where each R^{a′} is independently hydrogen, alkyl, haloalkyl, cycloalkyl, aryl, aralkyl, heterocycloalkyl, heteroaryl or heteroarylalkyl, and each R^{f′} is independently alkyl, haloalkyl, cycloalkyl, aryl, aralkyl, heterocycloalkyl, heteroaryl or heteroarylalkyl.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms, wherein an sp²-hybridized carbon or an sp³-hybridized carbon of the alkenyl residue is attached to the rest of the molecule by a single bond. The group may be in either the cis or trans conformation about the double bond(s) and should be understood to include both isomers. Examples include, but are not limited to, ethenyl (—CH═CH₂), 1-propenyl (—CH₂CH═CH₂), isopropenyl (—C(CH₃)═CH₂), butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted as described below, for example, with oxo, halogen, amino, nitrile, nitro, hydroxyl, haloalkyl, alkoxy, aryl, cycloalkyl, heterocycloalkyl, heteroaryl, and the like. Unless stated otherwise specifically in the specification, an alkenyl group is optionally substituted as described below by one or more of the following substituents: halo, cyano, nitro, oxo, thioxo, imino, oximo, trimethylsilanyl, —OR^{a′}, —SR^{a′}, —OC(O)R^{a′}, —OC(O)—OR^{f′}, —N(R^{a′})₂, —N⁺(R^{a′})₃, —C(O)R^{a′}, —C(O)OR^{a′}, —C(O)N(R^{a′})₂, —N(R^{a′})C(O)OR^{f′}, —OC(O)—N(R^{a′})₂, —N(R^{a′})C(O)R^{f′}, —N(R^{a′})S(O)ₜR^{f′} (where t is 1 or 2), —S(O)ₜOR^{a′} (where t is 1 or 2), —S(O)ₜR^{f′} (where t is 1 or 2) and —S(O)ₜN(R^{a′})₂ (where t is 1 or 2) where each R^{a′} is independently hydrogen, alkyl, haloalkyl, cycloalkyl, aryl, aralkyl, heterocycloalkyl, heteroaryl or heteroarylalkyl, and each R^{f′} is independently alkyl, haloalkyl, cycloalkyl, aryl, aralkyl, heterocycloalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. Unless stated otherwise specifically in the specification, an alkylene group is optionally substituted as described below by one or more of the following substituents: halo, cyano, nitro, oxo, thioxo, imino, oximo, trimethylsilanyl, —OR^{a′}, —SR^{a′}, —OC(O)R^{a′}, —OC(O)—OR^{f′}, —N(R^{a′})₂, —N⁺(R^{a′})₃, —C(O)R^{a′}, —C(O)OR^{a′}, —C(O)N(R^{a′})₂, —N(R^{a′})C(O)OR^{f′}, —OC(O)—N(R^{a′})₂, —N(R^{a′})C(O)R^{f′}, —N(R^{a′})S(O)ₜR^{f′} (where t is 1 or 2), —S(O)ₜOR^{a′} (where t is 1 or 2), —S(O)ₜR^{f′} (where t is 1 or 2) and —S(O)ₜN(R^{a′})₂ (where t is 1 or 2) where each R^{a′} is independently hydrogen, alkyl, haloalkyl, cycloalkyl, aryl, aralkyl, heterocycloalkyl, heteroaryl or heteroarylalkyl, and each R^{f′} is independently alkyl, haloalkyl, cycloalkyl, aryl, aralkyl, heterocycloalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. Unless stated otherwise specifically in the specification, an alkenylene group is optionally substituted as described below by one or more of the following substituents: halo, cyano, nitro, oxo, thioxo, imino, oximo, trimethylsilanyl, —OR^{a′}, —SR^{a′}, —OC(O)R^{a′}, —OC(O)—OR^{f′}, —N(R^{a′})₂, —N⁺(R^{a′})₃, —C(O)R^{a′}, —C(O)OR^{a′}, —C(O)N(R^{a′})₂, —N(R^{a′})C(O)OR^{f′}, —OC(O)—N(R^{a′})₂, —N(R^{a′})C(O)R^{f′}, —N(R^{a′})S(O)ₜR^{f′} (where t is 1 or 2), —S(O)ₜOR^{a′} (where t is 1 or 2), —S(O)ₜR^{f′} (where t is 1 or 2) and —S(O)ₜN(R^{a′})₂ (where t is 1 or 2) where each R^{a′} is independently hydrogen, alkyl, haloalkyl, cycloalkyl, aryl, aralkyl, heterocycloalkyl, heteroaryl or heteroarylalkyl, and each R^{f′} is independently alkyl, haloalkyl, cycloalkyl, aryl, aralkyl, heterocycloalkyl, heteroaryl or heteroarylalkyl.

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. Unless stated otherwise specifically in the specification, an alkynylene group is optionally substituted as described below by one or more of the following substituents: halo, cyano, nitro, oxo, thioxo, imino, oximo, trimethylsilanyl, —OR^{a′}, —SR^{a′}, —OC(O)R^{a′}, —OC(O)—OR^{f′}, —N(R^{a′})₂, —N⁺(R^{a′})₃, —C(O)R^{a′}, —C(O)OR^{a′}, —C(O)N(R^{a′})₂, —N(R^{a′})C(O)OR^{f′}, —OC(O)—N(R^{a′})₂, —N(R^{a′})C(O)R^{f′}, —N(R^{a′})S(O)ₜR^{f′} (where t is 1 or 2), —S(O)ₜOR^{a′} (where t is 1 or 2), —S(O)ₜR^{f′} (where t is 1 or 2) and —S(O)ₜN(R^{a′})₂ (where t is 1 or 2) where each R^{a′} is independently hydrogen, alkyl, haloalkyl, cycloalkyl, aryl, aralkyl, heterocycloalkyl, heteroaryl or heteroarylalkyl, and each R^{f′} is independently alkyl, haloalkyl, cycloalkyl, aryl, aralkyl, heterocycloalkyl, heteroaryl or heteroarylalkyl.

“Alkoxy” or “alkoxyl” refers to a radical bonded through an oxygen atom of the formula —O-alkyl, where alkyl is an alkyl chain as defined above.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon atoms unless otherwise specified (i.e., from 6 to 18 carbon atoms), where at least one of the rings in the ring system is fully unsaturated, (i.e., it contains a cyclic, delocalized (4n+2) π-electron system in accordance with the Hückel theory). The ring system from which aryl groups are derived include, but are not limited to, groups such as benzene, fluorene, indane, indene, tetralin and naphthalene. In some embodiments, the aryl is a C₆-C₁₀ aryl. In some embodiments, the aryl is a phenyl. Unless stated otherwise specifically in the specification, the term “aryl” or the prefix “ar-” (such as in “aralkyl”) is meant to include aryl radicals optionally substituted as described below by one or more substituents independently selected from alkyl, alkenyl, alkynyl, halo, haloalkyl, cyano, nitro, aryl, aralkyl, aralkenyl, aralkynyl, cycloalkyl, heterocycloalkyl, heteroaryl, heteroarylalkyl, —R^{b′}—OR^{a′}, —R^{b′}—SR^{a′}, —R^{b′}—OC(O)—R^{a′}, —R^{b′}—OC(O)—OR^{f′}, —R^{b′}—OC(O)—N(R^{a′})₂, —R^{b′}—N(R^{a′})₂, —R^{b′}—N⁺(R^{a′})₃, —R^{b′}—C(O)R^{a′}, —R^{b′}—C(O)OR^{a′}, —R^{b′}—C(O)N(R^{a′})₂, —R^{b′}—O—R^{c′}—C(O)N(R^{a′})₂, —R^{b′}—N(R^{a′})C(O)OR^{f′}, —R^{b′}—N(R^{a′})C(O)R^{a′}, —R^{b′}—N(R^{a′})S(O)ₜR^{f′} (where t is 1 or 2), —R^{b′}—S(O)ₜOR^{a′} (where t is 1 or 2), —R^{b′}—S(O)ₜR^{f′} (where t is 1 or 2) and —R^{b′}—S(O)ₜN(R^{a′})₂ (where t is 1 or 2), where each R^{a′} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R^{f′} is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b′} is independently a direct bond or a straight or branched alkylene or alkenylene chain, and R^{c′} is a straight or branched alkylene or alkenylene chain.

“Arylene” refers to a divalent radical derived from an “aryl” group as described above linking the rest of the molecule to a radical group. The arylene is attached to the rest of the molecule through a single bond and to the radical group through a single bond. In some embodiments, the arylene is a phenylene. Unless stated otherwise specifically in the specification, an arylene group is optionally substituted as described above for an aryl group.

“CHD1L” or “ALC1” are used interchangeably herein to refer to Chromodomain Helicase DNA Binding Protein 1 Like (CHD1L), HGNC: 1916 NCBI Gene: 9557 Ensembl: ENSG00000131778 OMIM®: 613039 UniProtKB/Swiss-Prot: Q86WJ1. CHD1L encodes a DNA helicase protein involved in DNA repair. The protein converts ATP to add poly(ADP-ribose) as it regulates chromatin relaxation following DNA damage.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom), bridged ring systems, and/or spirocyclic ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[1.1.1]pentyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, 7,7-dimethyl-bicyclo[2.2.1]heptanyl, and the like. Unless otherwise stated specifically in the specification, the term “cycloalkyl” is meant to include cycloalkyl radicals optionally substituted as described below by one or more substituents independently selected from alkyl, alkenyl, alkynyl, halo, haloalkyl, cyano, nitro, aryl, aralkyl, aralkenyl, aralkynyl, cycloalkyl, heterocycloalkyl, heteroaryl, heteroarylalkyl, —R^{b′}—OR^{a′}, —R^{b′}—SR^{a′}, —R^{b′}—OC(O)—R^{a′}, —R^{b′}—OC(O)—OR^{f′}, —R^{b′}—OC(O)—N(R^{a′})₂, —R^{b′}—N(R^{a′})₂, —R^{b′}—N⁺(R^{a′})₃, —R^{b′}—C(O)R^{a′}, —R^{b′}—C(O)OR^{a′}, —R^{b′}—C(O)N(R^{a′})₂, —R^{b′}—O—R^{c′}—C(O)N(R^{a′})₂, —R^{b′}—N(R^{a′})C(O)OR^{f′}, —R^{b′}—N(R^{a′})C(O)R^{a′}, —R^{b′}—N(R^{a′})S(O)ₜR^{f′} (where t is 1 or 2), —R^{b′}—S(O)ₜOR^{a′} (where t is 1 or 2), —R^{b′}—S(O)ₜR^{f′} (where t is 1 or 2) and —R^{b′}—S(O)ₜN(R^{a′})₂ (where t is 1 or 2), where each R^{a′} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R^{f′} is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b′} is independently a direct bond or a straight or branched alkylene or alkenylene chain, and R^{c′} is a straight or branched alkylene or alkenylene chain.

“Cycloalkylene” refers to a divalent radical derived from a “cycloalkyl” group as described above linking the rest of the molecule to a radical group. The cycloalkylene is attached to the rest of the molecule through a single bond and to the radical group through a single bond. Unless stated otherwise specifically in the specification, a cycloalkylene group is optionally substituted as described above for a cycloalkyl group.

“Halo” or “halogen” refers to bromo, chloro, fluoro or iodo. In some embodiments, halogen is fluoro or chloro. In some embodiments, halogen is chloro or bromo. In some embodiments, halogen is fluoro. In some embodiments, halogen is chloro. In some embodiments, halogen is bromo.

“Haloalkyl” refers to an alkyl radical, as defined above, that is substituted by one or more hydroxy radicals, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more halo radicals, as defined above, e.g., hydroxymethyl, 1-hydroxyethyl, 2-hydroxyethyl, 2-hydroxypropyl, 3-hydroxypropyl, 1,2-dihydroxyethyl, 2,3-dihydroxypropyl, 2,3,4,5,6-pentahydroxyhexyl, and the like.

“Heterocycloalkyl” refers to a stable 3- to 24-membered partially or fully saturated ring radical comprising 2 to 23 carbon atoms and from one to 8 heteroatoms selected from the group consisting of nitrogen, oxygen, and sulfur. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom), bridged ring systems, and/or spirocyclic ring systems; and the nitrogen, carbon or sulfur atoms in the heterocycloalkyl radical may be optionally oxidized; the nitrogen atom may be optionally quaternized. In some embodiments, the heterocycloalkyl is a 3- to 8-membered heterocycloalkyl. In some embodiments, the heterocycloalkyl is a 3- to 6-membered heterocycloalkyl. In some embodiments, the heterocycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. More preferably, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, the term “heterocycloalkyl” is meant to include heterocycloalkyl radicals as defined above that are optionally substituted by one or more substituents selected from alkyl, alkenyl, alkynyl, halo, fluoroalkyl, oxo, thioxo, cyano, nitro, aryl, aralkyl, aralkenyl, aralkynyl, cycloalkyl, heterocycloalkyl, heteroaryl, heteroarylalkyl, —R^{b′}—OR^{a′}, —R^{b′}—SR^{a′}, —R^{b′}—OC(O)—R^{a′}, —R^{b′}—OC(O)—OR^{f′}, —R^{b′}—OC(O)—N(R^{a′})₂, —R^{b′}—N(R^{a′})₂, —R^{b′}—N⁺(R^{a′})₃, —R^{b′}—C(O)R^{a′}, —R^{b′}—C(O)OR^{a′}, —R^{b′}—C(O)N(R^{a′})₂, —R^{b′}—O—R^{c′}—C(O)N(R^{a′})₂, —R^{b′}—N(R^{a′})C(O)OR^{f′}, —R^{b′}—N(R^{a′})C(O)R^{a′}, —R^{b′}—N(R^{a′})S(O)ₜR^{f′} (where t is 1 or 2), —R^{b′}—S(O)ₜOR^{a′} (where t is 1 or 2), —R^{b′}—S(O)ₜR^{f′} (where t is 1 or 2) and —R^{b′}—S(O)ₜN(R^{a′})₂ (where t is 1 or 2), where each R^{a′} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R^{f′} is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b′} is independently a direct bond or a straight or branched alkylene or alkenylene chain, and R^{c′} is a straight or branched alkylene or alkenylene chain.

“N-heterocycloalkyl” refers to a heterocycloalkyl radical as defined above containing at least one nitrogen and where the point of attachment of the heterocycloalkyl radical to the rest of the molecule is through a nitrogen atom in the heterocycloalkyl radical. An N-heterocycloalkyl radical is optionally substituted as described above for heterocycloalkyl radicals.

“C-heterocycloalkyl” refers to a heterocycloalkyl radical as defined above and where the point of attachment of the heterocycloalkyl radical to the rest of the molecule is through a carbon atom in the heterocycloalkyl radical. A C-heterocycloalkyl radical is optionally substituted as described above for heterocycloalkyl radicals.

“Heterocycloalkylene” refers to a divalent radical derived from a “heterocycloalkyl” group as described above linking the rest of the molecule to a radical group. The heterocycloalkylene is attached to the rest of the molecule through a single bond and to the radical group through a single bond. Unless stated otherwise specifically in the specification, a heterocycloalkylene group is optionally substituted as described above for a heterocycloalkyl group.

“Heteroaryl” refers to a radical derived from a 5- to 18-membered aromatic ring radical that comprises one to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. In some embodiments, the heteroaryl is a 5- to 10-membered heteroaryl. In some embodiments, the heteroaryl is a monocyclic heteroaryl, or a monocyclic 5- or 6-membered heteroaryl. In some embodiments, the heteroaryl is a 6,5-fused bicyclic heteroaryl. The heteroatom(s) in the heteroaryl radical is optionally oxidized. The carbon atom(s) in the heteroaryl is optionally oxidized. Two non-limiting examples of heteroaryl radicals that are oxidized and are encompassed by the term heteroaryl are pyridone and pyridine N-oxide. One or more nitrogen atoms, if present, are optionally quaternized. The heteroaryl is attached to the rest of the molecule through any atom of the ring(s). Unless stated otherwise specifically in the specification, the term “heteroaryl” is meant to include heteroaryl radicals as defined above that are optionally substituted by one or more substituents selected from alkyl, alkenyl, alkynyl, halo, haloalkyl, oxo, thioxo, cyano, nitro, aryl, aralkyl, aralkenyl, aralkynyl, cycloalkyl, heterocycloalkyl, heteroaryl, heteroarylalkyl, —R^{b′}—OR^{a′}, —R^{b′}—SR^{a′}, —R^{b′}—OC(O)—R^{a′}, —R^{b′}—OC(O)—OR^{f′}, —R^{b′}—OC(O)—N(R^{a′})₂, —R^{b′}—N(R^{a′})₂, —R^{b′}—N⁺(R^{a′})₃, —R^{b′}—C(O)R^{a′}, —R^{b′}—C(O)OR^{a′}, —R^{b′}—C(O)N(R^{a′})₂, —R^{b′}—O—R^{c′}—C(O)N(R^{a′})₂, —R^{b′}—N(R^{a′})C(O)OR^{f′}, —R^{b′}—N(R^{a′})C(O)R^{a′}, —R^{b′}—N(R^{a′})S(O)ₜR^{f′} (where t is 1 or 2), —R^{b′}—S(O)ₜOR^{a′} (where t is 1 or 2), —R^{b′}—S(O)ₜR^{f′} (where t is 1 or 2) and —R^{b′}—S(O)ₜN(R^{a′})₂ (where t is 1 or 2), where each R^{a′} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R^{f′} is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b′} is independently a direct bond or a straight or branched alkylene or alkenylene chain, and R^{c′} is a straight or branched alkylene or alkenylene chain.

“Heteroarylene” refers to a divalent radical derived from a “heteroaryl” group as described above linking the rest of the molecule to a radical group. The heteroarylene is attached to the rest of the molecule through a single bond and to the radical group through a single bond. Unless stated otherwise specifically in the specification, a heteroarylene group is optionally substituted as described above for a heteroaryl group.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be unsubstituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible.

The term “salt” or “pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts, and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts, and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2 dimethylaminoethanol, 2 diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N ethylpiperidine, polyamine resins, and the like. See Berge et al.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

As used herein, the phrase “therapeutically effective amount,” means the amount of a compound that, when administered to a patient in need, is sufficient to at least partially treat the disease or condition of the subject. The “therapeutically effective amount” will vary depending on the compound, the disease and its severity and the age, weight, adsorption, distribution, metabolism and excretion etc., of the patient to be treated.

As used herein, “treat,” “treatment” or “treating” refers to an approach for obtaining beneficial or desired results with respect to a disease, disorder, or medical condition including, but not limited to, a therapeutic benefit. In certain embodiments, treatment or treating involves administering a compound or composition described herein to a subject. A therapeutic benefit may include the eradication or amelioration of the underlying disorder being treated or a symptom thereof.

As used herein, the terms “subject” and “individual” are used interchangeably and refer to a mammal, such as a human or a non-human mammal (e.g., non-human primates, canines, equines, felines, porcines, bovines, ungulates, lagomorphs, rodents, and the like). In various embodiments, the subject can be a human (e.g., adult male, adult female, adolescent male, adolescent female, male child, female child). In some embodiments, the subject is under the care of a physician or other health worker in a hospital, as an outpatient, or other clinical context. In some embodiments, the subject may not be under the care or prescription of a physician or other health worker. In some embodiments, the subject is a “patient,” which refers to a subject that has or is diagnosed with a disease or a condition of the present disclosure.

As used herein, the phrase “a subject in need thereof” refers to a subject that has, or is at risk for developing, a pathology to be therapeutically treated with a composition (e.g., therapeutic composition) described herein.

The term “antagonist” as used herein refers to a modulator that binds to a target and blocks a biological response. By way of example, a CHD1L antagonist can be used to refer to a compound that exhibits an IC₅₀ with respect to CHD1L activity less than about 1 μM, about 2 μM, about 3 μM, about 4 μM, about 5 μM, about 6 μM, about 7 μM, about 8 μM, about 9 μM, or about 10 μM, as measured in a tumor cell viability assay. In some embodiments, CHD1L antagonist can be used to refer to a compound that exhibits an IC₅₀ with respect to CHD1L activity less than about 10 μM as measured in the tumor cell viability assay. In some embodiments, CHD1L antagonist can be used to refer to a compound that exhibits an IC₅₀ with respect to CHD1L activity less than about 1 μM as measured in the tumor cell viability assay. An antagonist has no activity in the absence of an agonist or inverse agonist but can block the activity of either, causing no change in the biological response.

As used herein, “IC₅₀” is intended to refer to the concentration of a substance (e.g., a compound or a drug) that is required for 50% inhibition of a biological process. For example, IC₅₀ refers to the half maximal (50%) inhibitory concentration (IC) of a substance as determined in a suitable assay. In some instances, an IC₅₀ is determined in an in vitro assay system. In some embodiments as used herein, IC₅₀ refers to the concentration of a modulator (e.g., a CHD1L antagonist) that is required for 50% inhibition of a receptor or a target enzyme (e.g., CHD1L).

As used herein, “synergy” or “synergistic,” as used herein, refer to the activity of two or more compounds wherein their combined activity (relative to a particular function, e.g., inhibition of tumor cell growth) is greater than the expected additive effect of their individual activities. For example, the expected additive effect can be defined according to Bliss synergy criteria. In accordance with the Bliss criteria, the effect of two or more compounds is equal to the sum of the effects of the individual drugs minus the multiplication of the effects of the individual drugs:

$$E 12 = E 1 + E 2 - E 1 ⋆ E 2$$

where E1 is the fractional inhibition by drug 1 (e.g., a CHD1L inhibitor), E2 is the fractional inhibition by drug 2 (e.g., a chemotherapy), and E12 is the expected fractional inhibition by the combination. % inhibition is calculated by multiplying fractional inhibition by 100%.
A Bliss synergy score is calculated by subtraction of the observed % inhibition from the calculated E12% inhibition:

Bliss independence score=(E12−fraction inhibition observed)*100%

The synergistic effect can apply to any of the properties discussed herein (e.g., inhibition CHD1L autoactivation, inhibition of CHD1L ATP-ase activity, decreasing an IC₅₀ of a chemotherapy, inhibition of the growth of cells, such as tumor cells, etc.).

As used herein, “Bliss synergy score” and “synergy score” are used interchangeably. The derivation of a Bliss energy score using the Bliss model is described at De Veroli et al. Bioinformatics, 2016, 32(18), 2866-8, the entirety of which is incorporated herein by this reference. When the synergy score is less than about −10, the interaction between the CHD1L inhibitor and the chemotherapy is likely to be antagonism. When the synergy score is between −10 and 10, the interaction between the CHD1L inhibitor and the chemotherapy is likely to be additivity. When the synergy score is greater than about 10, the interaction between the CHD1L inhibitor and the chemotherapy is likely to be synergy. For example, a synergy score of about 20 corresponds to a behavior of a compound that is about 20% above the expected behavior. In a tumor cell viability assay, a calculated synergy score of 20 indicates the efficacy of the combination of a CHD1L inhibitor and a chemotherapy is 20% more effective at reducing tumor cell viability than expected, thus showing synergy between the CHD1L inhibitor and chemotherapy rather than additive effect.

### NUMBERED EMBODIMENTS

Embodiment 1. A pharmaceutical composition for treating a cancer comprising: a chromodomain helicase/ATPase DNA binding protein 1-like gene (CHD1L) inhibitor; and a chemotherapy.

Embodiment 2. The pharmaceutical composition of Embodiment 1, wherein the chemotherapy comprises a PARP inhibitor.

Embodiment 3. The pharmaceutical composition of Embodiment 1 or Embodiment 2, wherein the chemotherapy comprises irinotecan, olaparib, doxorubicin, docetaxel, AZD5305, or 5-fluorouracil (5-FU), or a combination of two or more thereof.

Embodiment 4. The pharmaceutical composition of any one of Embodiments 1 to 3, wherein the composition comprises irinotecan.

Embodiment 5. The pharmaceutical composition of any one of Embodiments 1 to 4, wherein the chemotherapy is effective against colorectal cancer.

Embodiment 6. The pharmaceutical composition of any one of Embodiments 1 to 5, wherein the chemotherapy is effective against breast cancer.

Embodiment 7. The pharmaceutical composition of Embodiment 1, wherein the chemotherapy compound is effective against breast cancer.

Embodiment 8. A method of treating cancer in a subject in need thereof, the method comprising administering a therapeutically effective amount of a pharmaceutical composition comprising: a CHD1L inhibitor and a chemotherapy.

Embodiment 9. A method of treating cancer in a subject in need thereof, the method comprising administering a therapeutically effective amount of a CHD1L inhibitor, and administering a therapeutically effective amount of a chemotherapy.

Embodiment 10. The method of Embodiment 9, wherein the administering a therapeutically effective amount of a CHD1L inhibitor and the administering a therapeutically effective amount of a chemotherapy occur simultaneously.

Embodiment 11. The method of Embodiment 9, wherein the chemotherapy is not irinotecan.

Embodiment 12. The method of any one of Embodiments 9 to 11, wherein the chemotherapy comprises irinotecan, olaparib, doxorubicin, docetaxel, AZD5305, or 5-fluorouracil (5-FU), or a combination of two or more thereof.

Embodiment 13. The method of any one of Embodiments 9 to 12, wherein the chemotherapy is 5-fluorouracil.

Embodiment 14. The method of Embodiment 13, wherein the method further comprises inhibiting a Wnt signaling pathway.

Embodiment 15. A method of reducing a cytotoxicity of a chemotherapy, the method comprising co-administering the chemotherapy with a CHD1L inhibitor.

Embodiment 16. The method of Embodiment 15, wherein the administering the chemotherapy with a CHD1L decreases an IC₅₀ of the chemotherapy by at least about 100-fold to about 1000-fold.

Embodiment 17. A compound for treating a cancer, wherein the compound binds to an allosteric binding site of CHD1L.

Embodiment 18. The compound of Embodiment 17, wherein the allosteric binding site comprises a lysine and a glutamate.

Embodiment 19. The compound of Embodiment 17, wherein the CHD1L comprises a sequence having at least 80% sequence identity of any one of SEQ ID NOs: 1-40.

Embodiment 20. A method for treating a cancer, comprising administering a compound having the structure:

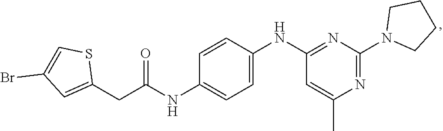

wherein the cancer is a colorectal cancer (CRC), triple negative breast cancer (TNBC), or both.

### EXAMPLES

### Example 1: Compound 6.11 Synergizes with Irinotecan/SN38 and 5FU in Promoting DNA Damage

Compound 6.11 was combined with irinotecan/SN38 or 5FU, measuring the well characterized biomarker of DNA damage (γ-H2AX) by immunofluorescence in SW620 or SUM149PT cells as indicated (FIG. 6). Compound 6.11 alone causes no significant DNA damage compared to vehicle. Compound 6.11 combined with SN38 significantly increased SN38 potency and magnitude of DNA damage compared to SN38 alone in CRC SW620 cells. The percentage of DNA damage for each single agent dose and combination was plotted on a contour map (FIG. 6A). Each shade on the contour map represents a range of % DNA damage where dark orange is 100% and white is 0% DNA damage. For example, the combination of 0.31 μM Compound 6.11 and 31.25 nM SN-38 elicit the same level of DNA damage as 125 nM SN-38 alone, increasing the potency for SN38 when combined with Compound 6.11 at the respective doses. Compound 6.11 combinations with 5FU to determine any synergistic effects was evaluated. As with SN38, the combination of Compound 6.11 and 5FU strongly synergized to induce 5FU-mediated DNA damage (FIG. 6B). The synergy between Compound 6.11 and SN38 or 5FU was measured at various concentrations and calculated using SynergyFinder and the Bliss model, and the synergy scores were used to generate 3D contour plots. Strong synergy (>10 synergy score) with Compound 6.11 when combined with SN38 or 5FU at all doses with a mean score of 24 and 23, respectively, was measured.

### Example 2: Compound 6.11 Synergizes with Irinotecan/SN38 or 5FU to Increase Cytotoxic Potency in CRC Tumor Organoids

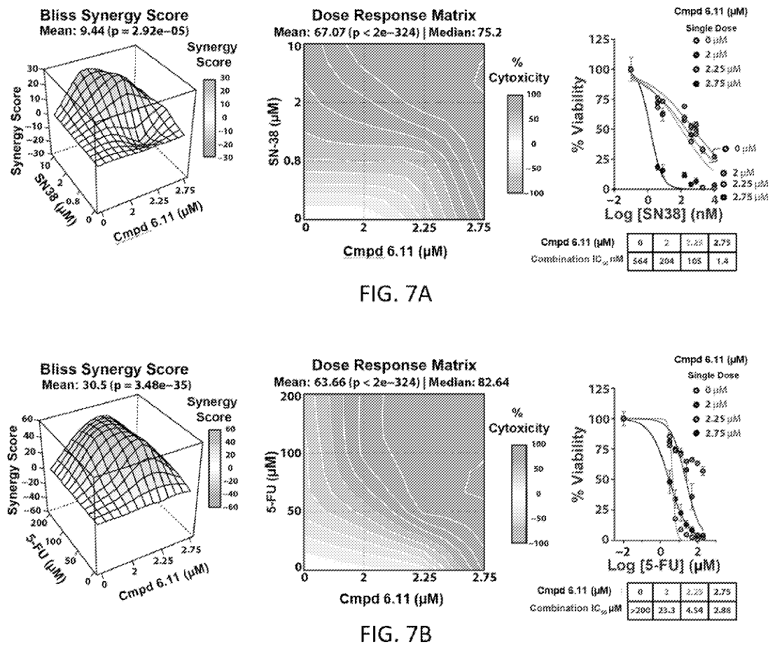

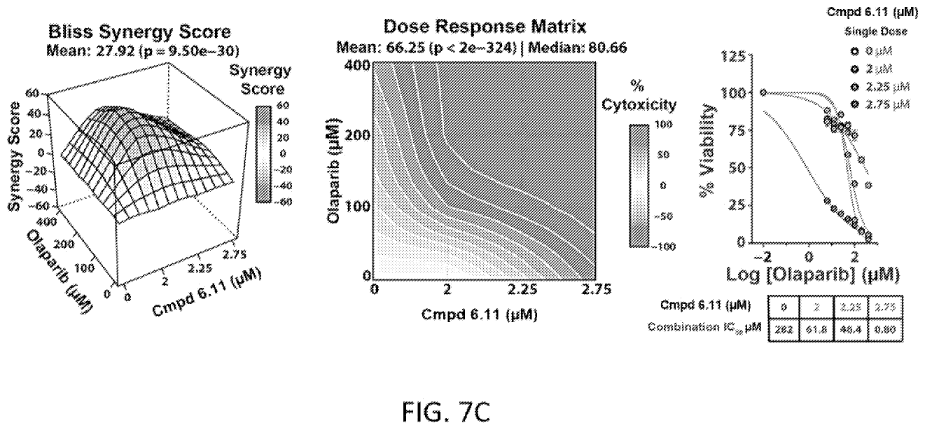

Synergy between Compound 6.11 and SN38 or 5FU at inducing CRC tumor organoid cell death was measured. SW620 cells were cultured as single tumor organoids per well in 96-well plates and treated with drugs for 72 h as reported. The IC₅₀ values were determined for SN38 or 5FU with and without Compound 6.11. The IC₅₀ value for SN38 alone in killing tumor organoids was ~600 nM, but when combined with Compound 6.11 (2.75 μM) strong synergy was measured (IC₅₀ ~1 nM), yielding an ~600-fold increase in cytotoxic potency compared to SN38 alone (FIG. 7A). 5FU alone had modest cytotoxic potency (IC₅₀>200 μM) under the conditions tested, but 5FU combined with Compound 6.11 (2.75 μM) produced strong synergy (~30 synergy score), increasing the IC₅₀ potency of 5FU to 2.88 μM, a 69-fold increase in potency compared to 5FU alone (FIG. 7B).

### Example 3: Compound 6.11 Synergizes with PARPi Olaparib in HR-Proficient Cells Independent of BRCA Mutation Status

PARPi treatment of BRCA1/2 mutant cancers produces a synthetic lethal effect to kill HR-deficient BRCA mutant cancers and that CHD1L is a factor promoting drug resistance to PARPi. CHD1Li synergized with the PARPi olaparib overcoming drug resistance in HCT116 BRCA2 mutant isolated EMT phenotypes (FIG. 5B). PARPi are not clinically used for predominantly HR-proficient cancers such as CRC. Thus, to investigate whether Compound 6.11 can synergize with PARPi HR-proficient tumor cells, combination studies with olaparib, measuring cytotoxicity in SW620 tumor organoids were conducted (FIG. 7C). Like 5FU, olaparib displayed a very high IC₅₀ value of 282 μM in killing SW620 tumor organoids, but Compound 6.11 synergized with olaparib improving the potency to 800 nM IC₅₀. This result is significant as Compound 6.11 enhanced the potency by ~355-fold, demonstrating nM efficacy in HR-proficient cells. Compound 6.11 and other CHD1Li described herein were shown to strongly synergize with olaparib to improve its antitumor potency.

### Example 4: Compound 6.11 Synergizes with Irinotecan/SN38 or 5FU in ATM Mutant CRC Tumor Organoids Overcoming MDR

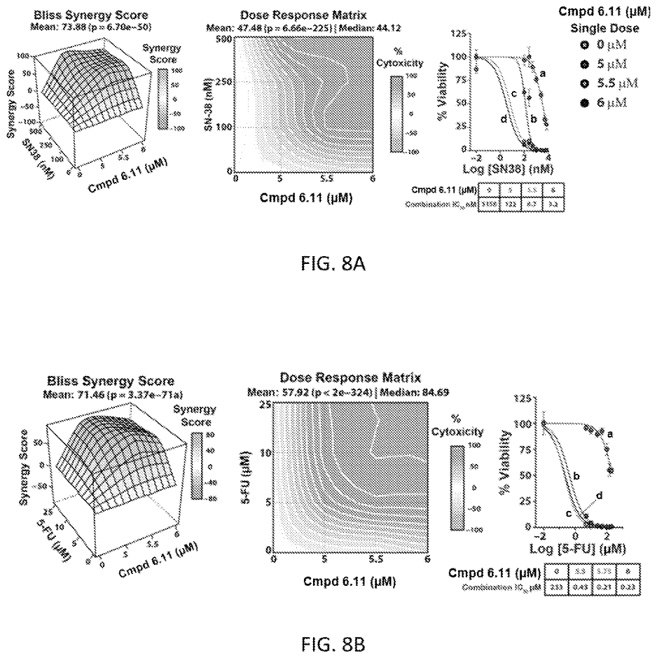

ATM is an important protein involved in the initial stages of the DNA damage response. Cancers with ATM inactivation mutations have been shown to be sensitive and responsive to DNA damaging chemotherapy. ATM mutations are important markers being developed to identify metastatic CRC (mCRC) patients that may be sensitive to DNA damaging chemotherapy. Thus, ATM mutant SW948 CRC tumor organoids were sensitive to irinotecan and potentially 5FU. To our surprise, SW948 tumor organoids displayed drug resistance to SN38 (IC₅₀=3156 nM) (FIG. 8A) compared to ATM wildtype (wt) CRC tumor organoids (IC₅₀=~600 nM) (FIG. 7A). Recent clinical studies demonstrate that mCRC patients with a heterogeneity score (HS)>100 are sensitive to chemotherapy and live significantly longer, but mCRC patients with an ATM mutation and HS<100 responded the same to chemotherapy as wt ATM mCRC cancer patients with no survival benefit. Thus, although SW948 CRC cells are ATM mutant they likely would have a HS<100 based on our results. However, since CHD1L is essential for the DNA damage response and DNA repair and that CHD1Li synergize with other drugs targeting DNA damage response and repair (e.g., PARPi olaparib), and without being bound by any particular theory, Compound 6.11 may elicit a synthetic lethal event with ATM mutant CRC cells. Thus, Compound 6.11 strongly synergized with SN38 and 5FU in ATM mutant SW948 tumor organoids. Compound 6.11 combined with SN38 gave a mean synergy score of ~74 (P<0.0001) and a maximum synergy score of nearly 100, improving the cytotoxic IC₅₀ potency of SN38 by 1,000-fold from 3,156 nM to 3.2 nM (FIG. 8A). Like SN38, Compound 6.11 strongly synergized with 5FU giving a mean synergy score of ~72 with a maximum synergy score of almost 82, while improving 5FU cytotoxic potency by 1,000-fold (FIG. 8B).

### Example 5: 5FU Induces CSC Stemness and MDR in CRC Via the Wnt Signaling Pathway and Compound 6.11 Inhibits this

5FU treatment of CRC cells, tumor organoids, and CRC mouse models induces the activation of the Wnt signaling pathway, promoting CSC cell expansion, MDR, and tumor recurrence. Cho et al also showed that inhibition of the Wnt pathway combined with 5FU suppressed the CSCs and reduced tumor growth. Importantly, Wnt pathway mutations leading to constitutive activation of TCF/LEF, are a well-known driver of CRC tumor progression, metastasis, and MDR. The Wnt/TCF pathway proves to be critical in EMT promotion of mesenchymal cells with increased CSC stemness in cancer.

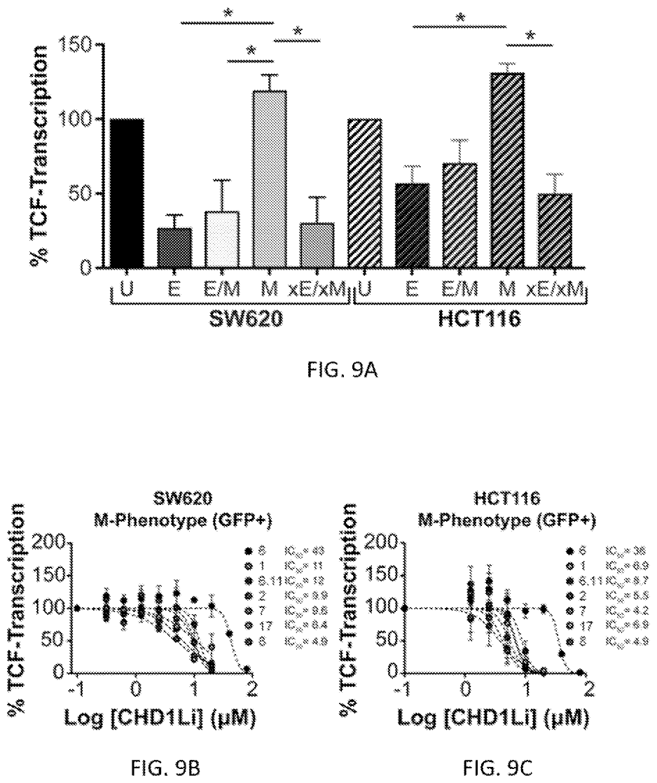

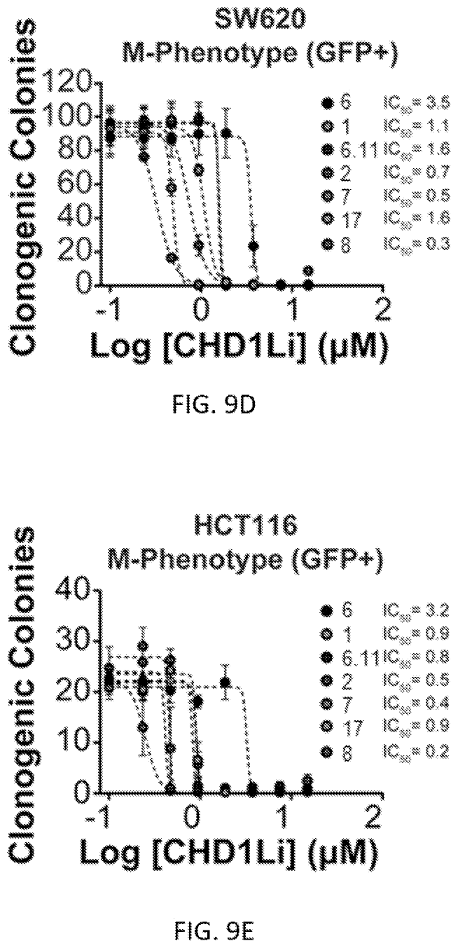

TCF/LEF-transcription acted as a master regulator of EMT and CSCs, particularly in CRC. Based on the results, CHD1L may be a component of the TCF/LEF transcription complex, which promotes a spectrum of quasi EMT phenotypes in CRC that display increased CSC stemness, metastatic potential, and MDR. Knockdown of CHD1L using shRNA or inhibition with CHD1Li induces the reversion of EMT to an epithelial state that is significantly less tumorigenic. In addition, CHD1L/TCF-transcription upregulation in isolated quasi mesenchymal cells (FIG. 9A) was shown, and that these cells have increased CSC stemness. Compound 6.11 and analogs show dose dependent inhibition of CHD1L-mediated TCF-transcription and CSC stemness (FIG. 9B-E). In summary, 5FU treatment upregulates the Wnt signaling pathway to induce EMT and CSCs that promote MDR and tumor recurrence. Inhibiting the Wnt pathway combined with 5FU abrogates expansion of EMT and CSCs to overcome MDR and tumor recurrence. Thus, Compound 6.11 strongly synergizes with 5FU causing CRC tumor organoid death (FIG. 9B), and Compound 6.11 inhibits CHD1L-mediated TCF-transcription to inhibit EMT and CSC stemness. Thus, Compound 6.11 likely synergizes with 5FU by inhibiting DNA synthesis, DNA repair, but also through inhibition of CHD1L-mediated Wnt/TCF-driven EMT and CSCs.

### Example 6: Compound 6.11 Synergizes with Irinotecan In Vivo

Compound 6.11 displays good PK and single agent efficacy in vivo. To confirm the in vitro combination results, in vivo combination studies with Compound 6.11 and irinotecan were conducted to determine if the combination was more effective than irinotecan alone, using irinotecan drug resistant SW620 M-phenotype mouse xenografts (FIGS. 5 & 10). The dose of Compound 6.11 was reduced to 5 mg/kg and administered intraperitoneally (IP) 1×/day, 7-days/week. Irinotecan was also decreased from a recently reported 100 mg/kg effective dose to 50 mg/kg dosed 1×/week. Mice were dosed with Compound 6.11 a week prior to their first irinotecan treatment to prime the animals for chemotherapy. The results indicate that irinotecan, when combined with Compound 6.11, is significantly more effective at inhibiting tumor growth and promoting survival, which increased mouse survival by about a month.

### Example 7: Compound 6.11 Displays No Toxicity in Mice

As a preliminary assessment of Compound 6.11 safety, a separate experiment treating nude mice with Compound 6.11 by oral gavage (PO) 1×/day 7-days/week at 100 mg/kg was conducted. 100 mg/kg is an effective dose for single agent efficacy when administered orally. In addition, mice were treated with irinotecan alone and combined with Compound 6.11. Organs and blood were harvested and sent to the Colorado State University Flint Animal Cancer Center for histopathological analysis by an independent certified veterinary pathologist with expertise in cancer chemotherapy. A complete blood count (CBC) chemistry and histology of liver, heart, and bone marrow tissues show no toxicity with Compound 6.11 alone or combined with irinotecan under tested conditions (FIG. 11).

Compound 6.11 represents CHD1Li and is currently the best-in-class clinical candidate that has the potential to significantly impact the treatment of cancer. Compound 6.11 displays excellent drug-like properties and antitumor efficacy as a single agent. Additionally, Compound 6.11 strongly synergizes with components of FOLFIRI and other targeted clinically relevant cancer drugs (e.g., olaparib). Successful completion of the proposed research will help position Compound 6.11 for the next stage of pre-clinical development, IND-enabling studies to achieve TRL5 level readiness.

### Example 8: Evaluate the Pharmacology & Efficiency of Compound 6.11 Alone and in Combination with FOLFIRI in CRC

Preclinical pharmacological studies in CRC tumor bearing nude mice. Assess toxicology safety and tolerability, including complete blood count (CBC) panel and histology of tissues (brain, heart, bone marrow, intestine, kidney, and liver). Determine the pharmacokinetics (PK) profile of Compound 6.11 alone and combined with each component of FOLFIRI. Analytical measurements include tumor volume, PK, PD maker for DNA damage, and organ toxicity (H&E histology). Toxicological and PK studies will determine the doses of Compound 6.11 and components of FOLFIRI that are safe and tolerable as a combination therapy in mice as well as optimal dosing and schedule.

Antitumor efficacy of Compound 6.11 alone and in combination with FOLFIRI using CRC xenografts. Conduct Compound 6.11 antitumor efficacy studies alone and in combination with each component of FOLFIRI (irinotecan and 5FU+LV) and FOLFIRI using nude mice CRC xenograft models. Analytical measurements include tumor volume by caliper and survival. Determine the efficacy of Compound 6.11 combined with components of FOLFIRI and FOLFIRI in mice.
A) Drug vehicle. Compound 6.11*HCl salt is readily soluble 30% PEG400 in water. Both 5FU/LV are also soluble in water, and irinotecan*HCl is soluble in 50% PEG400 in water. Compound 6.11*HCl and 5FU/LV are also soluble in 50% PEG400 saline solution. Thus, the vehicle that will be used for both phase 1 and phase 2 animal efficacy studies will be 50% PEG400 in sterile water (referred to herein in phase 1 and 2 as vehicle).
B) Toxicity study with Compound 6.11 and FOLFIRI. The safety of Compound 6.11 will be determined in combination with FOLFIRI in mice. The FOLFIRI treatment protocol to be used in mice is shown in Table 3 and was established in mouse colorectal cancer models. All drugs will be administered by intraperitoneal (IP) administration, with the exception of Compound 6.11, which will be dosed daily by oral gavage and carried out in male and female CD-1 mice. Groups will include vehicle, Compound 6.11 at 2 doses (50 and 100 mg/kg), FOLFIRI, FOLFIRI+Compound 6.11 50 mg/kg and FOLFIRI+Compound 6.11 at 100 mg/kg. Animals (8 per group, 4 males/4 females) will be monitored for body weight, body score index, food and water intake and blood will be collected weekly for complete blood count (CBC) analysis during the 4 weeks of drug treatment. Following 4 weeks of treatment, animals will be treated with 200 mg/kg 5-bromo-2′-deoxyuridine (Bardu) by IP injection 4 hours prior to sacrifice. Blood and tissues (brain, heart, bone marrow, intestine, kidney, and liver) will be collected, and samples stored in liquid nitrogen and formalin fixed for immunohistochemistry (IHC). Blood samples will be analyzed for CBC and chemistry panel by the Veterinary Diagnostic Center at Colorado State University. Tissue samples will be processed, and H&E stained for histopathological analysis. Intestine samples will be assessed for proliferation by IHC with anti-Bardu antibody staining as well as analysis by determining the villi length/crypt depth ratio. Safe combinations will be defined as those that do not cause significant changes in continuously measured factors (body weight, body score index, food and water intake, and CBC counts) or tissue specific pathology as compared to FOLFIRI-alone treated mice.

TABLE 3
Dosing Regimens

| Drug | Dose | Frequency |
|---|---|---|
| 5FU | 15 mg/kg | 3x Week |
| Leucovorin (LV) | 20 mg/kg | 3x Week |
| Irinotecan | 45 mg/kg | 2x Week |

C) Pharmacokinetic studies of FOLFIRI and Compound 6.11. To determine any potential drug interaction between the drugs in FOLFIRI and Compound 6.11, a pharmacokinetic (PK) study will be carried out comparing PK parameters of FOLFIRI and Compound 6.11 dosed alone and in combination. FOLFIRI will be dosed as described in Table 3 and Compound 6.11 as described in the toxicity studies at a dose of 50 mg/kg. Following drug dosing, plasma samples will be collected by cardiac stick under isoflurane anesthesia at 0.5-, 1-, 2-, 4-, 8- and 24-hours post-dosing. Each dosing group will include 24 CD-1 mice and include 2 males and 2 females at each time point. Samples will be stored at −80° C. until analysis. Drug levels (5FU, leucovorin, irinotecan/SN38, Compound 6.11) will be measured using validated tandem LCMS assays.

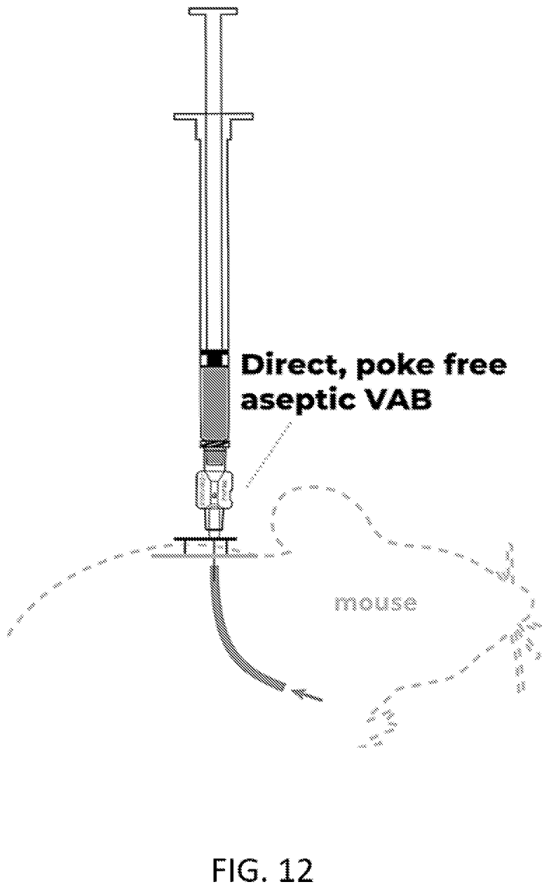

Compound 6.11, FOLFIRI and the combination (Compound 6.11+FOLFIRI) are tolerable in mice at the indicated doses. This is based both on the prior use of this FOLFIRI protocol in mice and our use of irinotecan and Compound 6.11 combinations safely in mice in previous studies (FIGS. 10 and 11) and preliminary studies using daily dosing of Compound 6.11 up to 100 mg/kg in mice with no significant toxicities. There are limited pharmacokinetic interactions between the drugs included in FOLFIRI and Compound 6.11 based on the known metabolic profiles of the agents included in FOLFIRI and microsomal studies carried out on Compound 6.11 metabolism.
D) Antitumor efficacy of Compound 6.11 alone and in combination with FOLFIRI using CRC xenografts. Quasi EMT phenotypes from a variety of cancer cell lines were isolated and demonstrated that isolated mesenchymal cells (M-phenotype) display significantly more tumorigenic and metastatic potential, and these cells are drug resistant to chemotherapy (FIG. 3). SW620 M-phenotype nude mouse xenografts was developed. The SQ620 M-phenotype nude mouse xenografts are relatively resistant to irinotecan alone but synergize with Compound 6.11, inhibiting tumor growth and promoting survival compared to irinotecan alone (FIG. 8).
E) Athymic nude (Foxn1^{nu}) mouse model with catheters and vascularized access button (VAB). Generally, components of FOLFIRI are administered to mice via intraperitoneal (IP) injections. IP injections can be stressful on mice and may cause peritonitis. In addition, FOLFIRI is administered to humans intravenously (IV). To eliminate any issues with IP administration and to better replicate human clinical treatment drugs are administered using nude male and female mice with surgically implanted jugular catheters and VAB ports commercially available from Charles River (FIG. 12).
F) Nude mouse flank xenografts. 48 male and 48 female athymic nude mice with jugular catheters and VABs, will be anesthetized with 3-5% inhaled isoflurane. Mice are inoculated subcutaneously into each flank with a 100 μL solution containing 50 μL RPMI-1640 media, 50 μL Matrigel™, and 2×10⁶ M-phenotype SW620 CRC cells. 7 days after tumor inoculation, mice will be randomized into 8 groups, vehicle, Compound 6.11, irinotecan, LV+5FU, FOLFIRI, Compound 6.11+irinotecan, Compound 6.11+LV-5FU, and Compound 6.11+FOLFIRI. The optimal dosing/schedule for each drug will be determined as above. Treatments will be administered IV through the VABs over the course of four weeks. Mouse weights and general mouse health will be monitored twice a week. Tumor volumes (TV) will be taken with caliper measurements in two dimensions and calculated using the formula TV=(L×W²)/0.5236. Mice will be sacrificed when tumors reach a maximum volume of 2000 mm³ individually, or the left and right tumors reached a combined total volume of 3000 mm³, and survival data will be collected. Mice will also be sacrificed if 15% or more weight loss is observed or other moribund criteria are met, but these mice will be excluded from the survival data.

SW620 M-phenotype mouse model was used as a model to test Compound 6.11 as a single agent and in combination with irinotecan (FIG. 10). Using the optimal dosing and schedule from above, the efficacy of Compound 6.11 alone, in combination with each component of FOLFIRI, and with FOLFIRI is determined. Results provide a Go decision, demonstrating that each drug alone displays antitumor activity but that the combination of Compound 6.11 with each component of FOLFIRI is significantly more effective at reducing tumor volume and increasing survival. Furthermore, Compound 6.11 combined with FOLFIRI is an effective combination. Compound 6.11 combinations may allow a reduction in FOLFIRI dosage while maintaining significant efficacy, which may prove beneficial in clinical use by limiting adverse effects of FOLFIRI.

### Example 9: Determine CRC Patient Populations Response to Compound 6.11 FOLFIRI Combination Therapies

Determine CRC patient populations that may benefit from Compound 6.11 FOLFIRI combinations. Select 8 patient samples with diverse genetic background and stage, including 4 responsive to FOLFIRI and 4 resistant to FOLFIRI. 8 patient samples are expanded in vivo and cultured ex vivo as patient derived tumor organoids (PDO) in 96-well plates arrayed as single PDOs per well. Measure the synergy of Compound 6.11 and FOLFIRI (irinotecan/SN38+5FU) as a triple combination. Analytical measurements include PDO viability, IC₅₀ dose responses, and Bliss synergy scores. Determine CRC patient populations that are sensitive to Compound 6.11+FOLFIRI combinations, including patients that are sensitive or resistant to FOLFIRI as well as early and late-stage metastatic CRC patients.

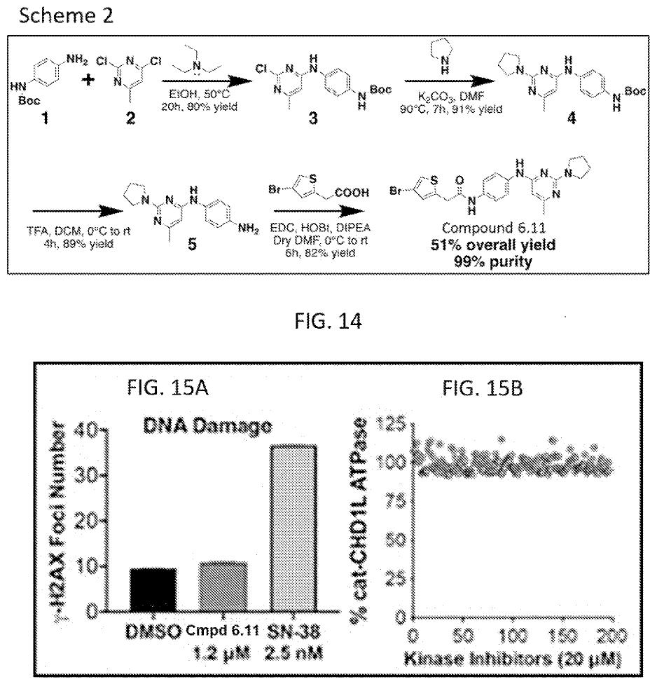

Determine the efficacy of Compound 6.11 FOLFIRI combination in CRC patient derived xenografts (PDX) mouse models. 4 patient samples in which Compound 6.11 FOLFIRI combination had the strongest synergy are expanded. Expanded patient samples are used to generate PDX models to conduct antitumor efficacy studies with Compound 6.11 combined with FOLFIRI. Compound 6.11 may benefit CRC patients if given before, during, and after FOLFIRI treatment. To investigate this, the most responsive PDX from Task 2 are used to test two combination groups: (1) Compound 6.11 before, during, and after FOLFIRI; and (2) Compound 6.11 during FOLFIRI only. Analytical measurements include tumor volume by caliper and survival. Efficacy of Compound 6.11 combinations with FOLFIRI in vivo are determined, while identifying CRC patient population(s) for early phase clinical trials.
A) Patient sample selection for patient derived organoids (PDO) and xenografts (PDX). To determine CRC patient populations that would benefit from Compound 6.11 and Compound 6.11 combined with FOLFIRI, PDO and PDX models were utilized. Currently, the University of Colorado Cancer Center (UCCC) GI tissue bank has >100 annotated IRB-approved prospective consented CRC patient samples with full medical records, including mutation status, stage of cancer, site of tumor sample resection (primary or metastatic), drug resistance, and tumor relapse information. Using this information, 8 patient samples with diverse genetic background mutations, stage of cancer, site of cancer (primary vs metastatic) were initially selected (Table 2). In addition, these samples show a range of sensitivity to irinotecan and 5FU as shown by their tumor growth inhibition index (FIG. 14). Without being bound by any theory, Compound 6.11 will benefit patients at any stage of CRC regardless of responsiveness to FOLFIRI, such as the 8 patient samples selected based on the response to irinotecan and 5FU, including 4 responsive and 4 resistant samples.

TABLE 4
Summary of Patient Sample Data.

| | | | | Muta- | |
|---|---|---|---|---|---|
| Patient | Tumor | Irinotecan | 5FU | tion | T, N, M |
| Sample | Site | Response | Response | Status | Status |
| CRC001 | Primary | 31.4% | 185.8% | KRAS | TX, NX, M1b |
| CRC006 | Metastatic | 78.7% | NA | KRAS, | TX, NX, M1c |
| | | | | APC | |
| CRC026 | Metastatic | 92.7% | 77.3% | NA | TX, NX, M1c |
| CRC040 | Primary | 17.3% | NA | NA | T2, N0, M0 |
| CRC042 | Primary | 104.3% | 107.8% | KRAS, | T3, N0, M1a |
| | | | | TP53 | |
| CRC102 | Metastatic | 12.2% | 113.1% | KRAS | TX, NX, M1c |
| CRC114 | Primary | 57.8% | NA | BRAF | T4a, N0, M0 |
| CRC125 | Metastatic | 58.8% | NA | NA | TX, N0, M1b |

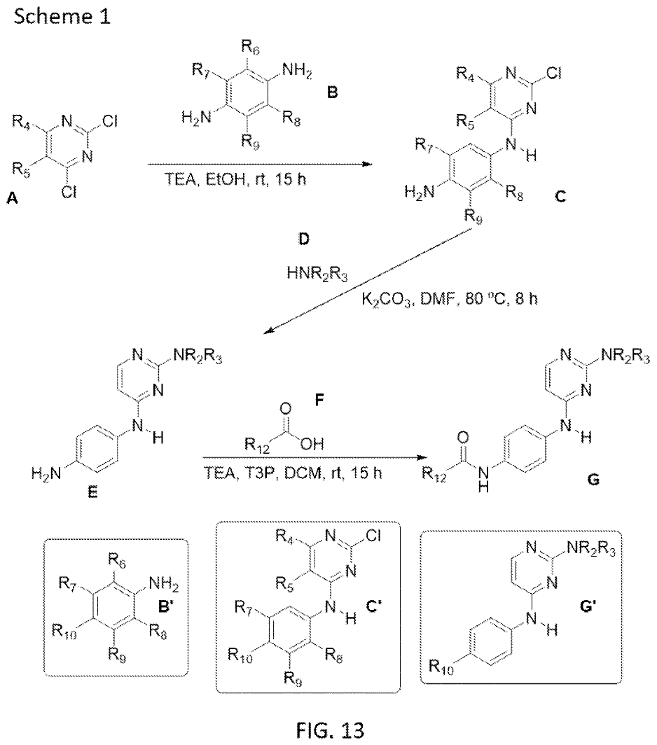

B) Expand 8 CRC patient samples for PDO testing and conduct single agent drug dose response efficacy studies. Viable frozen CRC patient samples “F0” will be expanded as reported by the UCCC GI tissue bank. Briefly, tumors are engrafted into nude mice via trocar subcutaneous injection “F1”, requiring 5 mice/patient sample. When the growing tumors reach 2,000 mm³ they will be harvested, cut into 3 mm³ pieces, and inoculated into “F2” mice for further expansion if needed. Tumor tissue is frozen or processed fresh to generate PDO or PDX models.
C) Automated PDO culture in 96-well plates. Fresh CRC patient samples will be cultured ex vivo as PDOs, modified from reported well established methods. Using a G3 Explorer automation platform (G3) equipped with a PFlex robotic arm and integrated with a Janus G3 liquid handier, centrifuge, cell incubator, and Envision plate reader. Using the Janus, 2,000 patient cells are added to ultra-low attachment (ULA) U-bottom plates in 100 μL of media per well. Plates are transferred to the centrifuge, spun at 1,000 rpm for 15 min to aggregate cells, sent back to the Janus, and coated with 25 μL of 10% Matrigel™ to achieve a final concentration of 2% Matrigel™ and sent to the G3 incubator for 72 h, forming uniform single PDOs per well (FIG. 13).
D) Single agent close responses in PDOs. Compound 6.11, 5FU, LV+5FU (for LV a single optimal dose that is effective with 5FU which will be used for the LV+5FU group is determined), irinotecan/SN38, and FOLFIRI will be tested as single agents with 8 doses over a range tailored to each drug to determine the IC₅₀ value of each drug using the FlexDrop IQ (PerkinElmer) drug printer on the G3 Explorer. The FlexDrop allows for efficient nanoliter and non-contact dispensing combination studies. The DMSO drug solvent concentration will be maintained at 0.5%, which has no effects on PDO viability. Additional doses will be added if needed to achieve a complete dose response. To measure PDO viability, excess media from each well is removed using the Janus, leaving the PDO in 40 μL, followed by the addition of 40 μL of 3D CellTiter-Glo (Promega) and shaken for 45 min. The contents of the well are transferred to white plates and sent to the Envision plate reader to measure luminescence.
E) PDO combination studies with Compound 6.11, each component of FOLFIRI and FOLFIRI. After determining the single agent efficacy for each drug, PDOs as described above will be generated to test the synergy between Compound 6.11, SN38, 5FU, 5FU+LV, and FOLFIRI. For two-drug combinations (Compound 6.11+SN38 and Compound 6.11+5FU) dose responses coupled with SynergyFinder and the Bliss model approach as shown in the preliminary data (FIGS. 6 & 7) are used to determine synergy scores. For more than two-drug combinations (Compound 6.11+5FU+LV and Compound 6.11+FOLFIRI), the IC₅₀ value of each drug is used, and the synergistic effects using the “response additivity approach” model is used. The response additivity approach assumes synergistic effects when the drug combination induces a greater response than the sum of the individual drug effects.

An array of 8 patient samples cultured as PDOs to test the synergy of Compound 6.11 with FOLFIRI and the components of FOLFIRI will be used. The patient samples will be diverse in oncogenic mutations, stage, site of tumor (primary vs metastatic), and sensitivity to FOLFIRI or components of FOLFIRI. Thus, the synergy between Compound 6.11 and each component of FOLFIRI will be determined but also that of FOLFIRI and gain insight into the types of CRC patients that may benefit from Compound 6.11. Based on preliminary data Compound 6.11 will be effective against most of the CRC PDOs and will synergize with FOLFIRI and the components of FOLFIRI. 4 patient samples (2 sensitive and 2 resistant to FOLFIRI) are selected that responded the best to Compound 6.11 and FOLFIRI combinations and confirm these results using PDX models.
F) Expand 4 CRC PDX models. Using the method in Part II and shown (FIG. 15), 4 CRC patient samples (2 sensitive and 2 resistant to FOLFIRI) as PDX models (5 mice/PDX=20 mice) will be included, particularly those that are the most responsive to Compound 6.11+FOLFIRI combinations in PDO studies.
G) In vivo antitumor efficacy studies with Compound 6.11 and FOLFIRI. Expanded patient tumors will be used to generate the 4 CRC PDX models, using nude mice with catheters and VAB as described above. These models will be used to validate the efficacy of Compound 6.11 combined with FOLFIRI. The 4 PDX models will be comprised of 4 groups per PDX model and will be randomized when tumors become palpable (~150 mm³), including vehicle, Compound 6.11, FOLFIRI, Compound 6.11+FOLFIRI. Considering a take rate of 70%, the study begins with 48 mice/PDX model, giving 12 mice per group for a total of 192 mice. All drugs will be administered IV through the VAB port. The optimal dose and scheduling for all drugs will be. Drug treatments will begin on day 14 and continue for 4-6 weeks. Mouse weights and general mouse health will be monitored twice a week. Tumor volume (TV) will be measured with calipers in two dimensions and calculated using the formula TV=(L×W²)/0.5236. Mice will be sacrificed under the criteria described above under Example 8(D).
H) Compound 6.11 pre-, concurrent-, and post-treatment PDX studies with FOLFIRI. Without being bound by any theory, Compound 6.11 may benefit CRC patients if given before, during, and after FOLFIRI treatment. To test this, the most responsive PDX model from Example 9(B) will be used to test 5 groups, including vehicle, Compound 6.11 concurrent treatment with FOLFIRI, and Compound 6.11 pre-, concurrent-, and post-treatment with FOLFIRI. Based on a 70% take rate, 12 mice per group, for a total of 36 mice, will be used. TV, survival, and animal health will be measured as in Example 9(B).

An array of 4 patient samples is used to generate PDX models to validate the synergy of Compound 6.11 with FOLFIRI and the components of FOLFIRI in vivo. Validation of the preliminary results and the PDO model synergy results using in vivo PDX models will provide strong rationale for further preclinical development.

### Example 10: Scale-Up Synthesis of CHD1Li

Optimizing the scale-up synthesis of CHD1Li will save in overall cost and time prior to outsourcing to a contract research organization (CRC) for GMP manufacturing. This synthesis was scaled up to achieve a 5 g scale in high overall yield (51%) and purity (99%) (Scheme 1, FIG. 13).

Method A: Synthesis of CHDLi Compounds. CHDLi compounds and derivatives thereof were prepared, for example, by the method illustrated in Scheme 1 (FIG. 13), as described in U.S. patent application Ser. No. 17/953,221, which is incorporated by reference herein in its entirety. Briefly, this three-step synthesis starts with selective aromatic nucleophilic substitution on the 4-position of a 2,4-dichloro-pyrimidine A (e.g., 2,4-dichloro-6-methylpyrimidine, where R₄ is methyl or 2,4-dichloro-5-fluoropyrimidine, where R₅ is fluorine) with a p-phenylenediamine B to form the intermediate C. Exemplary reaction conditions are shown in Scheme 1 where reactants are added with trimethylamine to ice cold ethanol and stirred at rt for 15 h. Chlorinated intermediate C is then reacted with any amine HNR₂R₃(D) by amination to generate intermediate E. Exemplary amination conditions are shown in Scheme 1, where reactants are reacted in DMF in the presence of K₂CO₃ at elevated temperature. Step three couples the R₁₀ group employing acid F to intermediate E. Various known synthetic methods can be employed to introduce a selected R₁₀ group, for example, cross coupling, click chemistry or substation reactions (e.g., SN2, aromatic, electrophilic). Scheme 1 illustrates coupling of the amine group of E with a selected carboxylic acid F to form R₁₀ which is —NH—CO—R₁₂ in compound G. Exemplary R₁₂ are aryl, aryl-substituted alkyl, heteroaryl and heteroaryl-substituted alkyl. Exemplary coupling conditions are illustrated in Scheme 5, where coupling proceeds in the presence of propylphosphonic anhydride (T3P) and triethylamine at room temperature to form the desired compound G. The illustrated method has been employed, for example, to prepare Compound 6.

Various substituted starting materials A, B, D and F are commercially available or can be prepared using known methods. In embodiments, aniline derivatives already substituted with R₁₀ (B′) can be used in place of p-phenylenediamine derivatives B to form a corresponding R₁₀-substituted intermediate C′. Carrying out step 2 of the illustrated reaction, by reacting intermediate C′ with D will result in desired corresponding compound G′ (where R₁₀ replaces R₁₂—CO—NH—). As will be appreciated by one of ordinary skill in the art, it may be useful to protect certain groups in the starting materials or intermediates during reactions shown to prevent undesired side-reactions. For example, ring N in reactants F may be protected with appropriate amine protecting groups. Use of appropriate protecting groups is generally routine in the art. A variety of primary or secondary amines (D) are commercially available or can be prepared by well-known methods. Alternatively, chlorinated intermediate C can be reacted with an appropriate nucleophile to add a selected —NR₂R₃ group at the 4-chloro position. For example, D can be a cyclic amine such as pyrrolidine. As another possible alternative, Suzuki coupling may be used to install an amine containing group by C—C bond formation. As another possible alternative, Buchwald-Hartwig cross coupling can be used to form carbon and amine bonds in such intermediates.

Method B: Synthesis of Compound 6.11. 50 g of N-boc-p-phenylenediamine 1 (240 mmol) will be added to a solution of 2,4-dichloro-6-methylpyrimidine 2, (39.12 g, 1 eq) and triethylamine (67 mL, 2 eq) in 250 mL absolute ethanol. After heating at 50° C. for 20 h, the reaction mixture is concentrated, and the precipitate is vacuum filtered and washed repeatedly with cold water to afford 3 as an off-white solid. Next, pyrrolidine (5 eq) is added to a solution of 3 (64 g, 190 mmol) and potassium carbonate (28 g, 1.05 eq) in 100 mL of DMF followed by heating the reaction mixture at 90° C. for 7 h. The cooled mixture is poured in an ice slurry and the resulting crystalline product is filtered and washed with ice cold water to give 4 in 91% as an off-white solid. The intermediate 4 (63 g, 171 mmol) was Boc deprotected to the free aniline 5 by treating with 50 mL trifluoroacetic acid (TFA) in dry DCM (100 mL) with stirring at 0° C. for 30 min then at room temperature for 4 h. The DCM is removed in vacuo followed by basifying the residue with saturated sodium bicarbonate solution. The resulting precipitate is filtered, dried, and recrystallized from ethyl acetate-diethyl ether to give 5 as cream-colored crystals in 89% yield. Finally, Compound 6.11 is prepared from amide coupling of 5 (40 g, 149 mmol) with 4-bromothiophene-2-acetic acid (1.2 eq) using 1.5 eq each of the coupling reagents EDC, HOBt and DIPEA in 100 mL of dry DMF under nitrogen. The reaction is poured over crushed ice, filtered, and crystalized from boiling ethanol adding a minimum amount of hot water to afford 57.7 g of Compound 6.11 in 82% yield, with an overall yield from 1 of 51% that is 99% pure by HPLC.

The 4-step synthesis of Compound 6.11 is simple and high yielding. There may be some challenges with the kilogram scale-up based on the partial water solubility of intermediates 3 and 5 in the aqueous workup. For example, and to our surprise, it was discovered from preparing Compound 6.11 analogs that intermediate 5 (Scheme 2) readily crystalizes from ethyl acetate/diethyl ether. Intermediate 5 may be crystallized or purified using a Teledyne Combiflash chromatography system. The order of synthetic intermediates is versatile in preparing Compound 6.11 and alternative approaches can be used. For example, the bromothiophene starting material can first be coupled to phenylenediamine 1, followed by Boc deprotection and reaction to the dichloropyrimidine 2. Importantly, these modifications will not add cost or extra steps to the synthesis compared to Scheme 1.

### Example 11: Binding of CHD1L Inhibitors to an Allosteric Binding Site in CHD1L

The mechanism of action (MOA) of Compound 6.11. Compound 6.11 demonstrated oral bioavailability (8 h half-life), achieved effective blood concentrations, and exhibits potent anti-tumor effects in mouse tumor xenografts. Furthermore, Compound 6.11 synergized with BC and CRC SOC in vitro and in vivo. Without being bound by any particular theory, the ability of CHD1L inhibitors to inhibit CHD1L and induce PARthanatos in tumor cells is presented in FIG. 4.

Compound 6.11 is an allosteric inhibitor that traps CHD1L onto chromatin. Compound 6.11 may bind to the allosteric site in CHD1L's C-ATPase domain (FIGS. 1A & 1B), supported by enzyme inhibition studies using cat-CHD1L (missing the macro domain) and molecular dynamics modeling. The N-ATPase domain houses the sole active ATP binding site, responsible for ATPase activity. In our structure-activity relationship (SAR) investigations, the aniline/pyrimidine was identified as a H-bond donor and pi-cation acceptor with Lys-272 (FIG. 1C). This interaction firmly anchors Compound 6.11 in the allosteric site, leading to potent nM dose-dependent inhibition of cat-CHD1L ATPase (FIG. 1D). Substituting the aniline with groups that disrupt H-bond interactions results in a loss of Compound 6.11's inhibitory activity. Additionally, a Lys-to-Ala (K272A) mutation was introduced, which preserves ATPase function but abolishes Compound 6.11's inhibitory activity (FIG. 1E). This data suggests Compound 6.11 binds to CHD1L's C-ATPase allosteric site. Compound 6.11 traps CHD1L onto nucleosomes using in situ subcellular fractionation, quantified through immunofluorescence. Compound 6.11 shows a dose-dependent increase in CHD1L trapping in BC and CRC (FIGS. 1F&1G). Conversely, SOC drugs for BC and CRC, such as olaparib (PARPi), Irinotecan/SN-38, and doxorubicin, do not trap CHD1L or inhibit CHD1L ATPase activity.

Mitigation of potential off-target effects. CHD1L's chromatin remodeling plays a vital role in DNA repair by exposing damage sites to repair proteins. Consequently, CHD1L's chromatin remodeling promotes MDR against DNA-damaging chemotherapy and PARPi. PARPi's anti-tumor effect relies on DNA damage through the trapping of PARP onto DNA. CHD1L's nucleosome sliding reduces PARP trapping, facilitating DNA repair and MDR. In our preliminary data, Compound 6.11 enhanced DNA damage and cell death in BC and CRC cells when used in combination with chemotherapy and PARPi. Despite trapping CHD1L onto chromatin, Compound 6.11 alone does not induce DNA damage (FIG. 15A). Kinase inhibitors are crucial in cancer and other diseases. Many kinase inhibitors share structural features, leading to indiscriminate off-target kinase inhibition due to conserved binding sites among kinases. Given that CHD1L is an ATPase, ~200 kinase inhibitors, including clinical drugs, for their ability to inhibit CHD1L ATPase were examined. Our findings reveal that none of these kinase inhibitors can effectively inhibit CHD1L ATPase even at a high concentration of 20 μM (FIG. 15B). Consequently, the ATP binding site and other binding sites in CHD1L are distinct, confirming that Compound 6.11's potent allosteric inhibition of CHD1L ATPase is selective, mitigating concerns about potential off-target interactions with cellular kinases.

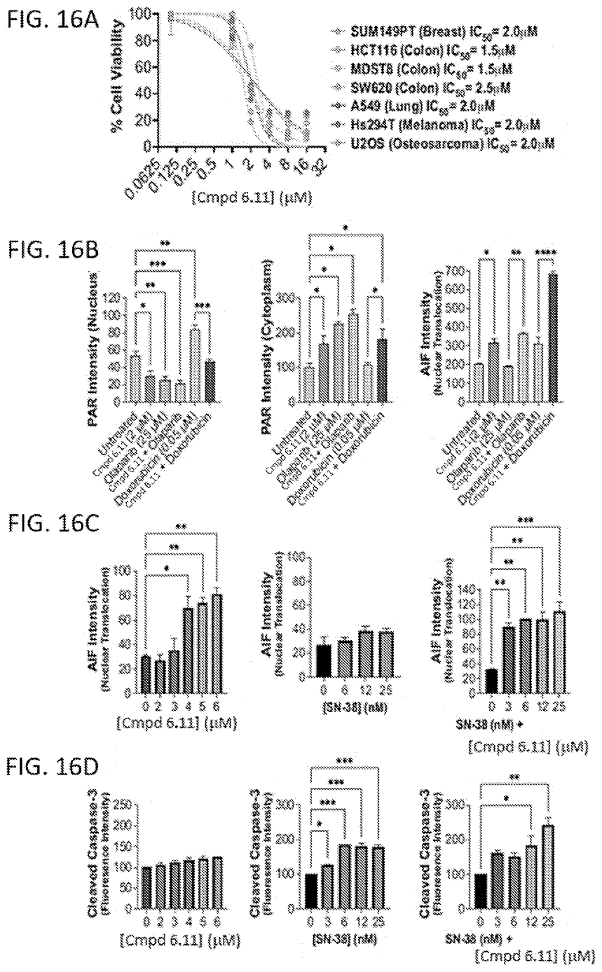

Compound 6.11 induces PARthanatos programmed cell death to kill BC & CRC cells. Compound 6.11 and other CHD1Li are potent inducers of cancer cell death (FIG. 16A). CHD1L is a master regulator of poly-ADP-ribose (PAR) mediated programmed cell death (PARthanatos) (FIGS. 16B&C). PARthanatos, is marked by PAR fragments that localize in cytoplasm, which releases apoptosis-inducing factor (AIF) from mitochondria followed by nuclear translocation and tumor cell death, as illustrated in FIG. 4 (top). CHD1L binds to PAR chains in the nucleus, protecting them from becoming fragmented. Without being bound by any particular theory, CHD1L may suppress PARthanatos to facilitate DNA repair and tumor cell survival. To determine this, PAR distribution in the cytoplasm and nucleus was measured. Both Compound 6.11 and olaparib increased cytoplasmic PAR, with their combination enhancing this effect. Doxorubicin did not elevate cytoplasmic PAR, but it increased nuclear PAR, which signals tumor cells to repair DNA and is a likely cause of MDR to doxorubicin. However, when combined with Compound 6.11, doxorubicin treatment shifted PAR to the cytoplasm, counteracting DNA repair and MDR.

AIF in the cytoplasm and nucleus was examined. Compound 6.11 did not affect cytoplasmic AIF but increased nuclear AIF alone and combined with olaparib, implying CHD1L's role in regulating PARthanatos. Interestingly, while olaparib increased cytoplasmic AIF, it did not enhance nuclear AIF, suggesting that PARP1/2 regulation of PAR is insufficient to trigger PARthanatos. Compound 6.11 combined with doxorubicin or irinotecan/SN-38 also resulted in AIF nuclear translocation. Finally, caspase-3 cleavage activation, an apoptosis marker, was measured (FIG. 16D). Compound 6.11 did not induce caspase-3 activation, but irinotecan/SN-38 did activate caspase-3, consistent with its known MOA. However, Compound 6.11 combined with SN-38 enhanced caspase-3 activation. In summary, Compound 6.11 promotes PARthanatos, displaying a unique MOA, which synergizes with chemotherapy and PARPi to enhance their efficacy and potency in killing BC and CRC tumor cells.

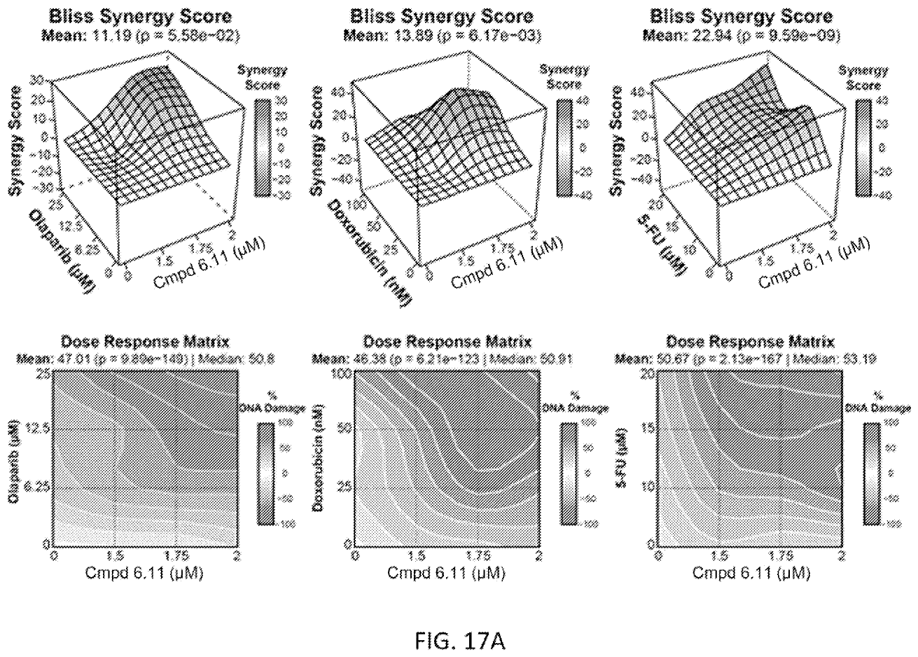

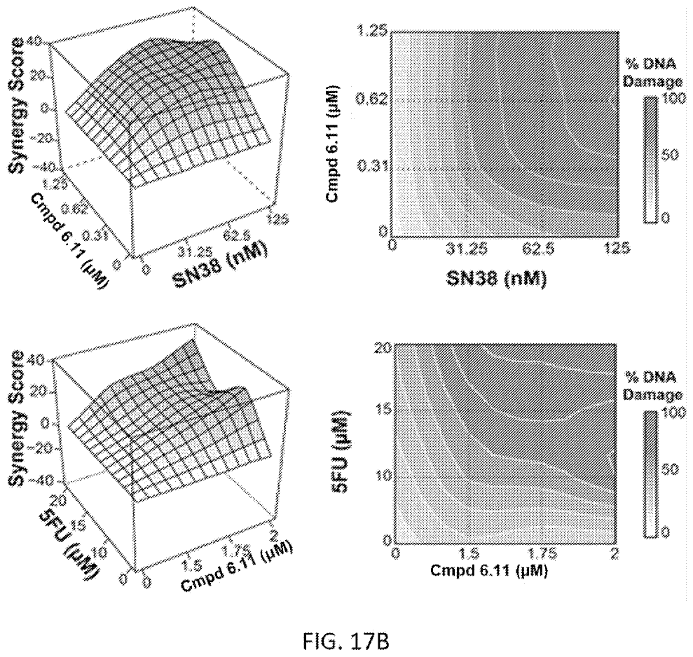

Compound 6 synergizes with SOC to enhance DNA damage. CHD1L plays an essential role in DNA damage response/repair (DDR) that promotes MDR. The data show synergy at enhancing DNA damage between Compound 6 and SOC for BC and CRC, including SN38, doxorubicin, 5FU, and olaparib. DNA damage was measured by immunofluorescent imaging, quantifying g-H2AX foci in SUM149PT and SW620 cells (FIG. 17). Synergy was measured using SynergyFinder and the Bliss model. This synergy is attributed to Compound 6's ability to trap CHD1L onto chromatin, preventing its chromatin remodeling and access to DNA damage sites by repair proteins.

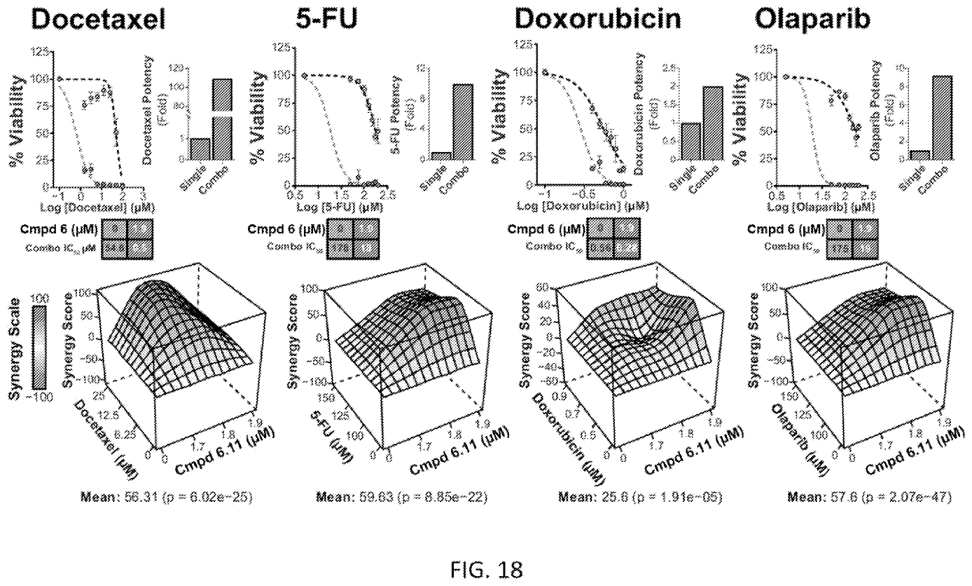

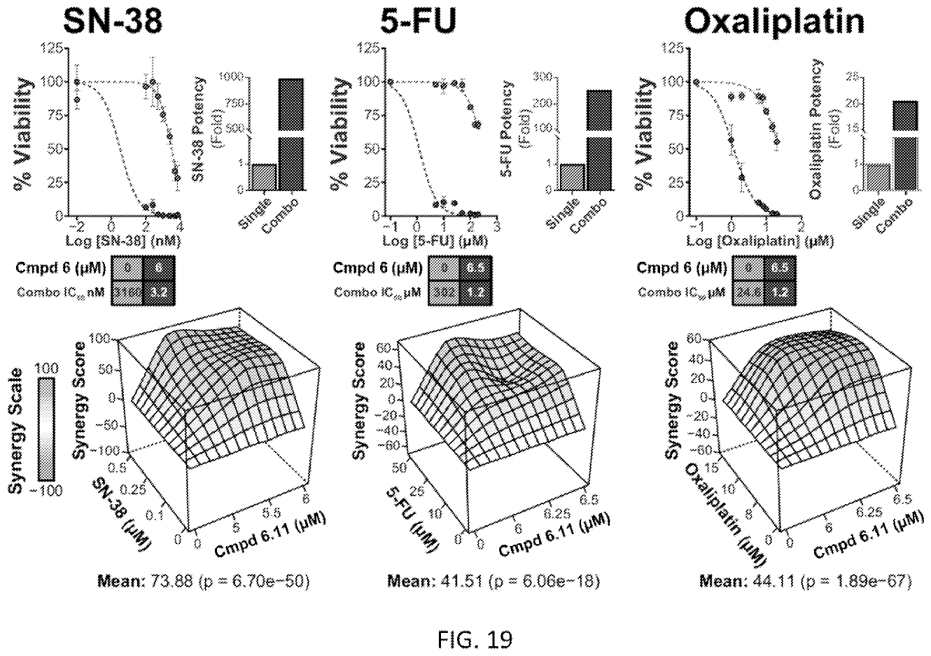

Compound 6 synergizes with SOC to enhance tumor organoid death. Compound 6 combined with docetaxel, 5-FU, doxorubicin, or olaparib synergized to induce SUM149PT BRCA1 mutant BC tumor organoid death (FIG. 18). Synergy was assessed using the Bliss model. A dose-dependent synergy was observed, with the strongest synergy occurring at 1.9 μM Compound 6. This resulted in a 10-fold increase in potency with Olaparib and 5-FU. A 20-fold increase in potency was observed with doxorubicin. Compound 6 combined with docetaxel provided a 110-fold increase in potency from 55 μM to 500 nM IC50. Next, Compound 6 was combined with SN-38, 5-FU, or oxaliplatin which also synergized to induce SW948 chemo resistant CRC tumor organoid cell death. (FIG. 19). The IC₅₀ value for SN-38 alone in killing tumor organoids was ~3 μM but combined with Compound 6 strong synergy was measured (IC50 ~3 nM), yielding a 1000-fold increase in cytotoxic potency compared to SN-38 alone. 5-FU alone had modest cytotoxicity (IC50 302 μM) but combinations with Compound 6 produced strong synergy, increasing the IC₅₀ potency of 5-FU to 1.2 μM, a nearly 300-fold increase in potency compared to 5-FU alone. Finally, Compound 6 enhanced oxaliplatin's potency by 20-fold. Altogether, Compound 6 is a versatile antitumor agent that strongly synergizes with SOC chemotherapy increasing their potency by tens of hundreds of fold.

### Example 12: Compound 6.11 for the Treatment of Breast Cancer and Ability to Induce PARthanatos by Trapping CHD1L and PARP onto Nucleosomes

Cell lines—SUM149PT, HCC1937 and MDA-MB-231 cell lines were purchased from ATCC or CU Anschutz Cell Technologies Shared Resource and were short-tandem repeat (STR)-profiled and mycoplasma-tested before use. SUM149PT cells were maintained in F12/Glutamax (Gibco) medium supplemented with 5% fetal bovine serum (FBS), 10 mM HEPES, 1 μg/mL hydrocortisone and 5 μg/mL insulin. HCC1937 and MDA-MB-231 cells were maintained in RPMI-1640 medium supplemented with 10% or 5% FBS, respectively. All the cell lines were kept in a humidified incubator at 37° C. and 5% CO₂.

Tumor organoid culture—Cell lines were cultured as tumor organoids by seeding 2,000 to 5,000 cells/well into uncoated 96 well U-bottom Ultra Low Attachment Microplates (Corning) using their corresponding medium. Cell aggregation was then promoted by centrifugation of the plates for 15 min at 1,000 rpm followed by the addition of Matrigel (Corning) to a 2-5% final concentration. Tumor organoids were maintained under standard cell culture conditions for 72 h prior to drug treatment.

Tumor organoid cytotoxicity—to assess cytotoxicity in tumor organoids, these were cultured as described before and treated for 72-96 h with the Compound 6.11 combined with olaparib, 5-fluorouracil (5-FU), doxorubicin, AZD5305 and docetaxel. Organoids were then transferred to a white solid bottom 96 well plate and incubated with an equal volume of CellTiter Glo 3D reagent (Promega) for 45 min on a shaker at 400 rpm. Luminescence was recorded using the EnVision plate reader (PerkinElmer) and values normalized to the controls using GraphPad Prism.

Immunofluorescence—DNA damage, PARylation and AIF translocation were assessed by immunofluorescence. SUM149PT cells were seeded into a 96 well PhenoPlate (PerkinElmer) and allowed to adhere overnight. Cells were then treated with Compound 6 or Compound 6.11 in combination with olaparib, 5-FLU, doxorubicin or AZD5305 for different timepoints. The medium was then aspirated, and cells were fixed with 4% paraformaldehyde for 15 min at room temperature (RT). After washing twice with PBS, cells were incubated with 0.3% Triton X-100 for 30 min followed by another washing step. To decrease unspecific binding of the antibodies, cells were blocked in 5% BSA or milk for 30 min at RT. Afterwards, cells were washed twice with PBS and incubated with the correspondent primary antibody with the following indicated dilutions, timepoints, and temperatures: anti-γH2AX, 1:400, overnight (O/N), 4° C.; anti-PAR, 1:1000, 2 h, RT; or anti-AIF, 1:100, O/N, 4° C. Cells were washed again and incubated with secondary antibody for 1-2 h at RT (goat anti-mouse or anti-rabbit AlexaFluor 647, 1:400). Finally, cells were washed, stained with Hoechst 33342 (1:1000), and imaged using a 40× water objective on the Phenix Plus High-Content Screening (ICS) System (PerkinElmer).

PARP1/2 and CHD1L trapping—PARP1, PARP2 and CHD1L trapping were assessed. SUM149PT cells were plated at a density of 20,000 cells per well into clear bottom black 96-well plates (PerkinElmer). Compound 6, olaparib, AZD5305 and doxorubicin were added at different doses and methyl methanesulfonate (MMS) to a final concentration of 0.01%. After 4 h incubation, cell media was aspirated and cells were treated for 10 min at 4° C. with cold cytoskeleton (CSK) buffer (10 mM PIPES pH=6.8, 300 mM sucrose, 200 mM NaCl, 3 mM MgCl₂) supplemented with 0.6% Triton X-100. Then, cells were washed with cold PBS and fixed for 15 min at −20° C. with ice-cold methanol, followed by incubation with blocking solution (5% BSA in PBS) for 1 h at RT. PARP1 (anti-PARP1 antibody, 1:2000, O/N, 4° C.), PARP2 (anti-PARP2, 1:1000, O/N, 4° C.) and CHD1L (anti-CHD1L, 1:1000, O/N, 4° C.) primary antibodies were then added in antibody dilution buffer (2% BSA in PBS) and incubated O/N at 4° C. Afterwards, cells were incubated with secondary antibodies (anti-mouse AlexaFluor 488 or anti-rabbit AlexaFluor 647) for 1 h at room temperature. Finally, cells were washed, stained with Hoechst 33342 (1:1000), and imaged using a 40× water objective on the Phenix Plus HCS System (PerkinElmer).

Cell cycle analysis by flow cytometry—to study the effect of Compound 6.11 and its combination with chemotherapeutic agents on cell cycle progression, 250,000 cells were seeded in 6 well plates and allowed to attach overnight. Cells were treated with different doses of Compound 6.11, olaparib, 5-FU or docetaxel and with their combinations for 24 h. Cells were then harvested by trypsinization, washed with PBS and fixed with 70% ice-cold ethanol for at least 2 h. The fixed cells were centrifuged at 300 g for 10 min, resuspended in PBS, centrifuged again, and stained in a 0.5 μg/mL DAPI solution in 0.1% Triton X-100-PBS for 30 min at RT. Samples were measured on a NovoCyte Penteon Cytometer (Agilent) and cell cycle distribution of single cells was analyzed using the automatic settings of FlowJo V10 (BD Biosciences).

CHD1L synthesis and purification—full-length (FL) CHD1L (residues 16-879 of SEQ ID NO: 1) and catalytic (cat) CHD1L (residues 16-619 of SEQ ID NO: 1) were expressed in Rosetta 2 (DE3) pLysS cells. LB/agar plates with kanamycin and chloramphenicol were streaked with 5 μL of a previous glycerol stock for 24 h. Primary cultures were made from plate colonies for 7 h in 100 mL LB broth with 34 μg/mL Chloramphenicol and 50 μg/mL Kanamycin A sulfate. Secondary cultures were grown in 2 L Terrific Broth with 34 μg/mL Chloramphenicol and 50 μg/mL Kanamycin A sulfate to OD⁶⁰⁰=1.6 (6 h). Cultures were induced with 0.5 mM IPTG at 18° C. for 18 h. Cells were harvested at 4,400 rpm for 40 minutes and frozen at −20° C. for 16 h. Frozen pellets were resuspended in a 4:1 mL/g ratio with Buffer 1A, comprised of 20 mM HEPES, pH 7.5, 500 mM NaCl, 50 mM KCl, 20 mM imidazole, 20 mM MgCl₂, 1 mM TCEP, 10% glycerol, and 500 μM PMSF. Resuspension occurred using a 1 kW La Reveuse smoothie blender. Cells were lysed by sonication, and cellular debris was removed by centrifugation at 15,000 rpm for 1 h. Supernatant was collected and refrigerated at 4° C. overnight before clarification by centrifugation at 15000 rpm for 45 min.

Supernatant was loaded onto an 8 mL Ni-NTA Qiagen resin column. Bound protein was washed with 100 mL Buffer A, 500 mL Buffer 1C (buffer A+10 mM ATP), then 100 mL Buffer A. Elution occurred using Buffer 1B (buffer A+500 mM imidazole) across a 20-500 mM imidazole gradient for 100 mL. Following Ni-NTA affinity purification, protein was dialyzed overnight against 4 L of Buffer 2A-cat comprised of 50 mM Tris, pH 7.5, 200 mM NaCl, 10% glycerol, and 1 mM DTT or 2A-FL comprised of 20 mM MES, pH 6.0, 300 mM NaCl, 10% glycerol, and 1 mM DTT.

Dialyzed protein was loaded onto 2×1 mL subtractive Q-FF Sepharose columns in addition to an 8 mL SP-FF Sepharose column. Bound protein was washed with 100 mL Buffer 2A-FL or 2A-cat. Subtractive Q-FF columns were then removed, after which elution occurred using Buffer 2B-FL (buffer 2A-FL+0.8 M NaCl) or Buffer 2B-cat (buffer 2A-cat+0.8 M NaCl) across a 100 mL gradient of 0.3-1.1 M NaCl. Following ion exchange purification, protein was concentrated to 5 mL using an Amicon Ultra centrifugal device.

Concentrated protein was loaded onto a HiLoad 26/600 Superdex 200 prep grade size exclusion column at 1 mL/min. Mobile phase used was Buffer 3A, comprised of 20 mM HEPES, 100 mM NaCl, 1 mM TCEP, pH 7.5, and 10% glycerol. After size exclusion purification, protein was pooled and concentrated for storage.

Nucleosome remodeling FRET assay—to study the CHD1L-mediated nucleosome remodeling and its inhibition by Compound 6.11 a Fluorescence Resonance Energy Transfer (FRET) assay was used. For that, mononucleosomes composed of a human histone octamer containing a Cy5-tagged H2A, wrapped by a Cy3-labelled DNA template (EpiCypher, Cat #16-4201) were used. The 10 μL reactions were prepared in black low volume 384-well microplates by mixing 20 nM of mononucleosomes, 20 nM of full length CHD1L, pre-incubated or not, for 10 min at 37° C. with Compound 6.11, 80 nM PARP1 (Abcam, ab123934) pre-incubated at 37° C. for 5 min with NAD⁺ and 2 mM ATP. Right after the addition of ATP the assay was monitored overtime using Cy3 excitation (531 nm) and simultaneous detection of Cy3 (595 nm) and Cy5 (685 nm) emission signals using an EnVision plate reader (PerkinElmer). Data are expressed as the ratio of Cy3/Cy5 signal overtime.

PARP1/2 enzymatic assay—the effect of Compound 6.11 and olaparib on PARP1 and PARP2 enzyme activity was tested using the PARP1/2 Colorimetric Assay Kit (BPS Bioscience, Cat #BPS-80580 or Cat #BPS-80581). First, a 96 well plate was coated with histones. Next, the biotinylated NAD⁺ substrate was incubated with an activated DNA template, the inhibitor to test and the PARP1/2 enzyme for 1 h at RT. After that, the sample plate was treated with streptavidin-RP for 30 min at RT, followed by addition of the horseradish peroxidase (HRP) substrate until the colorimetric signal was developed. The EnVision plate reader (PerkinElmer) was used to measure the absorbance at 450 nm and the results were plotted and analyzed using GraphPad Prism.

CHD1L ATPase assay measured by ADP-Glo assay—all reactions were carried out using white opaque 384 well microplates (PerkinElmer, Cat. No. 6007290). 25 nmol/L cat-CHD1L was preincubated with various doses of the inhibitor at 37° C. for 10 min before all other assay components were added to initiate the reaction. No enzyme control was used to measure the background. The reaction components include 200 nmol/L mononucleosome (Active Motif, Cat. No. 81770), 2 mmol/L DTT, and 10 μmol/L ATP (Promega) in a buffer containing 50 mmol/L Tris pH 7.5, 50 mmol/L NaCl, 5 mmol/L MgCl₂, and 5% glycerol to a total volume of 5 μL per well with 4 replicate wells per condition. The reaction was incubated at 37° C. for 1 h. The assay plate was brought to RT before 5 μL of ADP-glo™ Reagent (Promega, Cat. No. V9102) was added to remove unreacted ATP. The ATP depletion step was carried out at RT for 40 min before 10 μL of Kinase Detection Reagent was added and incubated for 30-60 min to convert the ADP produced from the enzyme assay back to ATP for luciferase rection to convert to light for detection. An EnVision plate reader (PerkinElmer) was used to detect luminescence from the ADP-glo assay and the enzyme activity was determined after background subtraction and normalization to enzyme only control.

Phosphatidylserine externalization and membrane integrity—phosphatidylserine externalization and membrane integrity kinetics were analyzed using the RealTime-Glo™ Annexin V Apoptosis and Necrosis Assay (Promega, JA1012) according to the manufacturer's instructions. 8,000 cells were seeded per well in 96 well solid bottom white plates (Corning) in 100 μL and allowed to attach overnight. Then, 75 μL of media were removed, followed by the addition of 25 μL of the test compounds at 4× desired final concentration and 50 μL of the 2× detection reagent. Luminescence and fluorescence were measured overtime using an EnVision plate reader (PerkinElmer). No-cell and medium-only controls were used for background correction.

Synergy evaluation—for the evaluation of the synergistic effect of Compound 6.11 in combination with chemotherapeutic agents or PARPi in causing cytotoxicity or DNA damage the Bliss synergistic score quantified by the SynergyFinder R package was used.

Statistical analysis—data were examined for statistical significance using GraphPad Prism 9, using one-way ANOVA tests, as indicated in the figure legends. All experiments were performed in 2-3 independent experimental replicates and data expressed as Mean±S.E.M. Significance levels were defined as followed: ns p>0.05, *p<0.05, **p<0.01, **p<0.001, ****p<0.0001. IC₅₀ values were calculated using GraphPad Prism 9, fitting the dose response curves to a nonlinear regression model.

### Results

CHD1L inhibitors synergize with standard-of-care chemotherapy and PARPi

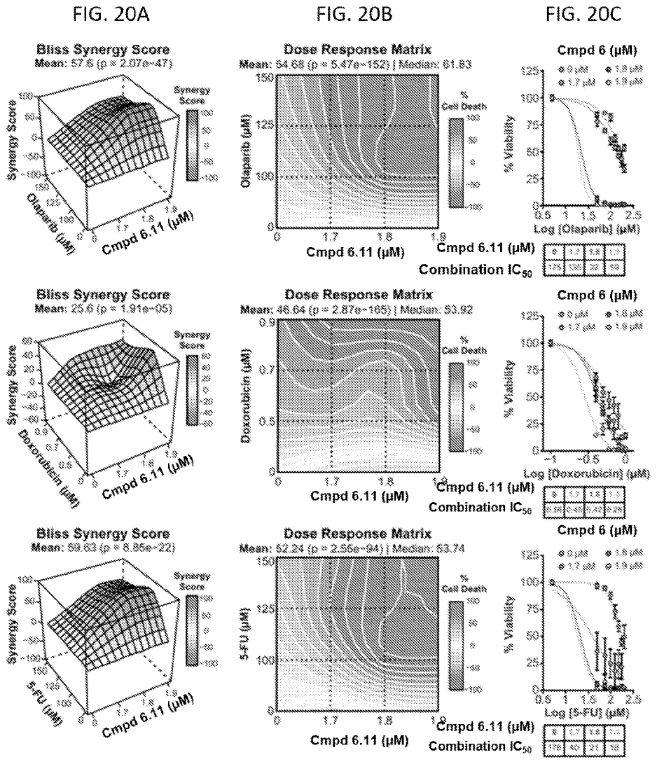

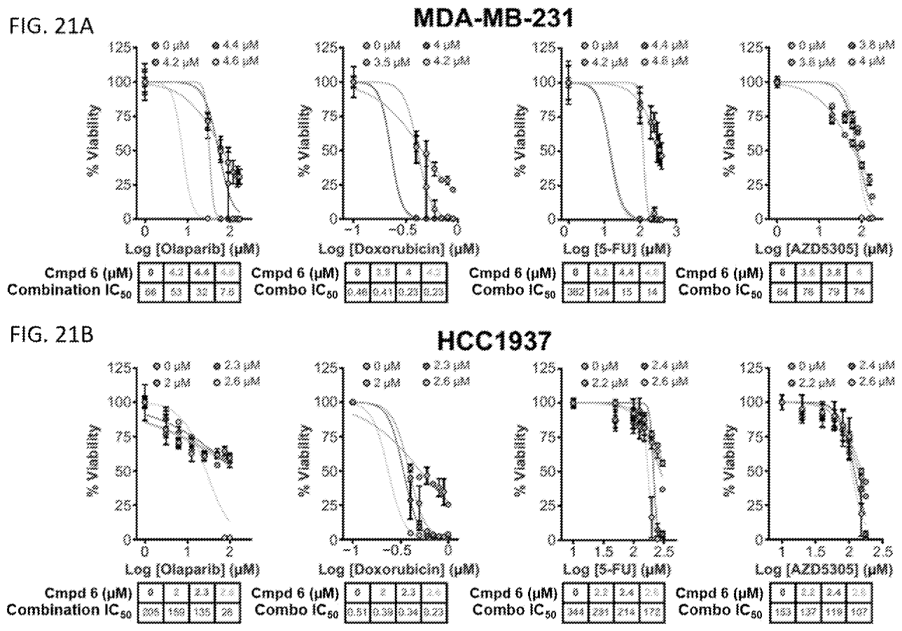

CHD1L inhibitors (CHD1Li) inhibit the ATPase activity of CHD1L, inducing the reversion of EMT and cell death in colorectal cancer and display antitumor activity in vivo. The cytotoxic effect of CHD1Li lead drug Compound 6 alone and in combination with SOC drugs used in the treatment of TNBC was assessed. Compound 6 is cytotoxic to TNBC tumor organoids, potently inhibiting the viability with half maximal inhibitory concentration (IC₅₀) values of 1.7 μM (BRCA1 mutant HR-deficient SUM149PT), 2.8 μM (BRCA1 mutant HR-deficient HCC1937), and 3.3 μM (BRCA wildtype HR-proficient MDA-MB-231). When combining non-lethal doses of Compound 6 with chemotherapy and PARPi a synergistic effect with all of them, both in BRCA1 mutant and HR-proficient cell models was observed (FIG. 21A, 21B). In SUM149PT tumor organoids, the combination improved the IC₅₀ potency of doxorubicin (topoisomerase II poison) by 2-fold from 0.56±0 12 μM to 0.28±0.01 μM (FIG. 20A-C). The synergistic effect was more pronounced when combining Compound 6 with 5-FU (inhibitor of DNA synthesis), improving the IC₅₀ potency of 5-FU by 10-fold with a Bliss synergy score of 59 (FIG. 20A-C).

Compound 6 also synergized with PARPi such as olaparib (PARP1/2 inhibitor) and AZD5305 (PARP1 selective inhibitor). The inhibition of PARP1/2 by olaparib, strongly synergized with CHD1L inhibition, improving the IC₅₀ potency of olaparib by 9-fold from 170±27 μM to 19±0.56 μM (FIG. 20A-C), while inhibition of only PARP1 by AZD5305 only improved its IC₅₀ potency by 2.7-fold in SUM149PT tumor organoids.

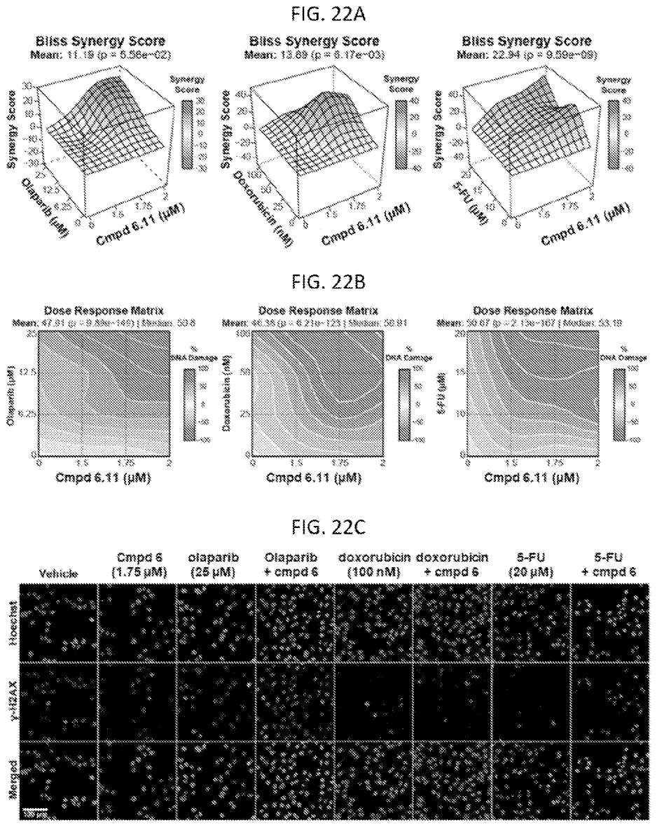

Inhibition of CHD1L enhances chemotherapy and PARPi-mediated DNA damage and cell cycle arrest. To understand the mechanism behind the cytotoxic synergistic effect between CHD1Li and TNBC therapies, the effect on the DDR, in which CHD1L plays an important role, was investigated. DNA damage was measured by immunofluorescence of the phosphorylation of the H2AX histone at the Ser-139 residue (γ-H2AX). This modification is required for the assembly of DNA repair proteins at sites of damaged chromatin, as well as for the activation of checkpoint proteins that arrest cell cycle progression. The treatment of HR-deficient SUM149PT cells with a range of doses of olaparib (0-25 μM), showed a maximum 5-fold increase in the number of γ-H2AX foci. PARP is mostly involved in DNA single stranded break (SSB) repair through the base excision repair (BER) pathway and when it is inhibited, and the replication fork encounters the SSBs, they progress to double stranded breaks (DSB) inducing γ-H2AX signaling. When combined with BRCA mutant HR-deficiency, PARPi cause synthetic lethality and cell death. In contrast, treatment with Compound 6 does not cause DNA damage, but when combined with olaparib an enhancement of PARP inhibition-mediated DNA damage (p<0.01l) was observed. This increase in the number of γ-H2AX foci is caused by a synergistic effect, showing a Bliss Synergy Score of 11.19 (FIG. 22A-C). Other drugs used in the treatment of TNBC, such as 5-FU and doxorubicin, also showed a synergistic increase in DNA damage when combined with Compound 6 (FIG. 22A-C).

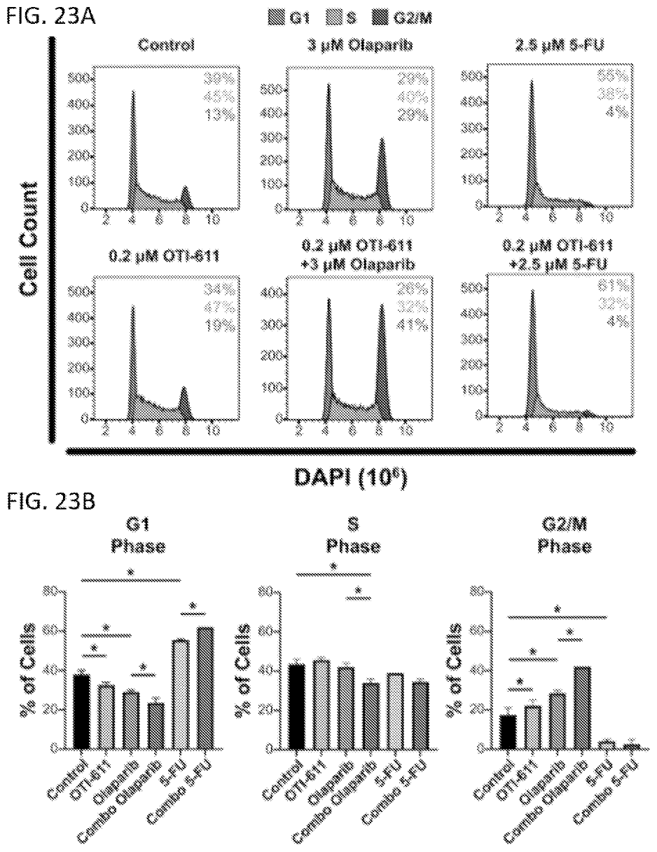

Upon generation of DNA lesions, DNA damage checkpoints are also activated, which can lead to an arrest of the cell cycle, restraining chromosome segregation until the damaged DNA has been repaired. To study the effect of TNBC therapies and inhibition of CHD1L on the cell cycle, cells were stained with DAPI, and the cell subpopulations in G1, S and G2/M phases were analyzed by flow cytometry. Treatment of SUM149PT cells for 24 hours with 0.2 μM of Compound 6 showed an increase in tetraploid DNA content, indicating an arrest in S and G2/M phases. Olaparib treatment caused a 2-fold increase in the percentage of cells in the G2/M phase, indicating that the DSB accumulation mediated by PARPi activates the G2/M checkpoint. In contrast, the number of cells in G1 phase increased by 16% with 5-FU treatment compared to the untreated cells, due to the inhibition of DNA synthesis through the S phase and activation of the G1 DNA damage checkpoint. Remarkably, the combination of these drugs with CHD1Li enhanced their effects, potentiating G2/M arrest caused by olaparib and G1 arrest caused by 5-FU (FIG. 23).

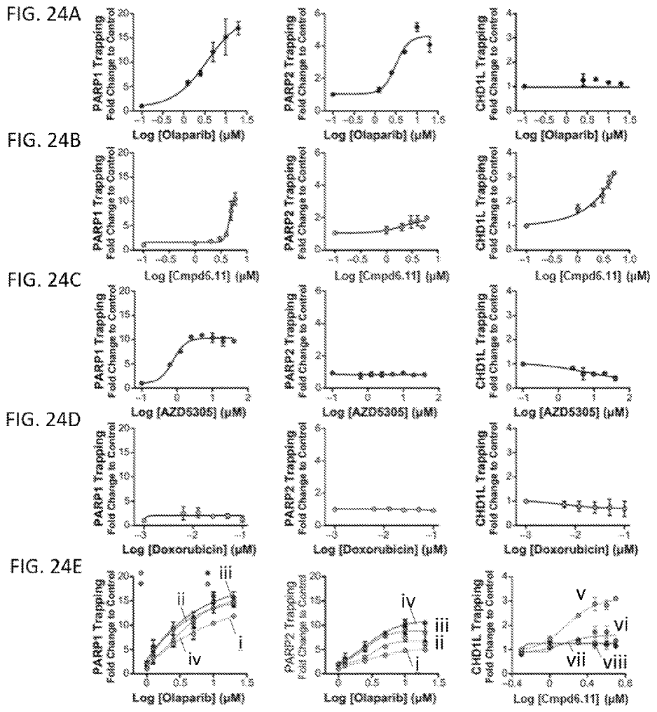

Inhibition of CHD1L traps PARP1, PARP2 and CHD1L at DNA damage sites. It is known that PARP-DNA complexes, caused by the inhibition of PARP auto-PARylation and its release from chromatin, are highly cytotoxic and are the driver of PARPi cytotoxicity, rather than unrepaired SSBs. Without being bound by any particular theory, PARP trapping could be another mechanism behind the synergistic effect of CHD1L and PARP inhibition. To study the accumulation of bound PARP1/2 on chromatin, in situ subcellular fractioning of SUM149PT cells co-treated with 0.01% methyl methanesulfonate (MMS) and the drug of interest was used, followed by immunofluorescence. Doxorubicin was used as a negative control to demonstrate recruitment of PARP1/2 to DNA damage sites independent from PARP trapping (FIG. 24D). Upon DNA damage caused by MMS, olaparib trapped in a dose-dependent manner both PARP1 and PARP2 on DNA, while the PARP1 selective inhibitor AZD5305, only increased PARP1 tapping (FIGS. 24A and 24C). The PARP1 trapping potency was observed to be higher for olaparib with a 15-fold increase compared to a 10-fold increase with AZD5305. Likewise, CHD1L inhibition also caused a dose-dependent trapping of PARP1/2 with a 10-fold and 2-fold increase in PARP1 and PARP2 trapping, respectively (FIG. 24B). In general, PARP2 trapping showed a lower increase than PARP1, which may be due to a lower abundance of PARP2 in cells. Thus, using doses of Compound 6.11 that do not cause PARP trapping, a significant increase in PARP1/2 trapping was observed when combined with olaparib compared to olaparib treatment alone (FIG. 24E). The degree to which Compound 6.11 could induce PARP1, PARP2, and CHD1L trapping with and without Olaparib was tested, and the results are shown in FIG. 24E. In the first panel, the amount of PARP1 trapping as a function of Olaparib concentration and Compound 6.11 concentration was measured. The following conditions were tested: (i) 0 μM Compound 6.11, (ii) 2 μM Compound 6.11, (iii) 3 μM Compound 6.11, (iv) 4 μM Compound 6.11, (v) 0 μM Olaparib, (vi) 2 μM Olaparib, (vii) 3 μM Olaparib, and (viii) 4 μM Olaparib.

Other studies on the role of CHD1L used cell lines with knockdown or knockouts of the gene encoding for CHD1L and have never been able to study the result of its enzymatic inhibition. By contrast, the instant example showed the interaction and trapping on chromatin of CHD1L upon DNA damage and PARP or CHD1L enzymatic inhibition. Doxorubicin was used as a control, proving that DNA damage does not cause a stable interaction of CHD1L and chromatin (FIG. 24D). PARPi olaparib and AZD5305 also did not show CHD1L trapping due to the inhibition of PARP and histone PARylation needed for CHD1L binding (FIGS. 24A and 24C). In contrast, the enzymatic inhibition of CHD1L by Compound 6 showed a dose-dependent increase in CHD1L trapping onto chromatin (FIG. 24B). Moreover, the combination of Compound 6 and olaparib showed a complete decrease in the CHD1L trapping caused by CHD1L inhibition, indicating a PAR-dependent mechanism for the activation of CHD1L and binding to nucleosomes (FIG. 24E). These results reveal that loss of CHD1L ATPase and nucleosome remodeling activity likely traps CHD1L on PARylated histones.

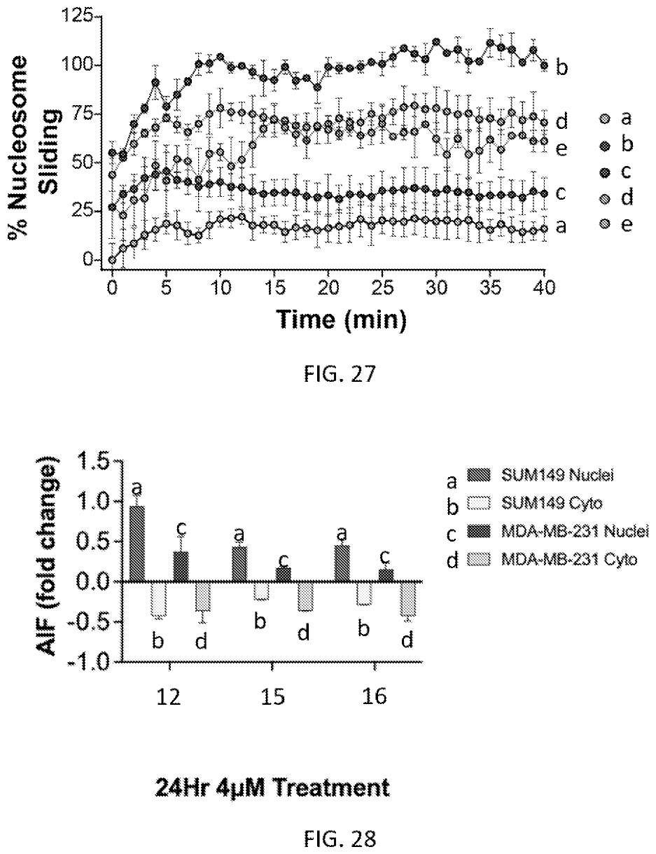

Inhibition of nucleosome remodeling by CHD1L inhibitors blocks DDR signaling. CHD1L's main role is chromatin remodeling and DNA relaxation, moving from a tightly condensed DNA to an accessible state allowing for DNA transcription and repair. Thus, the effect of CHD1L ATPase inhibition on its nucleosome remodeling activity using a FRET-based assay was used. The FRET assay measures the distancing of Cy3 on DNA and Cy5 on the H2A histone upon nucleosome sliding. Consistent with other literature report, the results demonstrate that nucleosome remodeling by CHD1L requires PARP1-mediated PARylation of nucleosomes, PAR binding to the CHD1L macro domain, and release of its autoinhibition. Thus, incubation of the nucleosomes with PARP1 (pre-incubated with NAD⁺), full length-CHD1L and ATP, showed a fast increase in the remodeling rate, starting at time 0 or the addition of ATP, and reaching a maximum after 30 minutes. The CHD1L ATPase domain hydrolyzes ATP to slide the nucleosomes and consequently our control with no ATP showed no remodeling. Interestingly, incubation with PARP1, NAD⁺ and ATP but no CHD1L, also showed some increase in the remodeling rate but was unstable in the time course of the experiment. When pre-incubating full length-CHD1L with 10 μM of Compound 6 for 10 minutes, a reduction of the remodeling rate was observed, demonstrating that the inhibition of the ATPase activity of CHD1L blocks its remodeling activity. The results are shown in FIG. 27. Briefly, (a) represents the presence of CHD1L and PARP1; (b) represents the presence of CHD1L, PARP1, and ATP; (c) represents the presence of CHD1L and ATP; (d) represents the presence of PARP1 and ATP; and (e) represents CHD1L, PARP1, ATP, and CHD1L inhibitor (Compound 6.11).

Since CHD1L inhibition did not cause DNA damage by γ-H2AX foci formation, inhibition of nucleosome remodeling and DNA relaxation could have an effect on the DDR. For that, SUM149PT cells were pre-treated with sub-lethal doses of Compound 6.11 for 4 hours, and then treated with Olaparib. The results showed that pre-treatment with CHD1Li significantly decreased the number of γ-H2AX foci caused by olaparib at low (5 μM) or high doses (25 μM), indicating that inhibition of CHD1L and subsequent nucleosome remodeling may prevent the DDR through inhibition of H2AX phosphorylation.

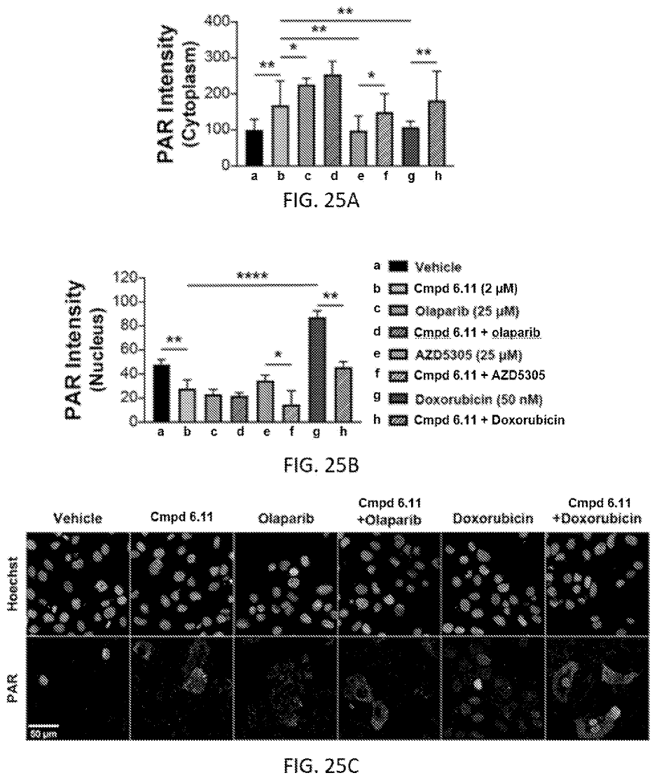

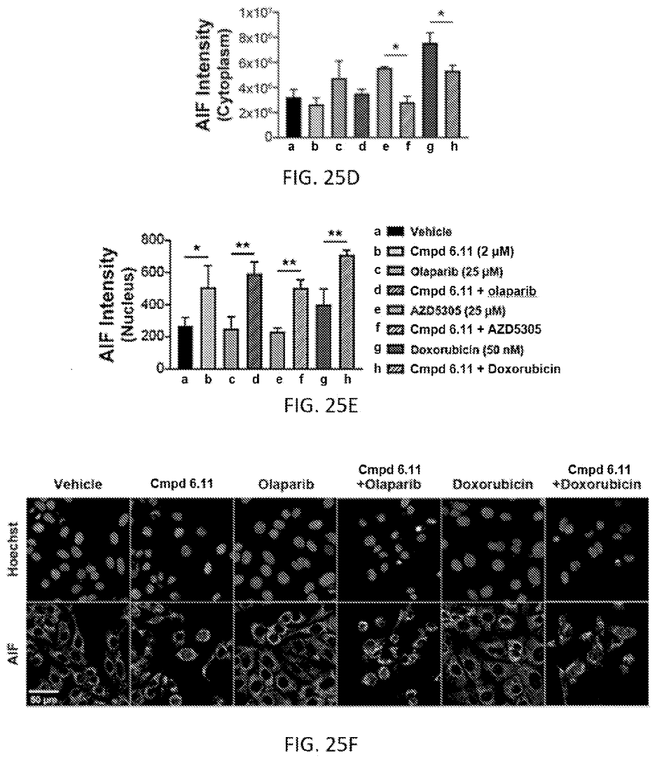

Inhibition of CHD1L promotes PAR translocation to the cytoplasm. The main role of PARP1 during the DDR is to detect and signal the recruitment of repair machinery to the sites of DNA damage. For that, PARP1/2 catalyze PARylation, which is a pivotal post-transcriptional protein modification, consisting of the addition of ADP-ribose units to the carboxyl group of acidic residues such as glutamate or aspartate on target proteins by using NAD⁺ as substrate. CHD1L is one of the proteins that requires binding to these PAR chains for its activation and subsequent chromatin remodeling. Using immunofluorescence of PAR, the effect of PARPi and CHD1Li on PARylation in SUM149PT cells was investigated. As expected, after a 4-hour treatment with the PARPi olaparib and AZD5305, an inhibition of nuclear PARylation caused by the blockade of the NAD⁺ binding site of PARP molecules was observed and an increase in cytoplasmic PAR in olaparib treated cells (FIG. 25A-C). In contrast, doxorubicin increased PARylation in the nucleus due to the increase in DNA damage and activation of DDR, while having no effect on cytoplasmic PAR (FIG. 25A-C). Interestingly, inhibition of CHD1L by treatment with Compound 6 caused the same effects as PARPi, and when combining them, this effect was potentiated. In addition, the effect of doxorubicin on nuclear PARylation was reverted by CHD1L inhibition, causing an increase of PAR in the cytoplasm (FIG. 25A-C).

To prove that Compound 6 is not directly inhibiting PARP1/2 and thus PARylation, enzyme assays were performed based on the capacity of PARP1/2 to add PAR to nucleosomes and itself, using NAD⁺. The results showed that olaparib inhibits both PARP1 and PARP2 and that Compound 6 does not have any effect on PARP1/2 activity. In contrast, Compound 6 potently inhibited the CHD1L ATPase enzymatic activity, while PARPi had no inhibitory effect. These results suggest that inhibition of CHD1L promotes translocation of PAR from the nucleus to the cytoplasm and is not a direct effect on PARylation by PARP1/2 inhibition.

It has been described that CHD1L binding to the PAR chain through its macro domain, protects the PAR chain from being hydrolyzed by PAR-Glycohydrolase (PARG), which is the main enzyme for PAR degradation. To further investigate this mechanism, cells were pre-treated for 2 hours with 1 μM of a PARG inhibitor (PDD00017273) prior to treatment with PARPi or CHD1Li. The inhibition of PARG did not have any effect on PARPi-mediated inhibition of nuclear PARylation. In contrast, pre-treatment with PARGi and therefore inhibition of PAR chain hydrolysis, kept the levels of nuclear PAR similar to control cells and inhibited PAR translocation to the cytoplasm mediated by CHD1L inhibition. Accordingly, CHD1L plays a key role in the homeostasis of PAR by protecting against PARG hydrolysis and Compound 6.11 inhibition of CHD1L promotes PARG-mediated hydrolysis and PAR translocation to the cytoplasm.

CHD1Li-mediated PAR translocation to the cytoplasm activates PARthanatos as a cell death mechanism. Parthanatos is a form a programmed cell death different from apoptosis and known to be mediated by PARP1 overactivation. The unique events occurring during parthanatos are: PARP1 rapid activation, accumulation of PAR and nuclear translocation of apoptosis inducing factor (AIF). Without being bound by any particular theory, accumulation of PAR in the cytoplasm caused by CHD1Li can also mediate parthanatos. To better understand the link between PAR translocation to the cytoplasm and cell death, immunofluorescence to measure the activation of caspase-3 and the subcellular localization of AIF after treatment with SOC drugs and Compound 6 in SUM49PT cells was used. After an 18-hour treatment with PARPi, olaparib or AZD5305, an increase in the AIF intensity in the cytoplasm was observed but no translocation of this protein to the nucleus (FIG. 25D-E) was observed. Without being bound by any particular theory, this might be caused by the mitochondrial outer membrane permeabilization, which triggers release of mitochondrial proteins like AIF or cytochrome c, activating caspase-3 by its cleavage and leading to apoptosis. When cells were treated with doxorubicin, activation of apoptosis by cleavage of caspase-3 was observed but also accompanied by some increase of AIF in the nucleus (FIG. 25D-E). Inhibition of CHD1L by treatment with Compound 6 caused a decrease in cytoplasmic AIF, while significantly increasing AIF in the nucleus (FIG. 25D-E), which is a key mediator and biomarker of parthanatos. Moreover, when combining CHD1L inhibition with SOC drugs, the cell death mechanism shifts from apoptosis to parthanatos, evidenced by a significant increase in AIF nuclear translocation and decrease in activation of caspase-3 (FIG. 25D-E).

PARthanatos might be accompanied by other events also occurring during apoptosis or necrosis like mitochondrial depolarization, phosphatidylserine (PS) externalization, loss of membrane integrity and large-scale DNA fragmentation. To better characterize this mechanism, PS externalization and membrane integrity were studied using a real-time multiplex assay that monitors Annexin V binding to PS and simultaneous loss of cell membrane integrity with a membrane impermeable DNA binding dye. PARPi and doxorubicin showed an apoptotic kinetic phenotype, characterized by an early increase in PS exposure on the outer plasma membrane and a late loss of membrane integrity. In contrast, CHD1Li caused a rapid and simultaneous increase of both when combined with SOC drugs, shifting the kinetics to a more non-apoptotic phenotype. Moreover, in cells stained with Hoechst 33342, treatment with Compound 6 caused large-scale fragmentation of DNA, which displays a different pattern of dead cell bodies compared to those caused by apoptosis (small-scale fragmentation).

### Example 13: CHD1L Inhibitors

The ability of compounds of Table 1 to trap CHD1L and PARP1 onto nucleosomes was determined using the same methods and assays described in Examples 11 and 12.

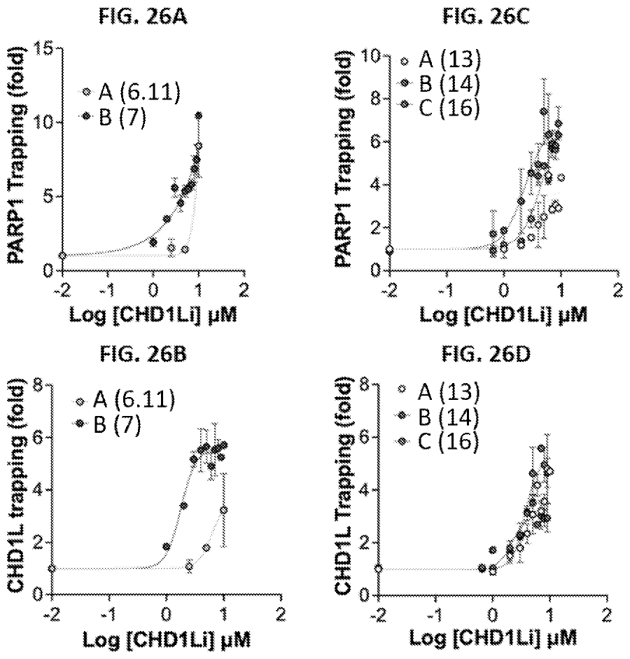

The ability of Compounds 6.11, 7, 13, 14, and 16 to trap CHD1L and PARP1 was determined using the procedure in Examples 11 and 12. Briefly, SW149PT cells were incubated Compounds 6.11, 7, 13, 14, and 16 at varying concentrations. After 24 hours, the cells were fixed and stained. The cells were exposed to anti-CHD1L and anti-PARP1 fluorescent antibodies, and the cells were subsequently imaged. FIG. 26A shows a graph of PARP1 trapping relative to a standard (immunofluorescence of cells exposed to doxorubicin as control) as a function of Compound 6.11 or Compound 7 concentration in micromolar. Compounds 6.11 and 7 were able to trap PARP1 by at least 5-fold. FIG. 26B illustrates a graph of CHD1L trapping relative to a standard (immunofluorescence of cells exposed to doxorubicin as control) relative to Compound 6.11 and Compound 7 concentration in micromolar. Compound 7 was able to trap CHD1L by about 5-fold relative to the baseline, and Compound 6.11 was able to trap CHD1L by about 3-fold relative to the baseline. FIG. 26C shows a graph of PARP1 trapping relative to a standard (immunofluorescence of cells exposed to doxorubicin as control) as a function of Compounds, 13, 14, and 16 concentration in micromolar. FIG. 26D illustrates a graph of CHD1L trapping relative to a standard (immunofluorescence of cells exposed to doxorubicin as control) relative to Compounds, 13, 14, and 16 concentration in micromolar. Without being bound by any particular theory, the mechanism of action of the tested CHD1L inhibitors was driven by CHD1Li inhibition of CHD1L ATPase activity preventing chromatin remodeling and CHD1L trapping onto PARylated nucleosomes. CHD1L was activated to promote DNA damage repair by PAR binding of autoPARylated PARP1/2 but also CHD1L PAR binding protected PAR chains from PARG-mediated hydrolysis, which helped PARP overcome PARP trapping. Thus, CHD1Li prevents CHD1L ATPase and chromatin remodeling, and PAR-binding leading to PARG-hydrolysis of PAR chains as the mechanism that traps PARP1/2.

The ability of Compounds 12, 15 and 16 to localize AIF was tested in SUM149 (breast cancer) and MDA-MB-231 (TNBC) cell lines. A comparison of the relative distribution of AIF in nuclei (a) versus cytoplasm (b) in SUM149 cells and nuclei (c) versus cytoplasm (d) in MDA-MB-231 cells in the presence of 4 μM Compounds 12, 15 and 16 are shown in FIG. 28. After treatment of Compound 12, AIF significantly localized in the nuclei in SUM149 cells, as shown by the almost 1-fold increase in AIF in the nucleus, compared to a decrease in AIF in the cytoplasm. After treatment with Compound 12, AIF localized in both the nuclei and remained in the cytoplasm in MDA-MB-231 cells. After treatment with Compound 15 in SUM149 cells, AIF tended to localize in the nuclei of SUM149 cells as compared to the cytoplasm, as shown by the about 0.5-fold increase in AIF in the nucleus, concomitant with an about 0.5 decrease in AIF in the cytoplasm. Similarly, AIF concentration in the cytoplasm of MDA-MB-231 cells decreased by about 0.5 after administration with Compound 15. After treatment with Compound 16, the relative concentration of AIF in the nuclei of SUM149 cells increased by about 0.5-fold, and the concentration of AIF in the cytoplasm decreased by about 0.4-fold. In MDA-MB-231 cells, the relative concentration of AIF in the cytoplasm decreased by about 0.5-fold, while the concentration of AIF increased in the nuclei by about 0.1-fold. Taken together, Compounds 12, 15, and 16 were shown to localized AIF in breast cancer cell lines SUM149 and MDA-MB-231, where a greater effect was observed in SUM149 cells. In MDA-MB-231 cells, Compounds 12, 15, and 16 greatly decreased the amount of AIF in the cytoplasm.

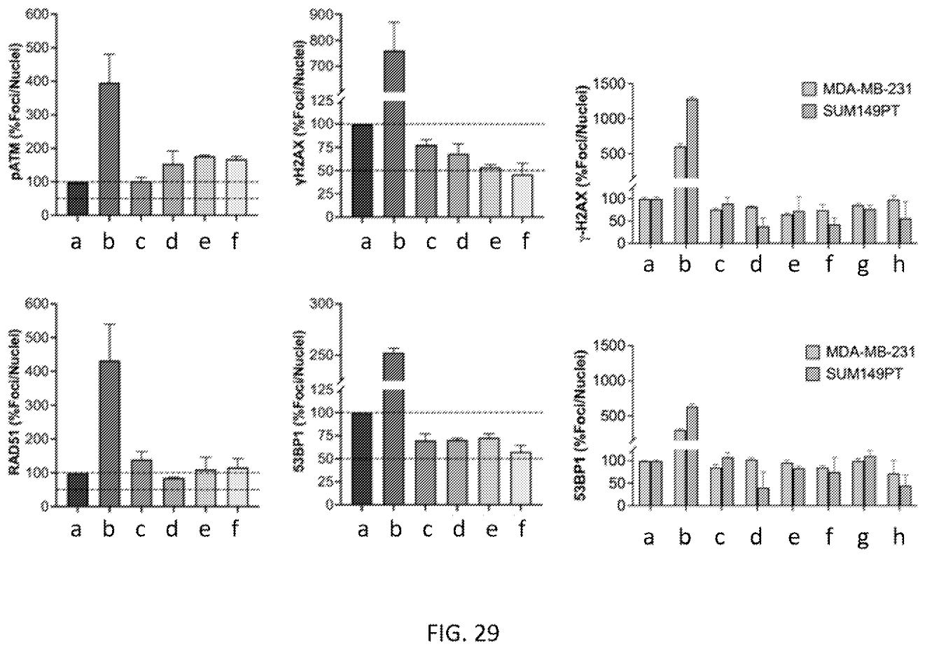

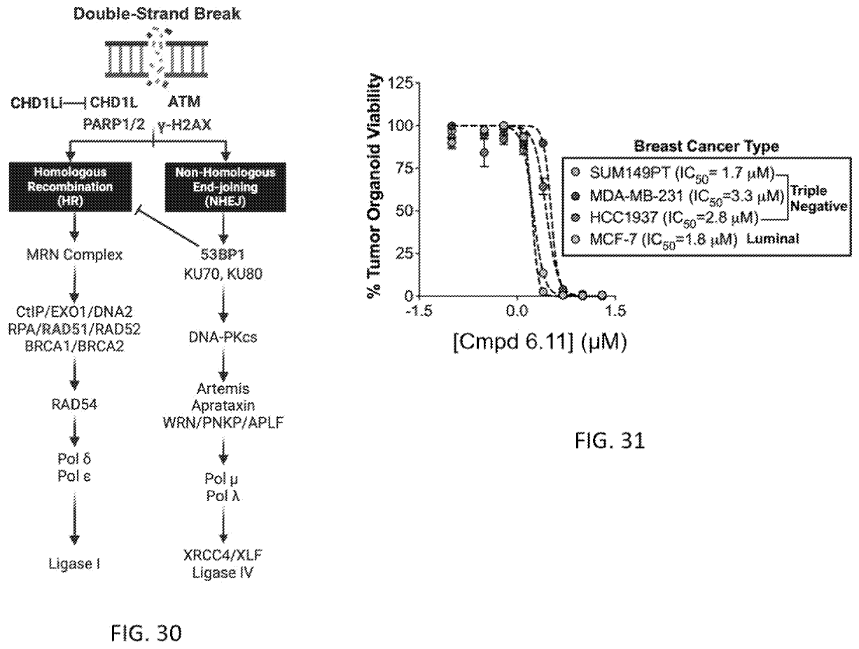

The ability of Compounds 6.11, 12, 14, and 16 to block DNA Damage Response (DDR) and Repair was tested by monitoring the relative amounts of pATM, RAD51, 53BP1, and γ-H2AX, which are biomarkers for the DDR (as illustrated in FIG. 30), and the results are presented in FIG. 29. Colo679 cells were treated with a (a) vehicle, (b) 2.5 μM etoposide, (c) 4 μM Compound 6.11, (d) 4 μM Compound 12, (e) 4 μM Compound 14, and (f) 4 μM Compound 16. SUM149 and MDA-MB-231 cells were treated with a (a) vehicle, (b) 2.5 μM etoposide, (c) 4 μM Compound 6.11, (d) 4 μM Compound 12, (e) 4 μM Compound 14, and (f) 4 μM Compound 16. Etoposide was used as a control to show upregulation of DDR pathway biomarkers. Regarding the effect on pATM, Compound 6.11 had minimal effect on the amount of pATM, whereas Compounds 12, 14, and 16 each increased the amount of pATM present. Regarding the effect on γ-H2AX, γ-H2AX concentrations decreased with treatment of Compounds 6.11, 12, 14, and 16. Regarding RAD51, administration of Compound 6.11 appeared to increase the amount of RAD51, indicating the homologous recombination pathway was activated. By contrast, administration of Compound 12 showed a decrease in RAD51, suggesting the homologous recombination pathway may not have been activated. Regarding 53BP1, Compounds 6.11, 12, 14, and 16 appeared to decrease the amount of 53BP1, indicating suppression or inactivation of the non-homologous end-joining (NHEJ) pathway. In the breast cancer cells (SUM149 and MDA-MB-231), administration of Compounds 6.11, 12, 14, and 16 showed overall decreases in the amounts of γ-H2AX and 53BP1 indicating suppression of the NHEJ pathway.

CHD1L inhibitor Compound 6.11 as a monotherapy for breast cancers. Triple negative breast cancer cell lines (SUM149PT, MDA-MB-231, HCC1937) and a luminal breast cancer cell line (MCF-7) were treated with Compound 6.11 at varying concentrations. The IC50 of Compound 6.11 in SUM149 PT cells was about 1.7 μM, in MDA-MB-231 cells was about 3.3 μM, in HCC1937 cells was about 2.8 μM, and in MCF-7 cells was about 1.8 μM, as shown in FIG. 31. The tumor organoid viability assay is as described in Example 9.

### Example 14: Combination Therapy of CHD1L Inhibitors and Chemotherapies in Breast Cancer Cells

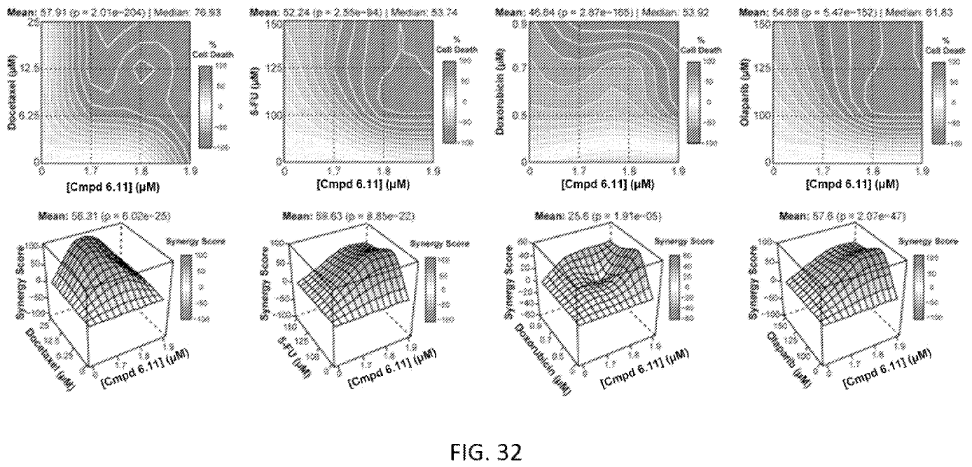

SUM149PT cells were treated with varying concentrations of Compound 6.11 (micromolar) and chemotherapies (docetaxel, 5-FU, doxorubicin, and Olaparib; micromolar), and the results are presented in FIG. 32. The Bliss synergy scores are presented and the synergy between Compound 6.11 and the chemotherapies in promoting SUM149PT cell death is presented in the contour graphs. The data indicate overall synergy between Compound 6.11 and docetaxel, 5-FU and Olaparib across all tested concentrations. The mean synergy score between Compound 6.11 and docetaxel was about 56, between Compound 6.11 and 5-FU was about 60, and Compound 6.11 and Olaparib was about 58, each of which are well above the threshold of 10, suggestive of high synergy between Compound 6.11 and the chemotherapies.

### Example 15: CHD1L Inhibitors for Treatment of Colorectal Cancer Using Colo678 Organoids

SUM149PT cells were treated with varying concentrations of Compound 6.11 (micromolar) and chemotherapies (docetaxel, 5-FU, doxorubicin, and Olaparib; micromolar), and the results are presented in FIG. 32. The Bliss synergy scores are presented and the synergy between Compound 6.11 and the chemotherapies in promoting SUM149PT cell death is presented in the contour graphs. The data indicate overall synergy between Compound 6.11 and docetaxel, 5-FU and Olaparib across all tested concentrations. The mean synergy score between Compound 6.11 and docetaxel was about 56, between Compound 6.11 and 5-FU was about 60, and Compound 6.11 and Olaparib was about 58, each of which are well above the threshold of 10, suggestive of high synergy between Compound 6.11 and the chemotherapies.

### Example 16: CHD1L Inhibitors for Treatment of Colorectal Cancers

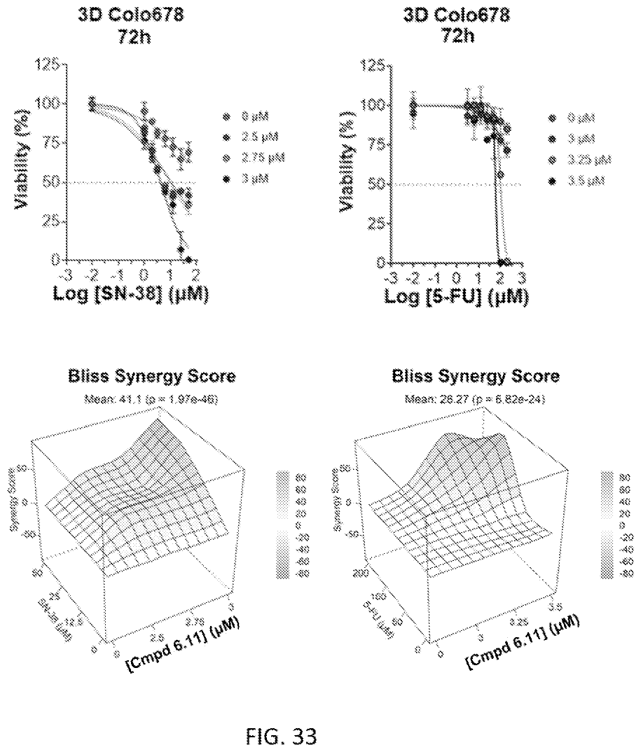

Colo678 cells were treated with Compounds 6.11, 12, 13, 20, 14, and 16 at varying concentrations in combination with SN-38 at varying concentrations, and the treatment was allowed to take for 72 hours. The results of the synergy experiments (described above in Examples 1-9) are presented in FIGS. 33-38.

The combination of Compound 6.11 and SN-38 was found to decrease the IC₅₀ of SN-38 as a function of Compound 6.11 concentration, as shown in FIG. 33 (top). The IC₅₀ of SN-38 in Colo678 cells in the absence of Compound 6.11 was about 185.9 μM. By contrast, the IC₅₀ of SN-38 when administered in the presence of Compound 6.11 decreased by an order of magnitude to 10.92 μM (when 2.5 μM Compound 6.11 present). The calculated mean synergy score was about 41, indicating synergy between Compound 6.11 and SN-38. Colo678 cells were also treated with Compound 6.11 and 5-FU, and the results are also shown in FIG. 33. A summary of the IC₅₀ values is presented in Table 5.

TABLE 5
IC₅₀ of SN-38 and 5-FU in Colo678 Cells
when Treated with Compound 6.11

| Concentration of | | Concentration of | |
|---|---|---|---|
| Compound 6.11 | IC₅₀ SN-38 | Compound 6.11 | IC₅₀ 5-FU |
| (μM) | (μM) | (μM) | (μM) |
| 0 | 185.9 | 0 | 3433 |
| 2.5 | 10.92 | 3 | 716.6 |
| 2.75 | 9.748 | 3.25 | 99.43 |
| 3 | 4.277 | 3.5 | 58.47 |

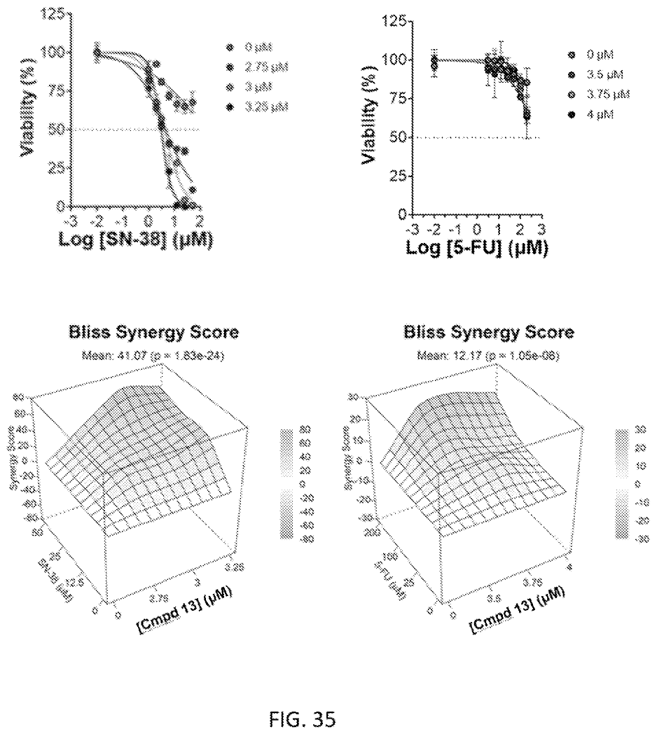

The combination of Compound 12 and SN-38 was found to decrease the IC₅₀ of SN-38 as a function of Compound 12 concentration, as shown in FIG. 35 (top, left). The combination of Compound 12 and 5-FU was found to decrease the IC₅₀ of 5-FU as a function of Compound 12 concentration, as shown in FIG. 35 (top, right). The calculated mean synergy score was about 26, indicating synergy between Compound 12 and SN-38. A summary of the IC₅₀ values is presented in Table 6.

TABLE 6
IC₅₀ of SN-38 and 5-FU in Colo678
Cells when Treated with Compound 12

| Concentration of | | Concentration of | |
|---|---|---|---|
| Compound 12 | IC₅₀ SN-38 | Compound 12 | IC₅₀ 5-FU |
| (μM) | (μM) | (μM) | (μM) |
| 0 | 71.58 | 0 | 75062 |
| 1 | 9.804 | 1 | 549.1 |
| 1.5 | 6.189 | 1.5 | 392.2 |
| 2 | 3.186 | 2 | 205.0 |

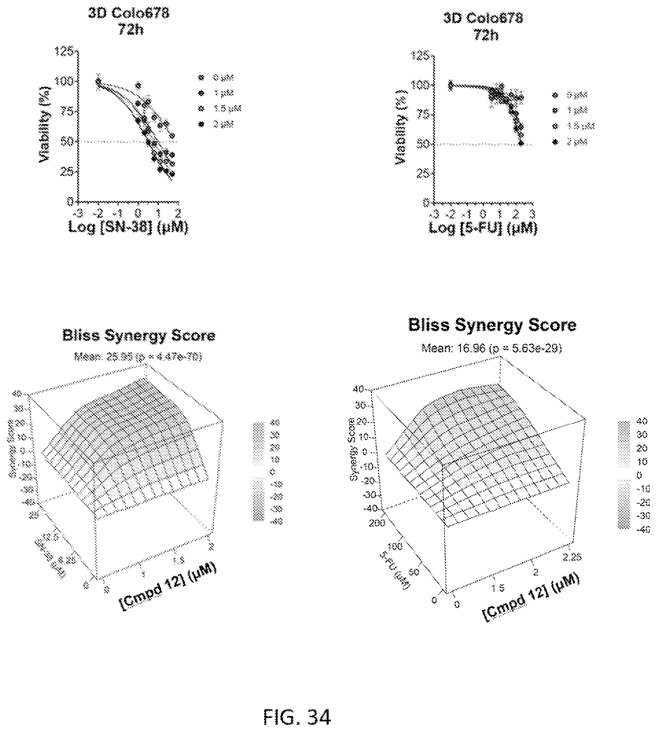

The combination of Compound 13 and SN-38 was found to decrease the IC₅₀ of SN-38 as a function of Compound 13 concentration, as shown in FIG. 34 (top, left). The combination of Compound 13 and 5-FU was found to decrease the IC₅₀ of 5-FU as a function of Compound 13 concentration, as shown in FIG. 34 (top, right). The calculated mean synergy score for the combination of Compound 13 and SN-38 was about 41. The calculated mean synergy score for the combination of Compound 13 and 5-FU was about 12, indicating synergy between Compound 13 and 5-FU. A summary of the IC₅₀ values is presented in Table 7.

TABLE 7
IC₅₀ of SN-38 and 5-FU in Colo678
Cells when Treated with Compound 13

| Concentration of | IC₅₀ | Concentration of | IC₅₀ |
|---|---|---|---|
| Compound 13 (μM) | SN-38 (μM) | Compound 13 (μM) | 5-FU (μM) |
| 0 | 149.4 | 0 | 7515 |
| 2.75 | 4.675 | 3.5 | 543.6 |
| 3 | 4.147 | 3.75 | 432.3 |
| 3.25 | 3.013 | 4 | 246.2 |

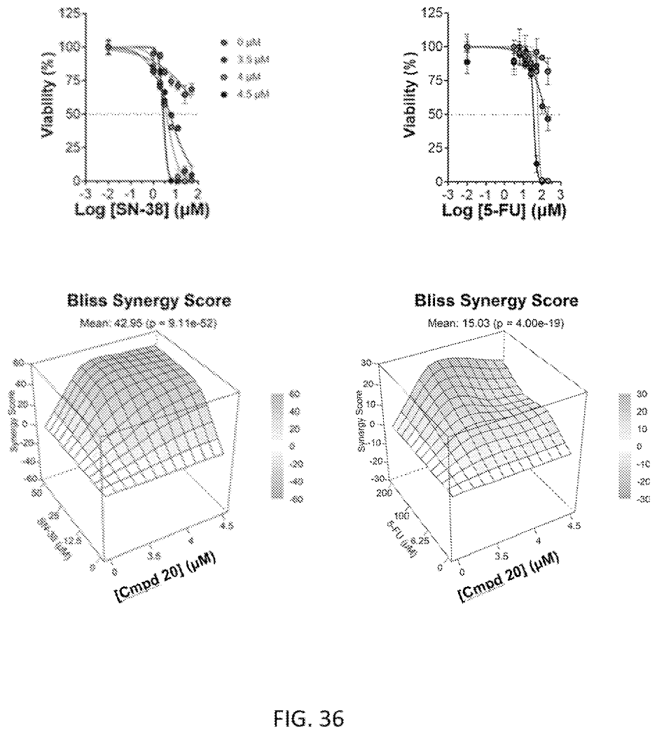

The combination of Compound 20 and SN-38 was found to decrease the IC₅₀ of SN-38 as a function of Compound 20 concentration, as shown in FIG. 36 (top, left). The combination of Compound 20 and 5-FU was found to decrease the IC₅₀ of 5-FU as a function of Compound 20 concentration, as shown in FIG. 36 (top, right). The calculated mean synergy score for the combination of Compound 20 and SN-38 was about 43. The calculated mean synergy score for the combination of Compound 20 and 5-FU was about 15, indicating synergy between Compound 20 and 5-FU. A summary of the IC₅₀ values is presented in Table 8.

TABLE 8
IC₅₀ of SN-38 and 5-FU in Colo678
Cells when Treated with Compound 20

| Concentration of | IC₅₀ | Concentration of | IC₅₀ |
|---|---|---|---|
| Compound 20 (μM) | SN-38 (μM) | Compound 20 (μM) | 5-FU (μM) |
| 0 | 163.2 | 0 | 14809 |
| 3.5 | 5.3940 | 3.5 | 170.2 |
| 4 | 3.723 | 4 | 60.01 |
| 4.5 | 2.746 | 4.5 | 34.48 |

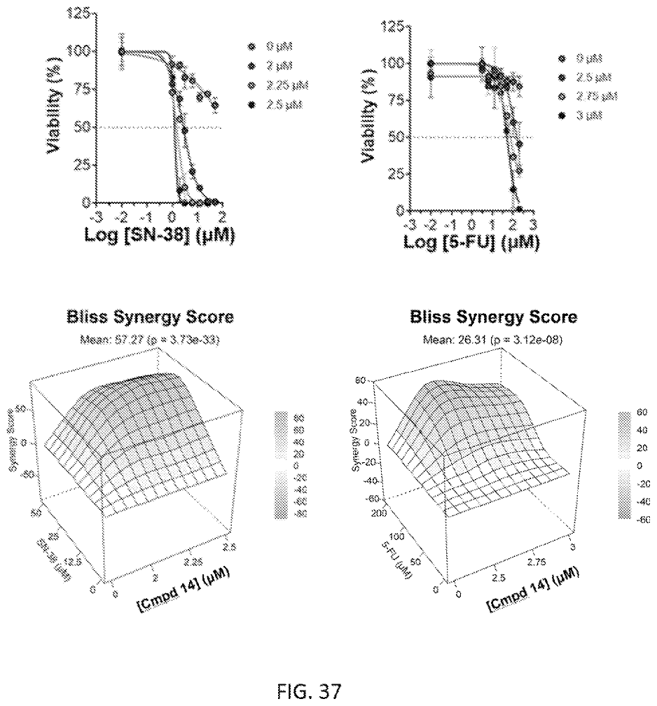

The combination of Compound 14 and SN-38 was found to decrease the IC₅₀ of SN-38 as a function of Compound 14 concentration, as shown in FIG. 37 (top, left). The combination of Compound 14 and 5-FU was found to decrease the IC₅₀ of 5-FU as a function of Compound 13 concentration, as shown in FIG. 37 (top, right). The calculated mean synergy score for the combination of Compound 14 and SN-38 was about 57. The calculated mean synergy score for the combination of Compound 14 and 5-FU was about 26, indicating synergy between Compound 14 and 5-FU. A summary of the IC₅₀ values is presented in Table 9.

TABLE 9
IC₅₀ of SN-38 and 5-FU in Colo678
Cells when Treated with Compound 14

| Concentration of | IC₅₀ | Concentration of | IC₅₀ |
|---|---|---|---|
| Compound 14 (μM) | SN-38 (μM) | Compound 14 (μM) | 5-FU (μM) |
| 0 | 171.4 | 0 | 0.000 |
| 2 | 2.759 | 2.5 | 148.8 |
| 2.25 | 1.489 | 2.75 | 74.65 |
| 2.5 | 1.246 | 3 | 50.39 |

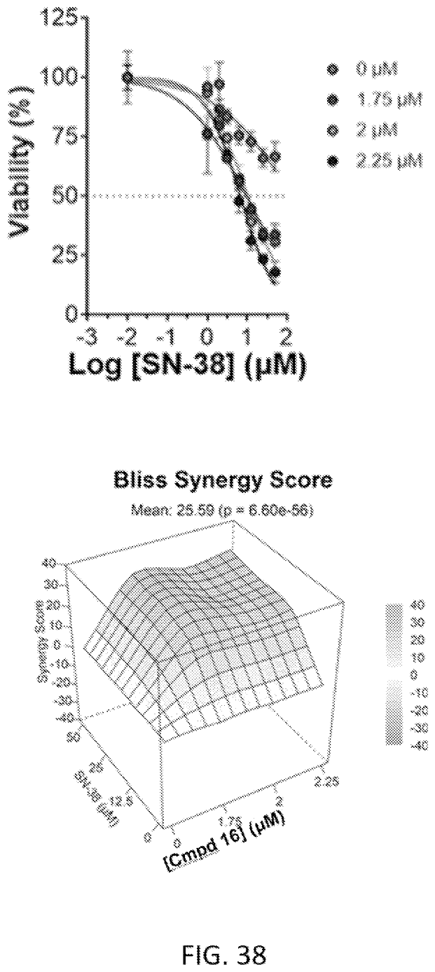

The combination of Compound 16 and SN-38 was found to decrease the IC₅₀ of SN-38 as a function of Compound 16 concentration, as shown in FIG. 38 (top). The calculated mean synergy score for the combination of Compound 16 and SN-38 was about 26. A summary of the IC₅₀ values is presented in Table 10.

TABLE 10
IC₅₀ of SN-38 in Colo678 Cells when Treated with Compound 16

| Concentration of Compound 16 (μM) | IC₅₀ SN-38 (μM) |
|---|---|
| 0 | 130.7 |
| 1.75 | 9.619 |
| 2 | 10.66 |
| 2.25 | 6.746 |

Compounds 6.11, 12, 13, 20, 14, and 16 were shown to decrease the IC₅₀ of SN-38 when the two were co-administered to Colo678 cells. The calculated mean synergy scores for each of the Compounds 6.11, 12, 13, 20, 14 and 16 show that CHD1L inhibitors were able to synergize strongly with SN-38 to decrease Colo678 cell viability.

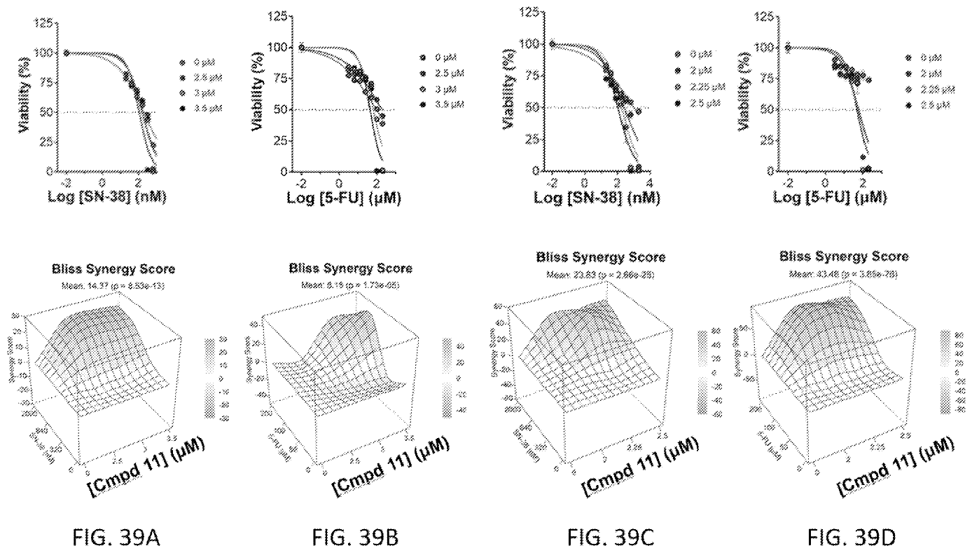

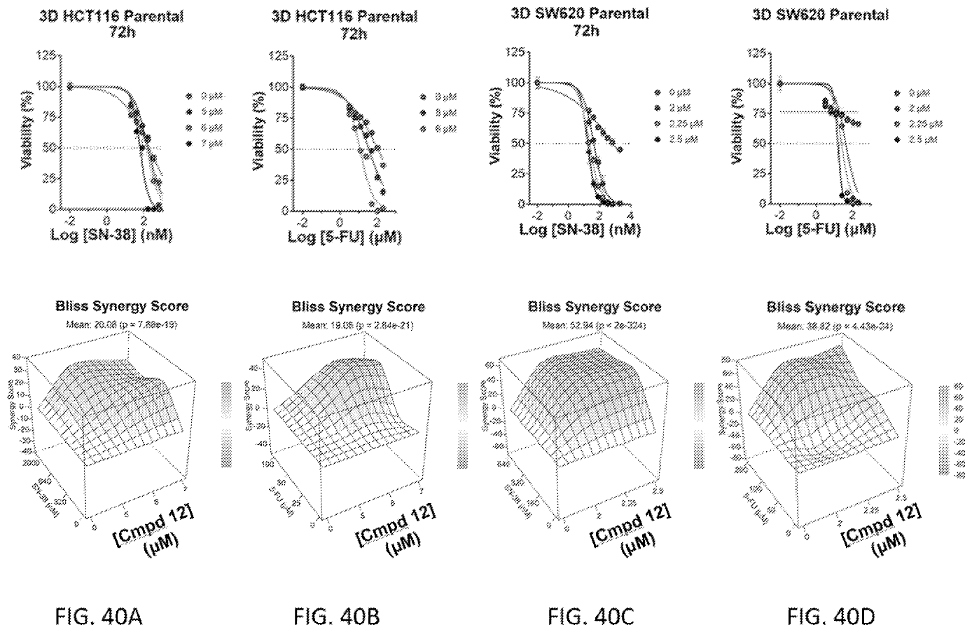

HCT116 Parental cells were treated with Compounds 11 and 12 at varying concentrations in combination with SN-38 at varying concentrations, and the treatment was allowed to take for 72 hours. The results of the synergy experiments (described above in Examples 1-9) are presented in FIGS. 39 and 40. SW620 Parental cells were treated with Compounds 11 and 12 at varying concentrations in combination with SN-38 at varying concentrations, and the treatment was allowed to take for 72 hours. The results of the synergy experiments (described above in Examples 1-9) are also presented in FIGS. 39 and 40.

The combination of Compound 11 and SN-38 was found to decrease the IC₅₀ of SN-38 as a function of Compound 11 concentration, as shown in FIG. 39A. The combination of Compound 11 and 5-FU was found to decrease the IC₅₀ of 5-FU as a function of Compound 11 concentration, as shown in FIG. 39B. The calculated mean synergy score for the combination of Compound 11 and SN-38 was about 14. The calculated mean synergy score for the combination of Compound 11 and 5-FU was about 8, indicating no synergy between Compound 11 and 5-FU. A summary of the IC₅₀ values is presented in Table 11A.

TABLE 11A
IC₅₀ of SN-38 and 5-FU in HCT116 Cells
when Treated with Compound 11

| Concentration of | IC₅₀ | Concentration of | IC₅₀ |
|---|---|---|---|
| Compound 11 (μM) | SN-38 (μM) | Compound 11 (μM) | 5-FU (μM) |
| 0 | 197.8 | 0 | 95.65 |
| 2.5 | 164.9 | 2.5 | 191.7 |
| 3 | 157.4 | 3 | 63.38 |
| 3.5 | 110.0 | 3.5 | 43.18 |

The combination of Compound 11 and SN-38 was found to decrease the IC₅₀ of SN-38 as a function of Compound 11 concentration in SW620 cells, as shown in FIG. 39C. The calculated mean synergy score for the combination of Compound 11 and SN-38 was about 24, indicating synergy between Compound 11 and SN-38. The combination of Compound 11 and 5-FU was found to decrease the IC₅₀ of 5-FU as a function of Compound 11 concentration in SW620 cells, as shown in FIG. 39D. The calculated mean synergy score for the combination of Compound 11 and 5-FU was about 43, indicating synergy between Compound 11 and 5-FU. A summary of the IC₅₀ values is presented in Table 11B.

TABLE 11B
IC₅₀ of SN-38 in SW620 Cells when Treated with Compound 11

| Concentration of | IC₅₀ | Concentration of | IC₅₀ |
|---|---|---|---|
| Compound 11 (μM) | SN-38 (μM) | Compound 11 (μM) | 5-FU (μM) |
| 0 | 989.33 | 0 | 20.78 |
| 2 | 260.9 | 2 | 59.51 |
| 2.25 | 178.3 | 2.25 | 54.40 |
| 2.5 | 106.3 | 2.5 | 47.69 |

The combination of Compound 12 and SN-38 was found to decrease the IC₅₀ of SN-38 as a function of Compound 12 concentration, as shown in FIG. 40A. The combination of Compound 12 and 5-FU was found to decrease the IC₅₀ of 5-FU as a function of Compound 12 concentration, as shown in FIG. 40B. The calculated mean synergy score for the combination of Compound 12 and SN-38 was about 20, indicating synergy between Compound 12 and 5-FU. The calculated mean synergy score for the combination of Compound 12 and 5-FU was about 19, indicating synergy between Compound 12 and 5-FU. A summary of the IC₅₀ values is presented in Table 12A.

TABLE 12A
IC₅₀ of SN-38 and 5-FU in HCT116 Cells
when Treated with Compound 12

| Concentration of | IC₅₀ | Concentration of | IC₅₀ |
|---|---|---|---|
| Compound 12 (μM) | SN-38 (μM) | Compound 12 (μM) | 5-FU (μM) |
| 0 | 183.3 | 0 | 102.7 |
| 5 | 165.6 | 5 | 34.55 |
| 6 | 139.2 | 6 | 11.52 |
| 7 | 60.26 | | |

The combination of Compound 12 and SN-38 was found to decrease the IC₅₀ of SN-38 as a function of Compound 12 concentration in SW620 cells, as shown in FIG. 40C. The calculated mean synergy score for the combination of Compound 12 and SN-38 was about 53, indicating synergy between Compound 12 and SN-38. The combination of Compound 12 and 5-FU was found to decrease the IC₅₀ of 5-FU as a function of Compound 12 concentration in SW620 cells, as shown in FIG. 40D. The calculated mean synergy score for the combination of Compound 12 and 5-FU was about 39, indicating synergy between Compound 12 and 5-FU. A summary of the IC₅₀ values is presented in Table 12.

TABLE 12B
IC₅₀ of SN-38 and 5-FU in SW620 Cells
when Treated with Compound 12

| Concentration of | IC₅₀ | Concentration of | IC₅₀ |
|---|---|---|---|
| Compound 12 (μM) | SN-38 (μM) | Compound 12 (μM) | 5-FU (μM) |
| 0 | 707.8 | 0 | 0.000 |
| 2 | 41.93 | 2 | 41.41 |
| 2.25 | 22.19 | 2.25 | 23.86 |
| 2.5 | 17.12 | 2.5 | 15.42 |

### Example 17: CHD1L Inhibitors for Treatment of Pancreatic Cancer

MiaPaca2 cells were used as a model for pancreatic cancer and were treated with varying concentrations of CHD1L inhibitors of the instant disclosure and SN-38. The cell viability assays and Bliss synergy analyses are as described above in Examples 1-9 and 11-12.

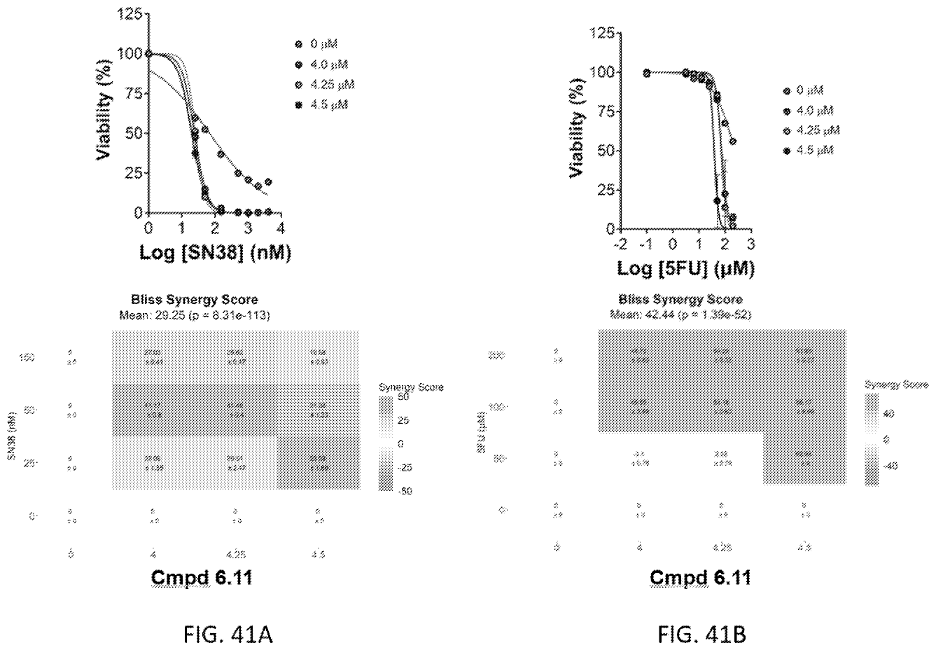

MiaPaca2 cells were treated with SN-38 and varying concentrations of Compound 6.11, and the ability of SN-38 and SN-38/Compound 6.11 to reduce MiaPaca2 cell viability. The IC₅₀ of SN-38 was found to decrease as a function of Compound 6.11 concentration, as shown in FIG. 41A and Table 13. The combination of Compound 6.11 and 5-FU was found to decrease the IC₅₀ of 5-FU as a function of Compound 6.11 concentration, as shown in FIG. 41B and Table 13. The calculated mean synergy score for the combination of Compound 6.11 and SN-38 was about 29, indicating synergy between Compound 6.11 and SN-38 to reduce MiaPaca2 cell viability. The calculated mean synergy score for the combination of Compound 6.11 and 5-FU was about 42, indicating synergy between Compound 6.11 and 5-FU. A summary of the IC₅₀ values is presented in Table 13.

TABLE 13
IC₅₀ of SN-38 and 5-FU in MiaPaca2 Cells
when Treated with Compound 6.11

| Concentration of | | Concentration of | |
|---|---|---|---|
| Compound 6.11 | IC₅₀ | Compound 6.11 | IC₅₀ |
| (μM) | SN-38 (μM) | (μM) | 5-FU (μM) |
| 0 | 68.35 | 0 | 238.0 |
| 4 | 24.03 | 4 | 74.43 |
| 4.25 | 25.41 | 4.25 | 68.66 |
| 4.5 | 19.79 | 4.5 | 38.31 |

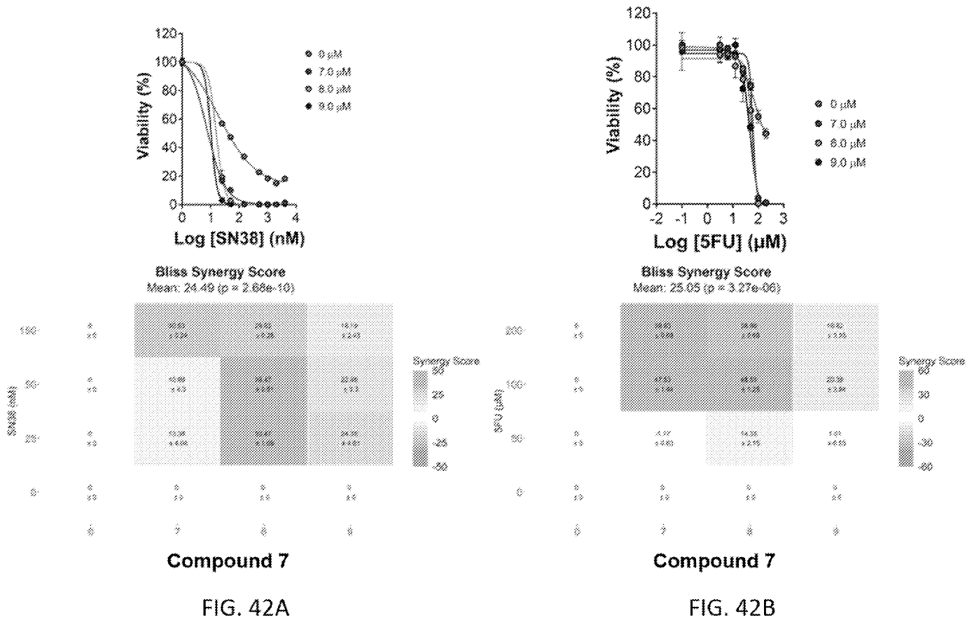

MiaPaca2 cells were treated with SN-38 and varying concentrations of Compound 7, and the ability of SN-38 and SN-38/Compound 7 to reduce MiaPaca2 cell viability. The IC₅₀ of SN-38 was found to decrease as a function of Compound 7 concentration, as shown in FIG. 42A and Table 14. The combination of Compound 7 and 5-FU was found to decrease the IC₅₀ of 5-FU as a function of Compound 7 concentration, as shown in FIG. 42B and Table 14. The calculated mean synergy score for the combination of Compound 7 and SN-38 was about 24.5, indicating synergy between Compound 7 and SN-38 to reduce MiaPaca2 cell viability. The calculated mean synergy score for the combination of Compound 7 and 5-FU was about 25, indicating synergy between Compound 7 and 5-FU. A summary of the IC₅₀ values is presented in Table 14.

TABLE 14
IC₅₀ of SN-38 and 5-FU in MiaPaca2
Cells when Treated with Compound 7

| Concentration of | IC₅₀ | Concentration of | IC₅₀ |
|---|---|---|---|
| Compound 7 (μM) | SN-38 (μM) | Compound 7 (μM) | 5-FU (μM) |
| 0 | 18.43 | 0 | 68.19 |
| 7 | 7.344 | 7 | 61.78 |
| 8 | 15.76 | 8 | 56.69 |
| 9 | 10.28 | 9 | 46.73 |

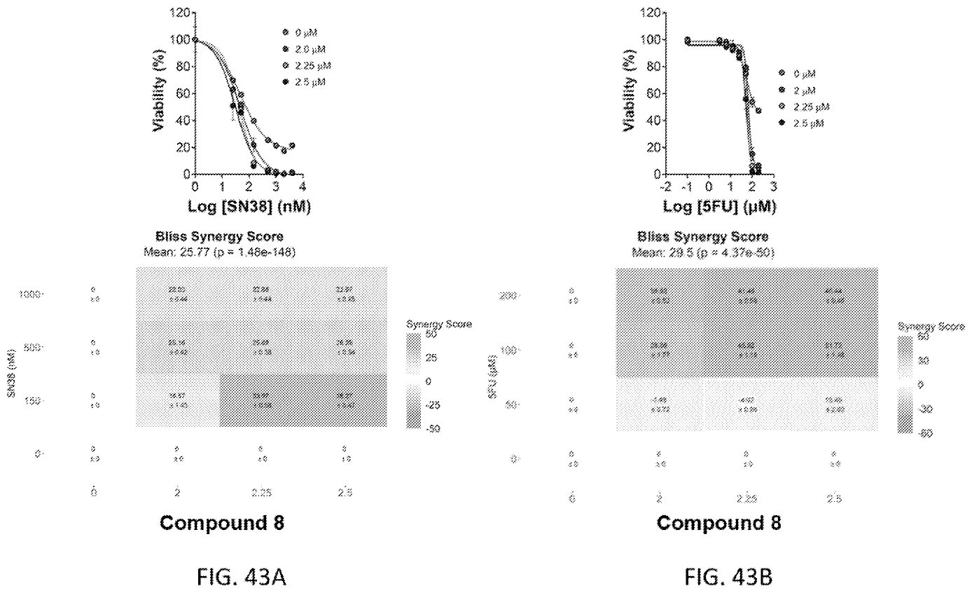

MiaPaca2 cells were treated with SN-38 and varying concentrations of Compound 8, and the ability of SN-38 and SN-38/Compound 8 to reduce MiaPaca2 cell viability. The IC₅₀ of SN-38 was found to decrease as a function of Compound 8 concentration, as shown in FIG. 43A and Table 15. The combination of Compound 8 and 5-FU was found to decrease the IC₅₀ of 5-FU as a function of Compound 8 concentration, as shown in FIG. 43B and Table 15. The calculated mean synergy score for the combination of Compound 8 and SN-38 was about 26, indicating synergy between Compound 8 and SN-38 to reduce MiaPaca2 cell viability. The calculated mean synergy score for the combination of Compound 8 and 5-FU was about 29.5, indicating synergy between Compound 8 and 5-FU. A summary of the IC₅₀ values is presented in Table 15.

TABLE 15
IC₅₀ of SN-38 and 5-FU in MiaPaca2
Cells when Treated with Compound 12

| Concentration of | IC₅₀ | Concentration of | IC₅₀ |
|---|---|---|---|
| Compound 8 (μM) | SN-38 (μM) | Compound 8 (μM) | 5-FU (μM) |
| 0 | 45.87 | 0 | 55.58 |
| 2 | 47.08 | 2 | 66.16 |
| 2.25 | 41.93 | 2.25 | 60.44 |
| 2.5 | 31.32 | 2.5 | 53.77 |

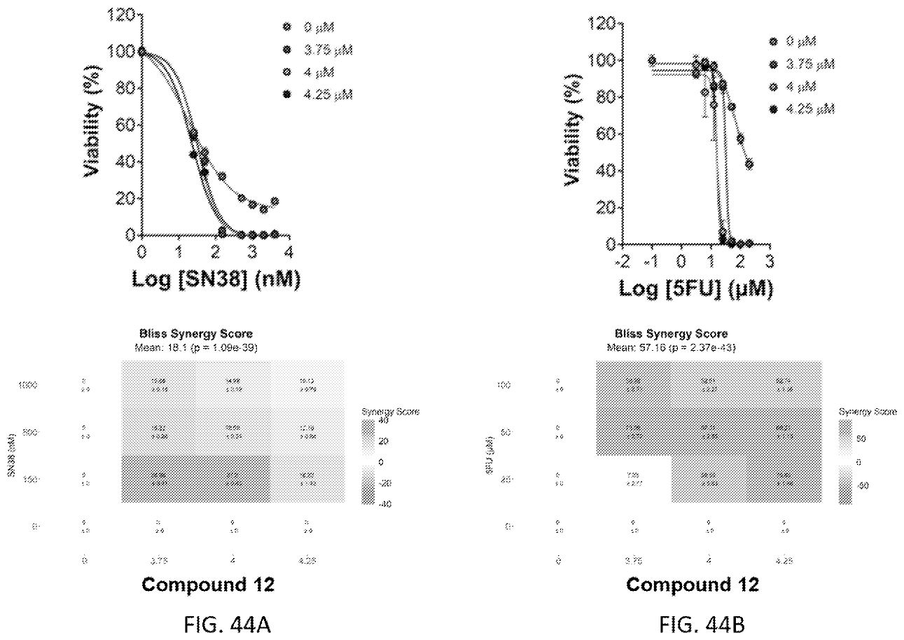

MiaPaca2 cells were treated with SN-38 and varying concentrations of Compound 12, and the ability of SN-38 and SN-38/Compound 12 to reduce MiaPaca2 cell viability. The IC₅₀ of SN-38 was found to decrease as a function of Compound 12 concentration, as shown in FIG. 44A and Table 16. The combination of Compound 12 and 5-FU was found to decrease the IC₅₀ of 5-FU as a function of Compound 12 concentration, as shown in FIG. 44B and Table 16. The calculated mean synergy score for the combination of Compound 12 and SN-38 was about 18, indicating synergy between Compound 12 and SN-38 to reduce MiaPaca2 cell viability. The calculated mean synergy score for the combination of Compound 12 and 5-FU was about 57, indicating synergy between Compound 12 and 5-FU. A summary of the IC₅₀ values is presented in Table 16.

TABLE 16
IC₅₀ of SN-38 and 5-FU in MiaPaca2
Cells when Treated with Compound 12

| Concentration of | IC₅₀ | Concentration of | IC₅₀ |
|---|---|---|---|
| Compound 12 (μM) | SN-38 (μM) | Compound 12 (μM) | 5-FU (μM) |
| 0 | 19.33 | 0 | 69.69 |
| 3.75 | 32.11 | 3.75 | 31.96 |
| 4 | 30.77 | 4 | 16.26 |
| 4.25 | 22.77 | 4.25 | 15.92 |

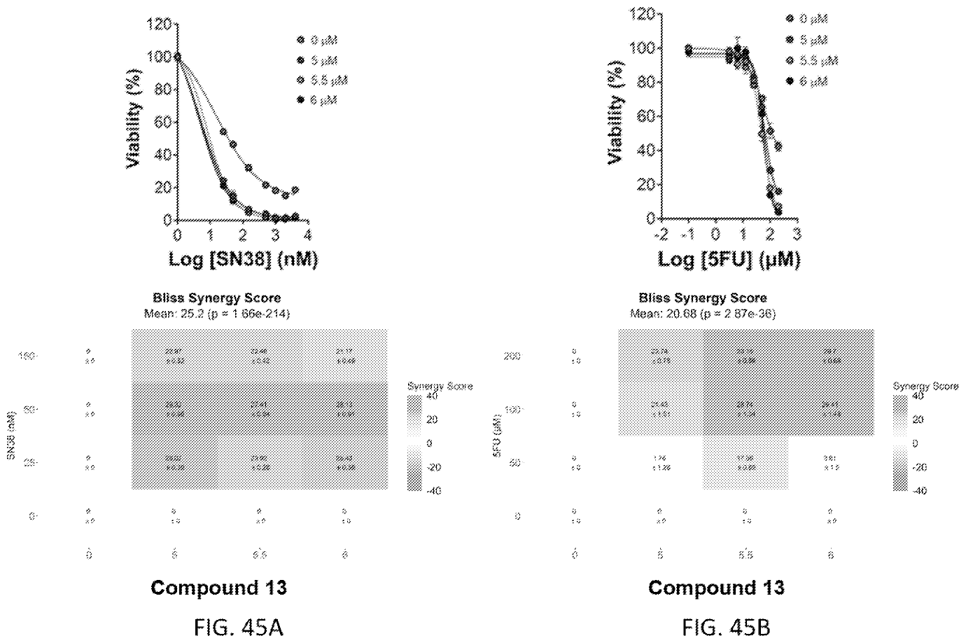

MiaPaca2 cells were treated with SN-38 and varying concentrations of Compound 13, and the ability of SN-38 and SN-38/Compound 13 to reduce MiaPaca2 cell viability. The IC₅₀ of SN-38 was found to decrease as a function of Compound 13 concentration, as shown in FIG. 45A and Table 17. The combination of Compound 13 and 5-FU was found to decrease the IC₅₀ of 5-FU as a function of Compound 13 concentration, as shown in FIG. 45B and Table 17. The calculated mean synergy score for the combination of Compound 13 and SN-38 was about 18, indicating synergy between Compound 13 and SN-38 to reduce MiaPaca2 cell viability. The calculated mean synergy score for the combination of Compound 13 and 5-FU was about 57, indicating synergy between Compound 13 and 5-FU. A summary of the IC₅₀ values is presented in Table 17.

TABLE 17
IC₅₀ of SN-38 and 5-FU in MiaPaca2
Cells when Treated with Compound 13

| Concentration of | IC₅₀ | Concentration of | IC₅₀ |
|---|---|---|---|
| Compound 13 (μM) | SN-38 (μM) | Compound 13 (μM) | 5-FU (μM) |
| 0 | 15.57 | 0 | 57.24 |
| 5 | 4.873 | 5 | 60.94 |
| 5.5 | 8.071 | 5.5 | 50.67 |
| 6 | 4.622 | 6 | 58.50 |

### Example 17: CHD1L Inhibitor Compound 13 as Combination Therapy for Breast Cancer

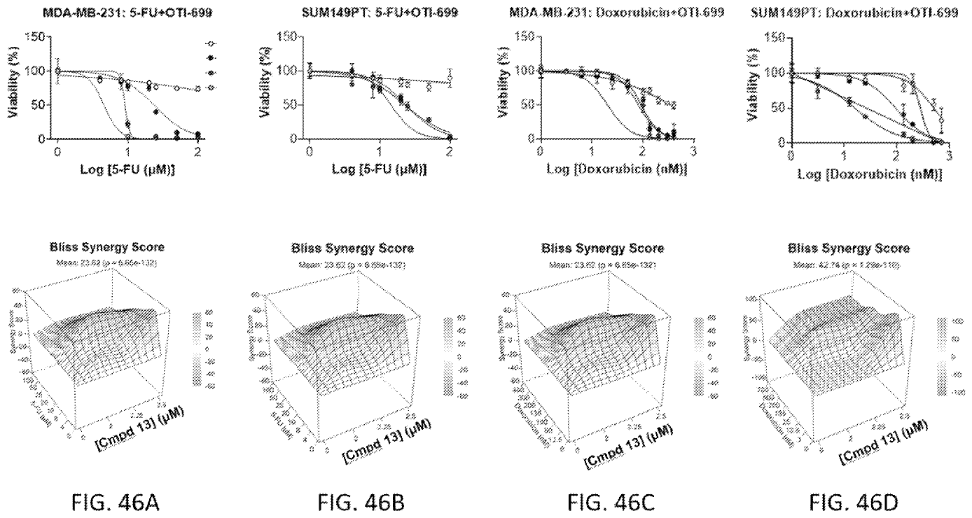

Compound 13 was administered to various model breast cancer organoid tissues including MDA-MB-231 (breast) and SUM149PT (breast) in combination with 5-FU, and the results are presented in FIGS. 46A and 46B, respectively. Compound 13 was administered to MDA-MB-231 (breast) and SUM149PT (breast) in combination with doxorubicin in FIGS. 46C and 46D, respectively. The measured IC₅₀ and calculated mean synergy scores are presented in Table 18.

TABLE 18
IC₅₀ of SN-38 and 5-FU when Treated with Compound 13

| | | | IC₅₀ | |
|---|---|---|---|---|
| Concentra- | IC₅₀ 5-FU | | doxorubicin | IC₅₀ |
| tion of | (μM) in | IC₅₀ 5-FU | (nM) in | doxorubicin |
| Compound | MDA- | (μM) in | MDA- | (μM) in |
| 13 (μM) | MB-231 | SUM149PT | MB-231 | SUM149PT |
| 0 | Resistant | Resistant | 345.9 | 505.4 |
| 2 | 23.5 | 23.8 | 92.2 | 94.4 |
| 2.25 | 9.1 | 23.0 | 81.5 | 16.0 |
| 2.5 | 4.6 | 14.4 | 20.2 | 279.2 |
| Mean | 23.62 | 23.62 | 23.62 | 42.74 |
| Synergy | | | | |
| Score | | | | |

Compound 13 was found to decrease the IC₅₀ of 5-FU and doxorubicin in both breast cancer cell lines. Further, the calculated mean synergy scores were at least 20 for all combinations of Compound 13 and 5-FU or doxorubicin, indicating synergy between the two.

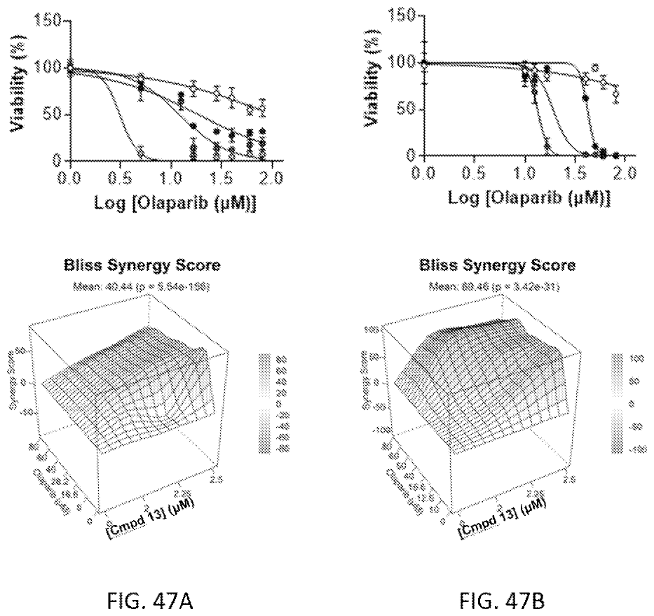

Compound 13 was administered to various model breast cancer organoid tissues including MDA-MB-231 (breast) and SUM149PT (breast) in combination with Olaparib, and the results are presented in FIGS. 47A and 47B, respectively. The measured IC₅₀ and calculated mean synergy scores are presented in Table 19.

TABLE 19
IC₅₀ of SN-38 and 5-FU when Treated with Compound 13

| Concentration of | IC₅₀ Olaparib (μM) | IC₅₀ Olaparib (μM) |
|---|---|---|
| Compound 13 (μM) | in MDA-MB-231 | in SUM149PT |
| 0 | 101.5 | 400.3 |
| 2 | 18.7 | 41.8 |
| 2.25 | 12.1 | 13.4 |
| 2.5 | 3.1 | 19.0 |
| Mean Synergy | 40.44 | 69.46 |
| Score | | |

Compound 13 was found to decrease the IC₅₀ of Olaparib in both breast cancer cell lines. Further, the calculated mean synergy scores were at least 40 for all combinations of Compound 13 and 5 Olaparib, indicating synergy between the two.

### Example 19: Pharmacokinetics in Mice

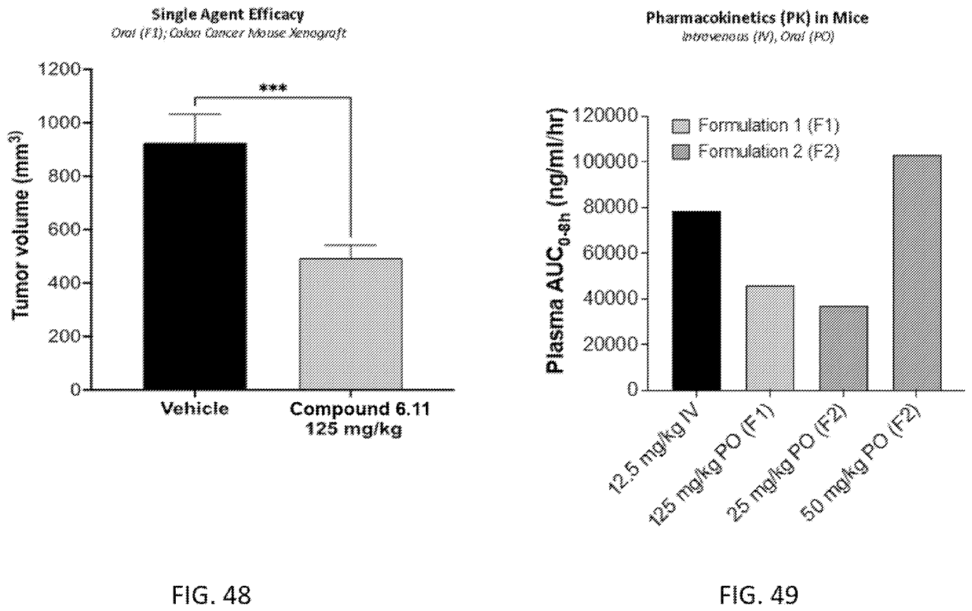

The efficacy of Compound 6.11 in reducing colon cancer in mice xenografts was assessed. A control colon cancer mouse xenograft was given a vehicle, and a second mouse xenograft was given 125 mg/kg Compound 6.11 in 10% DMSO and 90% PEG (all w/w %). The tumor volume was monitored over 30 days, and the tumor volume was measured after 30 days. The tumor was extracted from the mice, and the dimensions of the tumor were measured to provide a tumor volume in mm³. The result is presented in FIG. 48. A comparison of the tumor volume shows the formulation comprising Compound 6.11 reduced tumor size (and thus tumor growth) by about 50% in 30 days.

Pharmacokinetics in mice were assayed. A first batch of mice were intravenously administered a Compound 6.11 (12.5 mg/kg) administered intravenously. A second batch of mice were given a first formulation (F1) comprising 125 mg/kg (relative to mice weight) Compound 6.11 in 90% PEG and 10% DMSO (all w/w %). A third batch of mice were given a second formulation (F2) comprising 25 mg/kg (relative to mice weight) Compound 6.11, 30% PEG, 10% Tween-80, and 60% water (all w/w %). A fourth batch of mice were given the second formulation (F2) comprising 50 mg/kg Compound 6.11 in 30% PEG, 10% Tween-80, and 60% water (all w/w %). Plasma area under the curve (AUC) over 8 hours was measured to determine the amount of Compound 6.11 in plasma. The results are presented in FIG. 49. A comparison of the plasma AUC values (ng/mL/hr) illustrate the amount of Compound 6.11 in plasma greatly depended on the formulation composition.

### Example 20: Efficacy of CHD1Li in Mice

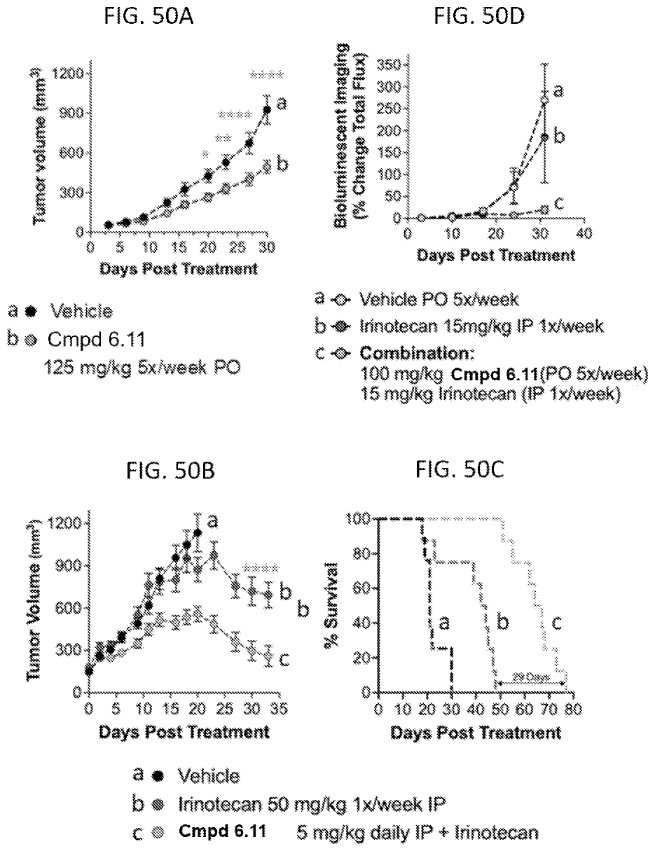

Colon cancer mice xenograft models were used to determine the efficacy of Compound 6.11 as a single agent and combination with irinotecan in reducing tumor volume and survival rates. FIG. 50A illustrates mice administered Compound 6.11 (125 mg/kg, 5×/week, oral administration) experienced a reduction in tumor volume by approximately 50% over the course of 30 days. FIG. 50B illustrates when mice were given Compound 6.11 and irinotecan as a combination therapy (50 mg/kg irinotecan, 1×/week; intraperitoneal administration) and Compound 6.11 (5 mg/kg daily, intraperitoneal administration) experienced a decrease in tumor volume of about 50% after 20 days, and tumor volume continued to decrease afterwards. Furthermore, the mice given the combination of both Compound 6.11 and irinotecan also showed improved survival rates, as evidenced by FIG. 50C. FIG. 50D illustrates a graph showing bioluminescent imaging (% change total flux) as a function of days post treatment of mice with irinotecan alone (15 mg/kg; 1×/week; intraperitoneal) and a combination of irinotecan (15 mg/kg irinotecan; 1×/week; intraperitoneal) and Compound 6.11 (100 mg/kg; 5×/week; oral). The data illustrate that administration with irinotecan reduces the metastatic tumor burden but administering the combination of irinotecan and Compound 6.11 significantly reduces the metastatic tumor burden to almost 0. Key: PO=oral; IP=intraperitoneal; ANOVA significance: *: P<0.05; **: P<0.0003; ***: P<0.0001.

### Example 21: In Silico Characterization of CHD1L Inhibitors and CHD1L Binding

The ability of compounds of Table 1 to bind to CHD1L was modeled, molecular docking data and parameters and ADME properties were calculated. The results are presented in Table 20. The higher the magnitude of the docking score, the tighter the CHD1L inhibitor binds to CHD1L. For example, of the compounds in Table 20, Compounds 7 and 18 bound to CHD1L more tightly than the other compounds of Table 20. Compounds 7 and 18 were also found to form 5 hydrogen bonds with residues within the active site, suggestive of stable interactions between these CHD1L inhibitors and CHD1L.

TABLE 20
MD simulations of docking of CHD1Li of Table 1 and calculated ADME properties.

| | Molecular docking data | | Calculated ADME properties | | | | |
|---|---|---|---|---|---|---|---|
| | Docking | Glide | | | | Solubility | |
| Compound No. | Score | Energy | MW | H-bonds | cLogP | (LogS) | PSA |
| Compound 6.11 | −5.7 | −55 | 472.4 | 4 | 5.7 | −8.9 | 71.0 |
| Compound 7 | −7.0 | −61 | 460.9 | 5 | 5.6 | −8.4 | 85.5 |
| Compound 8 | −6.4 | −59 | 472.9 | 4 | 5.9 | −8.4 | 78.3 |
| Compound 9 | −4.7 | −54 | 486.4 | 3 | 6.1 | −8.7 | 70.9 |
| Compound 10 | −5.7 | −52 | 376.4 | 5 | 5.0 | −7.1 | 81.7 |
| Compound 11 | −6.6 | −59 | 484.4 | 4 | 5.8 | −8.2 | 68.8 |
| Compound 12 | −6.2 | −53 | 441.3 | 5 | 4.4 | −7.1 | 88.1 |
| Compound 13 | −5.4 | 51 | 458.4 | 4 | 5.2 | −7.5 | 68.0 |
| Compound 14 | −5.4 | −48 | 459.4 | 5 | 4.3 | −6.9 | 81.4 |
| Compound 15 | −5.2 | −51 | 472.4 | 4 | 5.6 | −8.0 | 68.4 |
| Compound 16 | −7.3 | −58 | 460.9 | 5 | 5.8 | −8.5 | 81.6 |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of treating cancer, the method comprising: administering to a subject with the cancer a composition comprising:
a therapeutically effective amount of a chromodomain-helicase-DNA-binding protein 1-like (CHD1L) inhibitor having the structure of Formula (I): 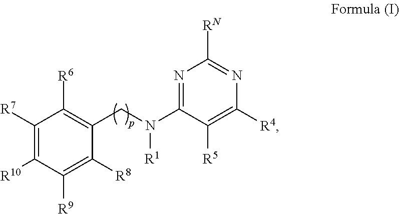
or a pharmaceutically acceptable salt thereof, wherein:
R^{N} is an optionally substituted 5- to 7-membered heterocycle optionally containing a second heteroatom selected from the of group N, S, and O;
R¹ is selected from hydrogen or C₁₋₃ alkyl;
each R⁴ and R⁵ is selected from hydrogen, C₁₋₃ alkyl, and halogen;
R⁶ is hydrogen or a halogen;
each R⁷, R⁸, and R⁹ is independently selected from hydrogen and C₁₋₃ alkyl, optionally substituted with C₁₋₃ alkyl or aryl;
R¹⁰ is NH—C(O)—(CH₂)ₘ—Ar, wherein
m is 0, 1, 2, 3, 4, or 5, and
Ar is aryl or heteroaryl optionally substituted with R^{a},
wherein each R^{a} is selected from hydrogen, C₁₋₃ alkyl, C₁₋₃ alkoxy, C₁₋₃ haloalkyl, or halogen, or two R^{a} together with the carbon to which they are bound form a 5- or 6-membered carbocycle, aryl, or heteroaryl optionally substituted with R^{a}; and
p is 0, 1, or 2,
wherein the cancer is breast cancer, melanoma, osteosarcoma, lung cancer, or pancreatic cancer.

2. The method of claim 1, wherein p is 0 or 1.

3. The method of claim 1 or 2, wherein R¹ is hydrogen.

4. The method of any one of claims 1 to 3, Ar is a 5- to 10-membered heterocycle.

5. The method of claim 4, wherein the 5- to 10-membered heterocycle is thiophenyl, furanyl, pyranyl, pyrrolyl, benzofuranyl, isobenzofuranyl, oxazolyl, indolyl, benzo[b]thiophenyl, or benzo[c]thiophenyl.

6. The method of any one of claims 1 to 5, wherein at least one of R⁶, R⁷, R⁸, or R⁹ is hydrogen.

7. The method of any one of claims 1 to 6, wherein Ar is substituted with at least one R^{a}.

8. The method of any one of claims 1 to 7, wherein R¹⁰ has the structure: 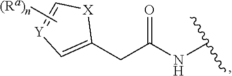
wherein
X is N, S, or O;
Y is N, S, or C;
R^{a} is selected from: hydrogen, C₁₋₃ alkyl, C₁₋₃ alkoxy, C₁₋₃ haloalkyl, or halogen; and
n is 0, 1, 2, or 3.

9. The method of claim 1, wherein the CHD1Li has the structure of Formula (II): 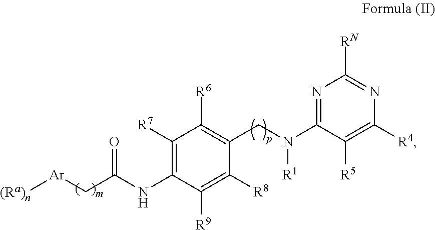
or a pharmaceutically acceptable salt thereof, wherein:
R^{N} is an optionally substituted 5- to 7-membered heterocycle optionally containing a second heteroatoms selected from the group N, S, or O;
R¹ is hydrogen or methyl;
R⁴ and R⁵ are each independently hydrogen, C₁₋₃ alkyl group, or halogen;
R⁶ is hydrogen or halogen;
R⁷, R⁸, and R⁹ are each independently hydrogen, C₁₋₃ alkyl, C₁₋₃ alkoxy, optionally substituted C₁₋₃ alkyl, or aryl;
Ar is an aryl or heteroaryl optionally substituted R^{a}, wherein
each R^{a} is selected from hydrogen, C₁₋₃ alkyl, C₁₋₃ alkoxy, C₁₋₃ haloalkyl, or halogen, or two R^{a} together with the carbon to which they are bound form a 5- or 6-membered carbocycle, aryl, or heteroaryl optionally substituted with R^{b}, and
each R^{b} is selected from C₁₋₃ alkyl, C₁₋₃ alkoxy, C₁₋₃ haloalkyl, or halogen;
p is 0 or 1;
m is 0, 1, 2, 3, 4, or 5; and
n is an integer from 0 to 9.

10. The method of claim 9, wherein Ar is a 5- to 10-membered heterocycle.

11. The method of claim 10, wherein the 5- to 10-membered heterocycle is thiophenyl, furanyl, pyranyl, pyrrolyl, benzofuranyl, isobenzofuranyl, oxazolyl, indolyl, benzo[b]thiophenyl, or benzo[c]thiophenyl.

12. The method of any one of claims claim 9 to 11, wherein R⁴ is methyl.

13. The method of any one of claims 9 to 12, wherein R¹ is hydrogen.

14. The method of any one of claims 9 to 13, wherein at least two of R⁵, R⁶, R⁷, R⁸, and R⁹ are hydrogen.

15. The method of any one of claims 9 to 14, wherein at least one of R⁵, R⁶, R⁷, R⁸, or R⁹ is halogen or C₁₋₃ alkoxy.

16. The method of any one of claims 9 to 15, wherein the CHD1L inhibitor has the structure of Formula (IIIA): 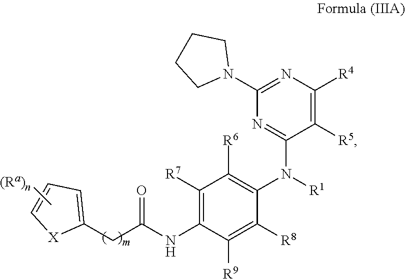
or a pharmaceutically acceptable salt thereof, wherein:
R¹ is hydrogen or methyl;
R⁴ and R⁵ are each independently hydrogen, C₁₋₃ alkyl group, or halogen;
R⁶ is hydrogen or a halogen;
R⁷, R⁸, and R⁹ are each independently hydrogen, C₁₋₃ alkyl, optionally substituted C₁₋₃ alkyl, or aryl;
each R^{a} is selected from hydrogen, C₁₋₃ alkyl, C₁₋₃ alkoxy, C₁₋₃ haloalkyl, or halogen, or two R^{a} together with the carbon to which they are bound form a 5- or 6-membered carbocycle, aryl, or heteroaryl optionally substituted with R^{b};
each R^{b} is selected from C₁₋₃ alkyl, C₁₋₃ alkoxy, C₁₋₃ haloalkyl, or halogen;
X is N, S, or O;
m is 0, 1, 2, 3, 4, or 5; and
n is 0, 1, 2, 3, or 4.

17. The method of claim 16, wherein R¹ is hydrogen.

18. The method of claim 16 or 17, wherein at least one of R⁶, R⁷, R⁸, or R⁹ is hydrogen.

19. The method of any one of claims 16 to 18, wherein m is 1, 2, 3, 4, or 5.

20. The method of any one of claims 16 to 19, wherein n is 1, 2, 3, or 4.

21. The method of any one of claims 16 to 20, wherein when n is 0, m is at least 1.

22. The method of any one of claims 16 to 21, wherein R^{a} is a C₁₋₃ alkyl or halogen.

23. The method of claim 21, wherein the halogen is bromo or chloro.

24. The method of any one of claims 16 to 23, wherein X is N.

25. The method of any one of claims 16 to 23, wherein X is S.

26. The method of any one of claims 16 to 23, wherein X is O.

27. The method of any one of claims 16 to 26, wherein at least one of R⁴ or R⁵ is methyl.

28. The method of claim 16, wherein CHD1L inhibitor having the structure of Formula (IIIA) is:

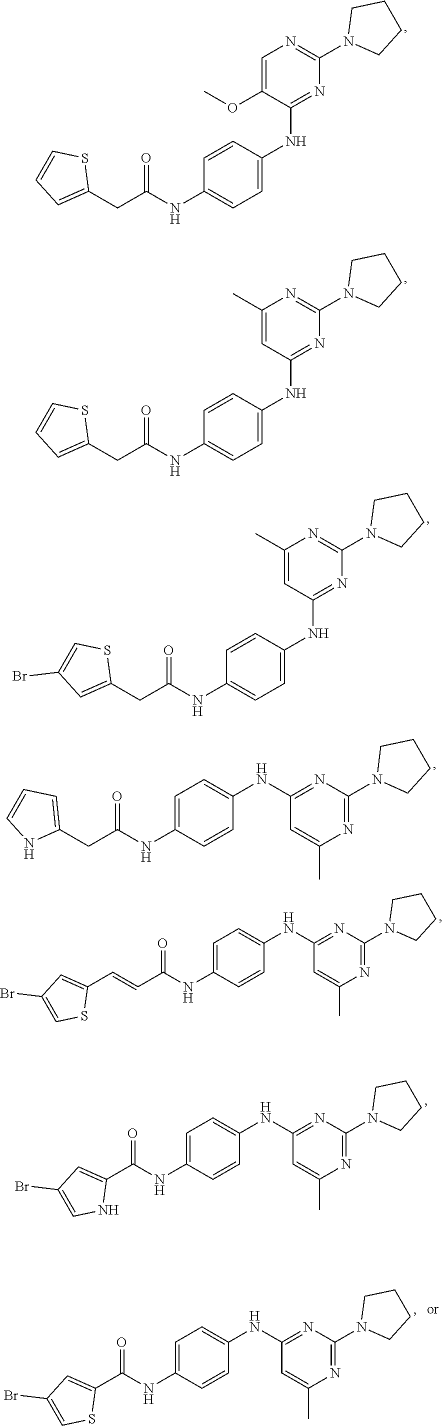

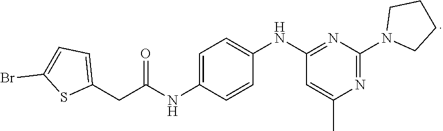

29. The method of claim 9, wherein the CHD1L inhibitor has the structure of Formula (IIIB): 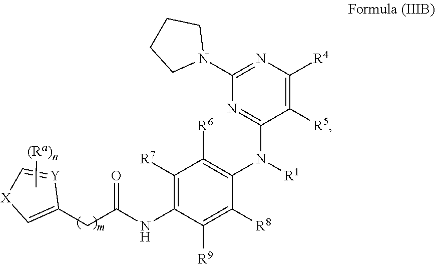
or a pharmaceutically acceptable salt thereof, wherein:
R¹ is hydrogen or methyl;
R⁴ and R⁵ are each independently hydrogen, C₁₋₃ alkyl group, or halogen;
R⁶ is hydrogen or a halogen;
R⁷, R⁸, and R⁹ are each independently hydrogen, C₁₋₃ alkyl, optionally substituted C₁₋₃ alkyl, or aryl;
each R^{a} is selected from hydrogen, C₁₋₃ alkyl, C₁₋₃ alkoxy, C₁₋₃ haloalkyl, or halogen, or two R^{a} together with the carbon to which they are bound form a 5- or 6-membered carbocycle, aryl, or heteroaryl optionally substituted with R^{b};
each R^{b} is selected from C₁₋₃ alkyl, C₁₋₃ alkoxy, C₁₋₃ haloalkyl, or halogen;
X is N, S, or O;
Y is C, N, or S;
m is 1, 2, 3, 4, or 5; and
n is 1, 2, 3, or 4.

30. The method of claim 29, wherein R¹ is hydrogen.

31. The method of claim 29 or 30, wherein at least one of R⁶, R⁷, R⁸, or R⁹ is hydrogen.

32. The method of any one of claims 29 to 31, wherein m is 1, 2, or 3.

33. The method of any one of claims 29 to 32, wherein n is 1, 2, 3, or 4.

34. The method of any one of claims 29 to 33, wherein R^{a} is a C₁₋₃ alkyl or halogen.

35. The method of claim 34, wherein the halogen is bromo or chloro.

36. The method of any one of claims 29 to 35, wherein X is N.

37. The method of any one of claims 29 to 35, wherein X is O.

38. The method of any one of claims 29 to 35, wherein X is S.

39. The method of claim 38, wherein Y is N, n is 1, and R^{a} is halogen.

40. The method of any one of claims 29 to 39, wherein at least one or R⁴ or R⁵ is methyl.

41. The method of claim 29, wherein the CHD1L inhibitor of Formula (IIIB) is:

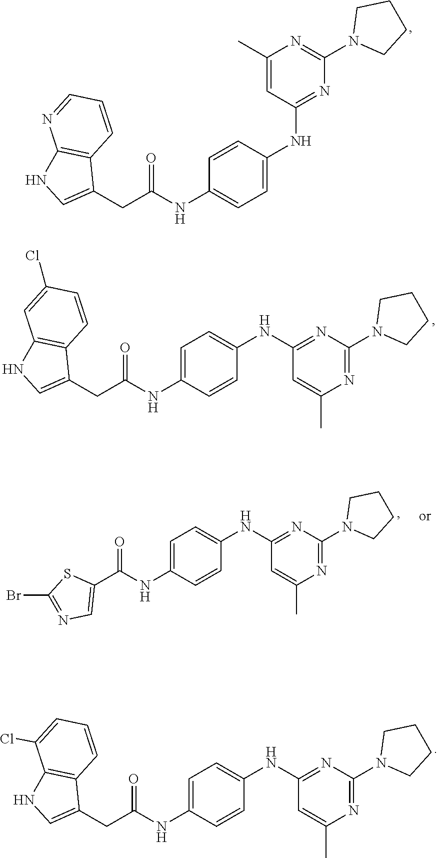 [SMILES: Cc1cc(Nc2ccc(NC(=O)Cc3c[nH]c4ncccc34)cc2)nc(N2CCCC2)n1 ; Cc1cc(Nc2ccc(NC(=O)Cc3c[nH]c4cc(Cl)ccc34)cc2)nc(N2CCCC2)n1 ; Cc1cc(Nc2ccc(NC(=O)c3cnc(Br)s3)cc2)nc(N2CCCC2)n1 ; Cc1cc(Nc2ccc(NC(=O)Cc3c[nH]c4c(Cl)cccc34)cc2)nc(N2CCCC2)n1]

42. The method of any one of claims 1 to 41, wherein the CHD1L inhibitor is a compound selected from Table 1.

43. The method of any one of claims 1 to 42, wherein the CHD1L inhibitor binds to an allosteric binding site comprising an N-terminus of a C-terminal ATP-ase domain of CHD1L.

44. The method of any one of claims 1 to 43, wherein the CHD1L inhibitor reduces an ATP-ase activity of CHD1L.

45. The method of any one of claims 1 to 44, wherein the CHD1L inhibitor interacts with a conserved lysine in the allosteric binding site.

46. The method of any one of claims 1 to 45, wherein the CHD1L comprises a sequence having at least 80% sequence identity SEQ ID NOs: 1-40 or Table 2.

47. The method of any one of claims 1 to 46, wherein the binding of CHD1L to an allosteric site of CHD1L inhibits chromatin remodeling.

48. The method of any one of claims 1 to 47, wherein the CHD1L inhibitor modulates poly-ADP-ribosylation (PAR) in a cancer cell.

49. The method of any one of claims 1 to 48, wherein administering the CHD1L inhibitor induces PAR-mediated programmed cell death (PARthanatos).

50. The method of any one of claims 1 to 49, wherein administering the CHD1L inhibitor prevents cell DNA repair in the tumor cell.

51. The method of any one of claims 1 to 50, wherein administering the CHD1L inhibitor induces PARthanatos and prevents cell DNA repair in the tumor cell.

52. The method of any one of claims 1 to 51, wherein administering the CHD1L inhibitor traps CHD1L within a nucleus of the tumor cell.

53. The method of any one of claims 1 to 52, wherein administering the CHD1L inhibitor inhibits a cell cycle of the tumor cell.

54. The method of any one of claims 1 to 53, wherein administering the CHD1L inhibitor inhibits chromatin remodeling.

55. The method of any one of claims 1 to 54, wherein administering the CHD1L inhibitor reduces tumor cell viability by about 20%, about 30%, about 40%, about 50%, about 60%, about 70%, about 80%, about 90%, about 95%, about 97%, about 99%, or about 100%, when the tumor cell viability is measured using an assay that measures ATP as an indicator of the tumor cell viability and generates a luminescence readout.

56. The method of claim 55, wherein assay comprises:
(a) introducing the CHD1L inhibitor and a detection reagent to organoids in a culture medium, wherein the detection reagent becomes luminescent when ATP in the organoids is extracted from the organoids;
(b) mixing the CHD1L inhibitor, the detection reagent and the organoids to produce a mixture under conditions sufficient to induce cell lysis and extract the ATP from the organoids;
(c) incubating the mixture to stabilize a luminescent signal; and
(d) recording luminescence to produce the luminescence readout.

57. The method of any one of claims 1 to 56, wherein the cancer displays multidrug resistance (MDR).

58. The method of any one of claims 1 to 57, wherein the cancer is breast cancer.

59. The method of any one of claims 1 to 57, wherein the cancer is lung cancer.

60. The method of any one of claims 1 to 57, wherein the cancer is melanoma.

61. The method of any one of claims 1 to 57, wherein the cancer is osteosarcoma.

62. The method of any one of claims 1 to 57, wherein the cancer is pancreatic cancer.

63. The method of any one of claims 1 to 61, further comprising administering a chemotherapy to the subject.

64. The method of claim 63, wherein a combination of the CHD1L inhibitor and the chemotherapy is therapeutically effective to treat the cancer.

65. The method of claim 63, wherein a combination of the CHD1L inhibitor potentiates or is synergistic with the chemotherapy.

66. The method of claim 63, wherein the cancer is resistant to the chemotherapy.

67. The method of any one of claims 61 to 66, wherein the chemotherapy is a PARP inhibitor.

68. The method of any one of claims 61 to 67, wherein the chemotherapy is a standard of care (SOC) chemotherapy for the cancer.

69. The method of any one of claims 61 to 68, wherein the chemotherapy comprises irinotecan, olaparib, doxorubicin, docetaxel, AZD5305, or 5-fluorouracil (5-FU), or a combination of two or more thereof.

70. The method of any one of claims 61 to 69, wherein the administration of the CHD1L inhibitor and the chemotherapy provides a Bliss synergy score of at least about 10, wherein the Bliss energy score is determined using a Bliss model.

71. The method of any one of claims 61 to 70, wherein the administration of the CHD1L inhibitor and the chemotherapy provides a Bliss synergy score of at least about 10 to about 60 using a Bliss model.

72. The method of any one of claims 61 to 71, wherein the administering the CHD1L inhibitor and the administering the chemotherapy are performed simultaneously.

73. The method of any one of claims 1 to 72, wherein the administration of the CHD1L inhibitor localizes apoptosis inducing factor (AIF) in the nucleus of the tumor cell.

74. The method of any one of claims 1 to 73, wherein the administration of the CHD1L inhibitor provides a higher concentration of AIF in the nucleus of the tumor cell relative to a concentration of AIF in a cytoplasm of the tumor cell.

75. The method of any one of claims 1 to 74, wherein the CHD1L inhibitor induces localization of PAR in the nucleus of the tumor cell.

76. A pharmaceutical composition for treating a cancer comprising:
a CHD1L inhibitor having the structure: 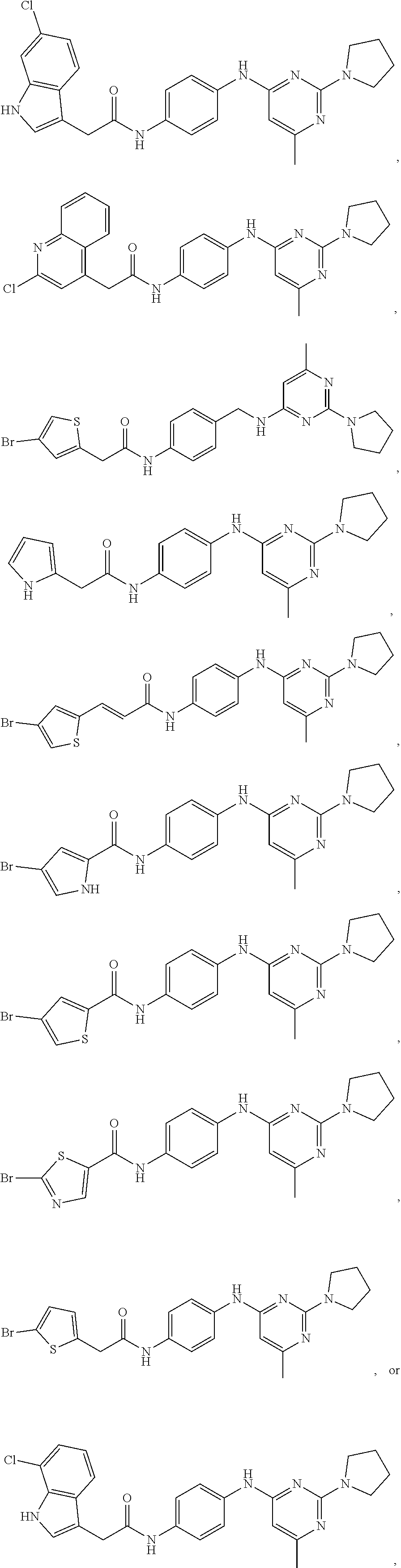
or a pharmaceutically acceptable salt thereof.

77. The pharmaceutical composition of claim 76, further comprising a chemotherapy.

78. The pharmaceutical composition of claim 77, wherein the chemotherapy comprises irinotecan, olaparib, doxorubicin, docetaxel, AZD5305, or 5-fluorouracil (5-FU), or a combination of two or more thereof.

79. The pharmaceutical composition of claim 77 or 78, wherein the chemotherapy is olaparib.

80. The pharmaceutical composition of claim 77 or 78, wherein the chemotherapy is irinotecan.

81. The pharmaceutical composition of claim 77 or 78, wherein the chemotherapy is doxorubicin.

82. The pharmaceutical composition of claim 77 or 78, wherein the chemotherapy is docetaxel.

83. The pharmaceutical composition of claim 77 or 78, wherein the chemotherapy is AZD5305.

84. The pharmaceutical composition of claim 77 or 78, wherein the chemotherapy is 5-fluorouracil (5-FU).

85. The pharmaceutical composition of claim 77, wherein the chemotherapy is effective against breast cancer.

86. The pharmaceutical composition of claim 77, wherein the chemotherapy is effective against colorectal cancer.

87. The pharmaceutical composition of claim 85, wherein the CHD1L inhibitor is a compound of Table 1.

88. The pharmaceutical composition of any one of claims 76 to 86, wherein the CHD1L inhibitor is 2-(6-chloro-1H-indol-3-yl)-N-(4-((6-methyl-2-(pyrrolidin-1-yl)pyrimidin-4-yl)amino)phenyl)acetamide.

89. The pharmaceutical composition of any one of claims 76 to 86, wherein the CHD1L inhibitor is 2-(2-chloroquinolin-4-yl)-N-(4-((6-methyl-2-(pyrrolidin-1-yl)pyrimidin-4-yl)amino)phenyl)acetamide.

90. The pharmaceutical composition of any one of claims 76 to 86, wherein the CHD1L inhibitor is 2-(4-bromothiophen-2-yl)-N-(4-(((6-methyl-2-(pyrrolidin-1-yl)pyrimidin-4-yl)amino)methyl)phenyl)acetamide.

91. The pharmaceutical composition of any one of claims 76 to 86, wherein the CHD1L inhibitor is N-(4-((6-methyl-2-(pyrrolidin-1-yl)pyrimidin-4-yl)amino)phenyl)-2-(1H-pyrrol-2-yl)acetamide.

92. The pharmaceutical composition of any one of claims 76 to 86, wherein the CHD1L inhibitor is (E)-3-(4-bromothiophen-2-yl)-N-(4-((6-methyl-2-(pyrrolidin-1-yl)pyrimidin-4-yl)amino)phenyl)acrylamide.

93. The pharmaceutical composition of any one of claims 76 to 86, wherein the CHD1L inhibitor is 4-bromo-N-(4-((6-methyl-2-(pyrrolidin-1-yl)pyrimidin-4-yl)amino)phenyl)-1H-pyrrole-2-carboxamide.

94. The pharmaceutical composition of any one of claims 76 to 86, wherein the CHD1L inhibitor is 4-bromo-N-(4-((6-methyl-2-(pyrrolidin-1-yl)pyrimidin-4-yl)amino)phenyl)thiophene-2-carboxamide.

95. The pharmaceutical composition of any one of claims 76 to 86, wherein the CHD1L inhibitor is 2-bromo-N-(4-((6-methyl-2-(pyrrolidin-1-yl)pyrimidin-4-yl)amino)phenyl)thiazole-5-carboxamide.

96. The pharmaceutical composition of any one of claims 76 to 86, wherein the CHD1L inhibitor is 2-(5-bromothiophen-2-yl)-N-(4-((6-methyl-2-(pyrrolidin-1-yl)pyrimidin-4-yl)amino)phenyl)acetamide.

97. The pharmaceutical composition of any one of claims 76 to 86, wherein the CHD1L inhibitor is 2-(7-chloro-1H-indol-3-yl)-N-(4-((6-methyl-2-(pyrrolidin-1-yl)pyrimidin-4-yl)amino)phenyl)acetamide.

98. The pharmaceutical composition of any one of claims 76 to 97, wherein the CHD1L inhibitor and the chemotherapy are formulated as separate dosages.

99. The pharmaceutical composition of any one of claims 76 to 98, wherein the CHD1L inhibitor and the chemotherapy are formulated to be administered simultaneously.

100. The pharmaceutical composition of any one of claim 98, wherein the CHD1L inhibitor and the chemotherapy are formulated to be administered sequentially.

101. The pharmaceutical composition of claim 100, wherein the CHD1L inhibitor is formulated to be administered prior to administration of the chemotherapy.

102. The pharmaceutical composition of claim 100, wherein the CHD1L inhibitor is formulated to be administered after administration of the chemotherapy.

103. The pharmaceutical composition of any one of claims 76 to 102, wherein the pharmaceutical composition is formulated to be administered orally or parenterally.

104. The pharmaceutical composition of claim 103, wherein the pharmaceutical composition is formulated to be administered orally.

105. The pharmaceutical composition of claim 103, wherein the pharmaceutical composition is formulated to be parenterally administered as an intradermal injection, subcutaneous injection, intramuscular injection, intraperitoneal injection, intraperitoneal infusion, or intravenous injection.

106. A method for treating cancer comprising administering a pharmaceutical composition of any one of claims 76 to 105.

107. The method of claim 106, wherein the cancer is colorectal cancer, breast cancer, osteosarcoma, melanoma, or lung cancer.

108. The method of claim 107, wherein the cancer is colorectal cancer.

109. The method of claim 107, wherein the cancer is breast cancer.

110. The method of claim 107, wherein the cancer is osteosarcoma.

111. The method of claim 107, wherein the cancer is melanoma.

112. The method of claim 107, wherein the cancer is lung cancer.

113. The method of any one of claims 106 to 112, wherein the pharmaceutical composition further comprises a chemotherapy.

114. The method of claim 113, wherein a combination of the CHD1L inhibitor and the chemotherapy is therapeutically effective to treat the cancer.

115. The method of claim 113, wherein the CHD1L inhibitor potentiates or is synergistic with the chemotherapy.

116. The method of claim 113, wherein the cancer is resistant to the chemotherapy, when the cancer is not treated with the CHD1L inhibitor.

117. The method of any one of claims 113 to 116, wherein the chemotherapy is a PARP inhibitor.

118. The method of any one of claims 113 to 117, wherein the chemotherapy is a standard of care (SOC) chemotherapy for the cancer.

119. The method of any one of claims 113 to 118, wherein the chemotherapy comprises irinotecan, olaparib, doxorubicin, docetaxel, AZD5305, or 5-fluorouracil (5-FU), or a combination of two or more thereof.

120. The method of any one of claims 113 to 119, wherein the administration of the CHD1L inhibitor and the chemotherapy provides a Bliss synergy score of at least about 10, wherein the Bliss energy score is determined using a Bliss model.

121. The method of any one of claims 113 to 120, wherein the administration of the CHD1L inhibitor and the chemotherapy provides a Bliss synergy score of at least about 10 to about 60 using a Bliss model.

122. The method of any one of claims 106 to 121, wherein the administration of the CHD1L inhibitor localizes apoptosis inducing factor (AIF) in the nucleus of the tumor cell.

123. The method of any one of claims 106 to 122, wherein the administration of the CHD1L inhibitor provides a higher concentration of AIF in the nucleus of the tumor cell relative to a concentration of AIF in a cytoplasm of the tumor cell.

124. The method of any one of claims 106 to 123, wherein the CHD1L inhibitor induces localization of PAR in the nucleus of the tumor cell.
